(19) United States
(12) Patent Application Publication
(10) Pub. No.: US 2006/0234906 A1
(43) Pub. Date: Oct. 19, 2006

(54) Structure of a complex of retinoblastoma protein bound to e2f, and uses thereof

(76) Inventor: Steven Gamblin, London (GB)

Correspondence Address: DLA PIPER RUDNICK GRAY CARY US, LLP, 4365 EXECUTIVE DRIVE, SUITE 1100, SAN DIEGO, CA, 92121-2133, US

(21) Appl. No.: 10/536,873

(22) PCT Filed: Nov. 27, 2003
(86) PCT No.: PCT/GB03/05158

§ 371 Date: Feb. 6, 2006

(30) Foreign Application Priority Data
Nov. 29, 2002 (GB) 0227910.7
Dec. 6, 2002 (GB) 02285538.5
Sep. 11, 2003 (GB) 0321300.6

(51) Int. Cl. C07K 14/72; G06F 19/00

(52) U.S. Cl. 514/2; 530/350; 702/19

(57) ABSTRACT

The present invention provides the crystal structure of pRb/E2F₍₄₀₉₋₄₂₆₎ as well as uses of the structure in identifying agents which modulate the binding between pRb and E2F and/or a pRb/E2F₍₄₀₉₋₄₂₆₎ complex, and thus are useful as pharmaceutical agents in the prevention or treatment of proliferative diseases.

The present invention relates to the crystal structure of pRb/E2F₍₄₀₉₋₄₂₆₎ as well as uses of the structure in identifying agents which modulate the binding between pRb and E2F and/or a pRb/E2F₍₄₀₉₋₄₂₆₎ complex, and thus are useful as pharmaceutical agents in the prevention or treatment of proliferative diseases.

The retinoblastoma tumour suppressor protein (pRb) regulates the cell cycle, sponsors differentiation and restrains apoptosis. Dysfunctional pRb is thought to be necessary for the development of most human malignancies.

pRb controls the cell cycle and apoptosis by acting as a negative regulator of transcription. It is now established that the growth-inhibitory effects of pRb are dependent on its regulation of the E2F family of transcription factors whose activity is necessary for the expression of genes involved in the G1 to S transition of the cell cycle and DNA replication. The transcriptional repression exerted by pRb over E2F responsive promoters involves at least three, distinct mechanisms. By binding to the transcriptional activation domain of E2F, pRb prevents it from recruiting components of the transcriptional apparatus and, once tethered to E2F promoters, pRb interacts with, and represses, other nearby transcription factors. Finally, pRb recruits protein factors to E2F promoters, such as histone deacetylases (HDACs) and histone methyltransferases (HMTases) that negatively regulate transcription by altering chromatin structure.

In addition to regulating entry into S-phase, it is thought that pRb is important in protecting differentiating cells from apoptosis. Certainly in many types of tissue, loss of pRb leads to apoptosis. This and other data has led to a model whereby the anti-apoptotic activity of pRb is mediated by its repression of certain E2F-dependent promoters. Unrepressed E2F is able to drive apoptosis by both p53-dependent and p53-independent mechanisms.

Although inactivation of the pRb pathway is thought to be widely involved in cellular transformation, there are examples of tumours where over-expression of functional pRb appears to be detrimental to successful clinical treatment. For example, adenocarcinoma of the pancreas is the fifth most common cause of cancer-related death in the Western world. It is particularly resistant to currently available forms of chemotherapy and radiation therapy. It is thought that this malignancy is able to evade apoptosis induced by treatment with chemotherapeutic drugs because of over-expression of pRb. It seems plausible that the protective effect of pRb over-expression against apoptosis is mediated by E2F. By blocking transcriptional activation by E2F, over-expression of pRb appears to render pancreatic cancer cells insensitive to chemotherapy.

As many of the anti-tumourigenic properties of pRb are mediated by its regulation of the E2F transcription factors, it would be beneficial to have a crystal structure of the pRb-binding fragment of E2F (E2F₍₄₀₉₋₄₂₆₎) in complex with the tumour suppressor protein. Such detailed knowledge of the molecular interactions between E2F and the A/B interface of pRb would enable the development of compounds that bind to pRb and inhibit complex formation. Such a compound, administered in parallel with conventional chemotherapy, would offer a means of enhancing treatment of proliferative diseases such as pancreatic cancer and perhaps related diseases.

Accordingly, the present invention provides the crystal structure of the primary pRb-binding fragment of E2F (E2F₍₄₀₉₋₄₂₆₎) in complex with the tumour suppressor protein pRb. The structure shows how E2F₍₄₀₉₋₄₂₆₎ binds at the interface of the A and B domains of the pocket of pRb making extensive interactions with conserved residues from both.

In order to address the regulation of the E2F transcription factor by pRb, the present inventors have determined the crystal structure of the complex of pRb_{AB} bound to the minimal binding region of E2F, namely E2F₍₄₀₉₋₄₂₆₎. The structure has important implications for the understanding of pRb/E2F function. The studies have quantified the contribution of the principal interaction made by E2F through residues 409-426 with pRb as well as that of a secondary interaction involving the marked box region of E2F. In both cases these interactions are with the pocket region of the tumour suppressor protein pRb.

The analysis of the crystal structure of pRb/E2F₍₄₀₉₋₄₂₆₎ suggests that E2F₍₄₀₉₋₄₂₆₎ acts as a sensor of the structural integrity of the pRb pocket. Accordingly, cells in many tissues should be protected against deleterious mutations in pRb because they will sponsor increased E2F transcriptional activation, and thus apoptosis. It seems particularly intriguing, therefore, that all tumour derived pRb mutants fail to bind to E2F suggesting that an intense selectionary pressure operates in many types of tissue in favour of cells which harbour defects in apoptosis once they have lost normal pRb function Perhaps the most notable exception to this process occurs in retinal cells, which are able to survive for some time with loss of pRb without acquiring other genetic alterations. Indeed, it has been suggested that these particular cells are distinguished by their ability to acquire survival signals from neighbouring cells and thus give rise to the eponymous retinoblastomas.

According to a first aspect, the present invention provides a crystal structure of the pRb/E2F₍₄₀₉₋₄₂₆₎ complex, characterised by the atomic co-ordinates of Annex 1.

Preferably the interactions between E2F₍₄₀₉₋₄₂₆₎ and pRb comprise one or more of the following interactions:

| E2F₍₄₀₉₋₄₂₆₎ residue | pRb residue |
|---|---|
| Leu₄₀₉ | Lys₅₄₈ |
| Tyr₄₁₁ | Glu₅₅₁ |
| Tyr₄₁₁ | Ile₅₃₂ |
| Tyr₄₁₁ | Glu₅₅₄ |
| His₄₁₂ | Arg₆₅₆ |
| His₄₁₂ | Lys₆₅₃ |
| Gly₄₁₄ | Glu₅₃₃ |
| Gly₄₁₄ | Lys₆₅₂ |
| Leu₄₁₅ | Leu₆₄₉ |
| Leu₄₁₅ | Glu₅₅₃ |
| Leu₄₁₅ | Lys₅₃₇ |
| Glu₄₁₇ | Lys₅₃₇ |
| Gly₄₁₈ | Arg₄₆₇ |
| Glu₄₁₉ | Thr₆₄₅ |
| Arg₄₂₂ | Glu₄₆₄ |
| Asp₄₂₃ | Arg₄₆₇ |
| Leu₄₂₄ | Lys₅₃₀ |
| Phe₄₂₅ | Phe₄₈₂ |
| Phe₄₂₅ | Lys₄₇₅ |

In a second aspect, the present invention provides a method to identify an agent which modulates the interaction between pRb and E2F₍₄₀₉₋₄₂₆₎, the method comprising:-
a) combining together pRb, E2F₍₄₀₉₋₄₂₆₎ and an agent, under conditions in which pRb and E2F₍₄₀₉₋₄₂₆₎ form a complex;
b) growing a crystal of any pRb/E2F₍₄₀₉₋₄₂₆₎ complex; and
c) analysing the crystal structure to determine whether the agent is an agent which modulates the interaction between pRb and E2F₍₄₀₉₋₄₂₆₎.

In the present invention, the term “modulates” is intended to refer to inhibiting, enhancing, destabilising and/or stabilising the interaction between pRb and E2F₍₄₀₉₋₄₂₆₎ and/or the formation of the pRb/E2F₍₄₀₉₋₄₂₆₎ complex and/or the stability of the complex after fomation.

“conditions in which pRb and E2F₍₄₀₉₋₄₂₆₎ can form a complex” are those conditions in which pRb and E2F₍₄₀₉₋₄₂₆₎ form a complex in the absence of an agent. Therefore the effect of the agent on the interaction between pRb and E2F₍₄₀₉₋₄₂₆₎ and complex formation can be assessed.

Growing a crystal of a pRb/E2F₍₄₀₉₋₄₂₆₎ complex in step b) can be performed using methods well known to the person skilled in the art, for example using methods described in Practical Protein Crystallography 1999, McRee, D. E., Academic Press, San Diego, Calif., USA; and also in Protein Crystallization Techniques, Strategies and Tips 1999, Bergfors, T. M., International University Line, Ca, USA.

In the method, the combining of the pRb, E2F₍₄₀₉₋₄₂₆₎ and agent may be in any order. The order may be combining pRb with the agent and then adding the E2F₍₄₀₉₋₄₂₆₎. Alternatively, the order may be combining E2F₍₄₀₉₋₄₂₆₎ with the agent and then adding pRb, or combining pRb with E2F₍₄₀₉₋₄₂₆₎ and then the agent. For example, the pRb may be combined with E2F₍₄₀₉₋₄₂₆₎ before soaking the complex in the agent, preferably in a solution of the agent. In this regard, two of the pRb, E2F₍₄₀₉₋₄₂₆₎ and agent may be co-crystalised before adding the pRb, E2F₍₄₀₉₋₄₂₆₎ or agent, as appropriate.

Preferably step c) comprises comparing the crystal structure to the crystal structure of the first aspect of the invention.

The agent may be selected using the three dimensional atomic co-ordinates of Annex 1.

In a third aspect, the present invention provides a method of identifying an agent that modulates a pRb/E2F₍₄₀₉₋₄₂₆₎ complex, comprising selecting an agent using the three-dimensional atomic coordinates of Annex 1.

Preferably, said selection is performed in conjunction with computer modeling.

Preferably the method comprises the further steps of:
a) contacting the selected agent with pRb and E2F₍₄₀₉₋₄₂₆₎ under conditions in which pRb and E2F₍₄₀₉₋₄₂₆₎ can form a complex; and
b) measuring the binding affinity of pRb to E2F₍₄₀₉₋₄₂₆₎ in the presence of the agent and comparing the binding affinity to that of pRb to E2F₍₄₀₉₋₄₂₆₎ when in the absence of the agent, wherein an agent modulates a pRb/E2F₍₄₀₉₋₄₂₆₎ complex when there is a change in the binding affinity of pRb to E2F₍₄₀₉₋₄₂₆₎ when in the presence of the agent.

The method may further comprise:
a) growing a supplementary crystal from a solution containing pRb and E2F₍₄₀₉₋₄₂₆₎ and the selected agent where said agent changes the binding affinity of the pRb/E2F₍₄₀₉₋₄₂₆₎ complex under conditions in which pRb and E2F₍₄₀₉₋₄₂₆₎ can form a complex;
b) determining the three-dimensional atomic co-ordinates of the supplementary crystal by X-ray diffraction using molecular replacement analysis;
c) comparing the three dimensional atomic co-ordinates with those for the crystal structure as defined in the first aspect of the invention; and
d) selecting a second generation agent using the three-dimensional atomic coordinates determined for the supplementary crystal.

Preferably, said selection is performed in conjunction with computer modeling.

In a fourth aspect there is provided a method of identifying an agent that modulates a pRb/E2F₍₄₀₉₋₄₂₆₎ complex, comprising:
a) contacting a selected agent with pRb and E2F₍₄₀₉₋₄₂₆₎ under conditions in which pRb and E2F₍₄₀₉₋₄₂₆₎ can form a complex; and
b) measuring the binding affinity of pRb to E2F₍₄₀₉₋₄₂₆₎ in the presence of the agent and comparing the binding affinity to that of pRb to E2F₍₄₀₉₋₄₂₆₎ when in the absence of the agent, wherein an agent modulates a pRb/E2F₍₄₀₉₋₄₂₆₎ complex when there is a change in the binding affinity of pRb to E2F₍₄₀₉₋₄₂₆₎ when in the presence of the agent.

There is a “change in the binding affinity” when the binding affinity either decreases or increases when in the presence of the agent. If a decrease is observed, the agent may be inhibiting the complex. If an increase is observed, the agent may be enhancing the complex.

The method of the fourth aspect may further comprise:
a) growing a supplementary crystal from a solution containing pRb and E2F₍₄₀₉₋₄₂₆₎ and the selected agent where said agent changes the binding affinity of the pRb/E2F₍₄₀₉₋₄₂₆₎ complex under conditions in which pRb and E2F₍₄₀₉₋₄₂₆₎ can form a complex;
b) determining the three-dimensional atomic coordinates of the supplementary crystal by X-ray diffraction using molecular replacement analysis;
c) comparing the three dimensional atomic co-ordinates with those for the crystal structure defined in the first aspect of the invention; and
d) selecting a second generation agent using the three-dimensional atomic coordinates determined for the supplementary crystal

Preferably, said selection is performed in conjunction with computer modeling.

In a fifth aspect, the present invention provides a method of identifying an agent that modulates a pRb/E2F₍₄₀₉₋₄₂₆₎ complex, comprising:
a) selecting an agent;
b) co-crystallizing pRb with the agent;
c) determining the three dimensional coordinates of the pRb-agent association by X-ray diffraction using molecular replacement analysis; and
d) comparing the three dimensional coordinates with those of the crystal structure claimed in claim 1.

In a sixth aspect, the present invention provides a method of identifying an agent that modulates a pRb/E2F₍₄₀₉₋₄₂₆₎ complex, comprising:
a) selecting an agent;
b) crystallizing pRb and soaking the agent into the crystal;
c) determining the three dimensional coordinates of the pRb-agent association by X-ray diffraction using molecular replacement analysis; and
d) comparing the three dimensional coordinates with those of the crystal structure claimed in claim 1.

In a seventh aspect, the present invention provides a method of identifying an agent that modulates a pRb/E2F₍₄₀₉₋₄₂₆₎ complex, comprising:
a) selecting an agent;
b) co-crystallizing pRb, E2F₍₄₀₉₋₄₂₆₎ and the agent;
c) determining the three dimensional coordinates of the pRb-E2F-agent association by X-ray diffraction using molecular replacement analysis; and
d) comparing the three dimensional coordinates with those of the crystal structure claimed in claim 1.

In an eighth aspect, the present invention provides a method of identifying an agent that modulates a pRb/E2F₍₄₀₉₋₄₂₆₎ complex, comprising:
a) selecting an agent;
b) co-crystallizing pRb and E2F₍₄₀₉₋₄₂₆₎ and soaking the agent into the crystal;
c) determining the three dimensional coordinates of the pRb-E2F-agent association by X-ray diffraction using molecular replacement analysis; and
d) comparing the three dimensional coordinates with those of the crystal structure claimed in claim 1.

Preferably the agent in the fifth, sixth, seventh or eighth aspect is selected using the three dimensional atomic co-ordinates of Annex 1. Preferably the method of the fifth, sixth, seventh or eighth aspect further comprises selecting a second generation agent using the three dimensional atomic coordinates determined. The second generation agent is preferably selected using the three dimensional atomic coordinates of Annex 1. The selection may be performed in conjunction with computer modeling.

Preferably the selected agent and/or the second generation agent, in the second, third, fourth, fifth, sixth, seventh and/or eighth aspects mimics a structural feature of E2F₍₄₀₉₋₄₂₆₎ when said E2F₍₄₀₉₋₄₂₆₎ is bound to pRb.

Preferably soaking refers to the pRb/E2F₍₄₀₉₋₄₂₆₎ complex being transferred to a solution containing the selected agent.

The method as defined in the third aspect preferably comprises the further steps of:
a) contacting the selected agent with a pRb/E2F₍₄₀₉₋₄₂₆₎ complex; and
b) determining whether the agent affects the stability of the complex.

Preferably the determination is with fluorescence polarization.

In a ninth aspect, the present invention provides a method of identifying an agent that modulates a pRb/E2F₍₄₀₉₋₄₂₆₎ complex, comprising:
a) contacting a fluorescently tagged E2F₍₄₀₉₋₄₂₆₎ peptide (E2F-fluoropeptide) with pRb to allow pRb/g2F-fluoropeptide complex formation;
b) detecting the fluorescence polarization;
c) adding a selected agent; and
d) detecting the fluorescence polarization in the presence of the agent.

In a tenth aspect, the present invention provides a method of identifying an agent that modulates a pRb/E2F₍₄₀₉₋₄₂₆₎ complex, comprising;
a) contacting a fluorescently tagged E2F₍₄₀₉₋₄₂₆₎ peptide (E2F-fluoropeptide) with pRb to allow pRb/E2F-fluoropeptide complex formation;
b) detecting the fluorescence polarization;
c) contacting a selected agent with pRb and E2F₍₄₀₉₋₄₂₆₎ peptide (E2F-fluoropeptide) under conditions in which pRb and E2F-fluoropeptide can form a complex;
d) detecting the fluorescence polarization; and
e) comparing the fluorescence polarization detected in b) and d).

Preferably the fluorescently tagged E2F peptide is selected using the three dimensional atomic co-ordinates of Annex 1.

Preferably a decrease in fluorescence polarization in the presence of the agent indicates that the agent destabilises the complex.

The methods of the ninth or tenth aspects may comprise the further step of adding untagged E2F₍₄₀₉₋₄₂₆₎ and detecting fluorescence polarization.

Preferably if fluorescence polarization decreases, on addition of the untagged E2F₍₄₀₉₋₄₂₆₎, the agent does not stabilise the complex.

Preferably if there is no substantial change in fluorescence polarization, on addition of the untagged E2F₍₄₀₉₋₄₂₆₎, the agent stabilises the complex.

Preferably the method further comprises:
a) contacting a fluorescently tagged E7 peptide (E7-fluoropeptide) with pRb to allow pRb/E7-fluoropeptide complex formation;
b) detecting the fluorescence polarization;
c) adding an agent that modulates PRb/E2F₍₄₀₉₋₄₂₆₎ complex; and
d) detecting the fluorescence polarization in the presence of the agent.

Alternatively the method may further comprise:
a) contacting a fluorescently tagged E7 peptide (E7-fluoropeptide) with pRb to allow pRb/E7-fluoropeptide complex formation;
b) detecting the fluorescence polarization;
c) contacting an agent that modulates pRb/E2F₍₄₀₉₋₄₂₆₎ complex with pRb and E7-fluoropeptide under conditions in which pRb and E7-fluoropeptide can from a complex;
d) detecting the fluorescence polarization; and
e) comparing the fluorescence polarization detected in b) and d).

Preferably a decrease in fluorescence polarization indicates that the agent also inhibits E7 binding to pRb. Such agents can then be removed from the method because the agents are identified as non-specific inhibitors. This identification of non-specific inhibitors can dramatically reduce the workload downstream of the assay method, for example in biochemical assays, thereby accelerating the hit to lead discovery process.

In addition ANS (aniline naphthalene sulphonic acid) reagent may be used to detect hydrophobic surfaces on pRb which become exposed as it unfolds. The fluorescence of ANS is highly sensitive to its environment. In solution there is virtually no fluorescence, whereas when bound to protein, such as pRb, it fluoresces highly. Changes in protein structure can alter the fluorescent signal of bound ANS due to changes in its environment to water. Therefore changes in pRb structure can be detected on addition of ANS and the agent that modulates pRb/E2F₍₄₀₉₋₄₂₆₎ complex. If the fluorescent signal alters on addition of the agent, the agent may be affecting the pRb structure. The use of ANS to monitor protein unfolding is known in the art (Semisotnov et al (1991) Biopolymers, 31(1), 119-128)

The binding affinities may be measured by isothermal titration calorimetry. Alternatively the binding affinities may be measured by Surface Plasmon Resonance (SPR).

In an eleventh aspect, the present invention provides an agent identified by a method according to the second, third, fourth, fifth, sixth, seventh, eighth, ninth and/or tenth aspects of the invention.

In a twelfth aspect, the present invention provides an agent, as set out according to the eleventh aspect of the invention, for use as an apoptosis promoting factor in the prevention or treatment of proliferative diseases.

Preferably the, or each selected agent is obtained from commercial sources or is synthesised. Preferably the agent is for use in preventing or treating cancer, which may be pancreatic cancer and related diseases.

In a thirteenth aspect, the present invention provides the use of an agent, which modulates a pRb/E2F₍₄₀₉₋₄₂₆₎ complex, identified by a method according to the second, third, fourth, fifth, sixth, seventh, eighth, ninth and/or tenth aspects of the present invention, in the manufacture of a medicament for the prevention or treatment of proliferative diseases.

The proliferative diseases may be cancer, preferably pancreatic cancer and related diseases.

In a fourteenth aspect, the present invention provides the use of the atomic co-ordinates of the crystal structure as set out according to the first aspect of the present invention, for identifying an agent that modulates a pRb/E2F₍₄₀₉₋₄₂₆₎ complex.

In a fifteenth aspect, the present invention provides computer readable media comprising a data storage material encoded with computer readable data, wherein said computer readable data comprises a set of atomic co-ordinates of the pRb/E2F₍₄₀₉₋₄₂₆₎ crystal structure according to Annex 1 recorded thereon.

Preferred features of each aspect of the invention are as for each of the other aspects mutatis mutandis.

The present invention will now be described, by way of example only, and with reference to the following figures, in which:

Annex I.

Atomic co-ordinates for crystal of pRb/B2F_{(409 426)} complex. In Annex 1 there is shown:

| Column Number | Description |
|---|---|
| 2 | Atom number |
| 3 | Atom type |
| 4 | Residue type |
| 5 | pRb domains (A or B) or E2F₍₄₀₉₋₄₂₆₎ (P) |
| 6 | Residue number |
| 7 | x co-ordinate of atom (Å) |
| 5 | y co-ordinate of atom (Å) |
| 9 | z co-ordinate of atom (Å) |
| 10 | Occupancy |
| 11 | B-factor (Å²) |

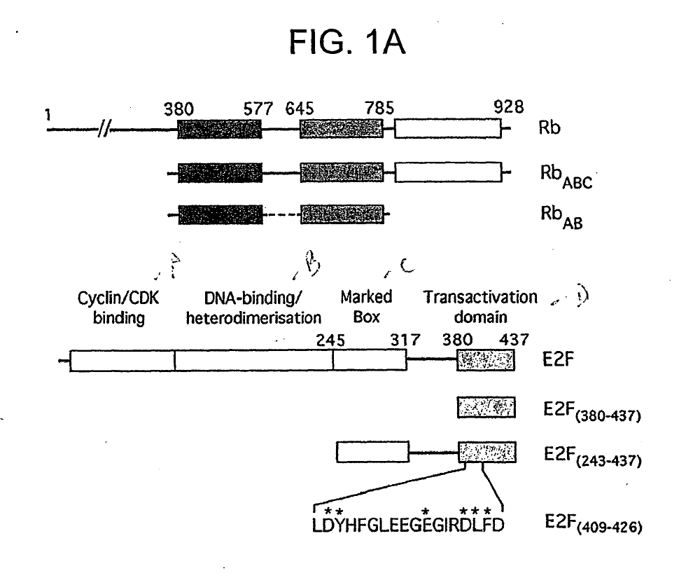

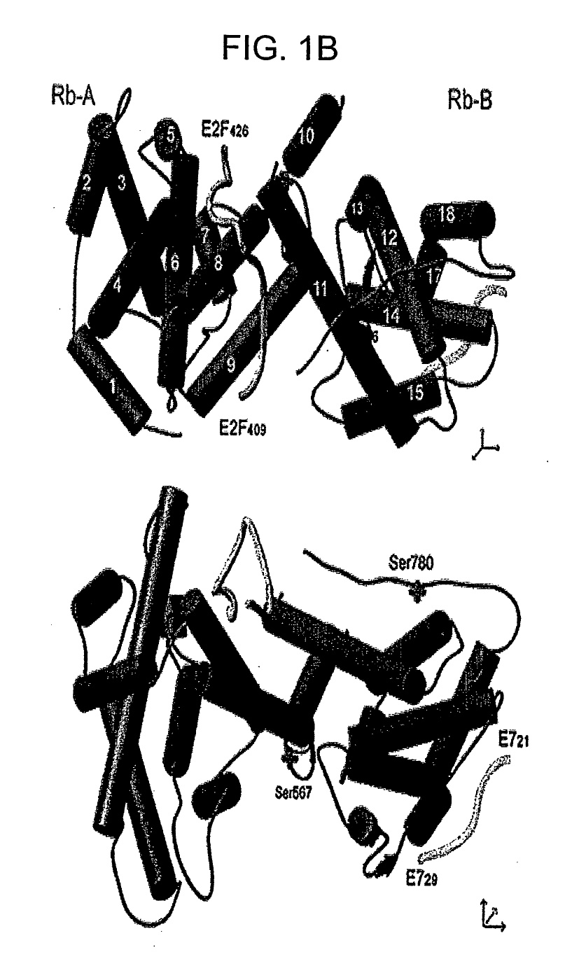

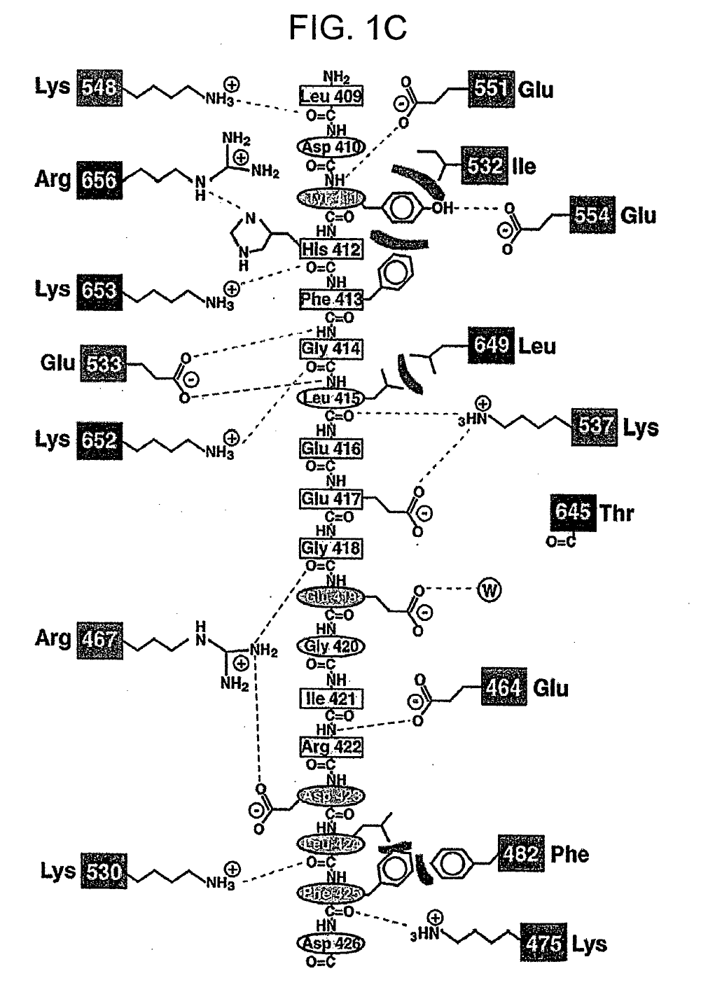

FIG. 1.

Structure of pRb/E2F.

(A) Schematic drawing of functional domains and protein constructs used for pRb, E2F. The shading used for the constructs in this panel match those used in subsequent figures.

(B) The structure of pRbsE2F₍₄₀₉₋₄₂₆₎, shown in two orthogonal views in Ribbons representation. The helices of the A domain are shown as a darker shade to those of the B domain. The main-chain trace of E2F is labelled.

(C) The interactions between E2F₍₄₀₉₋₄₂₆₎ and pRb_{AB} are shown schematically with the E2F peptide running down the centre. Residues of E2F that are conserved across the five family members are shown as ovals, while the five residue subset of these conserved residues whose mutation leads to disruption of the pRb/E2F interaction are shaded. Hydrogen-bond interactions are shown as broken lines, while hydrophobic contacts are indicated by bands. Residues from domain A of pRb are labelled with an asterisk and the other residues are from domain B. All of the pRb residues shown are either invariant or conserved across 27 species of pRb, p107 and p130.

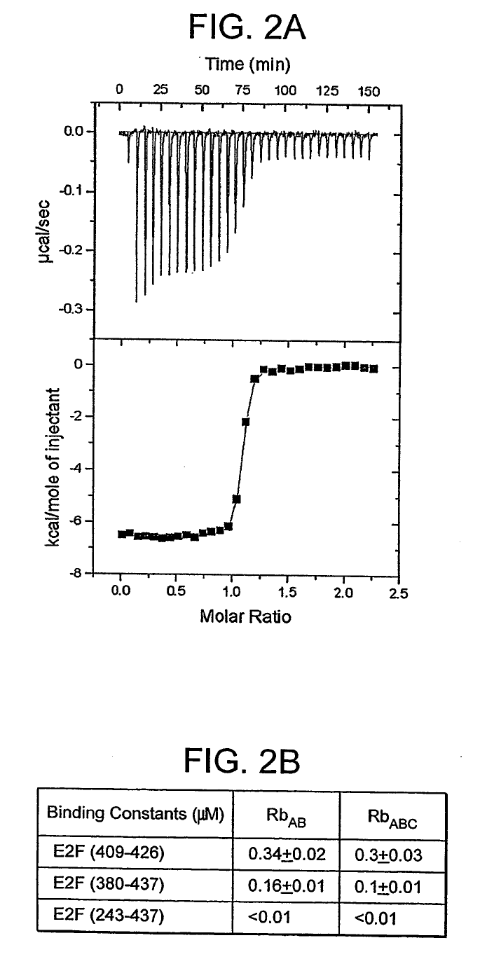

FIG. 2.

Isothermal Titration Calorimetry (ITC) measurements.

(A) The upper panel shows the raw data of an ITC experiment performed at 22° C. The lower panel shows the integrated heat changes, corrected for the heat of dilution, and the fitted curve based on a single site model. The panel represents the experiment where E2F₍₂₄₃₋₄₃₇₎ is titrated into Rb_{AB}.

(B) Summary of dissociation constants obtained by ITC measurements. The quoted errors are those produced by fitting the data to a two-state model. For the interaction of E2F₍₂₄₃₋₄₃₇₎ to Rb_{AB} and Rb_{ABC} the affinities are too high to measure reliably and we have therefore quoted the upper limits of the dissociation constants.

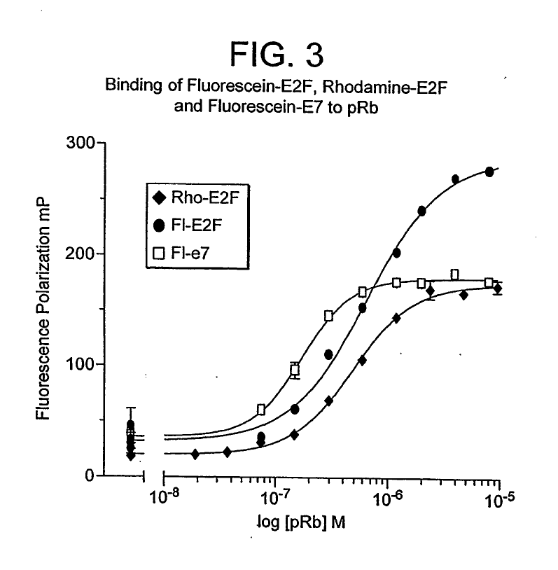

FIG. 3—Binding of Fluorescein-E2F, Rhodamine-E2F and Fluorescein-E7 to pRb

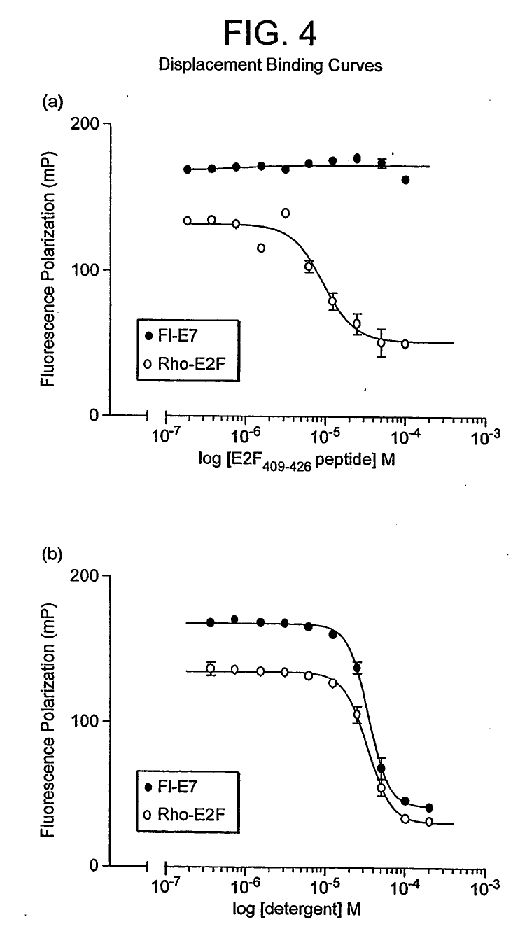

FIG. 4—Displacement binding curves: a) E2F₄₀₉₋₄₂₆ peptide; b) detergent

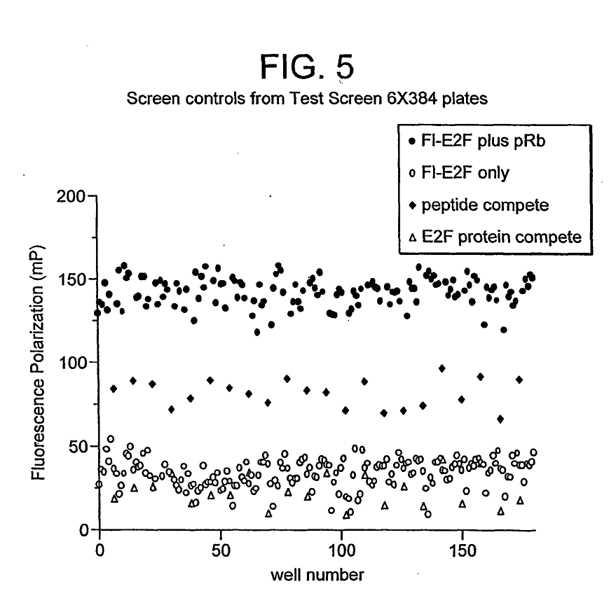

FIG. 5—Screen controls from test screen 6×384 plates

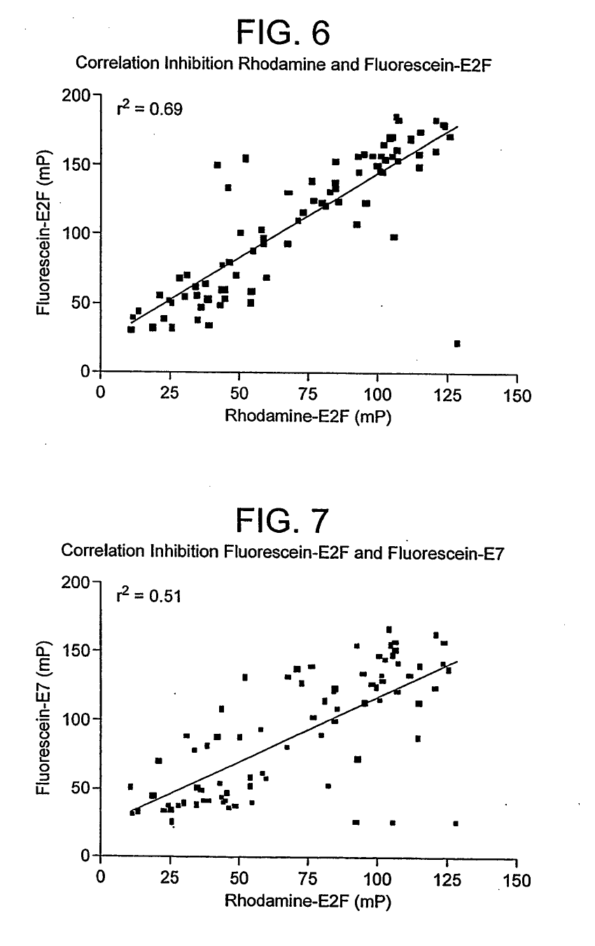

FIG. 6—Correlation between inhibition of Rhodamine and Fluorescein-E2F

FIG. 7—Correlation between inhibition of Fluorescein-E2F and Fluorescein-E7

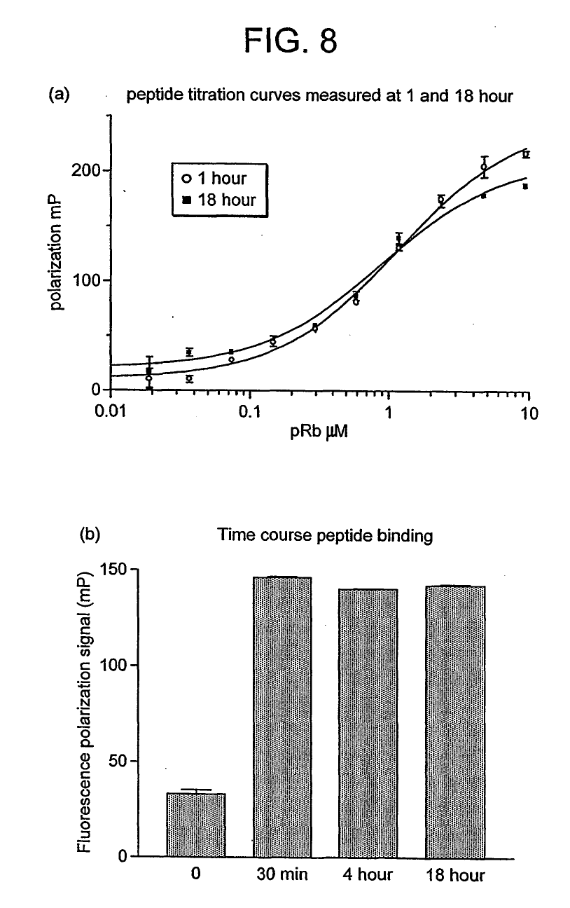

FIG. 8—a) Titration curves of rho-N-E2F (n=3); b) Time course of the change of fluorescence polarization signal with time taken from a test screen (mean±s.e.m., n=960)

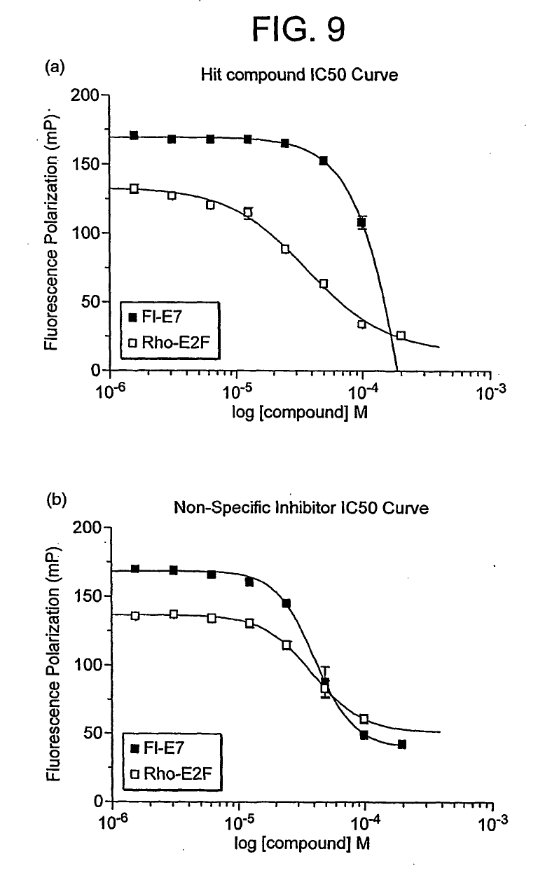

FIG. 9—IC50 curves determined for hits identified using the screening protocol described with reference to FIGS. 3 to 8: a) hit compound IC50 curve; b) non-specific inhibitor IC50 curve

### STRUCTURE DETERMINATION OF pRb/E2F

For crystallisation we used a pRb construct based on that previously described by Lee, J. O., Russo, A. A., and Pavletich, N. P. (1998). Structure of the retinoblastoma tumour-suppressor pocket domain bound to a peptide from HPV E7, Nature 391, 859-65, which has engineered thrombin cleavage sites at the ends of the flexible linker region between the A and B domains. Purification and proteolysis produces a final protein containing residues 372 to 589 of the A domain and 636 to 787 of the B domain (hereafter pRb_{AB}—FIG. 1A). Although these two domains are not covalently attached after thrombin treatment, they remain tightly associated. The removal of the A-B linker region facilitates crystallisation of pRb but does not alter its binding affinity for B2F. Crystals of the pRb/E2F₍₄₀₉₋₄₂₆₎ complex grew in a platelike habit with typical dimensions 200×200×10 μm³. Repeated attempts at data collection from flash-cooled crystals using synchrotron X-ray sources were thwarted by very high crystal mosaicity and poor data reduction statistics. The problem was overcome by using the micro-focus diffractometer on station ID13 at ESRF current experience and plans at EMBL and ESRF/ID13, Acta Crystallogr D 55, 1765-1770), currently the only such device installed at a synchrotron source. Using a 10×10 μm² aperture, data were collected from four separate and widely spaced volumes of a single crystal in order to minimise radiation damage. A total of 100, 1° oscillation images were collected using a MAR CCD detector. These data extended to a Bragg spacing of 2.5 Å with an overall R_{merge}=9.2%, and completeness of 87%. The structure was solved by molecular replacement and produced initial electron density maps in which the E2F peptide (E2F₍₄₀₉₋₄₂₆₎ could be readily located.

Protein Constructs.

Rb_{AB} was expressed as a GST-fusion protein in E. Coli using the pGEX-6P vector. The construct was engineered to contain a Prescission protease site at the N-terminus of Rb as well as two thrombin sites (LVPRGS) inserted at either end of the flexible A-B linker. Fusion protein was loaded onto a glutathione Sepharose 4B column before treatment with thrombin and Prescission protease. The resulting eluent was further purified using a Superdex 200 gel filtration column. Rb_{ABC} was expressed in E. coli with a C-terminal His-tag using pET-24. Crude lysate was first purified using an S-sepharose column followed by a Ni-NTA step before being run on a Superdex 200 gel filtration column. Recombinant human E2F1₍₂₄₃₋₄₃₇₎ was expressed in E. coli using pGEX-6P with an engineered Prescission protease site at the N-terminus of E2F. Crude lysate was bound onto a glutathione Sepharose 4B column prior to cleavage with the protease. The resulting eluent was further purified by gel filtration on a Superdex 75 column. E2F₍₄₀₉₋₄₂₆₎ and E2F₍₃₈₀₋₄₃₇₎ were synthetic peptides. HPV-16 E7₍₁₇₋₉₈₎ was prepared as described elsewhere (Clements, A. J., K, Mazzareli, J. M. Ricciardi, R. P. Marmorstein R. (2000). Oligomerization properties of the viral oncoproteins adenovirus E1A and human papillomavirus E7 and their complexes with the retinoblastoma protein., Biochemistry 39, 16033-16045).

Crystallography.

Plate-like crystals were grown by the hanging drop vapour diffusion method at 40° C. Rb_{AB} was mixed with the E2F-1 peptide at 1:2 molar ratio and concentrated to l5mg/ml. Hanging drops were formed by mixing 1 μl of protein solution with an equal volume of reservoir solution containing; 0.14M Na citrate, 26% PEG400, 4% n-propanol and 0.1M Tris at pH 7.8. Crystals were flash frozen in mother-liquor made up to 25% glycerol. Diffraction data were collected using the micro-focus diffractometer at ESRF and processed using the DENZO and SCALEPACK software (Otwinowski, Z. M., W. (1993). In Data Collection and Processing, L. I. Sawyer, N. Bailey, S., ed. (SERC Daresbury Laboratory), pp. 556-562). Molecular replacement calculations were carried out using Amore (CCP4, 1994) with 1GUX.bi;k as the search model. The final model contains co-ordinates for the protein which cover residues 379-578 of the A domain and 644-787 of the B domain of Rb and the entire E2F₍₄₀₉₋₄₂₆₎ peptide for the four copies present in the asymmetric unit together with 600 solvent molecules. The refined model has the following residuals; R_{work}=23.7%, R_{free}=28.7%, rmsd bonds=0.007 Å, rmsd angles=1.3°.

Structure of pRb/E2F Complex

The packing of the A and B domains generates a waist-like interface groove into which E2F₍₄₀₉₋₄₂₆₎ binds in a largely extended manner (FIG. 1B). The peptide makes contacts with residues from helices α4, α5, α6, α8 and α9 of domain A, and with α11 from domain B of pRb. Formation of the complex buries 2280 Å² of surface area, 1500 Å² of which are hydrophobic. The two end regions of the E2F₍₄₀₉₋₄₂₆₎ fragment make extensive contacts with pRb, while interactions made by the middle section of the E2F₍₄₀₉₋₄₂₆₎ fragment (residues 416 to 420) are relatively sparse (FIG. 1C). Overall, a high proportion of the hydrogen bond interactions between the two molecules involves the side chains of conserved pRb residues interacting with the main chain of E2F. Examination of the distribution of conserved residues over the surface of pRb, reveals that the majority are accounted for by the E2F binding site. There is a somewhat smaller cluster of conserved residues associated with the LxCxE binding site. Perhaps the most remarkable aspect of this analysis is that although pRb has been reported to associate with at least 110 cellular proteins perhaps 50 or more in a pocket-dependent manner, the E2F and LxCxE binding sites account for almost all of the conserved residues on its surface. There are two explanations that may partially account for these observations. Firstly, many of the reported binding partners of pRb have yet to be verified. Secondly, the LxCxE binding site is probably responsible for mediating the binding of many different proteins, such as HDAC, to pRb.

Since there are four copies of the pRb/E2F₍₄₀₉₋₄₂₆₎ complex in the asymmetric unit of our crystal form it is possible both to compare these four crystallographically independent copies of the pRb/E2F₍₄₀₉₋₄₂₆₎ complex and to compare them with the crystal structure of pRb/E7 without bond E2F (Lee et al., 1998 Supra). The first six residues at the N-terminus, the α3-α4 and α6-α7 loops adopt different conformations between the four copies in our asymmetric unit, while the variations across the rest of the structure between the four molecules is not significant. Comparison of the pRb structure in the presence and absence of bound E2F₍₄₀₉₋₄₂₆₎ shows that there is essentially no change in the relative orientation of the two lobes of the A/B pocket on E2F₍₄₀₉₋₄₂₆₎ binding nor any widespread changes in the structures of the individual domains. This comparison does reveal that the end of α4 and the connecting loop to α5 becomes ordered in the pRb/E2F₍₄₀₉₋₄₂₆₎ complex as two conserved residues (Glu464-pRb & Arg467-pRb located towards the end of α4 in our structure) interact with the E2F₍₄₀₉₋₄₂₆₎ peptide. Within the E2F₍₄₀₉₋₄₂₆₎ construct there are eight residues that are conserved across E2F's from all animal species (FIG. 1A). Amino-acid substitutions at five of these positions have been shown to lead to loss of binding to pRb but retention of E2F's trans-activation potential. The following description focuses on the structural role of these five residues. Tyr(411)-E2F appears to play an important role in peptide binding because its phenolic ring occupies a hydrophobic pocket created by Ile(536)-pRb, Ile(532)-pRb, Ile(547)-pRb and Phe(413)-E2F, while its hydroxyl group makes a hydrogen bond to the invariant Glu(554)-pRb. Towards the C-terminal part of the E2F peptide, Leu(424)-E2F and Phe(425)-E2F make several hydrophobic interactions, two of which involve conserved residues. Leu(424)-E2F makes contacts with the aliphatic portion of the side chain of Lys(530)-pRb and also packs against Leu(415)-E2F and Phe(425)-E2F. In addition, Phe(425)-E2F itself packs against Phe(482)pRb. Unlike the residues of E2F just discussed, the side-chains of Glu(419)-E2F and Asp(423)-E2F do not point into the groove formed between the A and B domains of pRb, but instead point away from it. Glu(419)-E2F hydrogen bonds through a water molecule with the main-chain carbonyl of Thr(645)-pRb; Asp(423)E2F forms a salt bridge with Arg(467)-pRb located at the end a4.

Finally, as described earlier, the crystal structure shows how E2F makes extensive contacts with largely conserved residues from both the A and B domains of the pocket and that the binding site for E2F is dependent on the structure of the interface between the two domains. This feature of the structure suggests that E2F acts as a sensor of the structural integrity of the pRb pocket. The position and nature of the E2F binding site make the binding of the transcription factor particularly sensitive to mutations in the pocket region of the tumour suppressor protein. The potential significance of these observations will be discussed later with regard to the normal role of pRb in protecting cells against E2F-mediated apoptosis.

Additional Determinants of pRb/E2F Function

It is clear from a number of studies that, although E2F₍₄₀₉₋₄₂₆₎ expressed as a Gal4 fusion protein is sufficient to recruit pRb and repress transcription, there are additional interactions made by the physiologically relevant E2F/DP heterodimer with pRb. Similarly, while the pocket domain is highly conserved, the most frequent site of deleterious mutation, and capable of transcriptional repression, it is not sufficient for the physiological function of pRb. In particular, the C-terminus of pRb is necessary for mediating growth arrest and recruitment of certain cyclin/cdk complexes as well as containing several of the residues whose phosphorylation leads to deactivation of pRb function. Therefore, in order to better understand the requirements for physiological pRb/E2F complex formation, we have made a series of constructs of the two proteins (FIG. 1A) and carried out binding measurements by isothermal titration calorimetry (ITC). We have also carried out a series of competition experiments to confirm qualitatively the interpretation of the ITC binding data.

Isothermal Titration Calorimetry.

Binding of the various E2F constructs to Rb_{AB} and Rb_{ABC} was measured by isothermal titration calorimetry using a MicroCal Omega VP-ITC machine (MicroCal Inc., Northampton, USA). The E2F constructs at a concentration between 100-150 μM were titrated into 12-15 μM Rb at a temperature of 22° C. Proteins were dialysed against 50 mM Tris pH 7.6, 100 mM NaCl and 1 mM TCEP. After subtraction of the dilution heats, calorimetric data was analysed using the evaluation software MicroCal Origin v5.0 (MicroCal Software Inc.). For all of the titrations, the stoichiometry of ligand binding to Rb was very close to 1.0. For E2F₍₂₄₃₋₄₃₇) binding to Rb, the binding affinity and the heat change associated with binding were such that we could only establish that binding was tighter than 10 nM. In order to verify that binding of this protein was similar for both Rb_{AB} and Rb_{ABC} we carried out competition experiments which showed approximately equal partition between the two different Rb proteins.

Competition Experiments.

The proteins used in these experiments were His₆-Rb_{ABC} (RESIDUES 380-929); MW 66.07 kDa, non-tagged Rb_{AB} (residues 372-787); MW 48.67 KDa, are His₆-Rb_{AB} (residues 376-792); MW 49.86 KDa, E2F₍₂₄₃₋₄₃₇₎; MW 21.45 KDa HPV E7 (residues 17-98); MW 9.38 KDa and E2F₍₄₀₉₋₄₂₆₎; MW 2.12 KDa. Protein concentrations were carefully determined by u.v. spectroscopy and confirmed by ITC titrations. The small acidic E2F proteins stain much weaker than Rb with Coomassie on SDS-PAGE. For all gel lanes contained a final Rb_{AB} concentration of ca. 7 μM. After equilibration with E2F₍₂₄₃₋₄₃₇) and E2F₍₄₀₉₋₄₂₆₎ the samples were loaded onto a 1.0 ml Ni column and washed with 7×0.5 ml of loading buffer (50 mM Tris pH 7.5, 200 mM NaCl & 10 mM Imidazole). The samples were then eluted with 7×0.5 ml elution buffer (50 mM Tris, pH 7.5, 200 mM NaCl, 200 mM Imidazole). After volume correction samples were boiled in SDS loading buffer and run on a 4-12% SDS PAGE. For the two pRb proteins and E2F₍₂₄₃₋₄₃₇₎ were mixed at 40 μM in a final volume of 0.5 ml. After equilibration for 2 hrs the mixture was loaded onto 1 ml Ni beads in a small column, washed with 7×0.5 ml of loading buffer (50 mM Tris, pH 7.5, 200 mM NaCl, 10 mM Imidazole), eluted using 7×0.5 ml elution buffer (50 mM Tris, pH 7.5, 200 mM NaCl, 200 mM Imidazole). Samples, after correcting for volume were boiled in SDS sample buffer and run on a 4-12% SDS gel.

A typical ITC experiment is shown in FIG. 2A and a summary of the affinity constants obtained for both pRb_{AB} and pRb_{ABC} interacting with three constructs of E2F are given in FIG. 2B. The two shorter E2F constructs bind to either pRb_{AB} or pRb_{ABC} with similar affinities. However, E2F₍₂₄₃₋₄₃₇₎ binds at least 16-fold stronger than either of the two shorter E2F fragments to both pRb_{AB} and Rb_{ABC}. Our ITC data therefore show that there are additional interactions of the A/B pocket of pRb with a region of E2F-1 outside of the transactivation domain. This result has been confirmed qualitatively by competition experiments which show that a 15-to 30-fold molar excess of the shorter E2F peptide is required to 50% compete with E2F₍₂₄₃₋₄₃₇) for binding to pRb. Our results are consistent with an earlier report that noted an interaction of pRb with the marked box region of E2F (residues 245-317). Taken together, these data demonstrate that the majority of the free energy of interaction between pRb and E2F is contributed by the 18-residue segment E2F₍₄₀₉₋₄₂₆₎ used in our structure analysis. There is an additional stabilising interaction between the marked box region of E2F and pRb, that contributes approximately 2 kcal mol⁻¹ to the overall free energy of complex formation, but is not sufficient on its own for complex formation.

As the binding constant for the interaction of E2F₍₂₄₃₋₄₃₇₎ with pRb_{AB} (or pRb_{ABC}) was too tight to determine reliably by ITC we performed a competition experiment to determine if this E2F construct bound preferentially to one or the other pRb construct. The results show approximately equal partitioning of E2F₍₂₄₃₋₄₃₇) between the two pRb species and demonstrates therefore, that the C-terminus of pRb does not participate in the binding to E2F-1 in isolation. This means that in addition to the interaction of E2F₍₄₀₉₋₄₂₆₎ with the pocket region of pRb there is an additional interaction, almost certainly involving the marked box region of E2F, that also binds to the pRb pocket. This data is consistent with the hypothesis that the approximately 10-fold stronger interaction of E2F/DP with pRb_{ABC} rather than pRb_{AB} reported previously arises through interactions of the DP component of the E2F/DP heterodimer with the C-terminus of pRb. This idea is strongly supported by the data from another study which shows that DP-1 interacts with pRb in a manner that does not require the structural integrity of the A/B pocket. Taken together, these data indicate that at least one of the functions of the C-terminus of pRb is to bring additional stabilisation to the interaction of the tumour suppressor with the heterodimeric E2F/DP transcription factors.

Use of Structure Atomic Co-Ordinates of Annex I

The atomic co-ordinates of Annex 1 are cartesian co-ordinates derived from the results obtained on diffraction of a monochromatic beam of X-rays by the atoms of the pRb/ E2F₍₄₀₉₋₂₆₎ complex in crystal form. The diffraction data was used to calculate electron density maps of the crystal. The electron density maps were then used to position the individual atoms of the pRb/ E2F₍₄₀₉₋₂₆₎ complex.

The determination of the three-dimensional structure of the pRb/E2F₍₄₀₉₋₄₂₆₎ complex provides basis for the design of new and specific agents that modulates formation of the complex and/or modulates the interaction between pRb and E2F₍₄₀₉₋₄₂₆₎. For example, computer modelling programs may be used to design different molecules expected to modulate formation of the pRb/E2F₍₄₀₉₋₄₂₆₎ complex and/or the interactions between pRb and E2F₍₄₀₉₋₄₂₆₎.

A candidate agent, may be any available compound. A commercial library of compound structures such as the Cambridge Structural Database would enable computer based in silico screening of the databases to enable compounds that may interact with, and/or modulate formation of, the complex to be identified.

Such libraries may be used to allow computer-based high throughput screening of many compounds in order to identify and select those agents with potential to modulate formation of the pRb/E2F₍₄₀₉₋₄₂₆₎ complex and/or the interaction between pRb and E2F₍₄₀₉₋₄₂₆₎.

In this regard, a potential modulating agent can be subjected to computer modelling with a docking program such as GRAM, DOCK or AUTODOCK (see Walters et al., Drug discovery Today, Vol. 3, No. 4, (1998), 160-178, and Dunbrack et al., Folding and Design, 2 (1997) 27-42) to identify and select potential agents. This can include computer fitting of potential modulating agents to the pRb/E2F₍₄₀₉₋₄₂₆₎ complex to ascertain how the agent, in terms of shape and structure, will bind to the complex.

Computer programs can be employed to estimate the interactions between the pRb,. E2F₍₄₀₉₋₄₂₆₎ and agent or pRb/E2F₍₄₀₉₋₄₂₆₎ complex and agent. These interactions may be attraction, repulsion, and steric hindrance of the two binding partners (e.g. the pRb/E2F₍₄₀₉₋₄₂₆₎ complex and a selected agent). A potential agent will be expected to be more potent if there is a tighter fit and fewer steric hindrances, and therefore greater attractive forces. It is advantageous for the agent to be specific to reduce interaction with other proteins. This could reduce the occurrence of side-effects due to additional interactions with other proteins.

Potential agents that have been designed or selected possible agents can then be screened for activity as set out in the second to tenth aspects above. The agents can be obtained from commercial sources or synthesised. The agent is then contacted with pRb/E2F₍₄₀₉₋₄₂₆₎ complex to determine the ability of the potential agent to modulate the formation of the complex. Alternatively the agent may be contacted with pRb and E2F₍₄₀₉₋₄₂₆₎ under conditions in which pRb and E2F₍₄₀₉₋₄₂₆₎ can form a complex (in the absence of agent), to determine the ability of the agent to modulate complex formation.

A complex of pRb/E2F₍₄₀₉₋₄₂₆₎ and said potential agent can then be formed such that the complex can be analysed by X-ray crystallography to determine the ability of the agent to modulate complex formation and/or the interaction between pRb and E2F₍₄₀₉₋₄₂₆₎.

The complex of pRb/E2F₍₄₀₉₋₄₂₆₎ and agent could be compared to that for pRb/E2F₍₄₀₉₋₄₂₆₎ alone with the three dimensional atomic co-ordinates in Annex 1.

Detailed structural information can then be obtained about the binding of the potential agent to the complex,. This will enable the structure or functionality of the potential agent to be altered to thereby to improve binding. The above steps may be repeated as may be required.

The agent-pRb/E2F₍₄₀₉₋₄₂₆₎ could be analysed by co-crystallising pRb/E2F₍₄₀₉₋₄₂₆₎ with the selected agent or soaking the agent into crystals of the pRb/E2F₍₄₀₉₋₄₂₆₎ complex; and then determining the three dimensional co-ordinates of the agent-complex by X-ray diffraction using molecular replacement analysis.

Therefore, the pRb/E2F₍₄₀₉₋₄₂₆₎-agent complexes can be crystallized and analysed using X-ray diffraction data obtained and processed, for example using the DENZO and SCALEPACK software (Otwinowksi, Z. M., W. (1993). Difference Fourier electron density maps can be calculated based on X-ray diffraction patterns of soaked or co-crystallised pRb/E2F₍₄₀₉₋₄₂₆₎ complex and the solved structure of uncomplexed agent. These maps can then be used to determine the structure of the agent bound to the pRb/E2F₍₄₀₉₋₄₂₆₎ and/or changes in the conformation of pRb/E2F₍₄₀₉₋₄₂₆₎ complex relative to the pRb/E2F₍₄₀₉₋₄₂₆₎ complex in the absence of agent.

The agent may be improved, for example by adding or removing functional groups, substituting groups or altering its shape in light of data obtained from agent bound to pRb/E2F₍₄₀₉₋₄₂₆₎ complex and/or agent bound to pRb. Such an improved agent may then be subjected to the methods of the invention.

Electron density maps can be calculated using programs such Amore from the CCP4 computing package (Collaborative Computational Project 4. The CCP4 Suite: Programs for Protein Crystallography, Acta Crystallographical, D50, (1994), 760-763).

The provision of computer readable media enables the atomic co-ordinates to be accessed to model the pRb/E2F₍₄₀₉₋₄₂₆₎ complex by, for example, RAMSOL (a publicly available computer software package which allows access and analysis of atomic co-ordinate data for structure determination and/or rational drug design).

In addition, structure factor data, derivable from the atomic co-ordinate data (see e.g. Blundell et al., in Protein Crystallography, Academic Press, New York, London and San Francisco, (1976)), can be used to enable difference Fourier electron density maps to be deduced.

Screening Assays

After an agent has been selected, its inhibitory effect on pRb/E2F₍₄₀₉₋₄₂₆₎ complex formation or ability to interact with the pRb/E2F₍₄₀₉₋₄₂₆₎ complex can be assessed with one or more of the methods of the invention.

For example, the crystal structure of the interaction of E2F₍₄₀₉₋₄₂₆₎ with pRb can be used to aid the design of a fluorescently tagged peptide for the use in a binding assay suitable for high throughput screening of low molecular weight compounds or peptide libraries. The fluorescent tag may be a fluorescein, rhodamine or some other commercially available tag chemically attached via a suitable amine or thiol group.

Binding could be measured by detecting fluorescence polarization in an homogeneous assay format (i.e. one in which all reagents are mixed in a single well, and reaction occurs in solution without wash steps). Fluorescence polarization technology is commonly applied in high throughput screening laboratories (ref: Sokhiam et al. (1999) Analytical Biochemistry, 275, 156-161. “Analysis of protein-peptide interaction by a miniaturised fluorescence polarization assay using cyclin-dependent kinase2/cyclin E as a model system.”)

Fluorescence polarization can be used to determine binding of a fluorescently-tagged small molecule (ligand or peptide) with a large molecule (receptor or protein) by detecting changes in the rotational velocity of the small molecule in the free and bound state. The rotational velocity is inversely proportional to the size of the molecule. Using a fluorescently tagged peptide and suitable optics the changes in rotational velocity upon binding to pRb can be measured as a differences in light emitted in parallel and perpendicular to a polarized excitation source. Fluorescence polarisation gives a measure of the proportion of fluorescently tagged peptide found in the bound state in a homogenous format.

In an assay method of the present invention, fluoro-peptide (E2F₍₄₀₉₋₄₂₆₎-fluoropeptide) bound to pRb will have a low rotational velocity and will appear stationary during the excitation period. Emitted light will be transmitted in parallel to the polarized incident light and the light detected will have a high polarization value. In contrast in the presence of an inhibitor of the interaction between pRb and E2F₍₄₀₉₋₄₂₆₎-fluoropeptide, the free E2F₍₄₀₉₋₄₂₆₎-fluoro-peptide will have a high rotational velocity and light will be transmitted in all directions. Emitted light will be detected both parallel and perpendicular to the polarized excitation source, and will have a low polarization value.

An example of the use of fluorescence polarisation is now described.

Data from a Fluorescence Polarisation (FP) screen configured for the interaction of pRb with E2F is presented. Fluorescein-tagged E2F peptide was used to screen 10,000 small drug like molecules. Hit confirmation strategies based on fluorescence interference and specificity were developed and compared.

Based on the crystal structure defined by the atomic co-ordinates in Annex 1, an FP screen was configured for the interaction of recombinant pRb A/B domains with E2F(409-426) peptide (see FIG. 1B). In addition, a second peptide binding site E7, see FIG. 1B), distant from the E2F binding pocket, was utilized as an internal control for non-specific inhibitors. Fluorophores in the form of fluorescein and rhodamine labelled peptides were synthesised and were used in a primary screen and hit confirmation.

Knowledge of the interaction of E2F and E7 peptides with pRb influenced the design of the fluoro-peptides used in the assay. The following peptides were synthesised, labelled and tested.
1. N-terminal amide link-age 5 carboxyfluorescein-E2F409-426, 18′mer. (fl-N-E2F18)
   LDYHFGLEEGEGIRDLFD
2. Rhodamine label at C-terminal cysteine E2F409-427, 19′mer. (Rh-C-E2F19)
   LDYHFGLEEGEGIRDLFDC
3. Rhodamine label at DDC substitution E2F409-426, 18′mer. (Rh-N2-E2F18)
   LCYHFGLEEGEGIRDLFD
4. N-terminal amide linkage Scarboxyfluorescein-E7, nonomer (Fl-E7)
   DLYCYEQLN

Peptides 1, 3 and 4 were used in the screen and subsequent hit confirmation assays.

Synthetic peptides were synthesised and fluoro-tagged using either N-terminal labelling with 5 carboxyfluorescein succinimidyl ester or cysteine labelling with single isomer tetramethylrhodamine-5-maleimide. Typical titration binding curves of pRb with the fluoro-labelled peptides are shown (mean±s.e.m, n=3) in FIG. 3. Fluorescein fluorescence measured at λexcite=485 and λemit=520 nm

Rhodamine fluorescence measured at λexcite=545 and λemit=580 nm

Measurements were made using BMG Fluorostar plate reader fitted with polarization optic. Fluorescein-E2F showed the greatest degree of polarization, and consequently the best signal to noise. It was chosen as the label of choice for a primary screen. Data were fitted to a one site binding model using Graphpad prism. Kd values of 450±70 and 380±50 nM were calculated for fluorescein and Rhodamine labelled E2F, which were similar to Kd determined for unlabelled peptide using isothermal calorimetry. Fluorescein-E7 showed tightest binding with Kd=130±20 nM,

The assay principle was validated using unlabelled E2F peptide to displace Fl-E2F without disrupting F1-E7 binding to pRb. Fluoro-tagged peptide (400 nM) was pre-incubated with pRb (1 μM ) and unlabelled peptide added at the concentrations shown. Displacement binding curves were plotted (FIG. 4a), and were fitted to a one site competition binding model using Graphpad prism curve fitting software. These curves were compared to the effects of a detergent-like compound (FIG. 4b), which causes gross structural changes and disrupts binding of both peptides.

The results show that labelled E2F (F1-E2F) does not displace E7, thereby validating the assay principle.

The assay was optimised in 384-well black-plates (Matrix) and automated using a Beckman Fx liquid handling robot. 1 μM pRb in 50 mM Tris HCl, pH7.0, 100 mM NaCl, 10 mM DTT, 0.05% NP-40 was mixed with 40 μM compound (4% DMSO) and 0.4 μM fluorescein-E2F (final concentrations). Controls from a test screen of 10,000 compounds are shown in FIG. 5.

Polarized and depolarized signal from fluorescein-E2F with and without pRb present are shown in FIG. 5 (solid and open circles respectively). Specific disruption of binding by E2F protein and peptide are also shown. Addition of E2F protein completely displaces F1-E2F (open triangle) and the signal is reduced to that of free fluoro-peptide alone. Addition of unlabelled-E2F at a concentration which gave 50% inhibition is clearly separated from the control populations. Hits were identified as compounds which reduced the polarization signal to less than mean-3sd of the fluorescein-E2F: pRb control.

Summary of Screen Data

| Assay Principle | | Fluorescence Polarization |
|---|---|---|
| Assay Automation | | Biomek Fx |
| Assay Detection | | BMG Polar Star Reader |
| Assay Parameters | Signal: noise | 6.9 |
| | Signal: background | 4.8 |
| | Z′ | 0.67 |
| Test Screen 10,000 | Z | 0.45 |
| | Hit rate | 0.93% |

Z factors are statistical factors well known by the skilled person in the art. The Z′ factor is defined by

$${Z}^{′} = 1 - \{ \frac{( 3 \times s . d . of positive control + 3 \times s . d of negative control )}{( mean of positive control - mean of negative control )} \}$$

In the present assay:-

positive control—fully polarized signal; pRb plus fluoro-tagged E2F peptide

negative control=depolarized signal from fluoro-tagged E2F peptide alone.

Z is calculated in much the same way except:

Positive control=polarized signal of pRb and fluoro-tagged E2F in presence of compounds.

Hit Confirmation:

Identification of Fluorescence Interfering Compounds.

A large proportion (37.5%) of the hits selected from the primary screen were coloured compounds which significantly altered the fluorescence intensity signal, and were potentially interfering with the assay. All hits were included in hit confirmation assays.

Hits were re-plated from master stocks and re-tested against fluorescein-E2F and rhodamine-E2F. A correlation (r²=0.69) between inhibition of fluorescein E2F and Rhodamine-E2F was observed (FIG. 6) with a hit confirmation rate of 78%. Notably, 60% of compounds which were potentially interfering with the fluorescein signal were inhibitors with Rhodamine-E2F assay, without affecting rhodamine fluorescence intensity signal. Suggesting that deselection of compounds on the basis of fluorescence interference can lead to loss of real inhibitors.

Finally the hits were tested against a second peptide binding site. Fluorescein-E7 peptide at 400 nM. The results were compared to inhibition of E2F and a scatter plot is shown in FIG. 7. A weak correlation was observed (r²=0.51), with 72% of the inhibitors of E2F also inhibiting fluorescein E7. These compounds were excluded as non-specific inhibitors and were not taken forward in subsequent biochemical assays.

Comparison of Hit Confirmation Strategies on 80 best hits selected from a Primary screen of 10,000 compounds.

Hit Confirmation rates

| Confirmation Test | % Primary Hits |
|---|---|
| 1. Inhibition in retest Fluorescein-E2F | 77.5 |
| 2. Fluorescence Interference | 37.5 |
| 3. Inhibition in retest Rhodamine-E2F | 62.5 |
| 4. Inhibition of Fluorescein-E7 | 58.5 |

The impact of selection strategy on number of compounds selected
for further biochemical study (eg IC50, isothermal calorimetry,
co-crystallisation)

| Strategy 1 | Strategy 2 | Strategy 3 |
|---|---|---|
| Tests 1 + 2 | Tests 1 + 3 | Tests 1 + 3 + 4 |
| Remove fluorescence | Select inhibitors active | E2F inhibitors but |
| interfering compounds | for both fluorescein- | not E7 inhibitors |
| | and rhodamine-B2F | |
| 36 | 50 | 14 |
| False Negatives | False Positives | Specific |
| | | Compounds only |

To demonstrate the stability and rapidity of binding equilibria of fluoro-peptide with pRb. The titration curves shown in FIGS. 8a and 8b are typical of several experiments and are of rho-N-E2F (n-3). The time course shown of the change of fluorescence polarization signal with time is taken from a test screen (mean±s.e.m., n=960).

pRb titration curves were performed in 96-well black plates, in a total reaction volume of 100 μL. Doubling dilutions from 10 μM stock of pRb were made in binding buffer (50 mM Tris HCl, pH7.0, 100 mM NaCl, 10 mM DTT, 0.05% NP-40) and 80 μL added in triplicate to wells. 20 μL of 2 μM fluoro-peptide was added and pipetted up and down to mix. The plate was read after 1 hr incubation at room temperature.

Compound interference was not a useful factor upon which to deselect compounds in an FP assay, and can lead to false negatives. The use of a second fluoro-label in hit confirmation avoids the loss of false negatives, but still includes false positives.

Screening of the hits against the second peptide site, E7, identified non-specific inhibitors, which caused gross structural changes to the protein. These were excluded from further biochemical testing. Identification of these non-specific inhibitors dramatically reduced the down stream work load.

The developed screening strategy rapidly identifies false negatives and positives (interfering and protein unfolding reagents) from the primary screen. This reduces the number of compounds to test in biochemical assays, thus saving both time and reagents which will accelerate the hit to lead discovery process.

ANS (aniline naphthalene sulphonic acid) reagent may be used to detect hydrophobic surfaces on pRb which become exposed as it unfolds. The fluorescence of ANS is highly sensitive to its environment. In solution there is virtually no fluorescence, whereas when bound to protein, such as pRb, it fluoresces highly. Changes in protein structure can alter the fluorescent signal of bound ANS due to changes in its environment to water. Therefore changes in pRb structure can be detected on addition of ANS and the agent that modulates pRb/E2F₍₄₀₉₋₄₂₆₎ complex. If the fluorescent signal alters on addition of the agent, the agent may be affecting the pRb structure. The use of ANS to monitor protein unfolding is known in the art (Semisotnov et al (1991) Biopolymers, 31(1), 119-128)

Biochemical assays could include IC50, isothermal calorimetry, and/or co-crystallisation.

In an example of an IC50 assay, reactions were performed in 96-well black plates in a total reaction volume of 100 μL. Compounds were dissolved in DMSO at a maximum concentration of 10 nM and doubling dilutions made in DMSO. 4 μL of diluted compound was mixed with 80 μL pRb (400 nM in binding buffer). The plate was incubated at room temperature for 15 min and then Rhodamine-E2F and fluorescein-E7 were added to give final concentrations of 400 nM each. Reactions were performed in triplicate. Plates were read after 1 hr. The results are shown in FIGS. 9a and 9b.

Accordingly, an assay method could include the following steps:
a) allow complex formation of pRb and E2F₍₄₀₉₋₄₂₆₎-fluoropeptide, and measure maximum fluorescence polarization; and
b) add a selected agent and detect whether there is a decrease in fluorescence polarization.

Alternatively, an assay method could include the steps:
a) allow complex formation of pRb and E2F₍₄₀₉₋₄₂₆₎-fluoropeptide in the presence and absence of a selected agent and measure the fluorescence polarization; and
b) compare the fluorescence polarization values.

Compounds which stabilise the pRb/E2F₍₄₀₉₋₄₂₆₎ complex could be assessed in a modification of the above method, involving competition binding of pRb by unlabelled E1F₍₄₀₉₋₄₂₆₎ and E2F₍₄₀₉₋₄₂₆₎-fluoropeptide.

In this regard an assay method could include the following steps:
a) allow complex formation of pRb/E2F₍₄₀₉₋₄₂₆₎-fluoropeptide, and measure max fluorescence polarization;
b) add a selected agent and measure fluorescence polarization—if no change in fluorescence polarization there is no disruption of complex;
c) add unlabeled E2F₍₄₀₉₋₄₂₆₎ and measure fluorescence polarization—expect displacement of E2F₍₄₀₉₋₄₂₆₎-fluoropeptide and a decrease in fluorescence polarization, but not if complex is stabilised by presence of the agent.

Alternatively, the pRb, E2F₍₄₀₉₋₄₂₆₎-fluoropeptide and agent could be added together before detecting fluorescence polarization. If fluorescence polarization is reduced to less than a predetermined value, the agent is determined to destabilize the complex, and vice versa.

The interactions could be confirmed by co-crystalisation of pRb/E2F₍₄₀₉₋₄₂₆₎ with the agent, and determination of the three dimensional atomic coordinates by X-ray diffraction and molecular replacement.

The E2F₍₄₀₉₋₄₂₆₎/pRb interaction can also be applied to heterogeneous assay formats (i.e. ones in which reagents are partitioned between a solid support and in solution, and wash steps are involved). This would involve the immobilization of pRb on microtitre plates, for example by antibody capture or metal ion chelation using His-tagged pRb and Nickel coated plates. E2F₍₄₀₉₋₄₂₆₎ peptide may be tagged with fluorescence as above and the fluorescence detected directly to determine binding. Alternatively, the peptide could be labelled with biotin and detected with streptavidin-horse radish peroxidase in an ELISA-like format.

Compounds which interact with the complex without altering association or disassociation of the complex could be identified by crystallographic means, unless the agent itself was tagged (radioactivity/fluorescence) and binding to the complex measured directly, e.g. fluorescence polarization as above or scintallation counting of an immuno-precipitate.

Alternatively, the agent can be added to pRb and E2F₍₄₀₉₋₄₂₆₎ under conditions in which pRb and E2F₍₄₀₉₋₂₆₎ can form a complex. This could result in the agent and complex co-crystallising. The binding affinities of pRb to E2F₍₄₀₉₋₂₆₎ in the pRb/E2F₍₄₀₉₋₂₆₎ complex in the presence and absence of the agent can then be compared to determine whether the agent inhibits complex formation. The three dimensional structure of the pRb/ E2F₍₄₀₉₋₄₂₆₎ —agent complex can also be solved (X-ray diffraction using molecular replacement analysis) to enable the associations in the new complex to be compared with those in the pRb/ E2F₍₄₀₉₋₄₂₆₎ complex (see Annex 1). As a further alternative the pRb/E2F₍₄₀₉₋₂₆₎ complex can be formed before soaking the complex in the presence of a selected agent. The binding affinities of pRb to E2F₍₄₀₉₋₂₆₎ can then be determined in the presence and absence of the agent. As before, the three dimensional structure of any pRb/ E2F₍₄₀₉₋₂₆₎ —agent complex could be solved.

The binding affinities can be measured using isothermal titration calorimetry. Alternatively, surface plasmon resonance (SPR) could be used such as that provided by Biacore AB.

Preferred features of each aspect of the invention are as for each of the other aspects mutatis mutandis. The prior art documents mentioned herein are incorporated to the fullest extent permitted by law.

Annex 1

REMARK the coordinates is one molecule from four molecules in an asymmetric
REMARK unit cell whithin the crystal:
REMARK a = 101.996 b = 158.548 c = 110.617 alpha = 90.00 beta = 93.70 gama = 90.00 C 2

| | | | | | | | | | | | |
|---|---|---|---|---|---|---|---|---|---|---|---|
| ATOM | 1 | N | MET | A | 379 | 13.261 | −15.752 | 30.447 | 1.00 | 45.11 | N |
| ATOM | 2 | CA | MET | A | 379 | 11.983 | −16.486 | 30.626 | 1.00 | 44.12 | C |
| ATOM | 3 | CB | MET | A | 379 | 11.935 | −17.082 | 32.026 | 1.00 | 44.57 | C |
| ATOM | 4 | CG | MET | A | 379 | 12.067 | −16.066 | 33.137 | 1.00 | 45.87 | C |
| ATOM | 5 | SD | MET | A | 379 | 12.458 | −16.814 | 34.740 | 1.00 | 52.60 | S |
| ATOM | 6 | CE | MET | A | 379 | 10.805 | −17.831 | 35.114 | 1.00 | 52.37 | C |
| ATOM | 7 | C | MET | A | 379 | 10.802 | −15.543 | 30.446 | 1.00 | 43.32 | C |
| ATOM | 8 | O | MET | A | 379 | 9.681 | −15.889 | 30.824 | 1.00 | 43.69 | O |
| ATOM | 9 | N | ASN | A | 380 | 11.069 | −14.348 | 29.909 | 1.00 | 41.45 | N |
| ATOM | 10 | CA | ASN | A | 380 | 10.043 | −13.347 | 29.646 | 1.00 | 39.85 | C |
| ATOM | 11 | CB | ASN | A | 380 | 10.641 | −11.934 | 29.700 | 1.00 | 39.85 | C |
| ATOM | 12 | CG | ASN | A | 380 | 10.867 | −11.446 | 31.134 | 1.00 | 40.80 | C |
| ATOM | 13 | OD1 | ASN | A | 380 | 9.924 | −11.442 | 31.937 | 1.00 | 40.97 | O |
| ATOM | 14 | ND2 | ASN | A | 380 | 12.115 | −11.037 | 31.461 | 1.00 | 36.52 | N |
| ATOM | 15 | C | ASN | A | 380 | 9.449 | −13.550 | 28.273 | 1.00 | 38.62 | C |
| ATOM | 16 | O | ASN | A | 380 | 10.144 | −14.006 | 27.355 | 1.00 | 38.16 | O |
| ATOM | 17 | N | THR | A | 381 | 8.174 | −13.193 | 28.126 | 1.00 | 36.87 | N |
| ATOM | 18 | CA | THR | A | 381 | 7.530 | −13.259 | 26.812 | 1.00 | 35.53 | C |
| ATOM | 19 | CB | THR | A | 381 | 6.303 | −14.214 | 26.805 | 1.00 | 35.83 | C |
| ATOM | 20 | OG1 | THR | A | 381 | 5.350 | −13.786 | 27.792 | 1.00 | 37.01 | O |
| ATOM | 21 | CG2 | THR | A | 381 | 6.717 | −15.621 | 27.249 | 1.00 | 35.34 | C |
| ATOM | 22 | C | THR | A | 381 | 7.123 | −11.901 | 26.289 | 1.00 | 33.36 | C |
| ATOM | 23 | O | THR | A | 381 | 6.745 | −11.028 | 27.043 | 1.00 | 32.65 | O |
| ATOM | 24 | N | ILE | A | 382 | 7.170 | −11.770 | 24.971 | 1.00 | 31.97 | N |
| ATOM | 25 | CA | ILE | A | 382 | 6.820 | −10.549 | 24.266 | 1.00 | 30.21 | C |
| ATOM | 26 | CB | ILE | A | 382 | 6.724 | −10.881 | 22.782 | 1.00 | 30.35 | C |
| ATOM | 27 | CG1 | ILE | A | 382 | 6.672 | −9.609 | 21.938 | 1.00 | 29.02 | C |
| ATOM | 28 | CD1 | ILE | A | 382 | 7.902 | −8.721 | 22.081 | 1.00 | 33.06 | C |
| ATOM | 29 | CG2 | ILE | A | 382 | 5.534 | −11.833 | 22.531 | 1.00 | 28.36 | C |
| ATOM | 30 | C | ILE | A | 382 | 5.498 | −10.020 | 24.767 | 1.00 | 29.89 | C |
| ATOM | 31 | O | ILE | A | 382 | 5.258 | −8.833 | 24.884 | 1.00 | 30.56 | O |
| ATOM | 32 | N | GLN | A | 383 | 4.638 | −10.942 | 25.092 | 1.00 | 29.58 | N |
| ATOM | 33 | CA | GLN | A | 383 | 3.305 | −10.639 | 25.574 | 1.00 | 29.76 | C |
| ATOM | 34 | CB | GLN | A | 383 | 2.535 | −11.945 | 25.820 | 1.00 | 29.93 | C |
| ATOM | 35 | CG | GLN | A | 383 | 1.103 | −11.705 | 26.237 | 1.00 | 34.71 | C |
| ATOM | 36 | CD | GLN | A | 383 | 0.261 | −12.955 | 26.137 | 1.00 | 39.96 | C |
| ATOM | 37 | OE1 | GLN | A | 383 | −0.945 | −12.917 | 26.383 | 1.00 | 39.55 | O |
| ATOM | 38 | NE2 | GLN | A | 383 | 0.895 | −14.071 | 25.754 | 1.00 | 43.51 | N |
| ATOM | 39 | C | GLN | A | 383 | 3.155 | −9.723 | 26.795 | 1.00 | 27.99 | C |
| ATOM | 40 | O | GLN | A | 383 | 2.247 | −8.901 | 26.822 | 1.00 | 27.26 | O |
| ATOM | 41 | N | GLN | A | 384 | 4.019 | −9.868 | 27.792 | 1.00 | 26.56 | N |
| ATOM | 42 | CA | GLN | A | 384 | 3.915 | −9.050 | 28.974 | 1.00 | 24.94 | C |
| ATOM | 43 | CB | GLN | A | 384 | 4.648 | −9.689 | 30.147 | 1.00 | 26.49 | C |
| ATOM | 44 | CG | GLN | A | 384 | 6.156 | −9.957 | 30.006 | 1.00 | 29.94 | C |
| ATOM | 45 | CD | GLN | A | 384 | 6.694 | −10.838 | 31.188 | 1.00 | 35.67 | C |
| ATOM | 46 | OE1 | GLN | A | 384 | 6.215 | −11.970 | 31.425 | 1.00 | 33.73 | O |
| ATOM | 47 | NE2 | GLN | A | 384 | 7.677 | −10.306 | 31.921 | 1.00 | 37.23 | N |
| ATOM | 48 | C | GLN | A | 384 | 4.371 | −7.617 | 28.771 | 1.00 | 23.54 | C |
| ATOM | 49 | O | GLN | A | 384 | 3.720 | −6.670 | 29.241 | 1.00 | 23.14 | O |
| ATOM | 50 | N | LEU | A | 385 | 5.466 | −7.419 | 28.053 | 1.00 | 21.30 | N |
| ATOM | 51 | CA | LEU | A | 385 | 5.894 | −6.053 | 27.840 | 1.00 | 19.42 | C |
| ATOM | 52 | CB | LEU | A | 385 | 7.244 | −5.976 | 27.128 | 1.00 | 20.05 | C |
| ATOM | 53 | CG | LEU | A | 385 | 7.786 | −4.583 | 26.770 | 1.00 | 20.09 | C |
| ATOM | 54 | CD1 | LEU | A | 385 | 7.807 | −3.617 | 27.986 | 1.00 | 16.89 | C |
| ATOM | 55 | CD2 | LEU | A | 385 | 9.205 | −4.708 | 26.201 | 1.00 | 22.07 | C |
| ATOM | 56 | C | LEU | A | 385 | 4.848 | −5.381 | 26.989 | 1.00 | 18.64 | C |
| ATOM | 57 | O | LEU | A | 385 | 4.598 | −4.175 | 27.168 | 1.00 | 17.76 | O |
| ATOM | 58 | N | MET | A | 386 | 4.253 | −6.140 | 26.050 | 1.00 | 16.78 | N |
| ATOM | 59 | CA | MET | A | 386 | 3.192 | −5.561 | 25.195 | 1.00 | 16.27 | C |
| ATOM | 60 | CB | MET | A | 386 | 2.769 | −6.488 | 24.033 | 1.00 | 16.13 | C |
| ATOM | 61 | CG | MET | A | 386 | 3.278 | −6.115 | 22.679 | 1.00 | 14.44 | C |
| ATOM | 62 | SD | MET | A | 386 | 2.985 | −7.361 | 21.343 | 1.00 | 20.61 | S |
| ATOM | 63 | CE | MET | A | 386 | 1.260 | −6.895 | 20.591 | 1.00 | 17.31 | C |
| ATOM | 64 | C | MET | A | 386 | 1.953 | −5.117 | 26.014 | 1.00 | 15.44 | C |
| ATOM | 65 | O | MET | A | 386 | 1.304 | −4.152 | 25.688 | 1.00 | 14.45 | O |
| ATOM | 66 | N | MET | A | 387 | 1.641 | −5.823 | 27.085 | 1.00 | 14.99 | N |
| ATOM | 67 | CA | MET | A | 387 | 0.500 | −5.451 | 27.907 | 1.00 | 15.36 | C |
| ATOM | 68 | CB | MET | A | 387 | 0.080 | −6.634 | 28.771 | 1.00 | 16.60 | C |
| ATOM | 69 | CG | MET | A | 387 | −0.663 | −7.709 | 28.039 | 1.00 | 21.28 | C |
| ATOM | 70 | SD | MET | A | 387 | −1.472 | −8.842 | 29.235 | 1.00 | 33.36 | S |
| ATOM | 71 | CE | MET | A | 387 | −0.081 | −9.970 | 29.834 | 1.00 | 29.39 | C |
| ATOM | 72 | C | MET | A | 387 | 0.815 | −4.229 | 28.810 | 1.00 | 13.51 | C |
| ATOM | 73 | O | MET | A | 387 | −0.019 | −3.369 | 29.075 | 1.00 | 12.21 | O |
| ATOM | 74 | N | ILE | A | 388 | 2.043 | −4.183 | 29.273 | 1.00 | 11.97 | N |
| ATOM | 75 | CA | ILE | A | 388 | 2.515 | −3.099 | 30.071 | 1.00 | 10.08 | C |
| ATOM | 76 | CB | ILE | A | 388 | 3.933 | −3.432 | 30.575 | 1.00 | 11.21 | C |
| ATOM | 77 | CG1 | ILE | A | 388 | 3.834 | −4.616 | 31.556 | 1.00 | 10.61 | C |
| ATOM | 78 | CD1 | ILE | A | 388 | 5.163 | −5.022 | 32.182 | 1.00 | 14.30 | C |
| ATOM | 79 | CG2 | ILE | A | 388 | 4.646 | −2.204 | 31.211 | 1.00 | 9.99 | C |
| ATOM | 80 | C | ILE | A | 388 | 2.484 | −1.844 | 29.271 | 1.00 | 8.68 | C |
| ATOM | 81 | O | ILE | A | 388 | 1.938 | −0.883 | 29.723 | 1.00 | 10.11 | O |
| ATOM | 82 | N | LEU | A | 389 | 3.028 | −1.833 | 28.060 | 1.00 | 7.85 | N |
| ATOM | 83 | CA | LEU | A | 389 | 3.047 | −0.600 | 27.248 | 1.00 | 6.29 | C |
| ATOM | 84 | CB | LEU | A | 389 | 4.037 | −0.694 | 26.100 | 1.00 | 5.90 | C |
| ATOM | 85 | CG | LEU | A | 389 | 5.546 | −0.953 | 26.392 | 1.00 | 5.99 | C |
| ATOM | 86 | CD1 | LEU | A | 389 | 6.365 | −0.770 | 25.087 | 1.00 | 2.00 | C |
| ATOM | 87 | CD2 | LEU | A | 389 | 6.148 | −0.133 | 27.549 | 1.00 | 4.06 | C |
| ATOM | 88 | C | LEU | A | 389 | 1.681 | −0.189 | 26.740 | 1.00 | 6.09 | C |
| ATOM | 89 | O | LEU | A | 389 | 1.326 | 0.998 | 26.635 | 1.00 | 5.28 | O |
| ATOM | 90 | N | ASN | A | 390 | 0.872 | −1.196 | 26.489 | 1.00 | 6.69 | N |
| ATOM | 91 | CA | ASN | A | 390 | −0.485 | −0.947 | 26.080 | 1.00 | 7.16 | C |
| ATOM | 92 | CB | ASN | A | 390 | −1.197 | −2.234 | 25.755 | 1.00 | 6.39 | C |
| ATOM | 93 | CG | ASN | A | 390 | −1.054 | −2.597 | 24.310 | 1.00 | 8.37 | C |
| ATOM | 94 | OD1 | ASN | A | 390 | −0.467 | −1.844 | 23.505 | 1.00 | 7.04 | O |
| ATOM | 95 | ND2 | ASN | A | 390 | −1.582 | −3.753 | 23.948 | 1.00 | 11.15 | N |
| ATOM | 96 | C | ASN | A | 390 | −1.269 | −0.146 | 27.084 | 1.00 | 7.15 | C |
| ATOM | 97 | O | ASN | A | 390 | −2.038 | 0.694 | 26.653 | 1.00 | 9.07 | O |
| ATOM | 98 | N | SER | A | 391 | −1.074 | −0.344 | 28.385 | 1.00 | 6.14 | N |
| ATOM | 99 | CA | SER | A | 391 | −1.849 | 0.461 | 29.338 | 1.00 | 7.59 | C |
| ATOM | 100 | CB | SER | A | 391 | −2.517 | −0.391 | 30.413 | 1.00 | 7.30 | C |
| ATOM | 101 | OG | SER | A | 391 | −1.555 | −1.006 | 31.241 | 1.00 | 6.73 | O |
| ATOM | 102 | C | SER | A | 391 | −1.091 | 1.626 | 30.005 | 1.00 | 7.70 | C |
| ATOM | 103 | O | SER | A | 391 | −1.696 | 2.512 | 30.608 | 1.00 | 6.83 | O |
| ATOM | 104 | N | ALA | A | 392 | 0.233 | 1.610 | 29.872 | 1.00 | 8.42 | N |
| ATOM | 105 | CA | ALA | A | 392 | 1.082 | 2.673 | 30.415 | 1.00 | 7.80 | C |
| ATOM | 106 | CB | ALA | A | 392 | 2.494 | 2.367 | 30.091 | 1.00 | 6.49 | C |
| ATOM | 107 | C | ALA | A | 392 | 0.695 | 4.043 | 29.860 | 1.00 | 8.43 | C |
| ATOM | 108 | O | ALA | A | 392 | 0.169 | 4.150 | 28.755 | 1.00 | 8.78 | 0 |
| ATOM | 109 | N | SER | A | 393 | 0.958 | 5.087 | 30.638 | 1.00 | 10.46 | N |
| ATOM | 110 | CA | SER | A | 393 | 0.655 | 6.482 | 30.310 | 1.00 | 11.12 | C |
| ATOM | 111 | CB | SER | A | 393 | 0.692 | 7.253 | 31.571 | 1.00 | 11.04 | C |
| ATOM | 112 | OG | SER | A | 393 | 0.415 | 8.586 | 31.290 | 1.00 | 15.76 | O |
| ATOM | 113 | C | SER | A | 393 | 1.651 | 7.157 | 29.385 | 1.00 | 12.00 | C |
| ATOM | 114 | O | SER | A | 393 | 2.838 | 6.842 | 29.408 | 1.00 | 13.27 | O |
| ATOM | 115 | N | ASP | A | 394 | 1.167 | 8.108 | 28.595 | 1.00 | 11.46 | N |
| ATOM | 116 | CA | ASP | A | 394 | 1.987 | 8.875 | 27.679 | 1.00 | 11.53 | C |
| ATOM | 117 | CB | ASP | A | 394 | 1.133 | 9.445 | 26.524 | 1.00 | 10.85 | C |
| ATOM | 118 | CG | ASP | A | 394 | 0.624 | 8.353 | 25.569 | 1.00 | 15.82 | C |
| ATOM | 119 | OD1 | ASP | A | 394 | 1.378 | 7.926 | 24.658 | 1.00 | 17.94 | O |
| ATOM | 120 | OD2 | ASP | A | 394 | −0.509 | 7.835 | 25.673 | 1.00 | 18.22 | O |
| ATOM | 121 | C | ASP | A | 394 | 2.686 | 10.030 | 28.391 | 1.00 | 11.56 | C |
| ATOM | 122 | O | ASP | A | 394 | 3.497 | 10.760 | 27.796 | 1.00 | 12.74 | O |
| ATOM | 123 | N | GLN | A | 395 | 2.383 | 10.206 | 29.656 | 1.00 | 10.33 | N |
| ATOM | 124 | CA | GLN | A | 395 | 2.922 | 11.325 | 30.369 | 1.00 | 10.45 | C |
| ATOM | 125 | CB | GLN | A | 395 | 1.740 | 12.142 | 30.905 | 1.00 | 11.83 | C |
| ATOM | 126 | CG | GLN | A | 395 | 0.976 | 12.872 | 29.792 | 1.00 | 14.78 | C |
| ATOM | 127 | CD | GLN | A | 395 | 1.927 | 13.681 | 28.846 | 1.00 | 17.31 | C |
| ATOM | 128 | OE1 | GLN | A | 395 | 1.612 | 13.884 | 27.668 | 1.00 | 13.52 | O |
| ATOM | 129 | NE2 | GLN | A | 395 | 3.073 | 14.156 | 29.388 | 1.00 | 15.32 | N |
| ATOM | 130 | C | GLN | A | 395 | 3.822 | 10.878 | 31.519 | 1.00 | 8.54 | C |
| ATOM | 131 | O | GLN | A | 395 | 3.698 | 9.784 | 32.001 | 1.00 | 7.56 | O |
| ATOM | 132 | N | PRO | A | 396 | 4.735 | 11.724 | 31.946 | 1.00 | 7.59 | N |
| ATOM | 133 | CA | PRO | A | 396 | 5.567 | 11.391 | 33.102 | 1.00 | 8.16 | C |
| ATOM | 134 | CB | PRO | A | 396 | 6.401 | 12.675 | 33.324 | 1.00 | 6.76 | C |
| ATOM | 135 | CG | PRO | A | 396 | 6.360 | 13.350 | 32.081 | 1.00 | 6.09 | C |
| ATOM | 136 | CD | PRO | A | 396 | 5.063 | 13.040 | 31.373 | 1.00 | 6.71 | C |
| ATOM | 137 | C | PRO | A | 396 | 4.665 | 11.077 | 34.336 | 1.00 | 8.69 | C |
| ATOM | 138 | O | PRO | A | 396 | 3.600 | 11.699 | 34.508 | 1.00 | 9.77 | O |
| ATOM | 139 | N | SER | A | 397 | 5.084 | 10.162 | 35.184 | 1.00 | 8.42 | N |
| ATOM | 140 | CA | SER | A | 397 | 4.221 | 9.811 | 36.311 | 1.00 | 9.96 | C |
| ATOM | 141 | CB | SER | A | 397 | 4.561 | 8.437 | 36.910 | 1.00 | 9.22 | C |
| ATOM | 142 | OG | SER | A | 397 | 5.712 | 8.496 | 37.719 | 1.00 | 6.50 | O |
| ATOM | 143 | C | SER | A | 397 | 4.341 | 10.829 | 37.393 | 1.00 | 10.78 | C |
| ATOM | 144 | O | SER | A | 397 | 5.208 | 11.660 | 37.349 | 1.00 | 10.33 | O |
| ATOM | 145 | N | GLU | A | 398 | 3.475 | 10.725 | 38.380 | 1.00 | 12.24 | N |
| ATOM | 146 | CA | GLU | A | 398 | 3.529 | 11.583 | 39.523 | 1.00 | 15.01 | C |
| ATOM | 147 | CB | GLU | A | 398 | 2.442 | 11.150 | 40.497 | 1.00 | 16.05 | C |
| ATOM | 148 | CG | GLU | A | 398 | 1.144 | 10.725 | 39.763 | 1.00 | 22.93 | C |
| ATOM | 149 | CD | GLU | A | 398 | 0.312 | 11.914 | 39.223 | 1.00 | 30.62 | C |
| ATOM | 150 | OE1 | GLU | A | 398 | −0.005 | 12.877 | 40.018 | 1.00 | 28.54 | O |
| ATOM | 151 | OE2 | GLU | A | 398 | −0.016 | 11.876 | 37.984 | 1.00 | 34.64 | O |
| ATOM | 152 | C | GLU | A | 398 | 4.906 | 11.482 | 40.171 | 1.00 | 14.63 | C |
| ATOM | 153 | O | GLU | A | 398 | 5.503 | 12.440 | 40.559 | 1.00 | 14.38 | O |
| ATOM | 154 | N | ASN | A | 399 | 5.422 | 10.286 | 40.251 | 1.00 | 15.36 | N |
| ATOM | 155 | CA | ASN | A | 399 | 6.715 | 10.093 | 40.828 | 1.00 | 15.54 | C |
| ATOM | 156 | CB | ASN | A | 399 | 6.949 | 8.607 | 40.993 | 1.00 | 17.66 | C |
| ATOM | 157 | CG | ASN | A | 399 | 7.545 | 8.279 | 42.313 | 1.00 | 23.36 | C |
| ATOM | 158 | OD1 | ASN | A | 399 | 8.739 | 8.543 | 42.555 | 1.00 | 29.18 | O |
| ATOM | 159 | ND2 | ASN | A | 399 | 6.719 | 7.744 | 43.213 | 1.00 | 26.01 | N |
| ATOM | 160 | C | ASN | A | 399 | 7.843 | 10.673 | 40.001 | 1.00 | 14.48 | C |
| ATOM | 161 | O | ASN | A | 399 | 8.811 | 11.219 | 40.573 | 1.00 | 14.83 | O |
| ATOM | 162 | N | LEU | A | 400 | 7.747 | 10.584 | 38.663 | 1.00 | 11.35 | N |
| ATOM | 163 | CA | LEU | A | 400 | 8.829 | 11.137 | 37.861 | 1.00 | 9.12 | C |
| ATOM | 164 | CB | LEU | A | 400 | 8.722 | 10.696 | 36.425 | 1.00 | 8.94 | C |
| ATOM | 165 | CG | LEU | A | 400 | 9.965 | 10.282 | 35.637 | 1.00 | 7.91 | C |
| ATOM | 166 | CD1 | LEU | A | 400 | 9.655 | 10.352 | 34.107 | 1.00 | 6.48 | C |
| ATOM | 167 | CD2 | LEU | A | 400 | 11.217 | 11.040 | 35.988 | 1.00 | 6.99 | C |
| ATOM | 168 | C | LEU | A | 400 | 8.933 | 12.675 | 37.981 | 1.00 | 7.77 | C |
| ATOM | 169 | O | LEU | A | 400 | 10.029 | 13.239 | 38.047 | 1.00 | 7.08 | O |
| ATOM | 170 | N | ILE | A | 401 | 7.783 | 13.331 | 38.001 | 1.00 | 7.02 | N |
| ATOM | 171 | CA | ILE | A | 401 | 7.672 | 14.766 | 38.198 | 1.00 | 6.74 | C |
| ATOM | 172 | CB | ILE | A | 401 | 6.182 | 15.161 | 38.154 | 1.00 | 6.15 | C |
| ATOM | 173 | CG1 | ILE | A | 401 | 5.669 | 15.045 | 36.732 | 1.00 | 7.60 | C |
| ATOM | 174 | CD1 | ILE | A | 401 | 6.360 | 16.024 | 35.724 | 1.00 | 6.89 | C |
| ATOM | 175 | CG2 | ILE | A | 401 | 5.968 | 16.588 | 38.665 | 1.00 | 4.00 | C |
| ATOM | 176 | C | ILE | A | 401 | 8.255 | 15.091 | 39.588 | 1.00 | 7.08 | C |
| ATOM | 177 | O | ILE | A | 401 | 8.872 | 16.141 | 39.834 | 1.00 | 6.41 | O |
| ATOM | 178 | N | SER | A | 402 | 8.112 | 14.141 | 40.479 | 1.00 | 6.89 | N |
| ATOM | 179 | CA | SER | A | 402 | 8.660 | 14.353 | 41.773 | 1.00 | 9.50 | C |
| ATOM | 180 | CB | SER | A | 402 | 8.347 | 13.166 | 42.680 | 1.00 | 10.22 | C |
| ATOM | 181 | OG | SER | A | 402 | 9.222 | 13.157 | 43.782 | 1.00 | 14.32 | O |
| ATOM | 182 | C | SER | A | 402 | 10.145 | 14.607 | 41.604 | 1.00 | 8.81 | C |
| ATOM | 183 | O | SER | A | 402 | 10.687 | 15.584 | 42.107 | 1.00 | 8.60 | O |
| ATOM | 184 | N | TYR | A | 403 | 10.799 | 13.744 | 40.852 | 1.00 | 9.20 | N |
| ATOM | 185 | CA | TYR | A | 403 | 12.231 | 13.941 | 40.585 | 1.00 | 9.67 | C |
| ATOM | 186 | CB | TYR | A | 403 | 12.780 | 12.753 | 39.816 | 1.00 | 9.50 | C |
| ATOM | 187 | CG | TYR | A | 403 | 13.035 | 11.498 | 40.641 | 1.00 | 10.54 | C |
| ATOM | 188 | CD1 | TYR | A | 403 | 14.106 | 11.434 | 41.555 | 1.00 | 11.31 | C |
| ATOM | 189 | CE1 | TYR | A | 403 | 14.355 | 10.307 | 42.276 | 1.00 | 9.55 | C |
| ATOM | 190 | CZ | TYR | A | 403 | 13.546 | 9.194 | 42.063 | 1.00 | 10.21 | C |
| ATOM | 191 | OH | TYR | A | 403 | 13.807 | 8.017 | 42.705 | 1.00 | 12.21 | O |
| ATOM | 192 | CE2 | TYR | A | 403 | 12.514 | 9.224 | 41.171 | 1.00 | 6.97 | C |
| ATOM | 193 | CD2 | TYR | A | 403 | 12.251 | 10.368 | 40.475 | 1.00 | 7.64 | C |
| ATOM | 194 | C | TYR | A | 403 | 12.579 | 15.264 | 39.824 | 1.00 | 9.18 | C |
| ATOM | 195 | O | TYR | A | 403 | 13.591 | 15.916 | 40.165 | 1.00 | 8.20 | O |
| ATOM | 196 | N | PHE | A | 404 | 11.751 | 15.634 | 38.836 | 1.00 | 7.17 | N |
| ATOM | 197 | CA | PHE | A | 404 | 11.953 | 16.876 | 38.133 | 1.00 | 8.77 | C |
| ATOM | 198 | CB | PHE | A | 404 | 10.904 | 17.114 | 36.997 | 1.00 | 9.18 | C |
| ATOM | 199 | CG | PHE | A | 404 | 10.887 | 16.043 | 35.902 | 1.00 | 6.14 | C |
| ATOM | 200 | CD1 | PHE | A | 404 | 11.894 | 15.068 | 35.828 | 1.00 | 4.71 | C |
| ATOM | 201 | CE1 | PHE | A | 404 | 11.866 | 14.087 | 34.869 | 1.00 | 2.70 | C |
| ATOM | 202 | CZ | PHE | A | 404 | 10.849 | 14.068 | 33.889 | 1.00 | 2.00 | C |
| ATOM | 203 | CE2 | PHE | A | 404 | 9.856 | 15.048 | 33.939 | 1.00 | 2.00 | C |
| ATOM | 204 | CD2 | PHE | A | 404 | 9.876 | 16.016 | 34.964 | 1.00 | 2.00 | C |
| ATOM | 205 | C | PHE | A | 404 | 11.909 | 18.081 | 39.104 | 1.00 | 10.04 | C |
| ATOM | 206 | O | PHE | A | 404 | 12.696 | 19.011 | 38.979 | 1.00 | 10.48 | O |
| ATOM | 207 | N | ASN | A | 405 | 10.991 | 18.073 | 40.060 | 1.00 | 10.29 | N |
| ATOM | 208 | CA | ASN | A | 405 | 10.927 | 19.164 | 41.005 | 1.00 | 11.87 | C |
| ATOM | 209 | CB | ASN | A | 405 | 9.546 | 19.150 | 41.687 | 1.00 | 13.31 | C |
| ATOM | 210 | CG | ASN | A | 405 | 8.446 | 19.650 | 40.757 | 1.00 | 14.99 | C |
| ATOM | 211 | OD1 | ASN | A | 405 | 7.272 | 19.493 | 41.026 | 1.00 | 18.52 | O |
| ATOM | 212 | ND2 | ASN | A | 405 | 8.844 | 20.265 | 39.670 | 1.00 | 12.65 | N |
| ATOM | 213 | C | ASN | A | 405 | 12.056 | 19.165 | 42.080 | 1.00 | 11.91 | C |
| ATOM | 214 | O | ASN | A | 405 | 12.255 | 20.149 | 42.813 | 1.00 | 10.19 | O |
| ATOM | 215 | N | ASN | A | 406 | 12.779 | 18.060 | 42.188 | 1.00 | 11.31 | N |
| ATOM | 216 | CA | ASN | A | 406 | 13.861 | 18.036 | 43.158 | 1.00 | 12.26 | C |
| ATOM | 217 | CB | ASN | A | 406 | 14.059 | 16.626 | 43.708 | 1.00 | 12.84 | C |
| ATOM | 218 | CG | ASN | A | 406 | 12.832 | 16.115 | 44.505 | 1.00 | 15.33 | C |
| ATOM | 219 | OD1 | ASN | A | 406 | 12.327 | 16.823 | 45.337 | 1.00 | 18.94 | O |
| ATOM | 220 | ND2 | ASN | A | 406 | 12.371 | 14.902 | 44.231 | 1.00 | 16.26 | N |
| ATOM | 221 | C | ASN | A | 406 | 15.190 | 18.520 | 42.617 | 1.00 | 12.01 | C |
| ATOM | 222 | O | ASN | A | 406 | 16.155 | 18.526 | 43.373 | 1.00 | 13.01 | O |
| ATOM | 223 | N | CYS | A | 407 | 15.245 | 18.845 | 41.316 | 1.00 | 10.65 | N |
| ATOM | 224 | CA | CYS | A | 407 | 16.454 | 19.284 | 40.629 | 1.00 | 9.58 | C |
| ATOM | 225 | CB | CYS | A | 407 | 16.358 | 19.051 | 39.114 | 1.00 | 9.61 | C |
| ATOM | 226 | SG | CYS | A | 407 | 16.101 | 17.366 | 38.388 | 1.00 | 7.04 | S |
| ATOM | 227 | C | CYS | A | 407 | 16.871 | 20.772 | 40.900 | 1.00 | 9.92 | C |
| ATOM | 228 | O | CYS | A | 407 | 16.056 | 21.660 | 41.100 | 1.00 | 10.76 | O |
| ATOM | 229 | N | THR | A | 408 | 18.163 | 21.036 | 40.929 | 1.00 | 9.20 | N |
| ATOM | 230 | CA | THR | A | 408 | 18.625 | 22.387 | 41.057 | 1.00 | 8.76 | C |
| ATOM | 231 | CB | THR | A | 408 | 20.132 | 22.403 | 40.955 | 1.00 | 9.46 | C |
| ATOM | 232 | OG1 | THR | A | 408 | 20.746 | 21.698 | 42.061 | 1.00 | 10.38 | O |
| ATOM | 233 | CG2 | THR | A | 408 | 20.691 | 23.859 | 41.004 | 1.00 | 8.49 | C |
| ATOM | 234 | C | THR | A | 408 | 18.050 | 23.190 | 39.890 | 1.00 | 8.65 | C |
| ATOM | 235 | O | THR | A | 408 | 17.807 | 24.356 | 40.027 | 1.00 | 9.13 | O |
| ATOM | 236 | N | VAL | A | 409 | 17.892 | 22.573 | 38.723 | 1.00 | 8.51 | N |
| ATOM | 237 | CA | VAL | A | 409 | 17.326 | 23.248 | 37.583 | 1.00 | 7.79 | C |
| ATOM | 238 | CB | VAL | A | 409 | 18.360 | 23.399 | 36.466 | 1.00 | 8.28 | C |
| ATOM | 239 | CG1 | VAL | A | 409 | 17.817 | 24.215 | 35.268 | 1.00 | 6.45 | C |
| ATOM | 240 | CG2 | VAL | A | 409 | 19.606 | 23.974 | 36.989 | 1.00 | 7.43 | C |
| ATOM | 241 | C | VAL | A | 409 | 16.213 | 22.344 | 37.104 | 1.00 | 8.17 | C |
| ATOM | 242 | O | VAL | A | 409 | 16.457 | 21.179 | 36.907 | 1.00 | 7.93 | O |
| ATOM | 243 | N | ASN | A | 410 | 15.007 | 22.876 | 36.914 | 1.00 | 7.49 | N |
| ATOM | 244 | CA | ASN | A | 410 | 13.864 | 22.062 | 36.530 | 1.00 | 8.42 | C |
| ATOM | 245 | CB | ASN | A | 410 | 12.566 | 22.786 | 36.929 | 1.00 | 8.29 | C |
| ATOM | 246 | CG | ASN | A | 410 | 11.361 | 21.860 | 36.935 | 1.00 | 7.78 | C |
| ATOM | 247 | OD1 | ASN | A | 410 | 11.027 | 21.254 | 35.930 | 1.00 | 17.24 | O |
| ATOM | 248 | ND2 | ASN | A | 410 | 10.706 | 21.767 | 38.037 | 1.00 | 2.36 | N |
| ATOM | 249 | C | ASN | A | 410 | 13.835 | 21.629 | 35.028 | 1.00 | 8.35 | C |
| ATOM | 250 | O | ASN | A | 410 | 13.852 | 22.453 | 34.141 | 1.00 | 7.62 | O |
| ATOM | 251 | N | PRO | A | 411 | 13.848 | 20.329 | 34.765 | 1.00 | 8.01 | N |
| ATOM | 252 | CA | PRO | A | 411 | 13.819 | 19.811 | 33.385 | 1.00 | 7.91 | C |
| ATOM | 253 | CB | PRO | A | 411 | 14.685 | 18.525 | 33.482 | 1.00 | 6.94 | C |
| ATOM | 254 | CG | PRO | A | 411 | 14.357 | 17.998 | 34.864 | 1.00 | 6.32 | C |
| ATOM | 255 | CD | PRO | A | 411 | 14.113 | 19.259 | 35.756 | 1.00 | 7.63 | C |
| ATOM | 256 | C | PRO | A | 411 | 12.470 | 19.497 | 32.786 | 1.00 | 8.05 | C |
| ATOM | 257 | O | PRO | A | 411 | 12.384 | 19.310 | 31.569 | 1.00 | 9.08 | O |
| ATOM | 258 | N | LYS | A | 412 | 11.422 | 19.458 | 33.581 | 1.00 | 8.49 | N |
| ATOM | 259 | CA | LYS | A | 412 | 10.086 | 19.154 | 33.046 | 1.00 | 9.75 | C |
| ATOM | 260 | CB | LYS | A | 412 | 9.052 | 19.504 | 34.066 | 1.00 | 9.19 | C |
| ATOM | 261 | CG | LYS | A | 412 | 7.744 | 18.856 | 33.805 | 1.00 | 12.96 | C |
| ATOM | 262 | CD | LYS | A | 412 | 6.730 | 19.221 | 34.859 | 1.00 | 20.30 | C |
| ATOM | 263 | CE | LYS | A | 412 | 5.921 | 20.478 | 34.478 | 1.00 | 22.28 | C |
| ATOM | 264 | NZ | LYS | A | 412 | 4.874 | 20.156 | 33.455 | 1.00 | 24.30 | N |
| ATOM | 265 | C | LYS | A | 412 | 9.717 | 19.867 | 31.717 | 1.00 | 10.16 | C |
| ATOM | 266 | O | LYS | A | 412 | 8.997 | 19.336 | 30.884 | 1.00 | 9.72 | O |
| ATOM | 267 | N | GLU | A | 413 | 10.253 | 21.055 | 31.526 | 1.00 | 10.93 | N |
| ATOM | 268 | CA | GLU | A | 413 | 9.971 | 21.858 | 30.354 | 1.00 | 12.98 | C |
| ATOM | 269 | CB | GLU | A | 413 | 10.335 | 23.314 | 30.655 | 1.00 | 14.77 | C |
| ATOM | 270 | CG | GLU | A | 413 | 9.876 | 24.252 | 29.569 | 1.00 | 22.12 | C |
| ATOM | 271 | CD | GLU | A | 413 | 8.366 | 24.221 | 29.395 | 1.00 | 30.57 | C |
| ATOM | 272 | OE1 | GLU | A | 413 | 7.756 | 23.307 | 30.039 | 1.00 | 29.00 | O |
| ATOM | 273 | OE2 | GLU | A | 413 | 7.827 | 25.111 | 28.609 | 1.00 | 33.99 | O |
| ATOM | 274 | C | GLU | A | 413 | 10.693 | 21.398 | 29.080 | 1.00 | 10.78 | C |
| ATOM | 275 | O | GLU | A | 413 | 10.082 | 21.129 | 28.015 | 1.00 | 10.26 | O |
| ATOM | 276 | N | SER | A | 414 | 12.000 | 21.334 | 29.166 | 1.00 | 8.08 | N |
| ATOM | 277 | CA | SER | A | 414 | 12.715 | 20.831 | 28.041 | 1.00 | 6.45 | C |
| ATOM | 278 | CB | SER | A | 414 | 14.174 | 20.915 | 28.308 | 1.00 | 5.87 | C |
| ATOM | 279 | OG | SER | A | 414 | 14.513 | 22.221 | 28.671 | 1.00 | 6.24 | O |
| ATOM | 280 | C | SER | A | 414 | 12.315 | 19.363 | 27.800 | 1.00 | 6.66 | C |
| ATOM | 281 | O | SER | A | 414 | 12.313 | 18.909 | 26.691 | 1.00 | 6.05 | O |
| ATOM | 282 | N | ILE | A | 415 | 11.998 | 18.602 | 28.836 | 1.00 | 7.08 | N |
| ATOM | 283 | CA | ILE | A | 415 | 11.537 | 17.282 | 28.494 | 1.00 | 9.05 | C |
| ATOM | 284 | CB | ILE | A | 415 | 11.925 | 16.069 | 29.384 | 1.00 | 8.83 | C |
| ATOM | 285 | CG1 | ILE | A | 415 | 13.023 | 16.338 | 30.443 | 1.00 | 5.60 | C |
| ATOM | 286 | CD1 | ILE | A | 415 | 13.107 | 15.477 | 31.689 | 1.00 | 2.00 | C |
| ATOM | 287 | CG2 | ILE | A | 415 | 12.878 | 15.445 | 28.216 | 1.00 | 17.75 | C |
| ATOM | 288 | C | ILE | A | 415 | 10.274 | 17.026 | 27.672 | 1.00 | 8.74 | C |
| ATOM | 289 | O | ILE | A | 415 | 10.253 | 16.110 | 26.813 | 1.00 | 7.52 | O |
| ATOM | 290 | N | LEU | A | 416 | 9.241 | 17.788 | 27.974 | 1.00 | 7.71 | N |
| ATOM | 291 | CA | LEU | A | 416 | 8.015 | 17.661 | 27.301 | 1.00 | 7.92 | C |
| ATOM | 292 | CB | LEU | A | 416 | 6.969 | 18.514 | 28.017 | 1.00 | 7.90 | C |
| ATOM | 293 | CG | LEU | A | 416 | 6.634 | 18.055 | 29.452 | 1.00 | 11.36 | C |
| ATOM | 294 | CD1 | LEU | A | 416 | 5.682 | 19.021 | 30.228 | 1.00 | 11.42 | C |
| ATOM | 295 | CD2 | LEU | A | 416 | 6.048 | 16.647 | 29.484 | 1.00 | 3.28 | C |
| ATOM | 296 | C | LEU | A | 416 | 8.200 | 18.184 | 25.872 | 1.00 | 7.77 | C |
| ATOM | 297 | O | LEU | A | 416 | 7.621 | 17.588 | 24.900 | 1.00 | 8.01 | O |
| ATOM | 298 | N | LYS | A | 417 | 8.960 | 19.286 | 25.732 | 1.00 | 5.60 | N |
| ATOM | 299 | CA | LYS | A | 417 | 9.069 | 19.937 | 24.417 | 1.00 | 4.70 | C |
| ATOM | 300 | CB | LYS | A | 417 | 9.624 | 21.362 | 24.537 | 1.00 | 6.05 | C |
| ATOM | 301 | CG | LYS | A | 417 | 8.714 | 22.296 | 25.374 | 1.00 | 7.81 | C |
| ATOM | 302 | CD | LYS | A | 417 | 9.342 | 23.738 | 25.642 | 1.00 | 10.54 | C |
| ATOM | 303 | CE | LYS | A | 417 | 9.498 | 24.562 | 24.365 | 1.00 | 9.12 | C |
| ATOM | 304 | NZ | LYS | A | 417 | 8.200 | 24.843 | 23.770 | 1.00 | 5.02 | N |
| ATOM | 305 | C | LYS | A | 417 | 9.864 | 19.111 | 23.462 | 1.00 | 3.25 | C |
| ATOM | 306 | O | LYS | A | 417 | 9.522 | 18.992 | 22.249 | 1.00 | 2.53 | O |
| ATOM | 307 | N | ARG | A | 418 | 10.838 | 18.424 | 24.027 | 1.00 | 2.00 | N |
| ATOM | 308 | CA | ARG | A | 418 | 11.675 | 17.530 | 23.239 | 1.00 | 2.82 | C |
| ATOM | 309 | CB | ARG | A | 418 | 12.913 | 17.084 | 24.007 | 1.00 | 2.07 | C |
| ATOM | 310 | CG | ARG | A | 418 | 13.990 | 16.222 | 23.291 | 1.00 | 3.70 | C |
| ATOM | 311 | CD | ARG | A | 418 | 15.353 | 16.381 | 23.949 | 1.00 | 3.44 | C |
| ATOM | 312 | NE | ARG | A | 418 | 15.052 | 16.283 | 25.337 | 1.00 | 9.38 | N |
| ATOM | 313 | CZ | ARG | A | 418 | 15.626 | 16.901 | 26.313 | 1.00 | 6.00 | C |
| ATOM | 314 | NH1 | ARG | A | 418 | 16.644 | 17.717 | 26.110 | 1.00 | 9.43 | N |
| ATOM | 315 | NH2 | ARG | A | 418 | 15.149 | 16.666 | 27.520 | 1.00 | 7.51 | N |
| ATOM | 316 | C | ARG | A | 418 | 10.873 | 16.374 | 22.687 | 1.00 | 3.56 | C |
| ATOM | 317 | O | ARG | A | 418 | 11.026 | 16.045 | 21.472 | 1.00 | 3.39 | O |
| ATOM | 318 | N | VAL | A | 419 | 10.022 | 15.771 | 23.546 | 1.00 | 2.74 | N |
| ATOM | 319 | CA | VAL | A | 419 | 9.148 | 14.671 | 23.100 | 1.00 | 2.74 | C |
| ATOM | 320 | CB | VAL | A | 419 | 8.360 | 14.001 | 24.269 | 1.00 | 3.32 | C |
| ATOM | 321 | CG1 | VAL | A | 419 | 7.306 | 13.052 | 23.758 | 1.00 | 2.00 | C |
| ATOM | 322 | CG2 | VAL | A | 419 | 9.276 | 13.300 | 25.281 | 1.00 | 3.25 | C |
| ATOM | 323 | C | VAL | A | 419 | 8.193 | 15.168 | 21.994 | 1.00 | 2.42 | C |
| ATOM | 324 | O | VAL | A | 419 | 8.034 | 14.535 | 20.971 | 1.00 | 3.50 | O |
| ATOM | 325 | N | LYS | A | 420 | 7.615 | 16.337 | 22.159 | 1.00 | 3.84 | N |
| ATOM | 326 | CA | LYS | A | 420 | 6.751 | 16.936 | 21.135 | 1.00 | 6.62 | C |
| ATOM | 327 | CB | LYS | A | 420 | 6.277 | 18.304 | 21.639 | 1.00 | 9.19 | C |
| ATOM | 328 | CG | LYS | A | 420 | 4.764 | 18.548 | 21.803 | 1.00 | 12.82 | C |
| ATOM | 329 | CD | LYS | A | 420 | 4.215 | 19.176 | 20.530 | 1.00 | 18.36 | C |
| ATOM | 330 | CE | LYS | A | 420 | 4.359 | 18.239 | 19.264 | 1.00 | 22.97 | C |
| ATOM | 331 | NZ | LYS | A | 420 | 4.073 | 18.902 | 17.861 | 1.00 | 14.81 | N |
| ATOM | 332 | C | LYS | A | 420 | 7.452 | 17.114 | 19.771 | 1.00 | 6.45 | C |
| ATOM | 333 | O | LYS | A | 420 | 6.947 | 16.694 | 18.733 | 1.00 | 5.83 | O |
| ATOM | 334 | N | ASP | A | 421 | 8.638 | 17.722 | 19.772 | 1.00 | 7.00 | N |
| ATOM | 335 | CA | ASP | A | 421 | 9.337 | 17.999 | 18.503 | 1.00 | 5.93 | C |
| ATOM | 336 | CB | ASP | A | 421 | 10.454 | 19.038 | 18.683 | 1.00 | 5.34 | C |
| ATOM | 337 | CG | ASP | A | 421 | 9.935 | 20.397 | 19.244 | 1.00 | 10.31 | C |
| ATOM | 338 | OD1 | ASP | A | 421 | 8.673 | 20.653 | 19.288 | 1.00 | 6.28 | O |
| ATOM | 339 | OD2 | ASP | A | 421 | 10.768 | 21.259 | 19.696 | 1.00 | 14.34 | O |
| ATOM | 340 | C | ASP | A | 421 | 9.890 | 16.750 | 17.838 | 1.00 | 5.07 | C |
| ATOM | 341 | O | ASP | A | 421 | 9.683 | 16.527 | 16.652 | 1.00 | 6.86 | O |
| ATOM | 342 | N | ILE | A | 422 | 10.620 | 15.923 | 18.552 | 1.00 | 3.56 | N |
| ATOM | 343 | CA | ILE | A | 422 | 11.120 | 14.727 | 17.945 | 1.00 | 2.48 | C |
| ATOM | 344 | CB | ILE | A | 422 | 11.830 | 13.913 | 18.957 | 1.00 | 2.93 | C |
| ATOM | 345 | CG1 | ILE | A | 422 | 13.031 | 14.638 | 19.545 | 1.00 | 2.54 | C |
| ATOM | 346 | CD1 | ILE | A | 422 | 13.975 | 15.166 | 18.435 | 1.00 | 5.66 | C |
| ATOM | 347 | CG2 | ILE | A | 422 | 12.172 | 12.511 | 18.367 | 1.00 | 2.78 | C |
| ATOM | 348 | C | ILE | A | 422 | 9.931 | 13.895 | 17.402 | 1.00 | 2.68 | C |
| ATOM | 349 | O | ILE | A | 422 | 9.990 | 13.371 | 16.355 | 1.00 | 2.00 | O |
| ATOM | 350 | N | GLY | A | 423 | 8.834 | 13.746 | 18.125 | 1.00 | 3.53 | N |
| ATOM | 351 | CA | GLY | A | 423 | 7.756 | 12.986 | 17.534 | 1.00 | 3.60 | C |
| ATOM | 352 | C | GLY | A | 423 | 7.330 | 13.576 | 16.190 | 1.00 | 5.15 | C |
| ATOM | 353 | O | GLY | A | 423 | 6.867 | 12.817 | 15.328 | 1.00 | 5.53 | O |
| ATOM | 354 | N | TYR | A | 424 | 7.468 | 14.901 | 16.007 | 1.00 | 3.70 | N |
| ATOM | 355 | CA | TYR | A | 424 | 6.999 | 15.558 | 14.812 | 1.00 | 4.56 | C |
| ATOM | 356 | CB | TYR | A | 424 | 6.996 | 17.095 | 14.956 | 1.00 | 4.66 | C |
| ATOM | 357 | CG | TYR | A | 424 | 6.643 | 17.772 | 13.661 | 1.00 | 2.59 | C |
| ATOM | 358 | CD1 | TYR | A | 424 | 5.339 | 17.825 | 13.223 | 1.00 | 2.76 | C |
| ATOM | 359 | CE1 | TYR | A | 424 | 5.024 | 18.381 | 12.022 | 1.00 | 3.68 | C |
| ATOM | 360 | CZ | TYR | A | 424 | 5.981 | 18.940 | 11.251 | 1.00 | 5.36 | C |
| ATOM | 361 | OH | TYR | A | 424 | 5.659 | 19.548 | 10.085 | 1.00 | 10.06 | O |
| ATOM | 362 | CE2 | TYR | A | 424 | 7.274 | 18.940 | 11.653 | 1.00 | 6.09 | C |
| ATOM | 363 | CD2 | TYR | A | 424 | 7.608 | 18.338 | 12.866 | 1.00 | 4.14 | C |
| ATOM | 364 | C | TYR | A | 424 | 7.946 | 15.174 | 13.665 | 1.00 | 4.75 | C |
| ATOM | 365 | O | TYR | A | 424 | 7.538 | 14.845 | 12.550 | 1.00 | 3.97 | O |
| ATOM | 366 | N | ILE | A | 425 | 9.208 | 15.194 | 13.996 | 1.00 | 5.11 | N |
| ATOM | 367 | CA | ILE | A | 425 | 10.246 | 14.814 | 13.086 | 1.00 | 6.31 | C |
| ATOM | 368 | CB | ILE | A | 425 | 11.575 | 15.256 | 13.706 | 1.00 | 7.12 | C |
| ATOM | 369 | CG1 | ILE | A | 425 | 11.591 | 16.788 | 13.651 | 1.00 | 8.77 | C |
| ATOM | 370 | CD1 | ILE | A | 425 | 12.912 | 17.423 | 13.998 | 1.00 | 11.84 | C |
| ATOM | 371 | CG2 | ILE | A | 425 | 12.770 | 14.644 | 13.013 | 1.00 | 6.38 | C |
| ATOM | 372 | C | ILE | A | 425 | 10.245 | 13.338 | 12.694 | 1.00 | 6.08 | C |
| ATOM | 373 | O | ILE | A | 425 | 10.494 | 13.013 | 11.505 | 1.00 | 6.94 | O |
| ATOM | 374 | N | PHE | A | 426 | 9.942 | 12.475 | 13.661 | 1.00 | 4.61 | N |
| ATOM | 375 | CA | PHE | A | 426 | 9.951 | 11.028 | 13.521 | 1.00 | 4.17 | C |
| ATOM | 376 | CB | PHE | A | 426 | 9.753 | 10.393 | 14.915 | 1.00 | 3.43 | C |
| ATOM | 377 | CG | PHE | A | 426 | 9.726 | 8.882 | 14.904 | 1.00 | 4.06 | C |
| ATOM | 378 | CD1 | PHE | A | 426 | 8.549 | 8.213 | 14.646 | 1.00 | 2.00 | C |
| ATOM | 379 | CE1 | PHE | A | 426 | 8.518 | 6.847 | 14.597 | 1.00 | 2.00 | C |
| ATOM | 380 | CZ | PHE | A | 426 | 9.642 | 6.104 | 14.835 | 1.00 | 2.49 | C |
| ATOM | 381 | CE2 | PHE | A | 426 | 10.861 | 6.767 | 15.101 | 1.00 | 5.74 | C |
| ATOM | 382 | CD2 | PHE | A | 426 | 10.903 | 8.140 | 15.104 | 1.00 | 2.77 | C |
| ATOM | 383 | C | PHE | A | 426 | 8.872 | 10.489 | 12.555 | 1.00 | 5.54 | C |
| ATOM | 384 | O | PHE | A | 426 | 9.126 | 9.583 | 11.779 | 1.00 | 5.57 | O |
| ATOM | 385 | N | LYS | A | 427 | 7.651 | 11.011 | 12.633 | 1.00 | 6.97 | N |
| ATOM | 386 | CA | LYS | A | 427 | 6.582 | 10.546 | 11.739 | 1.00 | 7.52 | C |
| ATOM | 387 | CB | LYS | A | 427 | 5.192 | 11.052 | 12.102 | 1.00 | 7.33 | C |
| ATOM | 388 | CG | LYS | A | 427 | 4.705 | 10.664 | 13.535 | 1.00 | 13.06 | C |
| ATOM | 389 | CD | LYS | A | 427 | 3.495 | 11.526 | 13.935 | 1.00 | 21.22 | C |
| ATOM | 390 | CE | LYS | A | 427 | 3.309 | 11.580 | 15.408 | 1.00 | 28.07 | C |
| ATOM | 391 | NZ | LYS | A | 427 | 4.036 | 12.688 | 16.156 | 1.00 | 28.68 | N |
| ATOM | 392 | C | LYS | A | 427 | 6.880 | 10.870 | 10.317 | 1.00 | 5.90 | C |
| ATOM | 393 | O | LYS | A | 427 | 6.715 | 10.025 | 9.441 | 1.00 | 6.60 | O |
| ATOM | 394 | N | GLU | A | 428 | 7.364 | 12.069 | 10.081 | 1.00 | 5.34 | N |
| ATOM | 395 | CA | GLU | A | 428 | 7.608 | 12.462 | 8.709 | 1.00 | 5.59 | C |
| ATOM | 396 | CB | GLU | A | 428 | 7.930 | 13.958 | 8.614 | 1.00 | 5.72 | C |
| ATOM | 397 | CG | CLU | A | 428 | 6.890 | 14.894 | 9.187 | 1.00 | 6.84 | C |
| ATOM | 398 | CD | CLU | A | 428 | 5.572 | 14.861 | 8.439 | 1.00 | 12.45 | C |
| ATOM | 399 | OE1 | GLU | A | 428 | 5.630 | 14.955 | 7.186 | 1.00 | 19.41 | O |
| ATOM | 400 | OE2 | GLU | A | 428 | 4.475 | 14.734 | 9.075 | 1.00 | 8.62 | O |
| ATOM | 401 | C | GLU | A | 428 | 8.720 | 11.563 | 8.091 | 1.00 | 4.41 | C |
| ATOM | 402 | O | GLU | A | 428 | 8.634 | 11.081 | 6.969 | 1.00 | 3.48 | O |
| ATOM | 403 | N | LYS | A | 429 | 9.745 | 11.339 | 8.883 | 1.00 | 4.31 | N |
| ATOM | 404 | CA | LYS | A | 429 | 10.831 | 10.497 | 8.509 | 1.00 | 4.32 | C |
| ATOM | 405 | CB | LYS | A | 429 | 11.939 | 10.610 | 9.514 | 1.00 | 4.54 | C |
| ATOM | 406 | CG | LYS | A | 429 | 12.655 | 11.990 | 9.432 | 1.00 | 5.96 | C |
| ATOM | 407 | CD | LYS | A | 429 | 14.004 | 12.046 | 10.143 | 1.00 | 5.92 | C |
| ATOM | 408 | CE | LYS | A | 429 | 14.709 | 13.432 | 9.940 | 1.00 | 5.19 | C |
| ATOM | 409 | NZ | LYS | A | 429 | 16.072 | 13.456 | 10.579 | 1.00 | 3.04 | N |
| ATOM | 410 | C | LYS | A | 429 | 10.404 | 9.085 | 8.301 | 1.00 | 4.70 | C |
| ATOM | 411 | O | LYS | A | 429 | 10.740 | 8.458 | 7.295 | 1.00 | 3.93 | O |
| ATOM | 412 | N | PHE | A | 430 | 9.557 | 8.607 | 9.197 | 1.00 | 6.56 | N |
| ATOM | 413 | CA | PHE | A | 430 | 9.124 | 7.229 | 9.124 | 1.00 | 7.38 | C |
| ATOM | 414 | CB | PHE | A | 430 | 8.236 | 6.911 | 10.323 | 1.00 | 7.54 | C |
| ATOM | 415 | CG | PHE | A | 430 | 7.907 | 5.450 | 10.464 | 1.00 | 8.61 | C |
| ATOM | 416 | CD1 | PHE | A | 430 | 7.066 | 4.830 | 9.564 | 1.00 | 11.42 | C |
| ATOM | 417 | CE1 | PHE | A | 430 | 6.769 | 3.531 | 9.687 | 1.00 | 12.86 | C |
| ATOM | 418 | CZ | PHE | A | 430 | 7.329 | 2.825 | 10.689 | 1.00 | 13.90 | C |
| ATOM | 419 | CE2 | PHE | A | 430 | 8.207 | 3.404 | 11.558 | 1.00 | 9.63 | C |
| ATOM | 420 | CD2 | PHE | A | 430 | 8.469 | 4.699 | 11.469 | 1.00 | 7.48 | C |
| ATOM | 421 | C | PHE | A | 430 | 8.411 | 7.061 | 7.772 | 1.00 | 7.72 | C |
| ATOM | 422 | O | PHE | A | 430 | 8.846 | 6.303 | 6.897 | 1.00 | 6.61 | O |
| ATOM | 423 | N | ALA | A | 431 | 7.334 | 7.807 | 7.602 | 1.00 | 8.28 | N |
| ATOM | 424 | CA | ALA | A | 431 | 6.583 | 7.787 | 6.355 | 1.00 | 10.41 | C |
| ATOM | 425 | CB | ALA | A | 431 | 5.660 | 8.974 | 6.307 | 1.00 | 9.96 | C |
| ATOM | 426 | C | ALA | A | 431 | 7.482 | 7.834 | 5.109 | 1.00 | 13.02 | C |
| ATOM | 427 | O | ALA | A | 431 | 7.248 | 7.119 | 4.125 | 1.00 | 11.76 | O |
| ATOM | 428 | N | LYS | A | 432 | 8.494 | 8.710 | 5.142 | 1.00 | 15.88 | N |
| ATOM | 429 | CA | LYS | A | 432 | 9.407 | 8.866 | 4.022 | 1.00 | 18.85 | C |
| ATOM | 430 | CB | LYS | A | 432 | 10.328 | 10.025 | 4.327 | 1.00 | 20.32 | C |
| ATOM | 431 | CG | LYS | A | 432 | 11.123 | 10.540 | 3.144 | 1.00 | 25.80 | C |
| ATOM | 432 | CD | LYS | A | 432 | 12.118 | 11.692 | 3.537 | 1.00 | 32.56 | C |
| ATOM | 433 | CE | LYS | A | 432 | 11.389 | 12.923 | 4.109 | 1.00 | 36.99 | C |
| ATOM | 434 | NZ | LYS | A | 432 | 12.281 | 14.009 | 4.701 | 1.00 | 35.79 | N |
| ATOM | 435 | C | LYS | A | 432 | 10.218 | 7.574 | 3.842 | 1.00 | 19.52 | C |
| ATOM | 436 | O | LYS | A | 432 | 10.266 | 7.003 | 2.783 | 1.00 | 19.79 | O |
| ATOM | 437 | N | ALA | A | 433 | 10.789 | 7.038 | 4.909 | 1.00 | 20.45 | N |
| ATOM | 438 | CA | ALA | A | 433 | 11.573 | 5.818 | 4.747 | 1.00 | 20.25 | C |
| ATOM | 439 | CB | ALA | A | 433 | 12.205 | 5.439 | 6.008 | 1.00 | 19.29 | C |
| ATOM | 440 | C | ALA | A | 433 | 10.685 | 4.696 | 4.273 | 1.00 | 20.97 | C |
| ATOM | 441 | O | ALA | A | 433 | 11.085 | 3.860 | 3.522 | 1.00 | 19.77 | O |
| ATOM | 442 | N | VAL | A | 434 | 9.471 | 4.656 | 4.772 | 1.00 | 23.20 | N |
| ATOM | 443 | CA | VAL | A | 434 | 8.589 | 3.571 | 4.450 | 1.00 | 24.82 | C |
| ATOM | 444 | CB | VAL | A | 434 | 7.473 | 3.514 | 5.426 | 1.00 | 23.74 | C |
| ATOM | 445 | CG1 | VAL | A | 434 | 6.429 | 2.537 | 4.929 | 1.00 | 26.52 | C |
| ATOM | 446 | CG2 | VAL | A | 434 | 7.992 | 3.073 | 6.705 | 1.00 | 26.22 | C |
| ATOM | 447 | C | VAL | A | 434 | 8.011 | 3.810 | 3.087 | 1.00 | 26.57 | C |
| ATOM | 448 | O | VAL | A | 434 | 7.026 | 4.512 | 2.937 | 1.00 | 28.03 | O |
| ATOM | 449 | N | GLY | A | 435 | 8.605 | 3.225 | 2.071 | 1.00 | 28.57 | N |
| ATOM | 450 | CA | GLY | A | 435 | 8.073 | 3.426 | 0.753 | 1.00 | 30.76 | C |
| ATOM | 451 | C | GLY | A | 435 | 7.687 | 4.898 | 0.666 | 1.00 | 32.32 | C |
| ATOM | 452 | O | GLY | A | 435 | 8.546 | 5.752 | 0.859 | 1.00 | 32.41 | O |
| ATOM | 453 | N | GLN | A | 436 | 6.401 | 5.184 | 0.440 | 1.00 | 32.95 | N |
| ATOM | 454 | CA | GLN | A | 436 | 5.958 | 6.530 | 0.236 | 1.00 | 34.18 | C |
| ATOM | 455 | CB | GLN | A | 436 | 6.058 | 6.779 | −1.265 | 1.00 | 35.09 | C |
| ATOM | 456 | CG | GLN | A | 436 | 7.501 | 6.823 | −1.799 | 1.00 | 38.55 | C |
| ATOM | 457 | CD | GLN | A | 436 | 7.698 | 6.064 | −3.115 | 1.00 | 40.82 | C |
| ATOM | 458 | OE1 | GLN | A | 436 | 8.670 | 6.317 | −3.836 | 1.00 | 42.49 | O |
| ATOM | 459 | NE2 | GLN | A | 436 | 6.778 | 5.145 | −3.431 | 1.00 | 41.77 | N |
| ATOM | 460 | C | GLN | A | 436 | 4.522 | 6.874 | 0.738 | 1.00 | 34.26 | C |
| ATOM | 461 | O | GLN | A | 436 | 3.675 | 5.955 | 0.911 | 1.00 | 34.87 | O |
| ATOM | 462 | N | GLY | A | 437 | 4.278 | 8.185 | 0.952 | 1.00 | 32.88 | N |
| ATOM | 463 | CA | GLY | A | 437 | 2.979 | 8.731 | 1.293 | 1.00 | 32.07 | C |
| ATOM | 464 | C | GLY | A | 437 | 2.649 | 9.129 | 2.729 | 1.00 | 31.84 | C |
| ATOM | 465 | O | GLY | A | 437 | 3.329 | 9.947 | 3.330 | 1.00 | 33.28 | O |
| ATOM | 466 | N | CYS | A | 438 | 1.569 | 8.559 | 3.264 | 1.00 | 30.09 | N |
| ATOM | 467 | CA | CYS | A | 438 | 1.081 | 8.811 | 4.618 | 1.00 | 28.02 | C |
| ATOM | 468 | CB | CYS | A | 438 | −0.349 | 9.405 | 4.591 | 1.00 | 28.75 | C |
| ATOM | 469 | SG | CYS | A | 438 | −1.524 | 8.660 | 5.796 | 1.00 | 30.36 | S |
| ATOM | 470 | C | CYS | A | 438 | 1.019 | 7.448 | 5.270 | 1.00 | 26.25 | C |
| ATOM | 471 | O | CYS | A | 438 | 0.383 | 6.535 | 4.724 | 1.00 | 24.75 | O |
| ATOM | 472 | N | VAL | A | 439 | 1.680 | 7.320 | 6.426 | 1.00 | 24.37 | N |
| ATOM | 473 | CA | VAL | A | 439 | 1.801 | 6.035 | 7.132 | 1.00 | 22.64 | C |
| ATOM | 474 | CB | VAL | A | 439 | 3.252 | 5.510 | 7.079 | 1.00 | 22.72 | C |
| ATOM | 475 | CG1 | VAL | A | 439 | 3.330 | 4.007 | 7.496 | 1.00 | 22.50 | C |
| ATOM | 476 | CG2 | VAL | A | 439 | 3.826 | 5.722 | 5.695 | 1.00 | 22.13 | C |
| ATOM | 477 | C | VAL | A | 439 | 1.303 | 6.064 | 8.591 | 1.00 | 21.76 | C |
| ATOM | 478 | O | VAL | A | 439 | 2.080 | 6.166 | 9.531 | 1.00 | 18.79 | O |
| ATOM | 479 | N | GLU | A | 440 | −0.016 | 5.952 | 8.749 | 1.00 | 21.55 | N |
| ATOM | 480 | CA | GLU | A | 440 | −0.623 | 5.986 | 10.061 | 1.00 | 21.30 | C |
| ATOM | 481 | CB | GLU | A | 440 | −2.104 | 5.639 | 9.974 | 1.00 | 22.04 | C |
| ATOM | 482 | CG | GLU | A | 440 | −2.941 | 6.602 | 9.153 | 1.00 | 23.35 | C |
| ATOM | 483 | CD | GLU | A | 440 | −4.398 | 6.142 | 9.025 | 1.00 | 27.57 | C |
| ATOM | 484 | OE1 | GLU | A | 440 | −4.964 | 5.665 | 10.046 | 1.00 | 29.99 | O |
| ATOM | 485 | OE2 | GLU | A | 440 | −5.002 | 6.237 | 7.914 | 1.00 | 27.95 | O |
| ATOM | 486 | C | GLU | A | 440 | 0.081 | 5.115 | 11.119 | 1.00 | 20.38 | C |
| ATOM | 487 | O | GLU | A | 440 | −0.247 | 5.193 | 12.283 | 1.00 | 21.06 | O |
| ATOM | 488 | N | ILE | A | 441 | 1.098 | 4.348 | 10.760 | 1.00 | 19.36 | N |
| ATOM | 489 | CA | ILE | A | 441 | 1.691 | 3.450 | 11.758 | 1.00 | 18.24 | C |
| ATOM | 490 | CB | ILE | A | 441 | 2.085 | 2.105 | 11.088 | 1.00 | 18.75 | C |
| ATOM | 491 | CG1 | ILE | A | 441 | 0.893 | 1.146 | 11.125 | 1.00 | 21.67 | C |
| ATOM | 492 | CD1 | ILE | A | 441 | −0.283 | 1.567 | 10.160 | 1.00 | 26.94 | C |
| ATOM | 493 | CG2 | ILE | A | 441 | 3.147 | 1.404 | 11.844 | 1.00 | 22.00 | C |
| ATOM | 494 | C | ILE | A | 441 | 2.832 | 4.072 | 12.555 | 1.00 | 16.39 | C |
| ATOM | 495 | O | ILE | A | 441 | 3.166 | 3.652 | 13.665 | 1.00 | 15.75 | O |
| ATOM | 496 | N | GLY | A | 442 | 3.440 | 5.089 | 11.968 | 1.00 | 14.93 | N |
| ATOM | 497 | CA | GLY | A | 442 | 4.514 | 5.798 | 12.581 | 1.00 | 13.46 | C |
| ATOM | 498 | C | GLY | A | 442 | 4.109 | 6.399 | 13.897 | 1.00 | 14.00 | C |
| ATOM | 499 | O | GLY | A | 442 | 4.862 | 6.323 | 14.833 | 1.00 | 16.06 | O |
| ATOM | 500 | N | SER | A | 443 | 2.940 | 7.002 | 13.990 | 1.00 | 13.14 | N |
| ATOM | 501 | CA | SER | A | 443 | 2.463 | 7.538 | 15.243 | 1.00 | 12.84 | C |
| ATOM | 502 | CB | SER | A | 443 | 1.049 | 8.104 | 15.060 | 1.00 | 12.90 | C |
| ATOM | 503 | OG | SER | A | 443 | 1.074 | 9.243 | 14.212 | 1.00 | 17.50 | O |
| ATOM | 504 | C | SER | A | 443 | 2.366 | 6.520 | 16.346 | 1.00 | 12.40 | C |
| ATOM | 505 | O | SER | A | 443 | 2.867 | 6.744 | 17.417 | 1.00 | 12.76 | O |
| ATOM | 506 | N | GLN | A | 444 | 1.694 | 5.409 | 16.075 | 1.00 | 12.47 | N |
| ATOM | 507 | CA | GLN | A | 444 | 1.407 | 4.381 | 17.069 | 1.00 | 12.70 | C |
| ATOM | 508 | CB | GLN | A | 444 | 0.527 | 3.245 | 16.467 | 1.00 | 12.40 | C |
| ATOM | 509 | CG | GLN | A | 444 | 0.280 | 1.987 | 17.407 | 1.00 | 17.53 | C |
| ATOM | 510 | CD | GLN | A | 444 | −1.153 | 1.909 | 18.097 | 1.00 | 20.42 | C |
| ATOM | 511 | OE1 | GLN | A | 444 | −1.419 | 2.577 | 19.145 | 1.00 | 19.65 | O |
| ATOM | 512 | NE2 | GLN | A | 444 | −2.048 | 1.106 | 17.506 | 1.00 | 16.04 | N |
| ATOM | 513 | C | GLN | A | 444 | 2.729 | 3.893 | 17.660 | 1.00 | 11.87 | C |
| ATOM | 514 | O | GLN | A | 444 | 2.833 | 3.634 | 18.875 | 1.00 | 10.62 | O |
| ATOM | 515 | N | ARG | A | 445 | 3.765 | 3.836 | 16.821 | 1.00 | 10.93 | N |
| ATOM | 516 | CA | ARG | A | 445 | 5.022 | 3.341 | 17.337 | 1.00 | 10.57 | C |
| ATOM | 517 | CB | ARG | A | 445 | 5.910 | 2.901 | 16.201 | 1.00 | 12.33 | C |
| ATOM | 518 | CG | ARG | A | 445 | 5.318 | 1.798 | 15.401 | 1.00 | 15.05 | C |
| ATOM | 519 | CD | ARG | A | 445 | 6.277 | 1.028 | 14.668 | 1.00 | 20.23 | C |
| ATOM | 520 | NE | ARG | A | 445 | 5.592 | 0.089 | 13.790 | 1.00 | 27.54 | N |
| ATOM | 521 | CZ | ARG | A | 445 | 6.170 | −0.577 | 12.776 | 1.00 | 29.64 | C |
| ATOM | 522 | NH1 | ARG | A | 445 | 7.471 | −0.444 | 12.508 | 1.00 | 26.97 | N |
| ATOM | 523 | NH2 | ARG | A | 445 | 5.432 | −1.411 | 12.047 | 1.00 | 32.65 | N |
| ATOM | 524 | C | ARG | A | 445 | 5.751 | 4.342 | 18.236 | 1.00 | 9.95 | C |
| ATOM | 525 | O | ARG | A | 445 | 6.220 | 3.973 | 19.354 | 1.00 | 9.16 | O |
| ATOM | 526 | N | TYR | A | 446 | 5.860 | 5.593 | 17.764 | 1.00 | 7.68 | N |
| ATOM | 527 | CA | TYR | A | 446 | 6.505 | 6.634 | 18.558 | 1.00 | 6.37 | C |
| ATOM | 528 | CB | TYR | A | 446 | 6.448 | 7.988 | 17.835 | 1.00 | 6.60 | C |
| ATOM | 529 | CG | TYR | A | 446 | 7.190 | 9.082 | 18.575 | 1.00 | 3.78 | C |
| ATOM | 530 | CD1 | TYR | A | 446 | 8.564 | 9.103 | 18.559 | 1.00 | 2.15 | C |
| ATOM | 531 | CE1 | TYR | A | 446 | 9.264 | 10.019 | 19.243 | 1.00 | 2.00 | C |
| ATOM | 532 | CZ | TYR | A | 446 | 8.646 | 10.974 | 19.966 | 1.00 | 2.00 | C |
| ATOM | 533 | OH | TYR | A | 446 | 9.472 | 11.916 | 20.558 | 1.00 | 2.42 | O |
| ATOM | 534 | CE2 | TYR | A | 446 | 7.293 | 11.020 | 20.022 | 1.00 | 2.00 | C |
| ATOM | 535 | CD2 | TYR | A | 446 | 6.533 | 10.064 | 19.311 | 1.00 | 2.00 | C |
| ATOM | 536 | C | TYR | A | 446 | 5.895 | 6.730 | 19.983 | 1.00 | 5.32 | C |
| ATOM | 537 | O | TYR | A | 446 | 6.611 | 6.820 | 20.978 | 1.00 | 3.64 | O |
| ATOM | 538 | N | LYS | A | 447 | 4.568 | 6.669 | 20.054 | 1.00 | 3.91 | N |
| ATOM | 539 | CA | LYS | A | 447 | 3.856 | 6.656 | 21.333 | 1.00 | 3.14 | C |
| ATOM | 540 | CB | LYS | A | 447 | 2.336 | 6.580 | 21.139 | 1.00 | 2.11 | C |
| ATOM | 541 | CG | LYS | A | 447 | 1.767 | 7.958 | 20.756 | 1.00 | 4.83 | C |
| ATOM | 542 | CD | LYS | A | 447 | 0.326 | 7.986 | 20.456 | 1.00 | 12.30 | C |
| ATOM | 543 | CE | LYS | A | 447 | −0.122 | 9.423 | 19.935 | 1.00 | 19.61 | C |
| ATOM | 544 | NZ | LYS | A | 447 | 0.615 | 9.946 | 18.721 | 1.00 | 25.13 | N |
| ATOM | 545 | C | LYS | A | 447 | 4.301 | 5.563 | 22.287 | 1.00 | 3.08 | C |
| ATOM | 546 | O | LYS | A | 447 | 4.442 | 5.807 | 23.476 | 1.00 | 2.00 | O |
| ATOM | 547 | N | LEU | A | 448 | 4.487 | 4.354 | 21.742 | 1.00 | 3.95 | N |
| ATOM | 548 | CA | LEU | A | 448 | 4.899 | 3.219 | 22.509 | 1.00 | 3.91 | C |
| ATOM | 549 | CB | LEU | A | 448 | 4.829 | 1.917 | 21.677 | 1.00 | 4.54 | C |
| ATOM | 550 | CG | LEU | A | 448 | 3.444 | 1.290 | 21.488 | 1.00 | 6.42 | C |
| ATOM | 551 | CD1 | LEU | A | 448 | 3.498 | 0.126 | 20.480 | 1.00 | 4.90 | C |
| ATOM | 552 | CD2 | LEU | A | 448 | 2.885 | 0.833 | 22.829 | 1.00 | 2.56 | C |
| ATOM | 553 | C | LEU | A | 448 | 6.327 | 3.501 | 22.949 | 1.00 | 3.00 | C |
| ATOM | 554 | O | LEU | A | 448 | 6.724 | 3.194 | 24.097 | 1.00 | 2.00 | O |
| ATOM | 555 | N | GLY | A | 449 | 7.089 | 4.088 | 22.029 | 1.00 | 2.24 | N |
| ATOM | 556 | CA | GLY | A | 449 | 8.440 | 4.523 | 22.383 | 1.00 | 3.49 | C |
| ATOM | 557 | C | GLY | A | 449 | 8.432 | 5.426 | 23.628 | 1.00 | 4.47 | C |
| ATOM | 558 | O | GLY | A | 449 | 9.127 | 5.100 | 24.600 | 1.00 | 5.55 | O |
| ATOM | 559 | N | VAL | A | 450 | 7.586 | 6.473 | 23.665 | 1.00 | 3.49 | N |
| ATOM | 560 | CA | VAL | A | 450 | 7.575 | 7.363 | 24.792 | 1.00 | 4.55 | C |
| ATOM | 561 | CB | VAL | A | 450 | 6.399 | 8.319 | 24.875 | 1.00 | 6.58 | C |
| ATOM | 562 | CG1 | VAL | A | 450 | 6.709 | 9.670 | 25.552 | 1.00 | 7.93 | C |
| ATOM | 563 | CG2 | VAL | A | 450 | 5.813 | 8.538 | 23.636 | 1.00 | 13.08 | C |
| ATOM | 564 | C | VAL | A | 450 | 7.157 | 6.676 | 26.043 | 1.00 | 3.61 | C |
| ATOM | 565 | O | VAL | A | 450 | 7.611 | 7.058 | 27.139 | 1.00 | 2.67 | O |
| ATOM | 566 | N | ARG | A | 451 | 6.181 | 5.784 | 25.926 | 1.00 | 2.00 | N |
| ATOM | 567 | CA | ARG | A | 451 | 5.688 | 5.160 | 27.117 | 1.00 | 2.00 | C |
| ATOM | 568 | CB | ARG | A | 451 | 4.415 | 4.350 | 26.864 | 1.00 | 2.00 | C |
| ATOM | 569 | CG | ARG | A | 451 | 3.265 | 5.172 | 26.369 | 1.00 | 2.00 | C |
| ATOM | 570 | CD | ARG | A | 451 | 2.114 | 4.324 | 25.965 | 1.00 | 5.40 | C |
| ATOM | 571 | NE | ARG | A | 451 | 0.956 | 5.069 | 25.455 | 1.00 | 10.15 | N |
| ATOM | 572 | CZ | ARG | A | 451 | −0.248 | 4.474 | 25.274 | 1.00 | 14.62 | C |
| ATOM | 573 | NH1 | ARG | A | 451 | −0.364 | 3.167 | 25.570 | 1.00 | 13.39 | N |
| ATOM | 574 | NH2 | ARG | A | 451 | −1.326 | 5.159 | 24.828 | 1.00 | 11.66 | N |
| ATOM | 575 | C | ARG | A | 451 | 6.801 | 4.316 | 27.772 | 1.00 | 2.00 | C |
| ATOM | 576 | O | ARG | A | 451 | 6.862 | 4.221 | 28.985 | 1.00 | 2.06 | O |
| ATOM | 577 | N | LEU | A | 452 | 7.694 | 3.779 | 26.968 | 1.00 | 2.00 | N |
| ATOM | 578 | CA | LEU | A | 452 | 8.787 | 2.978 | 27.445 | 1.00 | 2.00 | C |
| ATOM | 579 | CB | LEU | A | 452 | 9.493 | 2.241 | 26.276 | 1.00 | 2.00 | C |
| ATOM | 580 | CG | LEU | A | 452 | 9.745 | 0.778 | 26.453 | 1.00 | 3.02 | C |
| ATOM | 581 | CD1 | LEU | A | 452 | 10.674 | 0.419 | 25.328 | 1.00 | 5.79 | C |
| ATOM | 582 | CD2 | LEU | A | 452 | 10.172 | 0.204 | 27.937 | 1.00 | 2.00 | C |
| ATOM | 583 | C | LEU | A | 452 | 9.821 | 3.895 | 28.051 | 1.00 | 2.00 | C |
| ATOM | 584 | O | LEU | A | 452 | 10.339 | 3.642 | 29.139 | 1.00 | 2.00 | O |
| ATOM | 585 | N | TYR | A | 453 | 10.108 | 4.944 | 27.287 | 1.00 | 2.78 | N |
| ATOM | 586 | CA | TYR | A | 453 | 11.061 | 6.011 | 27.640 | 1.00 | 3.85 | C |
| ATOM | 587 | CB | TYR | A | 453 | 10.910 | 7.176 | 26.662 | 1.00 | 3.22 | C |
| ATOM | 588 | CG | TYR | A | 453 | 11.501 | 8.535 | 27.070 | 1.00 | 2.74 | C |
| ATOM | 589 | CD1 | TYR | A | 453 | 12.868 | 8.741 | 27.164 | 1.00 | 2.00 | C |
| ATOM | 590 | CE1 | TYR | A | 453 | 13.402 | 9.990 | 27.425 | 1.00 | 2.00 | C |
| ATOM | 591 | CZ | TYR | A | 453 | 12.543 | 11.062 | 27.581 | 1.00 | 2.98 | C |
| ATOM | 592 | OH | TYR | A | 453 | 12.996 | 12.386 | 27.820 | 1.00 | 2.14 | O |
| ATOM | 593 | CE2 | TYR | A | 453 | 11.177 | 10.869 | 27.486 | 1.00 | 2.00 | C |
| ATOM | 594 | CD2 | TYR | A | 453 | 10.675 | 9.642 | 27.209 | 1.00 | 2.00 | C |
| ATOM | 595 | C | TYR | A | 453 | 10.847 | 6.521 | 29.061 | 1.00 | 2.90 | C |
| ATOM | 596 | O | TYR | A | 453 | 11.761 | 6.551 | 29.867 | 1.00 | 2.00 | O |
| ATOM | 597 | N | TYR | A | 454 | 9.603 | 6.875 | 29.346 | 1.00 | 2.73 | N |
| ATOM | 598 | CA | TYR | A | 454 | 9.261 | 7.371 | 30.661 | 1.00 | 2.78 | C |
| ATOM | 599 | CB | TYR | A | 454 | 7.886 | 8.025 | 30.665 | 1.00 | 2.00 | C |
| ATOM | 600 | CG | TYR | A | 454 | 7.875 | 9.427 | 30.084 | 1.00 | 2.00 | C |
| ATOM | 601 | CD1 | TYR | A | 454 | 8.920 | 10.320 | 30.330 | 1.00 | 5.16 | C |
| ATOM | 602 | CE1 | TYR | A | 454 | 8.888 | 11.674 | 29.835 | 1.00 | 2.49 | C |
| ATOM | 603 | CZ | TYR | A | 454 | 7.841 | 12.047 | 29.107 | 1.00 | 2.99 | C |
| ATOM | 604 | OH | TYR | A | 454 | 7.794 | 13.303 | 28.578 | 1.00 | 3.52 | O |
| ATOM | 605 | CE2 | TYR | A | 454 | 6.795 | 11.146 | 28.853 | 1.00 | 4.23 | C |
| ATOM | 606 | CD2 | TYR | A | 454 | 6.842 | 9.862 | 29.328 | 1.00 | 2.00 | C |
| ATOM | 607 | C | TYR | A | 454 | 9.367 | 6.274 | 31.709 | 1.00 | 3.50 | C |
| ATOM | 608 | O | TYR | A | 454 | 9.906 | 6.509 | 32.803 | 1.00 | 4.53 | O |
| ATOM | 609 | N | ARG | A | 455 | 8.870 | 5.080 | 31.388 | 1.00 | 3.34 | N |
| ATOM | 610 | CA | ARG | A | 455 | 8.891 | 3.972 | 32.323 | 1.00 | 3.12 | C |
| ATOM | 611 | CB | ARG | A | 455 | 8.101 | 2.818 | 31.725 | 1.00 | 3.07 | C |
| ATOM | 612 | CG | ARG | A | 455 | 8.180 | 1.507 | 32.471 | 1.00 | 2.58 | C |
| ATOM | 613 | CD | ARG | A | 455 | 7.680 | 0.344 | 31.669 | 1.00 | 2.00 | C |
| ATOM | 614 | NE | ARG | A | 455 | 8.236 | −0.942 | 32.090 | 1.00 | 6.42 | N |
| ATOM | 615 | CZ | ARG | A | 455 | 7.807 | −1.662 | 33.179 | 1.00 | 11.99 | C |
| ATOM | 616 | NH1 | ARG | A | 455 | 8.348 | −2.862 | 33.469 | 1.00 | 9.20 | N |
| ATOM | 617 | NH2 | ARG | A | 455 | 6.802 | −1.218 | 33.939 | 1.00 | 9.54 | N |
| ATOM | 618 | C | ARG | A | 455 | 10.365 | 3.589 | 32.710 | 1.00 | 4.18 | C |
| ATOM | 619 | O | ARG | A | 455 | 10.681 | 3.275 | 33.841 | 1.00 | 5.50 | O |
| ATOM | 620 | N | VAL | A | 456 | 11.282 | 3.706 | 31.771 | 1.00 | 4.54 | N |
| ATOM | 621 | CA | VAL | A | 456 | 12.683 | 3.420 | 31.981 | 1.00 | 2.65 | C |
| ATOM | 622 | CB | VAL | A | 456 | 13.312 | 3.113 | 30.643 | 1.00 | 2.00 | C |
| ATOM | 623 | CG1 | VAL | A | 456 | 14.859 | 3.065 | 30.755 | 1.00 | 4.98 | C |
| ATOM | 624 | CG2 | VAL | A | 456 | 12.742 | 1.787 | 30.107 | 1.00 | 2.00 | C |
| ATOM | 625 | C | VAL | A | 456 | 13.432 | 4.511 | 32.717 | 1.00 | 2.66 | C |
| ATOM | 626 | O | VAL | A | 456 | 14.296 | 4.238 | 33.552 | 1.00 | 3.62 | O |
| ATOM | 627 | N | MET | A | 457 | 13.107 | 5.760 | 32.430 | 1.00 | 3.27 | N |
| ATOM | 628 | CA | MET | A | 457 | 13.752 | 6.909 | 33.073 | 1.00 | 2.00 | C |
| ATOM | 629 | CB | MET | A | 457 | 13.250 | 8.218 | 32.443 | 1.00 | 2.00 | C |
| ATOM | 630 | CG | MET | A | 457 | 13.849 | 9.459 | 33.049 | 1.00 | 2.00 | C |
| ATOM | 631 | SD | MET | A | 457 | 13.120 | 11.027 | 32.572 | 1.00 | 2.00 | S |
| ATOM | 632 | CE | MET | A | 457 | 13.434 | 10.941 | 30.705 | 1.00 | 2.00 | C |
| ATOM | 633 | C | MET | A | 457 | 13.454 | 6.866 | 34.580 | 1.00 | 2.43 | C |
| ATOM | 634 | O | MET | A | 457 | 14.312 | 7.094 | 35.376 | 1.00 | 2.00 | O |
| ATOM | 635 | N | GLU | A | 458 | 12.217 | 6.546 | 34.941 | 1.00 | 3.37 | N |
| ATOM | 636 | CA | GLU | A | 458 | 11.796 | 6.489 | 36.329 | 1.00 | 4.85 | C |
| ATOM | 637 | CB | GLU | A | 458 | 10.250 | 6.508 | 36.461 | 1.00 | 6.22 | C |
| ATOM | 638 | CG | GLU | A | 458 | 9.743 | 6.297 | 37.876 | 1.00 | 9.88 | C |
| ATOM | 639 | CD | GLU | A | 458 | 8.217 | 6.296 | 38.008 | 1.00 | 16.41 | C |
| ATOM | 640 | OE1 | GLU | A | 458 | 7.728 | 6.142 | 39.145 | 1.00 | 16.60 | O |
| ATOM | 641 | OE2 | GLU | A | 458 | 7.494 | 6.470 | 36.992 | 1.00 | 21.88 | O |
| ATOM | 642 | C | GLU | A | 458 | 12.397 | 5.295 | 37.024 | 1.00 | 3.62 | C |
| ATOM | 643 | O | GLU | A | 458 | 12.707 | 5.380 | 38.188 | 1.00 | 2.58 | O |
| ATOM | 644 | N | SER | A | 459 | 12.607 | 4.189 | 36.300 | 1.00 | 3.61 | N |
| ATOM | 645 | CA | SER | A | 459 | 13.252 | 3.018 | 36.912 | 1.00 | 2.73 | C |
| ATOM | 646 | CB | SER | A | 459 | 13.078 | 1.796 | 36.043 | 1.00 | 2.08 | C |
| ATOM | 647 | OG | SER | A | 459 | 13.733 | 0.633 | 36.548 | 1.00 | 2.00 | O |
| ATOM | 648 | C | SER | A | 459 | 14.736 | 3.330 | 37.168 | 1.00 | 3.70 | C |
| ATOM | 649 | O | SER | A | 459 | 15.297 | 2.986 | 38.201 | 1.00 | 2.47 | O |
| ATOM | 650 | N | MET | A | 460 | 15.368 | 4.020 | 36.230 | 1.00 | 4.07 | N |
| ATOM | 651 | CA | MET | A | 460 | 16.754 | 4.383 | 36.433 | 1.00 | 4.93 | C |
| ATOM | 652 | CB | MET | A | 460 | 17.361 | 5.054 | 35.178 | 1.00 | 4.86 | C |
| ATOM | 653 | CG | MET | A | 460 | 17.501 | 4.164 | 33.993 | 1.00 | 8.07 | C |
| ATOM | 654 | SD | MET | A | 460 | 18.246 | 4.959 | 32.523 | 1.00 | 13.32 | S |
| ATOM | 655 | CE | MET | A | 460 | 18.698 | 3.528 | 31.591 | 1.00 | 10.36 | C |
| ATOM | 656 | C | MET | A | 460 | 16.909 | 5.320 | 37.641 | 1.00 | 5.63 | C |
| ATOM | 657 | O | MET | A | 460 | 17.854 | 5.152 | 38.423 | 1.00 | 5.63 | O |
| ATOM | 658 | N | LEU | A | 461 | 16.023 | 6.318 | 37.767 | 1.00 | 5.49 | N |
| ATOM | 659 | CA | LEU | A | 461 | 16.142 | 7.295 | 38.821 | 1.00 | 6.56 | C |
| ATOM | 660 | CB | LEU | A | 461 | 15.134 | 8.436 | 38.604 | 1.00 | 6.42 | C |
| ATOM | 661 | CG | LEU | A | 461 | 15.377 | 9.443 | 37.446 | 1.00 | 6.02 | C |
| ATOM | 662 | CD1 | LEU | A | 461 | 14.169 | 10.331 | 37.283 | 1.00 | 2.12 | C |
| ATOM | 663 | CD2 | LEU | A | 461 | 16.678 | 10.249 | 37.573 | 1.00 | 2.00 | C |
| ATOM | 664 | C | LEU | A | 461 | 15.932 | 6.630 | 40.218 | 1.00 | 8.27 | C |
| ATOM | 665 | O | LEU | A | 461 | 16.665 | 6.892 | 41.159 | 1.00 | 7.16 | O |
| ATOM | 666 | N | LYS | A | 462 | 14.903 | 5.799 | 40.351 | 1.00 | 10.76 | N |
| ATOM | 667 | CA | LYS | A | 462 | 14.653 | 5.106 | 41.623 | 1.00 | 13.62 | C |
| ATOM | 668 | CB | LYS | A | 462 | 13.464 | 4.156 | 41.578 | 1.00 | 13.89 | C |
| ATOM | 669 | CG | LYS | A | 462 | 12.086 | 4.865 | 41.307 | 1.00 | 17.14 | C |
| ATOM | 670 | CD | LYS | A | 462 | 10.931 | 3.852 | 41.398 | 1.00 | 20.36 | C |
| ATOM | 671 | CE | LYS | A | 462 | 9.546 | 4.493 | 41.278 | 1.00 | 19.96 | C |
| ATOM | 672 | NZ | LYS | A | 462 | 8.469 | 3.509 | 41.729 | 1.00 | 17.73 | N |
| ATOM | 673 | C | LYS | A | 462 | 15.886 | 4.330 | 41.985 | 1.00 | 13.79 | C |
| ATOM | 674 | O | LYS | A | 462 | 16.346 | 4.384 | 43.137 | 1.00 | 15.92 | O |
| ATOM | 675 | N | SER | A | 463 | 16.491 | 3.733 | 40.987 | 1.00 | 12.72 | N |
| ATOM | 676 | CA | SER | A | 463 | 17.684 | 2.995 | 41.195 | 1.00 | 13.57 | C |
| ATOM | 677 | CB | SER | A | 463 | 18.055 | 2.288 | 39.926 | 1.00 | 14.00 | C |
| ATOM | 678 | OG | SER | A | 463 | 19.089 | 1.391 | 40.192 | 1.00 | 13.63 | O |
| ATOM | 679 | C | SER | A | 463 | 18.862 | 3.837 | 41.628 | 1.00 | 14.52 | C |
| ATOM | 680 | O | SER | A | 463 | 19.609 | 3.479 | 42.533 | 1.00 | 15.99 | O |
| ATOM | 681 | N | GLU | A | 464 | 19.070 | 4.958 | 40.971 | 1.00 | 14.97 | N |
| ATOM | 682 | CA | GLU | A | 464 | 20.196 | 5.767 | 41.321 | 1.00 | 14.47 | C |
| ATOM | 683 | CB | GLU | A | 464 | 20.352 | 6.912 | 40.359 | 1.00 | 13.37 | C |
| ATOM | 684 | CG | GLU | A | 464 | 21.027 | 6.509 | 39.072 | 1.00 | 15.00 | C |
| ATOM | 685 | CD | GLU | A | 464 | 22.427 | 5.984 | 39.244 | 1.00 | 13.58 | C |
| ATOM | 686 | OE1 | GLU | A | 464 | 22.561 | 5.003 | 39.965 | 1.00 | 17.87 | O |
| ATOM | 687 | OE2 | GLU | A | 464 | 23.388 | 6.526 | 38.654 | 1.00 | 12.54 | O |
| ATOM | 688 | C | GLU | A | 464 | 20.064 | 6.271 | 42.729 | 1.00 | 15.03 | C |
| ATOM | 689 | O | GLU | A | 464 | 21.057 | 6.362 | 43.440 | 1.00 | 13.80 | O |
| ATOM | 690 | N | GLU | A | 465 | 18.835 | 6.619 | 43.107 | 1.00 | 16.34 | N |
| ATOM | 691 | CA | GLU | A | 465 | 18.551 | 7.176 | 44.428 | 1.00 | 18.04 | C |
| ATOM | 692 | CB | GLU | A | 465 | 17.056 | 7.574 | 44.544 | 1.00 | 17.67 | C |
| ATOM | 693 | CG | GLU | A | 465 | 16.680 | 8.192 | 45.889 | 1.00 | 19.26 | C |
| ATOM | 694 | CD | GLU | A | 465 | 15.185 | 8.431 | 46.085 | 1.00 | 21.39 | C |
| ATOM | 695 | OE1 | GLU | A | 465 | 14.733 | 8.538 | 47.260 | 1.00 | 24.00 | O |
| ATOM | 696 | OE2 | GLU | A | 465 | 14.460 | 8.543 | 45.086 | 1.00 | 20.75 | O |
| ATOM | 697 | C | GLU | A | 465 | 18.981 | 6.218 | 45.554 | 1.00 | 18.80 | C |
| ATOM | 698 | O | GLU | A | 465 | 19.486 | 6.627 | 46.552 | 1.00 | 18.58 | O |
| ATOM | 699 | N | GLU | A | 466 | 18.818 | 4.922 | 45.356 | 1.00 | 21.36 | N |
| ATOM | 700 | CA | GLU | A | 466 | 19.154 | 3.978 | 46.399 | 1.00 | 23.94 | C |
| ATOM | 701 | CB | GLU | A | 466 | 18.257 | 2.740 | 46.301 | 1.00 | 24.78 | C |
| ATOM | 702 | CG | GLU | A | 466 | 18.800 | 1.694 | 45.376 | 1.00 | 31.30 | C |
| ATOM | 703 | CD | GLU | A | 466 | 18.024 | 0.404 | 45.467 | 1.00 | 37.63 | C |
| ATOM | 704 | OE1 | GLU | A | 466 | 17.805 | −0.006 | 46.641 | 1.00 | 39.08 | O |
| ATOM | 705 | OE2 | GLU | A | 466 | 17.638 | −0.157 | 44.382 | 1.00 | 36.89 | O |
| ATOM | 706 | C | GLU | A | 466 | 20.600 | 3.578 | 46.356 | 1.00 | 23.81 | C |
| ATOM | 707 | O | GLU | A | 466 | 21.106 | 2.912 | 47.247 | 1.00 | 23.07 | O |
| ATOM | 708 | N | ARG | A | 467 | 21.257 | 3.973 | 45.272 | 1.00 | 25.21 | N |
| ATOM | 709 | CA | ARG | A | 467 | 22.666 | 3.659 | 45.051 | 1.00 | 24.44 | C |
| ATOM | 710 | CB | ARG | A | 467 | 22.925 | 3.465 | 43.576 | 1.00 | 23.48 | C |
| ATOM | 711 | CG | ARG | A | 467 | 24.352 | 3.283 | 43.224 | 1.00 | 21.88 | C |
| ATOM | 712 | CD | ARG | A | 467 | 24.659 | 3.512 | 41.707 | 1.00 | 20.93 | C |
| ATOM | 713 | NE | ARG | A | 467 | 26.015 | 4.022 | 41.616 | 1.00 | 18.63 | N |
| ATOM | 714 | CZ | ARG | A | 467 | 26.367 | 5.089 | 40.982 | 1.00 | 15.74 | C |
| ATOM | 715 | NH1 | ARG | A | 467 | 25.481 | 5.760 | 40.281 | 1.00 | 12.88 | N |
| ATOM | 716 | NH2 | ARG | A | 467 | 27.641 | 5.467 | 41.027 | 1.00 | 18.41 | N |
| ATOM | 717 | C | ARG | A | 467 | 23.560 | 4.765 | 45.573 | 1.00 | 24.26 | C |
| ATOM | 718 | O | ARG | A | 467 | 24.669 | 4.516 | 45.988 | 1.00 | 25.68 | O |
| ATOM | 719 | N | LEU | A | 468 | 23.067 | 5.979 | 45.572 | 1.00 | 23.45 | N |
| ATOM | 720 | CA | LEU | A | 468 | 23.871 | 7.109 | 45.983 | 1.00 | 23.85 | C |
| ATOM | 721 | CB | LEU | A | 468 | 24.092 | 8.052 | 44.801 | 1.00 | 23.40 | C |
| ATOM | 722 | CG | LEU | A | 468 | 24.921 | 7.685 | 43.581 | 1.00 | 23.39 | C |
| ATOM | 723 | CD1 | LEU | A | 469 | 24.393 | 8.459 | 42.390 | 1.00 | 19.92 | C |
| ATOM | 724 | CD2 | LEU | A | 468 | 26.380 | 8.047 | 43.836 | 1.00 | 25.54 | C |
| ATOM | 725 | C | LEU | A | 468 | 23.174 | 7.919 | 47.087 | 1.00 | 24.22 | C |
| ATOM | 726 | O | LEU | A | 468 | 23.753 | 8.862 | 47.608 | 1.00 | 23.40 | O |
| ATOM | 727 | N | SER | A | 469 | 21.940 | 7.549 | 47.427 | 1.00 | 24.43 | N |
| ATOM | 728 | CA | SER | A | 469 | 21.160 | 8.357 | 48.310 | 1.00 | 25.52 | C |
| ATOM | 729 | CB | SER | A | 469 | 21.893 | 8.580 | 49.621 | 1.00 | 26.28 | C |
| ATOM | 730 | OG | SER | A | 469 | 22.034 | 7.352 | 50.339 | 1.00 | 24.93 | O |
| ATOM | 731 | C | SER | A | 469 | 21.022 | 9.644 | 47.500 | 1.00 | 26.91 | C |
| ATOM | 732 | O | SER | A | 469 | 21.227 | 9.627 | 46.285 | 1.00 | 28.46 | O |
| ATOM | 733 | N | ILE | A | 470 | 20.666 | 10.774 | 48.091 | 1.00 | 26.91 | N |
| ATOM | 734 | CA | ILE | A | 470 | 20.567 | 11.941 | 47.218 | 1.00 | 25.86 | C |
| ATOM | 735 | CB | ILE | A | 470 | 21.907 | 12.106 | 46.458 | 1.00 | 25.79 | C |
| ATOM | 736 | CG1 | ILE | A | 470 | 22.706 | 13.245 | 47.112 | 1.00 | 26.46 | C |
| ATOM | 737 | CD1 | ILE | A | 470 | 24.137 | 13.426 | 46.636 | 1.00 | 28.15 | C |
| ATOM | 738 | CG2 | ILE | A | 470 | 21.697 | 12.313 | 44.944 | 1.00 | 25.97 | C |
| ATOM | 739 | C | ILE | A | 470 | 19.406 | 11.815 | 46.260 | 1.00 | 25.84 | C |
| ATOM | 740 | O | ILE | A | 470 | 19.196 | 10.760 | 45.661 | 1.00 | 23.67 | O |
| ATOM | 741 | N | GLN | A | 471 | 18.600 | 12.878 | 46.174 | 1.00 | 26.30 | N |
| ATOM | 742 | CA | GLN | A | 471 | 17.516 | 12.894 | 45.210 | 1.00 | 27.70 | C |
| ATOM | 743 | CB | GLN | A | 471 | 16.221 | 13.383 | 45.835 | 1.00 | 28.63 | C |
| ATOM | 744 | CG | GLN | A | 471 | 15.514 | 12.376 | 46.723 | 1.00 | 32.75 | C |
| ATOM | 745 | CD | GLN | A | 471 | 16.285 | 12.086 | 48.043 | 1.00 | 38.95 | C |
| ATOM | 746 | OE1 | GLN | A | 471 | 16.536 | 13.000 | 48.845 | 1.00 | 36.46 | O |
| ATOM | 747 | NE2 | GLN | A | 471 | 16.656 | 10.801 | 48.257 | 1.00 | 41.47 | N |
| ATOM | 748 | C | GLN | A | 471 | 18.005 | 13.851 | 44.141 | 1.00 | 27.03 | C |
| ATOM | 749 | O | GLN | A | 471 | 19.174 | 13.799 | 43.776 | 1.00 | 28.39 | O |
| ATOM | 750 | N | ASN | A | 472 | 17.163 | 14.703 | 43.585 | 1.00 | 25.58 | N |
| ATOM | 751 | CA | ASN | A | 472 | 17.755 | 15.736 | 42.680 | 1.00 | 25.14 | C |
| ATOM | 752 | CB | ASN | A | 472 | 18.985 | 16.425 | 43.345 | 1.00 | 24.93 | C |
| ATOM | 753 | CG | ASN | A | 472 | 19.348 | 17.803 | 42.708 | 1.00 | 28.30 | C |
| ATOM | 754 | OD1 | ASN | A | 472 | 19.877 | 18.682 | 43.390 | 1.00 | 23.38 | O |
| ATOM | 755 | ND2 | ASN | A | 472 | 19.073 | 17.974 | 41.391 | 1.00 | 29.82 | N |
| ATOM | 756 | C | ASN | A | 472 | 18.118 | 15.198 | 41.256 | 1.00 | 22.63 | C |
| ATOM | 757 | O | ASN | A | 472 | 17.204 | 14.902 | 40.446 | 1.00 | 21.83 | O |
| ATOM | 758 | N | PHE | A | 473 | 19.419 | 15.043 | 40.990 | 1.00 | 19.00 | N |
| ATOM | 759 | CA | PHE | A | 473 | 19.885 | 14.539 | 39.654 | 1.00 | 17.56 | C |
| ATOM | 760 | CB | PHE | A | 473 | 19.095 | 13.278 | 39.175 | 1.00 | 15.51 | C |
| ATOM | 761 | CG | PHE | A | 473 | 19.295 | 12.095 | 40.041 | 1.00 | 14.08 | C |
| ATOM | 762 | CD1 | PHE | A | 473 | 18.379 | 11.788 | 41.031 | 1.00 | 14.53 | C |
| ATOM | 763 | CE1 | PHE | A | 473 | 18.603 | 10.715 | 41.900 | 1.00 | 11.36 | C |
| ATOM | 764 | CZ | PHE | A | 473 | 19.726 | 9.961 | 41.785 | 1.00 | 8.01 | C |
| ATOM | 765 | CE2 | PHE | A | 473 | 20.648 | 10.257 | 40.810 | 1.00 | 10.92 | C |
| ATOM | 766 | CD2 | PHE | A | 473 | 20.444 | 11.329 | 39.954 | 1.00 | 13.01 | C |
| ATOM | 767 | C | PHE | A | 473 | 19.944 | 15.623 | 38.527 | 1.00 | 15.68 | C |
| ATOM | 768 | O | PHE | A | 473 | 20.018 | 15.264 | 37.360 | 1.00 | 15.69 | O |
| ATOM | 769 | N | SER | A | 474 | 19.930 | 16.910 | 38.897 | 1.00 | 13.72 | N |
| ATOM | 770 | CA | SER | A | 474 | 19.840 | 18.016 | 37.936 | 1.00 | 13.11 | C |
| ATOM | 771 | CB | SER | A | 474 | 20.136 | 19.379 | 38.604 | 1.00 | 14.23 | C |
| ATOM | 772 | OG | SER | A | 474 | 19.705 | 20.550 | 37.894 | 1.00 | 6.19 | O |
| ATOM | 773 | C | SER | A | 474 | 20.702 | 17.877 | 36.691 | 1.00 | 13.44 | C |
| ATOM | 774 | O | SER | A | 474 | 20.221 | 18.062 | 35.597 | 1.00 | 13.49 | O |
| ATOM | 775 | N | LYS | A | 475 | 21.978 | 17.578 | 36.867 | 1.00 | 13.37 | N |
| ATOM | 776 | CA | LYS | A | 475 | 22.878 | 17.435 | 35.754 | 1.00 | 12.47 | C |
| ATOM | 777 | CB | LYS | A | 475 | 24.286 | 17.089 | 36.185 | 1.00 | 12.77 | C |
| ATOM | 778 | CG | LYS | A | 475 | 25.288 | 17.204 | 35.033 | 1.00 | 13.73 | C |
| ATOM | 779 | CD | LYS | A | 475 | 26.724 | 17.423 | 35.480 | 1.00 | 16.66 | C |
| ATOM | 780 | CE | LYS | A | 475 | 27.742 | 17.332 | 34.315 | 1.00 | 24.37 | C |
| ATOM | 781 | NZ | LYS | A | 475 | 27.400 | 18.006 | 32.937 | 1.00 | 23.53 | N |
| ATOM | 782 | C | LYS | A | 475 | 22.467 | 16.436 | 34.747 | 1.00 | 12.94 | C |
| ATOM | 783 | O | LYS | A | 475 | 22.438 | 16.736 | 33.530 | 1.00 | 14.20 | O |
| ATOM | 784 | N | LEU | A | 476 | 22.188 | 15.223 | 35.203 | 1.00 | 12.84 | N |
| ATOM | 785 | CA | LEU | A | 476 | 21.832 | 14.124 | 34.294 | 1.00 | 11.68 | C |
| ATOM | 786 | CB | LEU | A | 476 | 21.567 | 12.879 | 35.056 | 1.00 | 11.55 | C |
| ATOM | 787 | CG | LEU | A | 476 | 21.072 | 11.659 | 34.255 | 1.00 | 14.10 | C |
| ATOM | 788 | CD1 | LEU | A | 476 | 21.999 | 11.262 | 33.212 | 1.00 | 12.17 | C |
| ATOM | 789 | CD2 | LEU | A | 476 | 20.899 | 10.436 | 35.250 | 1.00 | 14.83 | C |
| ATOM | 790 | C | LEU | A | 476 | 20.597 | 14.474 | 33.514 | 1.00 | 11.27 | C |
| ATOM | 791 | O | LEU | A | 476 | 20.608 | 14.449 | 32.289 | 1.00 | 11.58 | O |
| ATOM | 792 | N | LEU | A | 477 | 19.554 | 14.876 | 34.234 | 1.00 | 9.67 | N |
| ATOM | 793 | CA | LEU | A | 477 | 18.271 | 15.210 | 33.633 | 1.00 | 7.27 | C |
| ATOM | 794 | CB | LEU | A | 477 | 17.211 | 15.256 | 34.724 | 1.00 | 6.59 | C |
| ATOM | 795 | CG | LEU | A | 477 | 17.000 | 13.934 | 35.430 | 1.00 | 6.22 | C |
| ATOM | 796 | CD1 | LEU | A | 477 | 15.877 | 14.102 | 36.533 | 1.00 | 2.69 | C |
| ATOM | 797 | CD2 | LEU | A | 477 | 16.667 | 12.908 | 34.367 | 1.00 | 2.00 | C |
| ATOM | 798 | C | LEU | A | 477 | 18.236 | 16.520 | 32.794 | 1.00 | 6.03 | C |
| ATOM | 799 | O | LEU | A | 477 | 17.234 | 16.807 | 32.164 | 1.00 | 5.74 | O |
| ATOM | 800 | N | ASN | A | 478 | 19.283 | 17.310 | 32.784 | 1.00 | 4.35 | N |
| ATOM | 801 | CA | ASN | A | 478 | 19.264 | 18.470 | 31.877 | 1.00 | 6.17 | C |
| ATOM | 802 | CB | ASN | A | 478 | 19.716 | 19.783 | 32.539 | 1.00 | 5.15 | C |
| ATOM | 803 | CG | ASN | A | 478 | 18.612 | 20.397 | 33.419 | 1.00 | 5.45 | C |
| ATOM | 804 | OD1 | ASN | A | 478 | 17.615 | 20.849 | 32.868 | 1.00 | 6.20 | O |
| ATOM | 805 | ND2 | ASN | A | 478 | 18.764 | 20.374 | 34.773 | 1.00 | 2.00 | N |
| ATOM | 806 | C | ASN | A | 478 | 20.057 | 18.257 | 30.562 | 1.00 | 6.46 | C |
| ATOM | 807 | O | ASN | A | 478 | 20.037 | 19.127 | 29.680 | 1.00 | 6.14 | O |
| ATOM | 808 | N | ASP | A | 479 | 20.712 | 17.098 | 30.461 | 1.00 | 5.99 | N |
| ATOM | 809 | CA | ASP | A | 479 | 21.572 | 16.766 | 29.360 | 1.00 | 7.55 | C |
| ATOM | 810 | CB | ASP | A | 479 | 22.520 | 15.643 | 29.755 | 1.00 | 7.69 | C |
| ATOM | 811 | CG | ASP | A | 479 | 23.519 | 15.344 | 28.706 | 1.00 | 12.13 | C |
| ATOM | 812 | OD1 | ASP | A | 479 | 23.206 | 15.086 | 27.511 | 1.00 | 18.57 | O |
| ATOM | 813 | OD2 | ASP | A | 479 | 24.708 | 15.317 | 28.984 | 1.00 | 21.87 | O |
| ATOM | 814 | C | ASP | A | 479 | 20.791 | 16.305 | 28.146 | 1.00 | 7.54 | C |
| ATOM | 815 | O | ASP | A | 479 | 20.205 | 15.236 | 28.165 | 1.00 | 8.94 | O |
| ATOM | 816 | N | ASN | A | 480 | 20.893 | 17.062 | 27.063 | 1.00 | 6.80 | N |
| ATOM | 817 | CA | ASN | A | 480 | 20.198 | 16.762 | 25.841 | 1.00 | 5.52 | C |
| ATOM | 818 | CB | ASN | A | 480 | 20.510 | 17.835 | 24.799 | 1.00 | 4.25 | C |
| ATOM | 819 | CG | ASN | A | 480 | 19.772 | 17.630 | 23.540 | 1.00 | 5.64 | C |
| ATOM | 820 | OD1 | ASN | A | 480 | 20.369 | 17.413 | 22.488 | 1.00 | 13.62 | O |
| ATOM | 821 | ND2 | ASN | A | 480 | 18.463 | 17.638 | 23.620 | 1.00 | 5.94 | N |
| ATOM | 822 | C | ASN | A | 480 | 20.522 | 15.352 | 25.332 | 1.00 | 4.85 | C |
| ATOM | 823 | O | ASN | A | 480 | 19.612 | 14.554 | 25.048 | 1.00 | 2.00 | O |
| ATOM | 824 | N | ILE | A | 481 | 21.812 | 15.051 | 25.262 | 1.00 | 5.91 | N |
| ATOM | 825 | CA | ILE | A | 481 | 22.281 | 13.726 | 24.790 | 1.00 | 6.95 | C |
| ATOM | 826 | CB | ILE | A | 481 | 23.805 | 13.604 | 24.829 | 1.00 | 7.62 | C |
| ATOM | 827 | CG1 | ILE | A | 481 | 24.480 | 14.691 | 23.972 | 1.00 | 8.74 | C |
| ATOM | 828 | CD1 | ILE | A | 481 | 24.208 | 14.429 | 22.466 | 1.00 | 17.85 | C |
| ATOM | 829 | CG2 | ILE | A | 481 | 24.205 | 12.265 | 24.282 | 1.00 | 7.84 | C |
| ATOM | 830 | C | ILE | A | 481 | 21.713 | 12.560 | 25.551 | 1.00 | 6.93 | C |
| ATOM | 831 | O | ILE | A | 481 | 21.364 | 11.555 | 24.932 | 1.00 | 8.37 | O |
| ATOM | 832 | N | PHE | A | 482 | 21.656 | 12.660 | 26.879 | 1.00 | 5.61 | N |
| ATOM | 833 | CA | PHE | A | 482 | 21.076 | 11.595 | 27.710 | 1.00 | 4.96 | C |
| ATOM | 834 | CB | PHE | A | 482 | 21.064 | 11.967 | 29.220 | 1.00 | 3.95 | C |
| ATOM | 835 | CG | PHE | A | 482 | 20.218 | 11.064 | 30.044 | 1.00 | 2.00 | C |
| ATOM | 836 | CD1 | PHE | A | 482 | 20.616 | 9.760 | 30.273 | 1.00 | 2.30 | C |
| ATOM | 837 | CE1 | PHE | A | 482 | 19.779 | 8.872 | 31.037 | 1.00 | 4.02 | C |
| ATOM | 838 | CZ | PHE | A | 482 | 18.537 | 9.353 | 31.571 | 1.00 | 6.39 | C |
| ATOM | 839 | CE2 | PHE | A | 482 | 18.140 | 10.701 | 31.307 | 1.00 | 3.09 | C |
| ATOM | 840 | CD2 | PHE | A | 482 | 18.986 | 11.517 | 30.569 | 1.00 | 2.00 | C |
| ATOM | 841 | C | PHE | A | 482 | 19.631 | 11.205 | 27.303 | 1.00 | 4.81 | C |
| ATOM | 842 | O | PHE | A | 482 | 19.379 | 10.044 | 27.144 | 1.00 | 4.61 | O |
| ATOM | 843 | N | HIS | A | 483 | 18.721 | 12.184 | 27.192 | 1.00 | 3.46 | N |
| ATOM | 844 | CA | HIS | A | 483 | 17.323 | 11.995 | 26.803 | 1.00 | 4.48 | C |
| ATOM | 845 | CB | HIS | A | 483 | 16.500 | 13.264 | 27.001 | 1.00 | 3.03 | C |
| ATOM | 846 | CG | HIS | A | 483 | 16.343 | 13.637 | 28.434 | 1.00 | 6.88 | C |
| ATOM | 847 | ND1 | HIS | A | 483 | 15.533 | 12.932 | 29.305 | 1.00 | 5.16 | N |
| ATOM | 848 | CE1 | HIS | A | 483 | 15.629 | 13.475 | 30.504 | 1.00 | 6.16 | C |
| ATOM | 849 | NE2 | HIS | A | 483 | 16.479 | 14.482 | 30.451 | 1.00 | 4.45 | N |
| ATOM | 850 | CD2 | HIS | A | 483 | 16.956 | 14.590 | 29.173 | 1.00 | 5.78 | C |
| ATOM | 851 | C | HIS | A | 483 | 17.210 | 11.610 | 25.339 | 1.00 | 4.68 | C |
| ATOM | 852 | O | HIS | A | 483 | 16.287 | 10.899 | 24.919 | 1.00 | 4.81 | O |
| ATOM | 853 | N | MET | A | 484 | 18.163 | 12.066 | 24.564 | 1.00 | 4.57 | N |
| ATOM | 854 | CA | MET | A | 484 | 18.143 | 11.733 | 23.163 | 1.00 | 5.49 | C |
| ATOM | 855 | CB | MET | A | 484 | 19.135 | 12.555 | 22.454 | 1.00 | 5.43 | C |
| ATOM | 856 | CG | MET | A | 484 | 18.627 | 13.127 | 21.223 | 1.00 | 13.21 | C |
| ATOM | 857 | SD | MET | A | 484 | 17.108 | 14.107 | 21.275 | 1.00 | 15.95 | S |
| ATOM | 858 | CE | MET | A | 484 | 17.888 | 15.586 | 21.607 | 1.00 | 23.47 | C |
| ATOM | 859 | C | MET | A | 484 | 18.512 | 10.264 | 23.026 | 1.00 | 4.72 | C |
| ATOM | 860 | O | MET | A | 484 | 17.927 | 9.590 | 22.188 | 1.00 | 4.49 | O |
| ATOM | 861 | N | SER | A | 485 | 19.399 | 9.758 | 23.892 | 1.00 | 2.84 | N |
| ATOM | 862 | CA | SER | A | 485 | 19.863 | 8.378 | 23.799 | 1.00 | 3.18 | C |
| ATOM | 863 | CB | SER | A | 485 | 21.196 | 8.206 | 24.505 | 1.00 | 4.17 | C |
| ATOM | 864 | OG | SER | A | 485 | 22.253 | 8.922 | 23.891 | 1.00 | 5.43 | O |
| ATOM | 865 | C | SER | A | 485 | 18.866 | 7.382 | 24.382 | 1.00 | 2.76 | C |
| ATOM | 866 | O | SER | A | 485 | 18.580 | 6.323 | 23.797 | 1.00 | 3.40 | O |
| ATOM | 867 | N | LEU | A | 486 | 18.291 | 7.753 | 25.499 | 1.00 | 2.70 | N |
| ATOM | 868 | CA | LEU | A | 486 | 17.294 | 6.932 | 26.173 | 1.00 | 2.83 | C |
| ATOM | 869 | CB | LEU | A | 486 | 16.795 | 7.645 | 27.456 | 1.00 | 2.69 | C |
| ATOM | 870 | CG | LEU | A | 486 | 16.678 | 6.926 | 28.798 | 1.00 | 5.49 | C |
| ATOM | 871 | CD1 | LEU | A | 486 | 15.484 | 7.415 | 29.632 | 1.00 | 2.00 | C |
| ATOM | 872 | CD2 | LEU | A | 486 | 16.643 | 5.426 | 28.695 | 1.00 | 7.92 | C |
| ATOM | 873 | C | LEU | A | 486 | 16.135 | 6.698 | 25.188 | 1.00 | 2.25 | C |
| ATOM | 874 | O | LEU | A | 486 | 15.750 | 5.566 | 24.970 | 1.00 | 2.40 | O |
| ATOM | 875 | N | LEU | A | 487 | 15.645 | 7.774 | 24.562 | 1.00 | 2.00 | N |
| ATOM | 876 | CA | LEU | A | 487 | 14.480 | 7.731 | 23.690 | 1.00 | 3.45 | C |
| ATOM | 877 | CB | LEU | A | 487 | 14.104 | 9.150 | 23.196 | 1.00 | 4.14 | C |
| ATOM | 878 | CG | LEU | A | 487 | 12.637 | 9.614 | 23.068 | 1.00 | 5.86 | C |
| ATOM | 879 | CD1 | LEU | A | 487 | 12.505 | 10.719 | 21.953 | 1.00 | 4.99 | C |
| ATOM | 880 | CD2 | LEU | A | 487 | 11.658 | 8.512 | 22.759 | 1.00 | 7.08 | C |
| ATOM | 881 | C | LEU | A | 487 | 14.718 | 6.785 | 22.477 | 1.00 | 3.80 | C |
| ATOM | 882 | O | LEU | A | 487 | 13.887 | 5.903 | 22.194 | 1.00 | 2.00 | O |
| ATOM | 883 | N | ALA | A | 488 | 15.873 | 6.983 | 21.838 | 1.00 | 3.28 | N |
| ATOM | 884 | CA | ALA | A | 488 | 16.299 | 6.192 | 20.698 | 1.00 | 4.99 | C |
| ATOM | 885 | CB | ALA | A | 488 | 17.695 | 6.594 | 20.270 | 1.00 | 5.66 | C |
| ATOM | 886 | C | ALA | A | 488 | 16.341 | 4.758 | 21.092 | 1.00 | 5.49 | C |
| ATOM | 887 | O | ALA | A | 488 | 15.806 | 3.885 | 20.441 | 1.00 | 6.07 | O |
| ATOM | 888 | N | CYS | A | 489 | 16.993 | 4.496 | 22.198 | 1.00 | 6.10 | N |
| ATOM | 889 | CA | CYS | A | 489 | 17.122 | 3.122 | 22.588 | 1.00 | 5.29 | C |
| ATOM | 890 | CB | CYS | A | 489 | 17.998 | 3.023 | 23.837 | 1.00 | 4.37 | C |
| ATOM | 891 | SG | CYS | A | 489 | 18.230 | 1.310 | 24.279 | 1.00 | 5.78 | S |
| ATOM | 892 | C | CYS | A | 489 | 15.738 | 2.513 | 22.729 | 1.00 | 5.44 | C |
| ATOM | 893 | O | CYS | A | 489 | 15.444 | 1.438 | 22.163 | 1.00 | 4.75 | O |
| ATOM | 894 | N | ALA | A | 490 | 14.888 | 3.233 | 23.462 | 1.00 | 5.61 | N |
| ATOM | 895 | CA | ALA | A | 490 | 13.503 | 2.829 | 23.673 | 1.00 | 5.43 | C |
| ATOM | 896 | CB | ALA | A | 490 | 12.815 | 3.780 | 24.604 | 1.00 | 6.08 | C |
| ATOM | 897 | C | ALA | A | 490 | 12.700 | 2.669 | 22.406 | 1.00 | 4.62 | C |
| ATOM | 898 | O | ALA | A | 490 | 11.860 | 1.779 | 22.335 | 1.00 | 6.60 | O |
| ATOM | 899 | N | LEU | A | 491 | 12.957 | 3.472 | 21.395 | 1.00 | 3.23 | N |
| ATOM | 900 | CA | LEU | A | 491 | 12.237 | 3.330 | 20.140 | 1.00 | 2.52 | C |
| ATOM | 901 | CB | LEU | A | 491 | 12.343 | 4.622 | 19.349 | 1.00 | 2.72 | C |
| ATOM | 902 | CG | LEU | A | 491 | 11.536 | 5.885 | 19.661 | 1.00 | 2.48 | C |
| ATOM | 903 | CD1 | LEU | A | 491 | 12.083 | 7.153 | 18.984 | 1.00 | 2.00 | C |
| ATOM | 904 | CD2 | LEU | A | 491 | 10.047 | 5.660 | 19.324 | 1.00 | 2.00 | C |
| ATOM | 905 | C | LEU | A | 491 | 12.851 | 2.178 | 19.332 | 1.00 | 3.77 | C |
| ATOM | 906 | O | LEU | A | 491 | 12.243 | 1.614 | 18.485 | 1.00 | 3.11 | O |
| ATOM | 907 | N | GLU | A | 492 | 14.109 | 1.865 | 19.555 | 1.00 | 5.20 | N |
| ATOM | 908 | CA | GLU | A | 492 | 14.694 | 0.850 | 18.745 | 1.00 | 7.70 | C |
| ATOM | 909 | CB | GLU | A | 492 | 16.201 | 0.864 | 18.909 | 1.00 | 7.57 | C |
| ATOM | 910 | CG | GLU | A | 492 | 16.964 | −0.236 | 18.193 | 1.00 | 10.36 | C |
| ATOM | 911 | CD | GLU | A | 492 | 16.581 | −0.371 | 16.735 | 1.00 | 12.45 | C |
| ATOM | 912 | OE1 | GLU | A | 492 | 16.431 | 0.631 | 16.081 | 1.00 | 17.37 | O |
| ATOM | 913 | OE2 | GLU | A | 492 | 16.433 | −1.468 | 16.213 | 1.00 | 14.99 | O |
| ATOM | 914 | C | GLU | A | 492 | 14.102 | −0.480 | 19.162 | 1.00 | 9.34 | C |
| ATOM | 915 | O | GLU | A | 492 | 13.919 | −1.384 | 18.346 | 1.00 | 9.42 | O |
| ATOM | 916 | N | VAL | A | 493 | 13.806 | −0.597 | 20.445 | 1.00 | 10.25 | N |
| ATOM | 917 | CA | VAL | A | 493 | 13.230 | −1.804 | 20.970 | 1.00 | 10.00 | C |
| ATOM | 918 | CB | VAL | A | 493 | 13.208 | −1.735 | 22.536 | 1.00 | 11.55 | C |
| ATOM | 919 | CG1 | VAL | A | 493 | 12.173 | −2.764 | 23.129 | 1.00 | 8.23 | C |
| ATOM | 920 | CG2 | VAL | A | 493 | 14.608 | −1.955 | 23.072 | 1.00 | 7.40 | C |
| ATOM | 921 | C | VAL | A | 493 | 11.802 | −1.988 | 20.466 | 1.00 | 10.30 | C |
| ATOM | 922 | O | VAL | A | 493 | 11.449 | −3.037 | 20.047 | 1.00 | 10.69 | O |
| ATOM | 923 | N | VAL | A | 494 | 11.016 | −0.926 | 20.434 | 1.00 | 10.67 | N |
| ATOM | 924 | CA | VAL | A | 494 | 9.637 | −1.000 | 20.006 | 1.00 | 10.15 | C |
| ATOM | 925 | CB | VAL | A | 494 | 8.838 | 0.337 | 20.293 | 1.00 | 9.64 | C |
| ATOM | 926 | CG1 | VAL | A | 494 | 7.530 | 0.329 | 19.610 | 1.00 | 6.54 | C |
| ATOM | 927 | CG2 | VAL | A | 494 | 8.558 | 0.545 | 21.852 | 1.00 | 6.43 | C |
| ATOM | 928 | C | VAL | A | 494 | 9.550 | −1.362 | 18.563 | 1.00 | 12.13 | C |
| ATOM | 929 | O | VAL | A | 494 | 8.754 | −2.219 | 18.178 | 1.00 | 12.62 | O |
| ATOM | 930 | N | MET | A | 495 | 10.363 | −0.722 | 17.742 | 1.00 | 13.37 | N |
| ATOM | 931 | CA | MET | A | 495 | 10.344 | −1.038 | 16.324 | 1.00 | 15.28 | C |
| ATOM | 932 | CB | MET | A | 495 | 11.097 | 0.035 | 15.531 | 1.00 | 14.65 | C |
| ATOM | 933 | CG | MET | A | 495 | 10.405 | 1.316 | 15.544 | 1.00 | 13.27 | C |
| ATOM | 934 | SD | MET | A | 495 | 11.314 | 2.329 | 14.534 | 1.00 | 18.39 | S |
| ATOM | 935 | CE | MET | A | 495 | 10.911 | 1.751 | 12.891 | 1.00 | 23.19 | C |
| ATOM | 936 | C | MET | A | 495 | 10.876 | −2.438 | 15.991 | 1.00 | 16.42 | C |
| ATOM | 937 | O | MET | A | 495 | 10.586 | −2.968 | 14.935 | 1.00 | 15.60 | O |
| ATOM | 938 | N | ALA | A | 496 | 11.688 | −2.993 | 16.884 | 1.00 | 18.74 | N |
| ATOM | 939 | CA | ALA | A | 496 | 12.290 | −4.310 | 16.687 | 1.00 | 21.16 | C |
| ATOM | 940 | CB | ALA | A | 496 | 13.319 | −4.623 | 17.755 | 1.00 | 20.54 | C |
| ATOM | 941 | C | ALA | A | 496 | 11.219 | −5.344 | 16.778 | 1.00 | 23.00 | C |
| ATOM | 942 | O | ALA | A | 496 | 11.094 | −6.267 | 15.922 | 1.00 | 22.03 | O |
| ATOM | 943 | N | THR | A | 497 | 10.445 | −5.221 | 17.836 | 1.00 | 24.64 | N |
| ATOM | 944 | CA | THR | A | 497 | 9.436 | −6.220 | 18.009 | 1.00 | 27.98 | C |
| ATOM | 945 | CB | THR | A | 497 | 8.657 | −5.942 | 19.223 | 1.00 | 27.33 | C |
| ATOM | 946 | OG1 | THR | A | 497 | 7.876 | −4.776 | 18.968 | 1.00 | 29.40 | O |
| ATOM | 947 | CG2 | THR | A | 497 | 9.609 | −5.593 | 20.366 | 1.00 | 25.14 | C |
| ATOM | 948 | C | THR | A | 497 | 8.512 | −6.334 | 16.784 | 1.00 | 30.44 | C |
| ATOM | 949 | O | THR | A | 497 | 8.060 | −7.443 | 16.457 | 1.00 | 30.05 | O |
| ATOM | 950 | N | TYR | A | 498 | 8.256 | −5.210 | 16.104 | 1.00 | 33.44 | N |
| ATOM | 951 | CA | TYR | A | 498 | 7.368 | −5.204 | 14.927 | 1.00 | 36.84 | C |
| ATOM | 952 | CB | TYR | A | 498 | 6.363 | −4.042 | 14.984 | 1.00 | 36.22 | C |
| ATOM | 953 | CG | TYR | A | 498 | 5.695 | −3.904 | 16.329 | 1.00 | 37.40 | C |
| ATOM | 954 | CD1 | TYR | A | 498 | 4.755 | −4.822 | 16.756 | 1.00 | 39.74 | C |
| ATOM | 955 | CE1 | TYR | A | 498 | 4.162 | −4.693 | 18.018 | 1.00 | 41.01 | C |
| ATOM | 956 | CZ | TYR | A | 498 | 4.520 | −3.631 | 18.828 | 1.00 | 38.06 | C |
| ATOM | 957 | OH | TYR | A | 498 | 3.974 | −3.454 | 20.077 | 1.00 | 38.69 | O |
| ATOM | 958 | CE2 | TYR | A | 498 | 5.443 | −2.731 | 18.404 | 1.00 | 35.90 | C |
| ATOM | 959 | CD2 | TYR | A | 498 | 6.019 | −2.865 | 17.183 | 1.00 | 35.68 | C |
| ATOM | 960 | C | TYR | A | 498 | 8.074 | −5.242 | 13.556 | 1.00 | 39.17 | C |
| ATOM | 961 | O | TYR | A | 498 | 7.561 | −4.693 | 12.563 | 1.00 | 40.64 | O |
| ATOM | 962 | N | SER | A | 499 | 9.233 | −5.887 | 13.478 | 1.00 | 41.51 | N |
| ATOM | 963 | CA | SER | A | 499 | 9.962 | −5.935 | 12.205 | 1.00 | 43.56 | C |
| ATOM | 964 | CB | SER | A | 499 | 11.290 | −6.704 | 12.338 | 1.00 | 43.58 | C |
| ATOM | 965 | OG | SER | A | 499 | 12.305 | −5.924 | 12.951 | 1.00 | 41.96 | O |
| ATOM | 966 | C | SER | A | 499 | 9.098 | −6.527 | 11.084 | 1.00 | 45.17 | C |
| ATOM | 967 | O | SER | A | 499 | 9.103 | −5.993 | 9.973 | 1.00 | 45.09 | O |
| ATOM | 968 | N | ARG | A | 500 | 8.382 | −7.631 | 11.378 | 1.00 | 46.76 | N |
| ATOM | 969 | CA | ARG | A | 500 | 7.476 | −8.292 | 10.409 | 1.00 | 48.01 | C |
| ATOM | 970 | CB | ARG | A | 500 | 7.233 | −9.738 | 10.797 | 1.00 | 47.71 | C |
| ATOM | 971 | CG | ARG | A | 500 | 6.773 | −10.619 | 9.631 | 1.00 | 49.22 | C |
| ATOM | 972 | CD | ARG | A | 500 | 5.473 | −11.420 | 9.915 | 1.00 | 50.50 | C |
| ATOM | 973 | NE | ARG | A | 500 | 4.297 | −10.547 | 10.020 | 1.00 | 52.41 | N |
| ATOM | 974 | CZ | ARG | A | 500 | 3.024 | −10.961 | 10.043 | 1.00 | 52.33 | C |
| ATOM | 975 | NH1 | ARG | A | 500 | 2.726 | −12.270 | 9.976 | 1.00 | 49.40 | N |
| ATOM | 976 | NH2 | ARG | A | 500 | 2.045 | −10.052 | 10.132 | 1.00 | 49.92 | N |
| ATOM | 977 | C | ARG | A | 500 | 6.124 | −7.563 | 10.321 | 1.00 | 48.87 | C |
| ATOM | 978 | O | ARG | A | 500 | 5.050 | −8.171 | 10.489 | 1.00 | 48.55 | O |
| ATOM | 979 | N | SER | A | 501 | 6.211 | −6.248 | 10.092 | 1.00 | 49.79 | N |
| ATOM | 980 | CA | SER | A | 501 | 5.058 | −5.356 | 9.992 | 1.00 | 50.64 | C |
| ATOM | 981 | CB | SER | A | 501 | 5.166 | −4.236 | 11.026 | 1.00 | 50.82 | C |
| ATOM | 982 | OG | SER | A | 501 | 4.077 | −3.328 | 10.893 | 1.00 | 51.88 | O |
| ATOM | 983 | C | SER | A | 501 | 4.924 | −4.738 | 8.587 | 1.00 | 50.90 | C |
| ATOM | 984 | O | SER | A | 501 | 3.844 | −4.752 | 7.983 | 1.00 | 50.85 | O |
| ATOM | 985 | N | SER | A | 508 | 9.297 | 1.112 | −0.283 | 1.00 | 57.14 | N |
| ATOM | 986 | CA | SER | A | 508 | 10.177 | 0.166 | 0.420 | 1.00 | 56.24 | C |
| ATOM | 987 | CB | SER | A | 508 | 10.072 | −1.239 | −0.219 | 1.00 | 56.13 | C |
| ATOM | 988 | OG | SER | A | 508 | 8.726 | −1.704 | −0.216 | 1.00 | 54.22 | O |
| ATOM | 989 | C | SER | A | 508 | 11.636 | 0.689 | 0.478 | 1.00 | 55.73 | C |
| ATOM | 990 | O | SER | A | 508 | 12.609 | −0.084 | 0.559 | 1.00 | 56.00 | O |
| ATOM | 991 | N | GLY | A | 509 | 11.773 | 2.013 | 0.436 | 1.00 | 54.44 | N |
| ATOM | 992 | CA | GLY | A | 509 | 13.075 | 2.644 | 0.499 | 1.00 | 52.95 | C |
| ATOM | 993 | C | GLY | A | 509 | 13.712 | 2.435 | 1.851 | 1.00 | 51.71 | C |
| ATOM | 994 | O | GLY | A | 509 | 14.026 | 3.403 | 2.552 | 1.00 | 51.34 | O |
| ATOM | 995 | N | THR | A | 510 | 13.924 | 1.160 | 2.179 | 1.00 | 50.64 | N |
| ATOM | 996 | CA | THR | A | 510 | 14.498 | 0.702 | 3.455 | 1.00 | 49.39 | C |
| ATOM | 997 | CB | THR | A | 510 | 16.036 | 0.527 | 3.429 | 1.00 | 50.13 | C |
| ATOM | 998 | OG1 | THR | A | 510 | 16.487 | 0.219 | 4.774 | 1.00 | 49.53 | O |
| ATOM | 999 | CG2 | THR | A | 510 | 16.783 | 1.834 | 3.003 | 1.00 | 49.41 | C |
| ATOM | 1000 | C | THR | A | 510 | 14.164 | 1.482 | 4.674 | 1.00 | 48.11 | C |
| ATOM | 1001 | O | THR | A | 510 | 14.292 | 2.708 | 4.691 | 1.00 | 48.23 | O |
| ATOM | 1002 | N | ASP | A | 511 | 13.774 | 0.760 | 5.719 | 1.00 | 46.68 | N |
| ATOM | 1003 | CA | ASP | A | 511 | 13.456 | 1.400 | 6.998 | 1.00 | 44.62 | C |
| ATOM | 1004 | CB | ASP | A | 511 | 12.053 | 1.024 | 7.442 | 1.00 | 45.05 | C |
| ATOM | 1005 | CG | ASP | A | 511 | 11.752 | 1.485 | 8.823 | 1.00 | 44.34 | C |
| ATOM | 1006 | OD1 | ASP | A | 511 | 11.716 | 2.709 | 9.062 | 1.00 | 47.00 | O |
| ATOM | 1007 | OD2 | ASP | A | 511 | 11.556 | 0.693 | 9.743 | 1.00 | 43.18 | O |
| ATOM | 1008 | C | ASP | A | 511 | 14.454 | 1.018 | 8.093 | 1.00 | 42.93 | C |
| ATOM | 1009 | O | ASP | A | 511 | 15.641 | 0.716 | 7.827 | 1.00 | 42.65 | O |
| ATOM | 1010 | N | LEU | A | 512 | 13.953 | 1.009 | 9.322 | 1.00 | 40.19 | N |
| ATOM | 1011 | CA | LEU | A | 512 | 14.792 | 0.764 | 10.492 | 1.00 | 37.10 | C |
| ATOM | 1012 | CB | LEU | A | 512 | 13.935 | 0.548 | 11.720 | 1.00 | 36.88 | C |
| ATOM | 1013 | CG | LEU | A | 512 | 14.573 | 0.486 | 13.093 | 1.00 | 38.65 | C |
| ATOM | 1014 | CD1 | LEU | A | 512 | 15.068 | −0.968 | 13.429 | 1.00 | 39.66 | C |
| ATOM | 1015 | CD2 | LEU | A | 512 | 15.676 | 1.573 | 13.278 | 1.00 | 39.27 | C |
| ATOM | 1016 | C | LEU | A | 512 | 15.743 | −0.390 | 10.273 | 1.00 | 34.87 | C |
| ATOM | 1017 | O | LEU | A | 512 | 15.760 | −1.031 | 9.220 | 1.00 | 34.35 | O |
| ATOM | 1018 | N | SER | A | 513 | 16.542 | −0.607 | 11.299 | 1.00 | 32.30 | N |
| ATOM | 1019 | CA | SER | A | 513 | 17.599 | −1.586 | 11.342 | 1.00 | 29.77 | C |
| ATOM | 1020 | CB | SER | A | 513 | 18.113 | −1.943 | 9.931 | 1.00 | 30.70 | C |
| ATOM | 1021 | OG | SER | A | 513 | 17.245 | −2.859 | 9.250 | 1.00 | 30.48 | O |
| ATOM | 1022 | C | SER | A | 513 | 18.597 | −0.743 | 12.154 | 1.00 | 26.48 | C |
| ATOM | 1023 | O | SER | A | 513 | 19.360 | 0.031 | 11.624 | 1.00 | 25.26 | O |
| ATOM | 1024 | N | PHE | A | 514 | 18.537 | −0.922 | 13.460 | 1.00 | 23.05 | N |
| ATOM | 1025 | CA | PHE | A | 514 | 19.332 | −0.177 | 14.402 | 1.00 | 19.60 | C |
| ATOM | 1026 | CB | PHE | A | 514 | 20.495 | −0.914 | 14.932 | 1.00 | 17.88 | C |
| ATOM | 1027 | CG | PHE | A | 514 | 21.410 | −0.038 | 15.659 | 1.00 | 13.56 | C |
| ATOM | 1028 | CD1 | PHE | A | 514 | 22.774 | −0.135 | 15.466 | 1.00 | 7.78 | C |
| ATOM | 1029 | CE1 | PHE | A | 514 | 23.601 | 0.675 | 16.127 | 1.00 | 7.61 | C |
| ATOM | 1030 | CZ | PHE | A | 514 | 23.077 | 1.632 | 17.011 | 1.00 | 10.61 | C |
| ATOM | 1031 | CE2 | PHE | A | 514 | 21.715 | 1.743 | 17.219 | 1.00 | 5.65 | C |
| ATOM | 1032 | CD2 | PHE | A | 514 | 20.893 | 0.919 | 16.539 | 1.00 | 7.37 | C |
| ATOM | 1033 | C | PHE | A | 514 | 19.831 | 1.160 | 13.883 | 1.00 | 19.98 | C |
| ATOM | 1034 | O | PHE | A | 514 | 19.175 | 2.200 | 14.198 | 1.00 | 21.97 | O |
| ATOM | 1035 | N | PRO | A | 515 | 20.917 | 1.189 | 13.071 | 1.00 | 16.87 | N |
| ATOM | 1036 | CA | PRO | A | 515 | 21.498 | 2.461 | 12.678 | 1.00 | 13.02 | C |
| ATOM | 1037 | CB | PRO | A | 515 | 22.506 | 2.056 | 11.613 | 1.00 | 13.72 | C |
| ATOM | 1038 | CG | PRO | A | 515 | 22.040 | 0.796 | 11.161 | 1.00 | 13.79 | C |
| ATOM | 1039 | CD | PRO | A | 515 | 21.587 | 0.062 | 12.404 | 1.00 | 16.21 | C |
| ATOM | 1040 | C | PRO | A | 515 | 20.449 | 3.495 | 12.185 | 1.00 | 11.10 | C |
| ATOM | 1041 | O | PRO | A | 515 | 20.673 | 4.693 | 12.490 | 1.00 | 11.58 | O |
| ATOM | 1042 | N | TRP | A | 516 | 19.356 | 3.097 | 11.514 | 1.00 | 8.08 | N |
| ATOM | 1043 | CA | TRP | A | 516 | 18.374 | 4.084 | 10.997 | 1.00 | 6.57 | C |
| ATOM | 1044 | CB | TRP | A | 516 | 17.119 | 3.393 | 10.446 | 1.00 | 6.20 | C |
| ATOM | 1045 | CG | TRP | A | 516 | 16.106 | 4.310 | 9.797 | 1.00 | 6.53 | C |
| ATOM | 1046 | CD1 | TRP | A | 516 | 16.196 | 4.932 | 8.538 | 1.00 | 6.10 | C |
| ATOM | 1047 | NE1 | TRP | A | 516 | 15.058 | 5.663 | 8.286 | 1.00 | 2.00 | N |
| ATOM | 1048 | CE2 | TRP | A | 516 | 14.229 | 5.565 | 9.354 | 1.00 | 2.00 | C |
| ATOM | 1049 | CD2 | TRP | A | 516 | 14.860 | 4.732 | 10.333 | 1.00 | 3.32 | C |
| ATOM | 1050 | CE3 | TRP | A | 516 | 14.213 | 4.528 | 11.555 | 1.00 | 2.00 | C |
| ATOM | 1051 | CZ3 | TRP | A | 516 | 12.976 | 5.088 | 11.741 | 1.00 | 2.00 | C |
| ATOM | 1052 | CH2 | TRP | A | 516 | 12.376 | 5.861 | 10.761 | 1.00 | 2.00 | C |
| ATOM | 1053 | CZ2 | TRP | A | 516 | 13.015 | 6.160 | 9.569 | 1.00 | 3.24 | C |
| ATOM | 1054 | C | TRP | A | 516 | 17.952 | 5.169 | 12.003 | 1.00 | 4.43 | C |
| ATOM | 1055 | O | TRP | A | 516 | 17.926 | 6.353 | 11.702 | 1.00 | 3.37 | O |
| ATOM | 1056 | N | ILE | A | 517 | 17.666 | 4.740 | 13.200 | 1.00 | 3.57 | N |
| ATOM | 1057 | CA | ILE | A | 517 | 17.185 | 5.585 | 14.267 | 1.00 | 4.13 | C |
| ATOM | 1058 | CB | ILE | A | 517 | 16.858 | 4.736 | 15.545 | 1.00 | 3.53 | C |
| ATOM | 1059 | CG1 | ILE | A | 517 | 15.951 | 5.447 | 16.492 | 1.00 | 3.79 | C |
| ATOM | 1060 | CD1 | ILE | A | 517 | 14.834 | 6.161 | 15.859 | 1.00 | 2.00 | C |
| ATOM | 1061 | CG2 | ILE | A | 517 | 18.075 | 4.328 | 16.251 | 1.00 | 2.00 | C |
| ATOM | 1062 | C | ILE | A | 517 | 18.212 | 6.609 | 14.585 | 1.00 | 6.38 | C |
| ATOM | 1063 | O | ILE | A | 517 | 17.848 | 7.674 | 15.015 | 1.00 | 8.32 | O |
| ATOM | 1064 | N | LEU | A | 518 | 19.497 | 6.317 | 14.379 | 1.00 | 7.30 | N |
| ATOM | 1065 | CA | LEU | A | 518 | 20.514 | 7.275 | 14.739 | 1.00 | 6.90 | C |
| ATOM | 1066 | CB | LEU | A | 518 | 21.899 | 6.677 | 14.639 | 1.00 | 7.32 | C |
| ATOM | 1067 | CG | LEU | A | 518 | 22.196 | 5.522 | 15.627 | 1.00 | 8.35 | C |
| ATOM | 1068 | CD1 | LEU | A | 518 | 23.684 | 5.126 | 15.647 | 1.00 | 2.50 | C |
| ATOM | 1069 | CD2 | LEU | A | 518 | 21.682 | 5.833 | 16.977 | 1.00 | 5.08 | C |
| ATOM | 1070 | C | LEU | A | 518 | 20.392 | 8.434 | 13.817 | 1.00 | 7.74 | C |
| ATOM | 1071 | O | LEU | A | 518 | 20.237 | 9.526 | 14.269 | 1.00 | 7.58 | O |
| ATOM | 1072 | N | ASN | A | 519 | 20.409 | 8.196 | 12.511 | 1.00 | 9.38 | N |
| ATOM | 1073 | CA | ASN | A | 519 | 20.366 | 9.250 | 11.525 | 1.00 | 11.13 | C |
| ATOM | 1074 | CB | ASN | A | 519 | 20.446 | 8.593 | 10.162 | 1.00 | 13.71 | C |
| ATOM | 1075 | CG | ASN | A | 519 | 21.098 | 9.498 | 9.094 | 1.00 | 23.00 | C |
| ATOM | 1076 | OD1 | ASN | A | 519 | 22.350 | 9.727 | 9.100 | 1.00 | 30.67 | O |
| ATOM | 1077 | ND2 | ASN | A | 519 | 20.268 | 10.018 | 8.169 | 1.00 | 22.67 | N |
| ATOM | 1078 | C | ASN | A | 519 | 19.046 | 10.037 | 11.701 | 1.00 | 10.46 | C |
| ATOM | 1079 | O | ASN | A | 519 | 19.004 | 11.240 | 11.786 | 1.00 | 11.31 | O |
| ATOM | 1080 | N | VAL | A | 520 | 17.949 | 9.344 | 11.879 | 1.00 | 9.13 | N |
| ATOM | 1081 | CA | VAL | A | 520 | 16.709 | 10.025 | 12.095 | 1.00 | 8.53 | C |
| ATOM | 1082 | CB | VAL | A | 520 | 15.620 | 8.949 | 12.461 | 1.00 | 10.43 | C |
| ATOM | 1083 | CG1 | VAL | A | 520 | 14.333 | 9.580 | 12.952 | 1.00 | 10.55 | C |
| ATOM | 1084 | CG2 | VAL | A | 520 | 15.317 | 8.122 | 11.201 | 1.00 | 13.73 | C |
| ATOM | 1085 | C | VAL | A | 520 | 16.771 | 11.115 | 13.191 | 1.00 | 6.90 | C |
| ATOM | 1086 | O | VAL | A | 520 | 16.164 | 12.190 | 13.044 | 1.00 | 3.70 | O |
| ATOM | 1087 | N | LEU | A | 521 | 17.443 | 10.792 | 14.314 | 1.00 | 5.11 | N |
| ATOM | 1088 | CA | LEU | A | 521 | 17.522 | 11.711 | 15.441 | 1.00 | 4.30 | C |
| ATOM | 1089 | CB | LEU | A | 521 | 17.357 | 10.946 | 16.736 | 1.00 | 4.31 | C |
| ATOM | 1090 | CG | LEU | A | 521 | 16.071 | 10.118 | 16.854 | 1.00 | 7.22 | C |
| ATOM | 1091 | CD1 | LEU | A | 521 | 15.983 | 9.289 | 18.178 | 1.00 | 7.33 | C |
| ATOM | 1092 | CD2 | LEU | A | 521 | 14.901 | 11.002 | 16.779 | 1.00 | 7.22 | C |
| ATOM | 1093 | C | LEU | A | 521 | 18.823 | 12.577 | 15.474 | 1.00 | 4.02 | C |
| ATOM | 1094 | O | LEU | A | 521 | 19.011 | 13.401 | 16.394 | 1.00 | 2.26 | O |
| ATOM | 1095 | N | ASN | A | 522 | 19.688 | 12.407 | 14.458 | 1.00 | 2.73 | N |
| ATOM | 1096 | CA | ASN | A | 522 | 20.955 | 13.075 | 14.488 | 1.00 | 2.77 | C |
| ATOM | 1097 | CB | ASN | A | 522 | 20.752 | 14.599 | 14.324 | 1.00 | 3.11 | C |
| ATOM | 1098 | CG | ASN | A | 522 | 21.989 | 15.287 | 13.874 | 1.00 | 2.00 | C |
| ATOM | 1099 | OD1 | ASN | A | 522 | 22.890 | 14.661 | 13.307 | 1.00 | 5.97 | O |
| ATOM | 1100 | ND2 | ASN | A | 522 | 22.010 | 16.571 | 13.998 | 1.00 | 2.15 | N |
| ATOM | 1101 | C | ASN | A | 522 | 21.802 | 12.771 | 15.773 | 1.00 | 2.00 | C |
| ATOM | 1102 | O | ASN | A | 522 | 22.511 | 13.624 | 16.287 | 1.00 | 3.48 | O |
| ATOM | 1103 | N | LEU | A | 523 | 21.783 | 11.539 | 16.200 | 1.00 | 2.26 | N |
| ATOM | 1104 | CA | LEU | A | 523 | 22.438 | 11.082 | 17.427 | 1.00 | 3.51 | C |
| ATOM | 1105 | CB | LEU | A | 523 | 21.428 | 10.220 | 18.177 | 1.00 | 2.69 | C |
| ATOM | 1106 | CG | LEU | A | 523 | 21.532 | 9.986 | 19.629 | 1.00 | 3.52 | C |
| ATOM | 1107 | CD1 | LEU | A | 523 | 20.849 | 8.528 | 19.873 | 1.00 | 3.71 | C |
| ATOM | 1108 | CD2 | LEU | A | 523 | 22.971 | 9.957 | 19.828 | 1.00 | 12.53 | C |
| ATOM | 1109 | C | LEU | A | 523 | 23.614 | 10.191 | 17.090 | 1.00 | 3.88 | C |
| ATOM | 1110 | O | LEU | A | 523 | 23.485 | 9.219 | 16.297 | 1.00 | 4.01 | O |
| ATOM | 1111 | N | LYS | A | 524 | 24.746 | 10.461 | 17.716 | 1.00 | 2.74 | N |
| ATOM | 1112 | CA | LYS | A | 524 | 25.903 | 9.601 | 17.474 | 1.00 | 2.84 | C |
| ATOM | 1113 | CB | LYS | A | 524 | 27.149 | 10.334 | 17.836 | 1.00 | 2.00 | C |
| ATOM | 1114 | CG | LYS | A | 524 | 27.149 | 11.724 | 17.147 | 1.00 | 2.00 | C |
| ATOM | 1115 | CD | LYS | A | 524 | 27.457 | 11.584 | 15.679 | 1.00 | 2.78 | C |
| ATOM | 1116 | CE | LYS | A | 524 | 27.418 | 12.949 | 14.980 | 1.00 | 7.61 | C |
| ATOM | 1117 | NZ | LYS | A | 524 | 27.359 | 12.847 | 13.479 | 1.00 | 11.33 | N |
| ATOM | 1118 | C | LYS | A | 524 | 25.885 | 8.202 | 18.109 | 1.00 | 3.34 | C |
| ATOM | 1119 | O | LYS | A | 524 | 25.280 | 7.964 | 19.167 | 1.00 | 3.82 | O |
| ATOM | 1120 | N | ALA | A | 525 | 26.506 | 7.261 | 17.441 | 1.00 | 3.11 | N |
| ATOM | 1121 | CA | ALA | A | 525 | 26.598 | 5.910 | 17.958 | 1.00 | 3.88 | C |
| ATOM | 1122 | CB | ALA | A | 525 | 27.316 | 5.015 | 16.961 | 1.00 | 2.69 | C |
| ATOM | 1123 | C | ALA | A | 525 | 27.273 | 5.816 | 19.369 | 1.00 | 5.48 | C |
| ATOM | 1124 | O | ALA | A | 525 | 26.858 | 5.010 | 20.264 | 1.00 | 5.29 | O |
| ATOM | 1125 | N | PHE | A | 526 | 28.325 | 6.610 | 19.571 | 1.00 | 5.00 | N |
| ATOM | 1126 | CA | PHE | A | 526 | 29.027 | 6.484 | 20.806 | 1.00 | 5.18 | C |
| ATOM | 1127 | CB | PHE | A | 526 | 30.353 | 7.286 | 20.777 | 1.00 | 5.32 | C |
| ATOM | 1128 | CG | PHE | A | 526 | 31.143 | 7.206 | 22.059 | 1.00 | 6.17 | C |
| ATOM | 1129 | CD1 | PHE | A | 526 | 31.814 | 6.036 | 22.377 | 1.00 | 2.97 | C |
| ATOM | 1130 | CE1 | PHE | A | 526 | 32.505 | 5.936 | 23.581 | 1.00 | 3.73 | C |
| ATOM | 1131 | CZ | PHE | A | 526 | 32.494 | 7.010 | 24.568 | 1.00 | 2.63 | C |
| ATOM | 1132 | CE2 | PHE | A | 526 | 31.822 | 8.187 | 24.280 | 1.00 | 3.05 | C |
| ATOM | 1133 | CD2 | PHE | A | 526 | 31.111 | 8.274 | 23.014 | 1.00 | 5.63 | C |
| ATOM | 1134 | C | PHE | A | 526 | 28.109 | 6.884 | 21.983 | 1.00 | 5.59 | C |
| ATOM | 1135 | O | PHE | A | 526 | 28.240 | 6.292 | 23.098 | 1.00 | 4.24 | O |
| ATOM | 1136 | N | ASP | A | 527 | 27.251 | 7.911 | 21.773 | 1.00 | 4.53 | N |
| ATOM | 1137 | CA | ASP | A | 527 | 26.313 | 8.356 | 22.846 | 1.00 | 4.18 | C |
| ATOM | 1138 | CB | ASP | A | 527 | 25.701 | 9.662 | 22.496 | 1.00 | 3.20 | C |
| ATOM | 1139 | CG | ASP | A | 527 | 26.718 | 10.775 | 22.359 | 1.00 | 5.73 | C |
| ATOM | 1140 | OD1 | ASP | A | 527 | 27.665 | 10.941 | 23.205 | 1.00 | 6.84 | O |
| ATOM | 1141 | OD2 | ASP | A | 527 | 26.598 | 11.619 | 21.454 | 1.00 | 9.35 | O |
| ATOM | 1142 | C | ASP | A | 527 | 25.187 | 7.375 | 23.150 | 1.00 | 4.67 | C |
| ATOM | 1143 | O | ASP | A | 527 | 24.739 | 7.280 | 24.286 | 1.00 | 6.22 | O |
| ATOM | 1144 | N | PHE | A | 528 | 24.737 | 6.649 | 22.136 | 1.00 | 4.38 | N |
| ATOM | 1145 | CA | PHE | A | 528 | 23.658 | 5.687 | 22.227 | 1.00 | 6.14 | C |
| ATOM | 1146 | CB | PHE | A | 528 | 23.358 | 5.130 | 20.803 | 1.00 | 7.25 | C |
| ATOM | 1147 | CG | PHE | A | 528 | 22.132 | 4.250 | 20.713 | 1.00 | 8.05 | C |
| ATOM | 1148 | CD1 | PHE | A | 528 | 20.924 | 4.782 | 20.311 | 1.00 | 6.37 | C |
| ATOM | 1149 | CE1 | PHE | A | 528 | 19.799 | 3.975 | 20.164 | 1.00 | 7.19 | C |
| ATOM | 1150 | CZ | PHE | A | 528 | 19.874 | 2.577 | 20.441 | 1.00 | 5.82 | C |
| ATOM | 1151 | CE2 | PHE | A | 528 | 21.068 | 2.038 | 20.784 | 1.00 | 5.09 | C |
| ATOM | 1152 | CD2 | PHE | A | 528 | 22.218 | 2.866 | 20.917 | 1.00 | 7.19 | C |
| ATOM | 1153 | C | PHE | A | 528 | 24.105 | 4.578 | 23.126 | 1.00 | 6.06 | C |
| ATOM | 1154 | O | PHE | A | 528 | 23.425 | 4.228 | 24.084 | 1.00 | 6.25 | O |
| ATOM | 1155 | N | TYR | A | 529 | 25.289 | 4.066 | 22.816 | 1.00 | 6.43 | N |
| ATOM | 1156 | CA | TYR | A | 529 | 25.938 | 3.007 | 23.562 | 1.00 | 6.94 | C |
| ATOM | 1157 | CB | TYR | A | 529 | 27.387 | 2.762 | 23.011 | 1.00 | 7.10 | C |
| ATOM | 1158 | CG | TYR | A | 529 | 28.385 | 2.752 | 24.090 | 1.00 | 7.19 | C |
| ATOM | 1159 | CD1 | TYR | A | 529 | 28.491 | 1.668 | 24.986 | 1.00 | 11.52 | C |
| ATOM | 1160 | CE1 | TYR | A | 529 | 29.389 | 1.711 | 26.088 | 1.00 | 10.35 | C |
| ATOM | 1161 | CZ | TYR | A | 529 | 30.151 | 2.858 | 26.287 | 1.00 | 13.78 | C |
| ATOM | 1162 | OH | TYR | A | 529 | 31.044 | 2.977 | 27.345 | 1.00 | 19.36 | O |
| ATOM | 1163 | CE2 | TYR | A | 529 | 30.068 | 3.915 | 25.405 | 1.00 | 11.15 | C |
| ATOM | 1164 | CD2 | TYR | A | 529 | 29.151 | 3.849 | 24.312 | 1.00 | 8.73 | C |
| ATOM | 1165 | C | TYR | A | 529 | 25.935 | 3.312 | 25.069 | 1.00 | 7.03 | C |
| ATOM | 1166 | O | TYR | A | 529 | 25.690 | 2.457 | 25.848 | 1.00 | 6.96 | O |
| ATOM | 1167 | N | LYS | A | 530 | 26.238 | 4.532 | 25.478 | 1.00 | 7.01 | N |
| ATOM | 1168 | CA | LYS | A | 530 | 26.239 | 4.848 | 26.893 | 1.00 | 7.93 | C |
| ATOM | 1169 | CB | LYS | A | 530 | 26.554 | 6.323 | 27.075 | 1.00 | 8.59 | C |
| ATOM | 1170 | CG | LYS | A | 530 | 27.919 | 6.736 | 26.434 | 1.00 | 12.70 | C |
| ATOM | 1171 | CD | LYS | A | 530 | 28.593 | 7.898 | 27.168 | 1.00 | 15.96 | C |
| ATOM | 1172 | CE | LYS | A | 530 | 27.983 | 9.237 | 26.949 | 1.00 | 16.66 | C |
| ATOM | 1173 | NZ | LYS | A | 530 | 28.524 | 10.243 | 27.977 | 1.00 | 21.28 | N |
| ATOM | 1174 | C | LYS | A | 530 | 24.930 | 4.531 | 27.635 | 1.00 | 7.04 | C |
| ATOM | 1175 | O | LYS | A | 530 | 24.894 | 4.454 | 28.847 | 1.00 | 6.48 | O |
| ATOM | 1176 | N | VAL | A | 531 | 23.871 | 4.272 | 26.892 | 1.00 | 6.56 | N |
| ATOM | 1177 | CA | VAL | A | 531 | 22.577 | 4.052 | 27.500 | 1.00 | 6.44 | C |
| ATOM | 1178 | CB | VAL | A | 531 | 21.516 | 4.971 | 26.840 | 1.00 | 7.07 | C |
| ATOM | 1179 | CG1 | VAL | A | 531 | 20.343 | 4.227 | 26.381 | 1.00 | 6.11 | C |
| ATOM | 1180 | CG2 | VAL | A | 531 | 21.123 | 6.035 | 27.787 | 1.00 | 5.82 | C |
| ATOM | 1181 | C | VAL | A | 531 | 22.123 | 2.598 | 27.510 | 1.00 | 6.24 | C |
| ATOM | 1182 | O | VAL | A | 531 | 21.231 | 2.236 | 28.299 | 1.00 | 5.96 | O |
| ATOM | 1183 | N | ILE | A | 532 | 22.781 | 1.762 | 26.700 | 1.00 | 5.58 | N |
| ATOM | 1184 | CA | ILE | A | 532 | 22.429 | 0.353 | 26.549 | 1.00 | 5.19 | C |
| ATOM | 1185 | CB | ILE | A | 532 | 23.238 | −0.250 | 25.456 | 1.00 | 4.47 | C |
| ATOM | 1186 | CG1 | ILE | A | 532 | 22.889 | 0.411 | 24.083 | 1.00 | 4.56 | C |
| ATOM | 1187 | CD1 | ILE | A | 532 | 23.696 | −0.133 | 22.877 | 1.00 | 2.00 | C |
| ATOM | 1188 | CG2 | ILE | A | 532 | 22.953 | −1.674 | 25.387 | 1.00 | 4.32 | C |
| ATOM | 1189 | C | ILE | A | 532 | 22.488 | −0.511 | 27.837 | 1.00 | 6.67 | C |
| ATOM | 1190 | O | ILE | A | 532 | 21.454 | −1.055 | 28.285 | 1.00 | 7.52 | O |
| ATOM | 1191 | N | GLU | A | 533 | 23.629 | −0.609 | 28.507 | 1.00 | 6.39 | N |
| ATOM | 1192 | CA | GLU | A | 533 | 23.677 | −1.499 | 29.691 | 1.00 | 6.52 | C |
| ATOM | 1193 | CB | GLU | A | 533 | 25.067 | −1.550 | 30.317 | 1.00 | 6.68 | C |
| ATOM | 1194 | CG | GLU | A | 533 | 25.272 | −2.671 | 31.320 | 1.00 | 11.17 | C |
| ATOM | 1195 | CD | GLU | A | 533 | 26.738 | −2.845 | 31.725 | 1.00 | 16.80 | C |
| ATOM | 1196 | OE1 | GLU | A | 533 | 27.562 | −2.996 | 30.800 | 1.00 | 19.44 | O |
| ATOM | 1197 | OE2 | GLU | A | 533 | 27.094 | −2.828 | 32.935 | 1.00 | 16.76 | O |
| ATOM | 1198 | C | GLU | A | 533 | 22.683 | −1.098 | 30.776 | 1.00 | 6.18 | C |
| ATOM | 1199 | O | GLU | A | 533 | 22.077 | −1.966 | 31.395 | 1.00 | 6.74 | O |
| ATOM | 1200 | N | SER | A | 534 | 22.497 | 0.211 | 30.998 | 1.00 | 5.64 | N |
| ATOM | 1201 | CA | SER | A | 534 | 21.585 | 0.679 | 32.030 | 1.00 | 4.84 | C |
| ATOM | 1202 | CB | SER | A | 534 | 21.769 | 2.187 | 32.279 | 1.00 | 4.45 | C |
| ATOM | 1203 | OG | SER | A | 534 | 23.070 | 2.400 | 32.781 | 1.00 | 5.10 | O |
| ATOM | 1204 | C | SER | A | 534 | 20.139 | 0.394 | 31.664 | 1.00 | 5.13 | C |
| ATOM | 1205 | O | SER | A | 534 | 19.329 | 0.055 | 32.511 | 1.00 | 4.57 | O |
| ATOM | 1206 | N | PHE | A | 535 | 19.828 | 0.544 | 30.377 | 1.00 | 5.26 | N |
| ATOM | 1207 | CA | PHE | A | 535 | 18.497 | 0.293 | 29.885 | 1.00 | 4.73 | C |
| ATOM | 1208 | CB | PHE | A | 535 | 18.453 | 0.649 | 28.394 | 1.00 | 4.00 | C |
| ATOM | 1209 | CG | PHE | A | 535 | 17.077 | 0.596 | 27.794 | 1.00 | 5.70 | C |
| ATOM | 1210 | CD1 | PHE | A | 535 | 16.418 | −0.623 | 27.601 | 1.00 | 6.27 | C |
| ATOM | 1211 | CE1 | PHE | A | 535 | 15.157 | −0.682 | 27.055 | 1.00 | 4.58 | C |
| ATOM | 1212 | CZ | PHE | A | 535 | 14.532 | 0.449 | 26.709 | 1.00 | 4.63 | C |
| ATOM | 1213 | CE2 | PHE | A | 535 | 15.156 | 1.683 | 26.907 | 1.00 | 7.49 | C |
| ATOM | 1214 | CD2 | PHE | A | 535 | 16.430 | 1.759 | 27.424 | 1.00 | 6.81 | C |
| ATOM | 1215 | C | PHE | A | 535 | 18.138 | −1.186 | 30.165 | 1.00 | 4.51 | C |
| ATOM | 1216 | O | PHE | A | 535 | 17.052 | −1.477 | 30.621 | 1.00 | 3.71 | O |
| ATOM | 1217 | N | ILE | A | 536 | 19.077 | −2.093 | 29.898 | 1.00 | 4.10 | N |
| ATOM | 1218 | CA | ILE | A | 536 | 18.874 | −3.495 | 30.168 | 1.00 | 4.80 | C |
| ATOM | 1219 | CB | ILE | A | 536 | 20.025 | −4.347 | 29.670 | 1.00 | 4.93 | C |
| ATOM | 1220 | CG1 | ILE | A | 536 | 20.014 | −4.414 | 28.161 | 1.00 | 2.00 | C |
| ATOM | 1221 | CD1 | ILE | A | 536 | 21.367 | −4.726 | 27.637 | 1.00 | 2.00 | C |
| ATOM | 1222 | CG2 | ILE | A | 536 | 19.894 | −5.757 | 30.160 | 1.00 | 2.46 | C |
| ATOM | 1223 | C | ILE | A | 536 | 18.664 | −3.836 | 31.638 | 1.00 | 6.22 | C |
| ATOM | 1224 | O | ILE | A | 536 | 17.956 | −4.791 | 31.910 | 1.00 | 7.20 | O |
| ATOM | 1225 | N | LYS | A | 537 | 19.245 | −3.082 | 32.571 | 1.00 | 7.13 | N |
| ATOM | 1226 | CA | LYS | A | 537 | 19.039 | −3.360 | 34.010 | 1.00 | 7.81 | C |
| ATOM | 1227 | CB | LYS | A | 537 | 20.210 | −2.954 | 34.920 | 1.00 | 8.38 | C |
| ATOM | 1228 | CG | LYS | A | 537 | 21.589 | −3.503 | 34.638 | 1.00 | 11.82 | C |
| ATOM | 1229 | CD | LYS | A | 537 | 22.582 | −2.777 | 35.588 | 1.00 | 20.54 | C |
| ATOM | 1230 | CE | LYS | A | 537 | 24.072 | −2.945 | 35.157 | 1.00 | 26.43 | C |
| ATOM | 1231 | NZ | LYS | A | 537 | 25.029 | −2.886 | 36.319 | 1.00 | 26.23 | N |
| ATOM | 1232 | C | LYS | A | 537 | 17.853 | −2.665 | 34.540 | 1.00 | 6.91 | C |
| ATOM | 1233 | O | LYS | A | 537 | 17.360 | −3.015 | 35.570 | 1.00 | 7.82 | O |
| ATOM | 1234 | N | ALA | A | 538 | 17.402 | −1.626 | 33.873 | 1.00 | 6.92 | N |
| ATOM | 1235 | CA | ALA | A | 538 | 16.250 | −0.901 | 34.370 | 1.00 | 5.49 | C |
| ATOM | 1236 | CB | ALA | A | 538 | 16.297 | 0.503 | 33.897 | 1.00 | 4.12 | C |
| ATOM | 1237 | C | ALA | A | 538 | 14.926 | −1.537 | 33.966 | 1.00 | 6.38 | C |
| ATOM | 1238 | O | ALA | A | 538 | 13.957 | −1.305 | 34.626 | 1.00 | 6.33 | O |
| ATOM | 1239 | N | GLU | A | 539 | 14.879 | −2.296 | 32.870 | 1.00 | 7.70 | N |
| ATOM | 1240 | CA | GLU | A | 539 | 13.655 | −2.885 | 32.354 | 1.00 | 8.69 | C |
| ATOM | 1241 | CB | GLU | A | 539 | 13.550 | −2.681 | 30.821 | 1.00 | 10.15 | C |
| ATOM | 1242 | CG | GLU | A | 539 | 12.180 | −3.034 | 30.261 | 1.00 | 10.13 | C |
| ATOM | 1243 | CD | GLU | A | 539 | 11.089 | −2.643 | 31.275 | 1.00 | 14.92 | C |
| ATOM | 1244 | OE1 | GLU | A | 539 | 10.984 | −1.404 | 31.629 | 1.00 | 14.92 | O |
| ATOM | 1245 | OE2 | GLU | A | 539 | 10.377 | −3.560 | 31.747 | 1.00 | 11.46 | O |
| ATOM | 1246 | C | GLU | A | 539 | 13.662 | −4.373 | 32.559 | 1.00 | 9.87 | C |
| ATOM | 1247 | O | GLU | A | 539 | 14.241 | −5.145 | 31.725 | 1.00 | 11.40 | O |
| ATOM | 1248 | N | GLY | A | 540 | 12.982 | −4.820 | 33.599 | 1.00 | 8.74 | N |
| ATOM | 1249 | CA | GLY | A | 540 | 12.926 | −6.240 | 33.832 | 1.00 | 8.45 | C |
| ATOM | 1250 | C | GLY | A | 540 | 12.031 | −7.044 | 32.889 | 1.00 | 8.50 | C |
| ATOM | 1251 | O | GLY | A | 540 | 12.013 | −8.249 | 32.981 | 1.00 | 8.50 | O |
| ATOM | 1252 | N | ASN | A | 541 | 11.296 | −6.412 | 31.987 | 1.00 | 8.51 | N |
| ATOM | 1253 | CA | ASN | A | 541 | 10.420 | −7.177 | 31.125 | 1.00 | 9.67 | C |
| ATOM | 1254 | CB | ASN | A | 541 | 9.033 | −6.558 | 31.090 | 1.00 | 9.96 | C |
| ATOM | 1255 | CG | ASN | A | 541 | 8.315 | −6.684 | 32.421 | 1.00 | 12.87 | C |
| ATOM | 1256 | OD1 | ASN | A | 541 | 7.510 | −7.576 | 32.607 | 1.00 | 15.20 | O |
| ATOM | 1257 | ND2 | ASN | A | 541 | 8.603 | −5.782 | 33.346 | 1.00 | 11.13 | N |
| ATOM | 1258 | C | ASN | A | 541 | 10.911 | −7.378 | 29.706 | 1.00 | 9.56 | C |
| ATOM | 1259 | O | ASN | A | 541 | 10.126 | −7.663 | 28.823 | 1.00 | 10.01 | O |
| ATOM | 1260 | N | LEU | A | 542 | 12.207 | −7.232 | 29.472 | 1.00 | 9.76 | N |
| ATOM | 1261 | CA | LEU | A | 542 | 12.717 | −7.373 | 28.095 | 1.00 | 9.10 | C |
| ATOM | 1262 | CB | LEU | A | 542 | 14.075 | −6.684 | 27.941 | 1.00 | 8.09 | C |
| ATOM | 1263 | CG | LEU | A | 542 | 14.095 | −5.179 | 27.973 | 1.00 | 8.97 | C |
| ATOM | 1264 | CD1 | LEU | A | 542 | 15.492 | −4.668 | 28.053 | 1.00 | 9.22 | C |
| ATOM | 1265 | CD2 | LEU | A | 542 | 13.403 | −4.622 | 26.759 | 1.00 | 9.80 | C |
| ATOM | 1266 | C | LEU | A | 542 | 12.852 | −8.848 | 27.746 | 1.00 | 8.30 | C |
| ATOM | 1267 | O | LEU | A | 542 | 13.338 | −9.615 | 28.540 | 1.00 | 8.47 | O |
| ATOM | 1268 | N | THR | A | 543 | 12.463 | −9.247 | 26.547 | 1.00 | 8.14 | N |
| ATOM | 1269 | CA | THR | A | 543 | 12.631 | −10.650 | 26.147 | 1.00 | 7.80 | C |
| ATOM | 1270 | CB | THR | A | 543 | 11.922 | −10.978 | 24.831 | 1.00 | 6.70 | C |
| ATOM | 1271 | OG1 | THR | A | 543 | 12.469 | −10.196 | 23.781 | 1.00 | 4.89 | O |
| ATOM | 1272 | CG2 | THR | A | 543 | 10.487 | −10.605 | 24.853 | 1.00 | 7.19 | C |
| ATOM | 1273 | C | THR | A | 543 | 14.089 | −11.004 | 25.939 | 1.00 | 9.04 | C |
| ATOM | 1274 | O | THR | A | 543 | 14.967 | −10.139 | 25.938 | 1.00 | 9.69 | O |
| ATOM | 1275 | N | ARG | A | 544 | 14.325 | −12.283 | 25.690 | 1.00 | 9.66 | N |
| ATOM | 1276 | CA | ARG | A | 544 | 15.657 | −12.785 | 25.472 | 1.00 | 10.93 | C |
| ATOM | 1277 | CB | ARG | A | 544 | 15.645 | −14.329 | 25.540 | 1.00 | 11.59 | C |
| ATOM | 1278 | CG | ARG | A | 544 | 16.929 | −15.067 | 25.014 | 1.00 | 10.88 | C |
| ATOM | 1279 | CD | ARG | A | 544 | 16.848 | −16.618 | 25.167 | 1.00 | 14.78 | C |
| ATOM | 1280 | NE | ARG | A | 544 | 15.749 | −17.233 | 24.413 | 1.00 | 13.02 | N |
| ATOM | 1281 | CZ | ARG | A | 544 | 15.747 | −17.391 | 23.074 | 1.00 | 18.91 | C |
| ATOM | 1282 | NH1 | ARG | A | 544 | 16.775 | −16.942 | 22.355 | 1.00 | 23.09 | N |
| ATOM | 1283 | NH2 | ARG | A | 544 | 14.719 | −17.965 | 22.422 | 1.00 | 17.48 | N |
| ATOM | 1284 | C | ARG | A | 544 | 16.117 | −12.289 | 24.106 | 1.00 | 11.05 | C |
| ATOM | 1285 | O | ARG | A | 544 | 17.262 | −11.979 | 23.932 | 1.00 | 10.33 | O |
| ATOM | 1286 | N | GLU | A | 545 | 15.186 | −12.211 | 23.167 | 1.00 | 11.63 | N |
| ATOM | 1287 | CA | GLU | A | 545 | 15.455 | −11.772 | 21.819 | 1.00 | 13.95 | C |
| ATOM | 1288 | CB | GLU | A | 545 | 14.246 | −12.016 | 20.946 | 1.00 | 13.76 | C |
| ATOM | 1289 | CG | GLU | A | 545 | 13.860 | −13.495 | 20.890 | 1.00 | 21.85 | C |
| ATOM | 1290 | CD | GLU | A | 545 | 13.715 | −14.208 | 22.292 | 1.00 | 28.21 | C |
| ATOM | 1291 | OE1 | GLU | A | 545 | 13.028 | −13.699 | 23.251 | 1.00 | 22.99 | O |
| ATOM | 1292 | OE2 | GLU | A | 545 | 14.307 | −15.320 | 22.427 | 1.00 | 29.40 | O |
| ATOM | 1293 | C | GLU | A | 545 | 15.772 | −10.298 | 21.757 | 1.00 | 14.02 | C |
| ATOM | 1294 | O | GLU | A | 545 | 16.548 | −9.871 | 20.921 | 1.00 | 14.84 | O |
| ATOM | 1295 | N | MET | A | 546 | 15.154 | −9.530 | 22.646 | 1.00 | 13.93 | N |
| ATOM | 1296 | CA | MET | A | 546 | 15.330 | −8.086 | 22.725 | 1.00 | 13.00 | C |
| ATOM | 1297 | CB | MET | A | 546 | 14.159 | −7.478 | 23.524 | 1.00 | 13.31 | C |
| ATOM | 1298 | CG | MET | A | 546 | 13.948 | −5.997 | 23.287 | 1.00 | 19.54 | C |
| ATOM | 1299 | SD | MET | A | 546 | 13.855 | −5.454 | 21.439 | 1.00 | 29.26 | S |
| ATOM | 1300 | CE | MET | A | 546 | 15.547 | −5.068 | 21.117 | 1.00 | 21.36 | C |
| ATOM | 1301 | C | MET | A | 546 | 16.706 | −7.727 | 23.314 | 1.00 | 10.12 | C |
| ATOM | 1302 | O | MET | A | 546 | 17.354 | −6.831 | 22.867 | 1.00 | 7.74 | O |
| ATOM | 1303 | N | ILE | A | 547 | 17.154 | −8.475 | 24.313 | 1.00 | 9.83 | N |
| ATOM | 1304 | CA | ILE | A | 547 | 18.459 | −8.235 | 24.923 | 1.00 | 8.79 | C |
| ATOM | 1305 | CB | ILE | A | 547 | 18.601 | −9.080 | 26.159 | 1.00 | 7.97 | C |
| ATOM | 1306 | CG1 | ILE | A | 547 | 17.582 | −8.632 | 27.190 | 1.00 | 7.88 | C |
| ATOM | 1307 | CD1 | ILE | A | 547 | 17.608 | −9.481 | 28.521 | 1.00 | 7.89 | C |
| ATOM | 1308 | CG2 | ILE | A | 547 | 19.986 | −8.952 | 26.827 | 1.00 | 8.85 | C |
| ATOM | 1309 | C | ILE | A | 547 | 19.476 | −8.603 | 23.875 | 1.00 | 8.59 | C |
| ATOM | 1310 | O | ILE | A | 547 | 20.432 | −7.911 | 23.585 | 1.00 | 9.01 | O |
| ATOM | 1311 | N | LYS | A | 548 | 19.203 | −9.683 | 23.224 | 1.00 | 8.48 | N |
| ATOM | 1312 | CA | LYS | A | 548 | 20.060 | −10.106 | 22.157 | 1.00 | 8.58 | C |
| ATOM | 1313 | CB | LYS | A | 548 | 19.527 | −11.417 | 21.580 | 1.00 | 8.46 | C |
| ATOM | 1314 | CG | LYS | A | 548 | 20.646 | −12.366 | 21.115 | 1.00 | 14.13 | C |
| ATOM | 1315 | CD | LYS | A | 548 | 20.837 | −13.592 | 22.053 | 1.00 | 20.90 | C |
| ATOM | 1316 | CE | LYS | A | 548 | 21.254 | −13.189 | 23.487 | 1.00 | 24.85 | C |
| ATOM | 1317 | NZ | LYS | A | 548 | 21.477 | −14.338 | 24.469 | 1.00 | 25.80 | N |
| ATOM | 1318 | C | LYS | A | 548 | 20.211 | −9.024 | 21.088 | 1.00 | 7.39 | C |
| ATOM | 1319 | O | LYS | A | 548 | 21.321 | −8.759 | 20.627 | 1.00 | 7.65 | O |
| ATOM | 1320 | N | HIS | A | 549 | 19.100 | −8.402 | 20.689 | 1.00 | 5.99 | N |
| ATOM | 1321 | CA | HIS | A | 549 | 19.118 | −7.426 | 19.629 | 1.00 | 3.72 | C |
| ATOM | 1322 | CB | HIS | A | 549 | 17.703 | −7.050 | 19.213 | 1.00 | 2.50 | C |
| ATOM | 1323 | CG | HIS | A | 549 | 17.656 | −5.973 | 18.172 | 1.00 | 4.11 | C |
| ATOM | 1324 | ND1 | HIS | A | 549 | 18.258 | −6.102 | 16.932 | 1.00 | 2.57 | N |
| ATOM | 1325 | CE1 | HIS | A | 549 | 18.083 | −4.979 | 16.253 | 1.00 | 2.00 | C |
| ATOM | 1326 | NE2 | HIS | A | 549 | 17.394 | −4.134 | 17.003 | 1.00 | 2.00 | N |
| ATOM | 1327 | CD2 | HIS | A | 549 | 17.121 | −4.725 | 18.206 | 1.00 | 2.00 | C |
| ATOM | 1328 | C | HIS | A | 549 | 19.919 | −6.219 | 20.099 | 1.00 | 4.21 | C |
| ATOM | 1329 | O | HIS | A | 549 | 20.773 | −5.673 | 19.357 | 1.00 | 2.85 | O |
| ATOM | 1330 | N | LEU | A | 550 | 19.698 | −5.856 | 21.366 | 1.00 | 4.44 | N |
| ATOM | 1331 | CA | LEU | A | 550 | 20.395 | −4.711 | 21.962 | 1.00 | 5.58 | C |
| ATOM | 1332 | CB | LEU | A | 550 | 19.851 | −4.379 | 23.369 | 1.00 | 4.96 | C |
| ATOM | 1333 | CG | LEU | A | 550 | 18.486 | −3.698 | 23.396 | 1.00 | 6.21 | C |
| ATOM | 1334 | CD1 | LEU | A | 550 | 18.194 | −3.255 | 24.820 | 1.00 | 9.90 | C |
| ATOM | 1335 | CD2 | LEU | A | 550 | 18.512 | −2.473 | 22.485 | 1.00 | 8.87 | C |
| ATOM | 1336 | C | LEU | A | 550 | 21.912 | −4.937 | 21.991 | 1.00 | 5.86 | C |
| ATOM | 1337 | O | LEU | A | 550 | 22.693 | −4.018 | 21.757 | 1.00 | 5.64 | O |
| ATOM | 1338 | N | GLU | A | 551 | 22.318 | −6.167 | 22.289 | 1.00 | 5.89 | N |
| ATOM | 1339 | CA | GLU | A | 551 | 23.714 | −6.493 | 22.297 | 1.00 | 7.35 | C |
| ATOM | 1340 | CB | GLU | A | 551 | 23.897 | −7.847 | 22.907 | 1.00 | 7.43 | C |
| ATOM | 1341 | CG | GLU | A | 551 | 25.312 | −8.358 | 22.922 | 1.00 | 11.11 | C |
| ATOM | 1342 | CD | GLU | A | 551 | 25.376 | −9.858 | 23.196 | 1.00 | 12.97 | C |
| ATOM | 1343 | OE1 | GLU | A | 551 | 25.783 | −10.217 | 24.325 | 1.00 | 13.82 | O |
| ATOM | 1344 | OE2 | GLU | A | 551 | 25.015 | −10.655 | 22.287 | 1.00 | 12.23 | O |
| ATOM | 1345 | C | GLU | A | 551 | 24.347 | −6.418 | 20.897 | 1.00 | 7.94 | C |
| ATOM | 1346 | O | GLU | A | 551 | 25.475 | −5.935 | 20.720 | 1.00 | 8.12 | O |
| ATOM | 1347 | N | ARG | A | 552 | 23.616 | −6.849 | 19.888 | 1.00 | 8.62 | N |
| ATOM | 1348 | CA | ARG | A | 552 | 24.128 | −6.750 | 18.529 | 1.00 | 10.18 | C |
| ATOM | 1349 | CB | ARG | A | 552 | 23.144 | −7.403 | 17.595 | 1.00 | 12.41 | C |
| ATOM | 1350 | CG | ARG | A | 552 | 23.649 | −7.583 | 16.190 | 1.00 | 20.85 | C |
| ATOM | 1351 | CD | ARG | A | 552 | 24.954 | −8.372 | 16.069 | 1.00 | 27.00 | C |
| ATOM | 1352 | NE | ARG | A | 552 | 25.734 | −7.818 | 14.969 | 1.00 | 30.08 | N |
| ATOM | 1353 | CZ | ARG | A | 552 | 27.060 | −7.851 | 14.950 | 1.00 | 34.49 | C |
| ATOM | 1354 | NH1 | ARG | A | 552 | 27.664 | −8.435 | 15.999 | 1.00 | 30.27 | N |
| ATOM | 1355 | NH2 | ARG | A | 552 | 27.773 | −7.319 | 13.901 | 1.00 | 34.69 | N |
| ATOM | 1356 | C | ARG | A | 552 | 24.352 | −5.292 | 18.076 | 1.00 | 9.36 | C |
| ATOM | 1357 | O | ARG | A | 552 | 25.304 | −5.020 | 17.357 | 1.00 | 9.30 | O |
| ATOM | 1358 | N | CYS | A | 553 | 23.467 | −4.381 | 18.496 | 1.00 | 7.47 | N |
| ATOM | 1359 | CA | CYS | A | 553 | 23.615 | −2.972 | 18.243 | 1.00 | 7.51 | C |
| ATOM | 1360 | CB | CYS | A | 553 | 22.387 | −2.146 | 18.736 | 1.00 | 7.23 | C |
| ATOM | 1361 | SG | CYS | A | 553 | 20.754 | −2.427 | 17.964 | 1.00 | 7.13 | S |
| ATOM | 1362 | C | CYS | A | 553 | 24.890 | −2.480 | 18.971 | 1.00 | 7.59 | C |
| ATOM | 1363 | O | CYS | A | 553 | 25.704 | −1.706 | 18.435 | 1.00 | 7.17 | O |
| ATOM | 1364 | N | GLU | A | 554 | 25.084 | −2.959 | 20.180 | 1.00 | 6.68 | N |
| ATOM | 1365 | CA | GLU | A | 554 | 26.265 | −2.557 | 20.905 | 1.00 | 8.06 | C |
| ATOM | 1366 | CB | GLU | A | 554 | 26.211 | −3.111 | 22.321 | 1.00 | 7.22 | C |
| ATOM | 1367 | CG | GLU | A | 554 | 27.140 | −2.424 | 23.280 | 1.00 | 5.98 | C |
| ATOM | 1368 | CD | GLU | A | 554 | 26.917 | −2.886 | 24.688 | 1.00 | 11.28 | C |
| ATOM | 1369 | OE1 | GLU | A | 554 | 26.704 | −4.112 | 24.903 | 1.00 | 15.10 | O |
| ATOM | 1370 | OE2 | GLU | A | 554 | 26.930 | −2.034 | 25.602 | 1.00 | 14.58 | O |
| ATOM | 1371 | C | GLU | A | 554 | 27.576 | −2.999 | 20.274 | 1.00 | 8.25 | C |
| ATOM | 1372 | O | GLU | A | 554 | 28.592 | −2.329 | 20.318 | 1.00 | 9.11 | O |
| ATOM | 1373 | N | HIS | A | 555 | 27.561 | −4.184 | 19.737 | 1.00 | 8.65 | N |
| ATOM | 1374 | CA | HIS | A | 555 | 28.736 | −4.736 | 19.112 | 1.00 | 7.90 | C |
| ATOM | 1375 | CB | HIS | A | 555 | 28.487 | −6.229 | 18.855 | 1.00 | 6.62 | C |
| ATOM | 1376 | CG | HIS | A | 555 | 28.603 | −7.083 | 20.083 | 1.00 | 4.39 | C |
| ATOM | 1377 | ND1 | HIS | A | 555 | 28.242 | −8.418 | 20.099 | 1.00 | 2.00 | N |
| ATOM | 1378 | CE1 | HIS | A | 555 | 28.523 | −8.929 | 21.283 | 1.00 | 2.00 | C |
| ATOM | 1379 | NE2 | HIS | A | 555 | 29.024 | −7.971 | 22.050 | 1.00 | 2.00 | N |
| ATOM | 1380 | CD2 | HIS | A | 555 | 29.094 | −6.808 | 21.319 | 1.00 | 2.05 | C |
| ATOM | 1381 | C | HIS | A | 555 | 29.067 | −3.985 | 17.817 | 1.00 | 8.31 | C |
| ATOM | 1382 | O | HIS | A | 555 | 30.235 | −3.850 | 17.416 | 1.00 | 8.87 | O |
| ATOM | 1383 | N | ARG | A | 556 | 28.046 | −3.483 | 17.145 | 1.00 | 7.61 | N |
| ATOM | 1384 | CA | ARG | A | 556 | 28.312 | −2.784 | 15.910 | 1.00 | 7.11 | C |
| ATOM | 1385 | CB | ARG | A | 556 | 27.068 | −2.745 | 15.032 | 1.00 | 7.58 | C |
| ATOM | 1386 | CG | ARG | A | 556 | 26.830 | −4.086 | 14.419 | 1.00 | 12.81 | C |
| ATOM | 1387 | CD | ARG | A | 556 | 25.422 | −4.476 | 14.251 | 1.00 | 19.23 | C |
| ATOM | 1388 | NE | ARG | A | 556 | 24.910 | −3.879 | 13.043 | 1.00 | 22.82 | N |
| ATOM | 1389 | CZ | ARG | A | 556 | 23.632 | −3.796 | 12.766 | 1.00 | 25.22 | C |
| ATOM | 1390 | NH1 | ARG | A | 556 | 22.735 | −4.276 | 13.647 | 1.00 | 23.17 | N |
| ATOM | 1391 | NH2 | ARG | A | 556 | 23.255 | −3.226 | 11.619 | 1.00 | 23.51 | N |
| ATOM | 1392 | C | ARG | A | 556 | 28.790 | −1.417 | 16.218 | 1.00 | 5.89 | C |
| ATOM | 1393 | O | ARG | A | 556 | 29.447 | −0.835 | 15.400 | 1.00 | 6.78 | O |
| ATOM | 1394 | N | ILE | A | 557 | 28.480 | −0.890 | 17.404 | 1.00 | 4.95 | N |
| ATOM | 1395 | CA | ILE | A | 557 | 28.975 | 0.438 | 17.777 | 1.00 | 3.96 | C |
| ATOM | 1396 | CB | ILE | A | 557 | 28.195 | 0.966 | 18.957 | 1.00 | 4.35 | C |
| ATOM | 1397 | CG1 | ILE | A | 557 | 26.718 | 1.166 | 18.578 | 1.00 | 3.64 | C |
| ATOM | 1398 | CD1 | ILE | A | 557 | 25.860 | 1.516 | 19.797 | 1.00 | 2.00 | C |
| ATOM | 1399 | CG2 | ILE | A | 557 | 28.762 | 2.324 | 19.538 | 1.00 | 2.19 | C |
| ATOM | 1400 | C | ILE | A | 557 | 30.462 | 0.279 | 18.067 | 1.00 | 5.06 | C |
| ATOM | 1401 | O | ILE | A | 557 | 31.301 | 1.023 | 17.582 | 1.00 | 3.84 | O |
| ATOM | 1402 | N | MET | A | 558 | 30.774 | −0.732 | 18.862 | 1.00 | 6.34 | N |
| ATOM | 1403 | CA | MET | A | 558 | 32.137 | −1.072 | 19.214 | 1.00 | 7.02 | C |
| ATOM | 1404 | CB | MET | A | 558 | 32.147 | −2.170 | 20.277 | 1.00 | 7.34 | C |
| ATOM | 1405 | CG | MET | A | 558 | 31.501 | −1.673 | 21.619 | 1.00 | 12.35 | C |
| ATOM | 1406 | SD | MET | A | 558 | 31.502 | −2.949 | 22.900 | 1.00 | 17.81 | S |
| ATOM | 1407 | CE | MET | A | 558 | 30.101 | −3.923 | 22.556 | 1.00 | 14.43 | C |
| ATOM | 1408 | C | MET | A | 558 | 33.009 | −1.491 | 18.054 | 1.00 | 5.34 | C |
| ATOM | 1409 | O | MET | A | 558 | 34.222 | −1.284 | 18.091 | 1.00 | 4.60 | O |
| ATOM | 1410 | N | GLU | A | 559 | 32.417 | −2.102 | 17.040 | 1.00 | 3.43 | N |
| ATOM | 1411 | CA | GLU | A | 559 | 33.269 | −2.609 | 15.983 | 1.00 | 2.79 | C |
| ATOM | 1412 | CB | GLU | A | 559 | 32.673 | −3.807 | 15.250 | 1.00 | 3.49 | C |
| ATOM | 1413 | CG | GLU | A | 559 | 31.360 | −3.495 | 14.562 | 1.00 | 6.17 | C |
| ATOM | 1414 | CD | GLU | A | 559 | 30.720 | −4.665 | 13.825 | 1.00 | 9.46 | C |
| ATOM | 1415 | OE1 | GLU | A | 559 | 30.780 | −5.820 | 14.299 | 1.00 | 6.19 | O |
| ATOM | 1416 | OE2 | GLU | A | 559 | 30.122 | −4.376 | 12.742 | 1.00 | 13.37 | O |
| ATOM | 1417 | C | GLU | A | 559 | 33.642 | −1.514 | 15.017 | 1.00 | 2.46 | C |
| ATOM | 1418 | O | GLU | A | 559 | 34.777 | −1.515 | 14.553 | 1.00 | 3.03 | O |
| ATOM | 1419 | N | SER | A | 560 | 32.722 | −0.585 | 14.708 | 1.00 | 2.00 | N |
| ATOM | 1420 | CA | SER | A | 560 | 32.999 | 0.509 | 13.765 | 1.00 | 2.00 | C |
| ATOM | 1421 | CB | SER | A | 560 | 32.848 | 0.074 | 12.287 | 1.00 | 2.00 | C |
| ATOM | 1422 | OG | SER | A | 560 | 31.566 | −0.370 | 11.966 | 1.00 | 2.00 | O |
| ATOM | 1423 | C | SER | A | 560 | 32.339 | 1.845 | 13.960 | 1.00 | 2.00 | C |
| ATOM | 1424 | O | SER | A | 560 | 33.008 | 2.816 | 14.009 | 1.00 | 2.06 | O |
| ATOM | 1425 | N | LEU | A | 561 | 31.020 | 1.902 | 14.097 | 1.00 | 2.00 | N |
| ATOM | 1426 | CA | LEU | A | 561 | 30.323 | 3.156 | 14.220 | 1.00 | 2.20 | C |
| ATOM | 1427 | CB | LEU | A | 561 | 28.835 | 2.896 | 14.518 | 1.00 | 2.33 | C |
| ATOM | 1428 | CG | LEU | A | 561 | 27.969 | 2.019 | 13.593 | 1.00 | 4.31 | C |
| ATOM | 1429 | CD1 | LEU | A | 561 | 26.528 | 1.785 | 14.215 | 1.00 | 2.00 | C |
| ATOM | 1430 | CD2 | LEU | A | 561 | 27.927 | 2.524 | 12.106 | 1.00 | 2.00 | C |
| ATOM | 1431 | C | LEU | A | 561 | 30.959 | 4.193 | 15.219 | 1.00 | 2.00 | C |
| ATOM | 1432 | O | LEU | A | 561 | 31.119 | 5.361 | 14.911 | 1.00 | 2.00 | O |
| ATOM | 1433 | N | ALA | A | 562 | 31.326 | 3.756 | 16.393 | 1.00 | 2.00 | N |
| ATOM | 1434 | CA | ALA | A | 562 | 31.879 | 4.693 | 17.354 | 1.00 | 2.83 | C |
| ATOM | 1435 | CB | ALA | A | 562 | 31.979 | 4.095 | 18.810 | 1.00 | 2.00 | C |
| ATOM | 1436 | C | ALA | A | 562 | 33.259 | 5.109 | 16.884 | 1.00 | 3.11 | C |
| ATOM | 1437 | O | ALA | A | 562 | 33.863 | 5.921 | 17.530 | 1.00 | 3.18 | O |
| ATOM | 1438 | N | TRP | A | 563 | 33.768 | 4.535 | 15.806 | 1.00 | 2.21 | N |
| ATOM | 1439 | CA | TRP | A | 563 | 35.073 | 4.973 | 15.316 | 1.00 | 3.35 | C |
| ATOM | 1440 | CB | TRP | A | 563 | 36.036 | 3.777 | 15.101 | 1.00 | 2.70 | C |
| ATOM | 1441 | CG | TRP | A | 563 | 36.208 | 2.881 | 16.308 | 1.00 | 3.50 | C |
| ATOM | 1442 | CD1 | TRP | A | 563 | 35.434 | 1.796 | 16.638 | 1.00 | 4.08 | C |
| ATOM | 1443 | NE1 | TRP | A | 563 | 35.881 | 1.214 | 17.793 | 1.00 | 2.00 | N |
| ATOM | 1444 | CE2 | TRP | A | 563 | 36.974 | 1.908 | 18.231 | 1.00 | 2.92 | C |
| ATOM | 1445 | CD2 | TRP | A | 563 | 37.222 | 2.948 | 17.292 | 1.00 | 2.00 | C |
| ATOM | 1446 | CE3 | TRP | A | 563 | 38.273 | 3.804 | 17.524 | 1.00 | 2.65 | C |
| ATOM | 1447 | CZ3 | TRP | A | 563 | 39.083 | 3.601 | 18.648 | 1.00 | 3.77 | C |
| ATOM | 1448 | CH2 | TRP | A | 563 | 38.839 | 2.532 | 19.547 | 1.00 | 2.00 | C |
| ATOM | 1449 | CZ2 | TRP | A | 563 | 37.786 | 1.694 | 19.378 | 1.00 | 2.00 | C |
| ATOM | 1450 | C | TRP | A | 563 | 34.969 | 5.825 | 14.016 | 1.00 | 3.80 | C |
| ATOM | 1451 | O | TRP | A | 563 | 35.925 | 6.044 | 13.306 | 1.00 | 2.92 | O |
| ATOM | 1452 | N | LEU | A | 564 | 33.777 | 6.253 | 13.660 | 1.00 | 4.71 | N |
| ATOM | 1453 | CA | LEU | A | 564 | 33.712 | 7.145 | 12.484 | 1.00 | 5.58 | C |
| ATOM | 1454 | CB | LEU | A | 564 | 32.264 | 7.290 | 11.965 | 1.00 | 4.26 | C |
| ATOM | 1455 | CG | LEU | A | 564 | 31.566 | 6.068 | 11.390 | 1.00 | 4.77 | C |
| ATOM | 1456 | CD1 | LEU | A | 564 | 30.004 | 6.356 | 11.192 | 1.00 | 3.32 | C |
| ATOM | 1457 | CD2 | LEU | A | 564 | 32.200 | 5.550 | 10.050 | 1.00 | 2.00 | C |
| ATOM | 1458 | C | LEU | A | 564 | 34.290 | 8.532 | 12.858 | 1.00 | 4.78 | C |
| ATOM | 1459 | O | LEU | A | 564 | 34.350 | 8.918 | 14.034 | 1.00 | 4.33 | O |
| ATOM | 1460 | N | SER | A | 565 | 34.677 | 9.272 | 11.849 | 1.00 | 4.86 | N |
| ATOM | 1461 | CA | SER | A | 565 | 35.281 | 10.573 | 12.036 | 1.00 | 5.62 | C |
| ATOM | 1462 | CB | SER | A | 565 | 35.653 | 11.162 | 10.720 | 1.00 | 6.03 | C |
| ATOM | 1463 | OG | SER | A | 565 | 36.397 | 10.281 | 9.913 | 1.00 | 9.07 | O |
| ATOM | 1464 | C | SER | A | 565 | 34.368 | 11.553 | 12.711 | 1.00 | 6.16 | C |
| ATOM | 1465 | O | SER | A | 565 | 34.856 | 12.519 | 13.301 | 1.00 | 7.64 | O |
| ATOM | 1466 | N | ASP | A | 566 | 33.058 | 11.357 | 12.600 | 1.00 | 5.01 | N |
| ATOM | 1467 | CA | ASP | A | 566 | 32.118 | 12.194 | 13.356 | 1.00 | 4.61 | C |
| ATOM | 1468 | CB | ASP | A | 566 | 30.751 | 12.235 | 12.667 | 1.00 | 4.01 | C |
| ATOM | 1469 | CG | ASP | A | 566 | 30.043 | 10.859 | 12.633 | 1.00 | 7.60 | C |
| ATOM | 1470 | OD1 | ASP | A | 566 | 30.719 | 9.768 | 12.582 | 1.00 | 12.23 | O |
| ATOM | 1471 | OD2 | ASP | A | 566 | 28.798 | 10.766 | 12.592 | 1.00 | 6.42 | O |
| ATOM | 1472 | C | ASP | A | 566 | 31.929 | 11.762 | 14.835 | 1.00 | 4.59 | C |
| ATOM | 1473 | O | ASP | A | 566 | 31.067 | 12.257 | 15.518 | 1.00 | 6.37 | O |
| ATOM | 1474 | N | SER | A | 567 | 32.752 | 10.892 | 15.365 | 1.00 | 3.80 | N |
| ATOM | 1475 | CA | SER | A | 567 | 32.472 | 10.379 | 16.678 | 1.00 | 4.60 | C |
| ATOM | 1476 | CB | SER | A | 567 | 32.849 | 8.890 | 16.709 | 1.00 | 4.41 | C |
| ATOM | 1477 | OG | SER | A | 567 | 32.554 | 8.341 | 17.982 | 1.00 | 5.48 | O |
| ATOM | 1478 | C | SER | A | 567 | 33.115 | 11.092 | 17.875 | 1.00 | 4.42 | C |
| ATOM | 1479 | O | SER | A | 567 | 34.332 | 11.220 | 17.963 | 1.00 | 4.31 | O |
| ATOM | 1480 | N | PRO | A | 568 | 32.300 | 11.423 | 18.867 | 1.00 | 4.48 | N |
| ATOM | 1481 | CA | PRO | A | 568 | 32.786 | 12.087 | 20.083 | 1.00 | 4.54 | C |
| ATOM | 1482 | CB | PRO | A | 568 | 31.550 | 12.119 | 20.963 | 1.00 | 4.15 | C |
| ATOM | 1483 | CG | PRO | A | 568 | 30.425 | 12.026 | 20.031 | 1.00 | 4.02 | C |
| ATOM | 1484 | CD | PRO | A | 568 | 30.859 | 11.141 | 18.927 | 1.00 | 3.92 | C |
| ATOM | 1485 | C | PRO | A | 568 | 33.922 | 11.300 | 20.746 | 1.00 | 4.80 | C |
| ATOM | 1486 | O | PRO | A | 568 | 34.778 | 11.860 | 21.382 | 1.00 | 5.03 | O |
| ATOM | 1487 | N | LEU | A | 569 | 33.962 | 9.997 | 20.550 | 1.00 | 5.56 | N |
| ATOM | 1488 | CA | LEU | A | 569 | 35.065 | 9.185 | 21.063 | 1.00 | 5.83 | C |
| ATOM | 1489 | CB | LEU | A | 569 | 34.953 | 7.839 | 20.393 | 1.00 | 5.39 | C |
| ATOM | 1490 | CG | LEU | A | 569 | 35.662 | 6.643 | 21.012 | 1.00 | 8.02 | C |
| ATOM | 1491 | CD1 | LEU | A | 569 | 36.514 | 5.968 | 20.035 | 1.00 | 7.12 | C |
| ATOM | 1492 | CD2 | LEU | A | 569 | 36.445 | 7.041 | 22.339 | 1.00 | 9.13 | C |
| ATOM | 1493 | C | LEU | A | 569 | 36.523 | 9.780 | 20.895 | 1.00 | 6.51 | C |
| ATOM | 1494 | O | LEU | A | 569 | 37.338 | 9.766 | 21.816 | 1.00 | 7.17 | O |
| ATOM | 1495 | N | PHE | A | 570 | 36.860 | 10.317 | 19.739 | 1.00 | 6.86 | N |
| ATOM | 1496 | CA | PHE | A | 570 | 38.216 | 10.808 | 19.528 | 1.00 | 7.23 | C |
| ATOM | 1497 | CB | PHE | A | 570 | 38.501 | 11.136 | 18.035 | 1.00 | 5.23 | C |
| ATOM | 1498 | CG | PHE | A | 570 | 38.424 | 9.923 | 17.163 | 1.00 | 6.73 | C |
| ATOM | 1499 | CD1 | PHE | A | 570 | 39.368 | 8.914 | 17.301 | 1.00 | 7.09 | C |
| ATOM | 1500 | CE1 | PHE | A | 570 | 39.276 | 7.757 | 16.572 | 1.00 | 7.82 | C |
| ATOM | 1501 | CZ | PHE | A | 570 | 38.233 | 7.573 | 15.679 | 1.00 | 7.51 | C |
| ATOM | 1502 | CE2 | PHE | A | 570 | 37.301 | 8.556 | 15.528 | 1.00 | 6.38 | C |
| ATOM | 1503 | CD2 | PHE | A | 570 | 37.377 | 9.719 | 16.292 | 1.00 | 5.05 | C |
| ATOM | 1504 | C | PHE | A | 570 | 38.524 | 11.975 | 20.418 | 1.00 | 9.18 | C |
| ATOM | 1505 | O | PHE | A | 570 | 39.718 | 12.193 | 20.765 | 1.00 | 10.62 | O |
| ATOM | 1506 | N | ASP | A | 571 | 37.494 | 12.747 | 20.782 | 1.00 | 10.38 | N |
| ATOM | 1507 | CA | ASP | A | 571 | 37.678 | 13.891 | 21.705 | 1.00 | 12.23 | C |
| ATOM | 1508 | CB | ASP | A | 571 | 36.503 | 14.908 | 21.636 | 1.00 | 12.92 | C |
| ATOM | 1509 | CG | ASP | A | 571 | 36.747 | 16.079 | 20.592 | 1.00 | 19.58 | C |
| ATOM | 1510 | OD1 | ASP | A | 571 | 35.738 | 16.784 | 20.221 | 1.00 | 22.58 | O |
| ATOM | 1511 | OD2 | ASP | A | 571 | 37.892 | 16.388 | 20.102 | 1.00 | 24.68 | O |
| ATOM | 1512 | C | ASP | A | 571 | 37.838 | 13.320 | 23.139 | 1.00 | 11.69 | C |
| ATOM | 1513 | O | ASP | A | 571 | 38.703 | 13.723 | 23.898 | 1.00 | 9.05 | O |
| ATOM | 1514 | N | LEU | A | 572 | 37.023 | 12.322 | 23.447 | 1.00 | 12.36 | N |
| ATOM | 1515 | CA | LEU | A | 572 | 37.111 | 11.657 | 24.705 | 1.00 | 14.85 | C |
| ATOM | 1516 | CB | LEU | A | 572 | 36.028 | 10.621 | 24.800 | 1.00 | 16.18 | C |
| ATOM | 1517 | CG | LEU | A | 572 | 35.809 | 10.127 | 26.233 | 1.00 | 21.52 | C |
| ATOM | 1518 | CD1 | LEU | A | 572 | 35.722 | 11.348 | 27.185 | 1.00 | 21.46 | C |
| ATOM | 1519 | CD2 | LEU | A | 572 | 34.536 | 9.247 | 26.291 | 1.00 | 23.88 | C |
| ATOM | 1520 | C | LEU | A | 572 | 38.461 | 10.963 | 24.850 | 1.00 | 15.08 | C |
| ATOM | 1521 | O | LEU | A | 572 | 38.945 | 10.761 | 25.954 | 1.00 | 15.22 | O |
| ATOM | 1522 | N | ILE | A | 573 | 39.068 | 10.567 | 23.745 | 1.00 | 15.07 | N |
| ATOM | 1523 | CA | ILE | A | 573 | 40.376 | 9.968 | 23.850 | 1.00 | 15.55 | C |
| ATOM | 1524 | CB | ILE | A | 573 | 40.645 | 9.090 | 22.628 | 1.00 | 14.55 | C |
| ATOM | 1525 | CG1 | ILE | A | 573 | 39.959 | 7.746 | 22.767 | 1.00 | 12.31 | C |
| ATOM | 1526 | CD1 | ILE | A | 573 | 39.857 | 6.981 | 21.393 | 1.00 | 6.14 | C |
| ATOM | 1527 | CG2 | ILE | A | 573 | 42.098 | 8.825 | 22.425 | 1.00 | 12.90 | C |
| ATOM | 1528 | C | ILE | A | 573 | 41.435 | 11.088 | 23.983 | 1.00 | 17.30 | C |
| ATOM | 1529 | O | ILE | A | 573 | 42.414 | 10.949 | 24.685 | 1.00 | 18.35 | O |
| ATOM | 1530 | N | LYS | A | 574 | 41.261 | 12.192 | 23.290 | 1.00 | 17.39 | N |
| ATOM | 1531 | CA | LYS | A | 574 | 42.272 | 13.190 | 23.373 | 1.00 | 18.15 | C |
| ATOM | 1532 | CB | LYS | A | 574 | 42.087 | 14.242 | 22.296 | 1.00 | 16.97 | C |
| ATOM | 1533 | CG | LYS | A | 574 | 43.280 | 15.079 | 22.098 | 1.00 | 15.24 | C |
| ATOM | 1534 | CD | LYS | A | 574 | 43.072 | 16.241 | 21.114 | 1.00 | 15.89 | C |
| ATOM | 1535 | CE | LYS | A | 574 | 44.287 | 17.213 | 21.136 | 1.00 | 14.11 | C |
| ATOM | 1536 | NZ | LYS | A | 574 | 44.345 | 18.197 | 20.020 | 1.00 | 9.92 | N |
| ATOM | 1537 | C | LYS | A | 574 | 42.251 | 13.802 | 24.760 | 1.00 | 20.35 | C |
| ATOM | 1538 | O | LYS | A | 574 | 43.302 | 13.946 | 25.401 | 1.00 | 20.46 | O |
| ATOM | 1539 | N | GLN | A | 575 | 41.059 | 14.149 | 25.224 | 1.00 | 22.46 | N |
| ATOM | 1540 | CA | GLN | A | 575 | 40.876 | 14.743 | 26.532 | 1.00 | 24.95 | C |
| ATOM | 1541 | CB | GLN | A | 575 | 39.394 | 14.789 | 26.845 | 1.00 | 25.51 | C |
| ATOM | 1542 | CG | GLN | A | 575 | 39.020 | 15.353 | 28.210 | 1.00 | 31.74 | C |
| ATOM | 1543 | CD | GLN | A | 575 | 37.544 | 15.802 | 28.301 | 1.00 | 36.07 | C |
| ATOM | 1544 | OE1 | GLN | A | 575 | 36.639 | 14.979 | 28.181 | 1.00 | 37.48 | O |
| ATOM | 1545 | NE2 | GLN | A | 575 | 37.315 | 17.110 | 28.535 | 1.00 | 37.16 | N |
| ATOM | 1546 | C | GLN | A | 575 | 41.569 | 13.876 | 27.560 | 1.00 | 26.44 | C |
| ATOM | 1547 | O | GLN | A | 575 | 42.042 | 14.356 | 28.570 | 1.00 | 26.79 | O |
| ATOM | 1548 | N | SER | A | 576 | 41.660 | 12.579 | 27.291 | 1.00 | 28.20 | N |
| ATOM | 1549 | CA | SER | A | 576 | 42.287 | 11.655 | 28.238 | 1.00 | 28.79 | C |
| ATOM | 1550 | CB | SER | A | 576 | 41.622 | 10.312 | 28.144 | 1.00 | 28.17 | C |
| ATOM | 1551 | OG | SER | A | 576 | 42.636 | 9.371 | 27.953 | 1.00 | 27.25 | O |
| ATOM | 1552 | C | SER | A | 576 | 43.775 | 11.452 | 28.024 | 1.00 | 29.76 | C |
| ATOM | 1553 | O | SER | A | 576 | 44.506 | 11.186 | 28.946 | 1.00 | 29.92 | O |
| ATOM | 1554 | N | LYS | A | 577 | 44.210 | 11.566 | 26.785 | 1.00 | 31.31 | N |
| ATOM | 1555 | CA | LYS | A | 577 | 45.610 | 11.424 | 26.438 | 1.00 | 32.44 | C |
| ATOM | 1556 | CB | LYS | A | 577 | 45.756 | 11.456 | 24.924 | 1.00 | 32.43 | C |
| ATOM | 1557 | CG | LYS | A | 577 | 46.759 | 10.514 | 24.390 | 1.00 | 31.18 | C |
| ATOM | 1558 | CD | LYS | A | 577 | 46.252 | 9.097 | 24.601 | 1.00 | 28.82 | C |
| ATOM | 1559 | CE | LYS | A | 577 | 47.095 | 8.118 | 23.806 | 1.00 | 29.90 | C |
| ATOM | 1560 | NZ | LYS | A | 577 | 46.930 | 6.691 | 24.174 | 1.00 | 25.38 | N |
| ATOM | 1561 | C | LYS | A | 577 | 46.462 | 12.556 | 27.028 | 1.00 | 33.67 | C |
| ATOM | 1562 | O | LYS | A | 577 | 47.676 | 12.565 | 26.875 | 1.00 | 33.03 | O |
| ATOM | 1563 | N | ASP | A | 578 | 45.820 | 13.534 | 27.663 | 1.00 | 35.46 | N |
| ATOM | 1564 | CA | ASP | A | 578 | 46.552 | 14.659 | 28.239 | 1.00 | 36.42 | C |
| ATOM | 1565 | CB | ASP | A | 578 | 45.949 | 16.006 | 27.817 | 1.00 | 36.95 | C |
| ATOM | 1566 | CG | ASP | A | 578 | 46.096 | 16.280 | 26.331 | 1.00 | 38.84 | C |
| ATOM | 1567 | OD1 | ASP | A | 578 | 47.060 | 15.800 | 25.685 | 1.00 | 41.17 | O |
| ATOM | 1568 | OD2 | ASP | A | 578 | 45.279 | 16.976 | 25.708 | 1.00 | 42.67 | O |
| ATOM | 1569 | C | ASP | A | 578 | 46.556 | 14.554 | 29.751 | 1.00 | 36.86 | C |
| ATOM | 1573 | N | SER | B | 644 | 36.636 | 9.547 | 33.813 | 1.00 | 13.34 | N |
| ATOM | 1574 | CA | SER | B | 644 | 35.896 | 9.256 | 32.537 | 1.00 | 13.52 | C |
| ATOM | 1575 | CB | SER | B | 644 | 36.738 | 9.648 | 31.317 | 1.00 | 14.13 | C |
| ATOM | 1576 | OG | SER | B | 644 | 35.927 | 9.799 | 30.153 | 1.00 | 17.99 | O |
| ATOM | 1577 | C | SER | B | 644 | 35.367 | 7.811 | 32.424 | 1.00 | 12.17 | C |
| ATOM | 1578 | O | SER | B | 644 | 35.897 | 6.982 | 31.701 | 1.00 | 12.64 | O |
| ATOM | 1579 | N | THR | B | 645 | 34.293 | 7.546 | 33.159 | 1.00 | 10.72 | N |
| ATOM | 1580 | CA | THR | B | 645 | 33.630 | 6.265 | 33.190 | 1.00 | 8.91 | C |
| ATOM | 1581 | CB | THR | B | 645 | 32.330 | 6.374 | 34.033 | 1.00 | 9.09 | C |
| ATOM | 1582 | OG1 | THR | B | 645 | 32.625 | 6.834 | 35.352 | 1.00 | 8.17 | O |
| ATOM | 1583 | CG2 | THR | B | 645 | 31.728 | 4.968 | 34.256 | 1.00 | 9.39 | C |
| ATOM | 1584 | C | THR | B | 645 | 33.274 | 5.785 | 31.770 | 1.00 | 7.52 | C |
| ATOM | 1585 | O | THR | B | 645 | 33.531 | 4.625 | 31.379 | 1.00 | 5.65 | O |
| ATOM | 1586 | N | SER | B | 646 | 32.676 | 6.694 | 31.028 | 1.00 | 6.11 | N |
| ATOM | 1587 | CA | SER | B | 646 | 32.310 | 6.460 | 29.633 | 1.00 | 6.99 | C |
| ATOM | 1588 | CB | SER | B | 646 | 31.925 | 7.758 | 28.930 | 1.00 | 7.51 | C |
| ATOM | 1589 | OG | SER | B | 646 | 32.996 | 8.717 | 29.121 | 1.00 | 12.87 | O |
| ATOM | 1590 | C | SER | B | 646 | 33.444 | 5.841 | 28.850 | 1.00 | 4.97 | C |
| ATOM | 1591 | O | SER | B | 646 | 33.254 | 4.809 | 28.273 | 1.00 | 3.36 | O |
| ATOM | 1592 | N | LEU | B | 647 | 34.606 | 6.477 | 28.859 | 1.00 | 4.50 | N |
| ATOM | 1593 | CA | LEU | B | 647 | 35.742 | 5.969 | 28.104 | 1.00 | 6.01 | C |
| ATOM | 1594 | CB | LEU | B | 647 | 36.846 | 6.981 | 28.058 | 1.00 | 6.07 | C |
| ATOM | 1595 | CG | LEU | B | 647 | 38.046 | 6.586 | 27.214 | 1.00 | 5.64 | C |
| ATOM | 1596 | CD1 | LEU | B | 647 | 37.711 | 6.400 | 25.743 | 1.00 | 2.00 | C |
| ATOM | 1597 | CD2 | LEU | B | 647 | 39.120 | 7.685 | 27.433 | 1.00 | 3.27 | C |
| ATOM | 1598 | C | LEU | B | 647 | 36.327 | 4.653 | 28.634 | 1.00 | 7.62 | C |
| ATOM | 1599 | O | LEU | B | 647 | 36.803 | 3.822 | 27.846 | 1.00 | 7.75 | O |
| ATOM | 1600 | N | SER | B | 648 | 36.335 | 4.459 | 29.958 | 1.00 | 7.31 | N |
| ATOM | 1601 | CA | SER | B | 648 | 36.813 | 3.191 | 30.458 | 1.00 | 7.43 | C |
| ATOM | 1602 | CB | SER | B | 648 | 37.232 | 3.312 | 31.940 | 1.00 | 8.38 | C |
| ATOM | 1603 | OG | SER | B | 648 | 36.099 | 3.311 | 32.774 | 1.00 | 12.14 | O |
| ATOM | 1604 | C | SER | B | 648 | 35.783 | 2.054 | 30.271 | 1.00 | 6.33 | C |
| ATOM | 1605 | O | SER | B | 648 | 36.135 | 0.897 | 30.113 | 1.00 | 6.45 | O |
| ATOM | 1606 | N | LEU | B | 649 | 34.499 | 2.359 | 30.278 | 1.00 | 5.67 | N |
| ATOM | 1607 | CA | LEU | B | 649 | 33.485 | 1.304 | 30.061 | 1.00 | 4.38 | C |
| ATOM | 1608 | CB | LEU | B | 649 | 32.075 | 1.863 | 30.294 | 1.00 | 3.95 | C |
| ATOM | 1609 | CG | LEU | B | 649 | 30.936 | 0.916 | 30.609 | 1.00 | 3.72 | C |
| ATOM | 1610 | CD1 | LEU | B | 649 | 30.121 | 0.646 | 29.371 | 1.00 | 8.06 | C |
| ATOM | 1611 | CD2 | LEU | B | 649 | 31.444 | −0.408 | 31.175 | 1.00 | 2.00 | C |
| ATOM | 1612 | C | LEU | B | 649 | 33.590 | 0.812 | 28.646 | 1.00 | 4.70 | C |
| ATOM | 1613 | O | LEU | B | 649 | 33.553 | −0.418 | 28.356 | 1.00 | 6.51 | O |
| ATOM | 1614 | N | PHE | B | 650 | 33.796 | 1.774 | 27.755 | 1.00 | 3.18 | N |
| ATOM | 1615 | CA | PHE | B | 650 | 33.888 | 1.510 | 26.366 | 1.00 | 2.00 | C |
| ATOM | 1616 | CB | PHE | B | 650 | 33.940 | 2.828 | 25.556 | 1.00 | 2.00 | C |
| ATOM | 1617 | CG | PHE | B | 650 | 33.947 | 2.604 | 24.106 | 1.00 | 2.00 | C |
| ATOM | 1618 | CD1 | PHE | B | 650 | 32.763 | 2.357 | 23.427 | 1.00 | 2.00 | C |
| ATOM | 1619 | CE1 | PHE | B | 650 | 32.792 | 2.111 | 22.035 | 1.00 | 2.00 | C |
| ATOM | 1620 | CZ | PHE | B | 650 | 33.988 | 2.062 | 21.329 | 1.00 | 2.00 | C |
| ATOM | 1621 | CE2 | PHE | B | 650 | 35.175 | 2.284 | 21.992 | 1.00 | 2.00 | C |
| ATOM | 1622 | CD2 | PHE | B | 650 | 35.163 | 2.543 | 23.394 | 1.00 | 2.00 | C |
| ATOM | 1623 | C | PHE | B | 650 | 35.103 | 0.619 | 26.111 | 1.00 | 2.00 | C |
| ATOM | 1624 | O | PHE | B | 650 | 34.994 | −0.415 | 25.474 | 1.00 | 2.37 | O |
| ATOM | 1625 | N | TYR | B | 651 | 36.266 | 1.031 | 26.556 | 1.00 | 2.00 | N |
| ATOM | 1626 | CA | TYR | B | 651 | 37.452 | 0.270 | 26.268 | 1.00 | 3.35 | C |
| ATOM | 1627 | CB | TYR | B | 651 | 38.731 | 0.988 | 26.767 | 1.00 | 3.37 | C |
| ATOM | 1628 | CG | TYR | B | 651 | 39.419 | 1.741 | 25.662 | 1.00 | 4.03 | C |
| ATOM | 1629 | CD1 | TYR | B | 651 | 40.287 | 1.076 | 24.801 | 1.00 | 5.03 | C |
| ATOM | 1630 | CE1 | TYR | B | 651 | 40.882 | 1.737 | 23.744 | 1.00 | 7.04 | C |
| ATOM | 1631 | CZ | TYR | B | 651 | 40.633 | 3.096 | 23.506 | 1.00 | 3.43 | C |
| ATOM | 1632 | OH | TYR | B | 651 | 41.249 | 3.679 | 22.433 | 1.00 | 3.35 | O |
| ATOM | 1633 | CE2 | TYR | B | 651 | 39.780 | 3.780 | 24.300 | 1.00 | 2.37 | C |
| ATOM | 1634 | CD2 | TYR | B | 651 | 39.147 | 3.087 | 25.408 | 1.00 | 2.56 | C |
| ATOM | 1635 | C | TYR | B | 651 | 37.335 | −1.135 | 26.856 | 1.00 | 4.60 | C |
| ATOM | 1636 | O | TYR | B | 651 | 37.557 | −2.180 | 26.169 | 1.00 | 3.86 | O |
| ATOM | 1637 | N | LYS | B | 652 | 36.925 | −1.165 | 28.109 | 1.00 | 5.25 | N |
| ATOM | 1638 | CA | LYS | B | 652 | 36.770 | −2.429 | 28.776 | 1.00 | 7.08 | C |
| ATOM | 1639 | CB | LYS | B | 652 | 36.116 | −2.190 | 30.135 | 1.00 | 9.45 | C |
| ATOM | 1640 | CG | LYS | B | 652 | 36.103 | −3.327 | 31.124 | 1.00 | 9.85 | C |
| ATOM | 1641 | CD | LYS | B | 652 | 35.793 | −2.711 | 32.483 | 1.00 | 8.46 | C |
| ATOM | 1642 | CE | LYS | B | 652 | 34.602 | −3.426 | 33.172 | 1.00 | 11.02 | C |
| ATOM | 1643 | NZ | LYS | B | 652 | 34.713 | −4.916 | 33.235 | 1.00 | 10.84 | N |
| ATOM | 1644 | C | LYS | B | 652 | 35.971 | −3.380 | 27.910 | 1.00 | 6.24 | C |
| ATOM | 1645 | O | LYS | B | 652 | 36.417 | −4.472 | 27.586 | 1.00 | 6.42 | O |
| ATOM | 1646 | N | LYS | B | 653 | 34.803 | −2.965 | 27.484 | 1.00 | 6.01 | N |
| ATOM | 1647 | CA | LYS | B | 653 | 34.039 | −3.842 | 26.566 | 1.00 | 5.79 | C |
| ATOM | 1648 | CB | LYS | B | 653 | 32.592 | −3.355 | 26.388 | 1.00 | 6.75 | C |
| ATOM | 1649 | CG | LYS | B | 653 | 31.683 | −3.588 | 27.623 | 1.00 | 6.98 | C |
| ATOM | 1650 | CD | LYS | B | 653 | 30.444 | −2.803 | 27.531 | 1.00 | 9.41 | C |
| ATOM | 1651 | CE | LYS | B | 653 | 29.457 | −3.240 | 28.518 | 1.00 | 9.56 | C |
| ATOM | 1652 | NZ | LYS | B | 653 | 29.069 | −4.552 | 28.071 | 1.00 | 16.84 | N |
| ATOM | 1653 | C | LYS | B | 653 | 34.665 | −4.068 | 25.177 | 1.00 | 5.15 | C |
| ATOM | 1654 | O | LYS | B | 653 | 34.680 | −5.204 | 24.665 | 1.00 | 5.64 | O |
| ATOM | 1655 | N | VAL | B | 654 | 35.172 | −3.033 | 24.528 | 1.00 | 3.96 | N |
| ATOM | 1656 | CA | VAL | B | 654 | 35.796 | −3.311 | 23.240 | 1.00 | 4.21 | C |
| ATOM | 1657 | CB | VAL | B | 654 | 36.340 | −2.025 | 22.582 | 1.00 | 3.09 | C |
| ATOM | 1658 | CG1 | VAL | B | 654 | 37.138 | −2.320 | 21.425 | 1.00 | 4.47 | C |
| ATOM | 1659 | CG2 | VAL | B | 654 | 35.261 | −1.175 | 22.068 | 1.00 | 3.53 | C |
| ATOM | 1660 | C | VAL | B | 654 | 36.923 | −4.399 | 23.425 | 1.00 | 5.38 | C |
| ATOM | 1661 | O | VAL | B | 654 | 37.162 | −5.194 | 22.517 | 1.00 | 5.50 | O |
| ATOM | 1662 | N | TYR | B | 655 | 37.606 | −4.433 | 24.579 | 1.00 | 4.72 | N |
| ATOM | 1663 | CA | TYR | B | 655 | 38.644 | −5.454 | 24.769 | 1.00 | 6.51 | C |
| ATOM | 1664 | CB | TYR | B | 655 | 39.505 | −5.204 | 26.019 | 1.00 | 6.11 | C |
| ATOM | 1665 | CG | TYR | B | 655 | 40.456 | −4.016 | 25.925 | 1.00 | 9.57 | C |
| ATOM | 1666 | CD1 | TYR | B | 655 | 41.074 | −3.656 | 24.712 | 1.00 | 10.61 | C |
| ATOM | 1667 | CE1 | TYR | B | 655 | 41.943 | −2.567 | 24.654 | 1.00 | 12.73 | C |
| ATOM | 1668 | CZ | TYR | B | 655 | 42.195 | −1.858 | 25.839 | 1.00 | 13.88 | C |
| ATOM | 1669 | OH | TYR | B | 655 | 43.039 | −0.758 | 25.867 | 1.00 | 17.21 | O |
| ATOM | 1670 | CE2 | TYR | B | 655 | 41.577 | −2.206 | 27.021 | 1.00 | 8.69 | C |
| ATOM | 1671 | CD2 | TYR | B | 655 | 40.725 | −3.241 | 27.055 | 1.00 | 9.00 | C |
| ATOM | 1672 | C | TYR | B | 655 | 38.074 | −6.888 | 24.825 | 1.00 | 6.04 | C |
| ATOM | 1673 | O | TYR | B | 655 | 38.605 | −7.806 | 24.221 | 1.00 | 6.92 | O |
| ATOM | 1674 | N | ARG | B | 656 | 36.985 | −7.059 | 25.528 | 1.00 | 5.36 | N |
| ATOM | 1675 | CA | ARG | B | 656 | 36.402 | −8.358 | 25.720 | 1.00 | 6.12 | C |
| ATOM | 1676 | CB | ARG | B | 656 | 35.157 | −8.238 | 26.641 | 1.00 | 5.83 | C |
| ATOM | 1677 | CG | ARG | B | 656 | 34.955 | −9.400 | 27.555 | 1.00 | 9.85 | C |
| ATOM | 1678 | CD | ARG | B | 656 | 33.527 | −9.895 | 27.730 | 1.00 | 14.72 | C |
| ATOM | 1679 | NE | ARG | B | 656 | 33.278 | −10.215 | 29.135 | 1.00 | 19.73 | N |
| ATOM | 1680 | CZ | ARG | B | 656 | 32.174 | −10.816 | 29.568 | 1.00 | 23.51 | C |
| ATOM | 1681 | NH1 | ARG | B | 656 | 31.217 | −11.188 | 28.707 | 1.00 | 26.59 | N |
| ATOM | 1682 | NH2 | ARG | B | 656 | 32.021 | −11.056 | 30.851 | 1.00 | 19.55 | N |
| ATOM | 1683 | C | ARG | B | 656 | 36.008 | −8.885 | 24.362 | 1.00 | 5.96 | C |
| ATOM | 1684 | O | ARG | B | 656 | 36.360 | −9.999 | 24.006 | 1.00 | 5.87 | O |
| ATOM | 1685 | N | LEU | B | 657 | 35.272 | −8.056 | 23.612 | 1.00 | 6.37 | N |
| ATOM | 1686 | CA | LEU | B | 657 | 34.794 | −8.416 | 22.293 | 1.00 | 6.12 | C |
| ATOM | 1687 | CB | LEU | B | 657 | 34.122 | −7.226 | 21.632 | 1.00 | 5.46 | C |
| ATOM | 1688 | CG | LEU | B | 657 | 32.951 | −7.388 | 20.640 | 1.00 | 7.16 | C |
| ATOM | 1689 | CD1 | LEU | B | 657 | 33.118 | −6.470 | 19.460 | 1.00 | 2.00 | C |
| ATOM | 1690 | CD2 | LEU | B | 657 | 32.700 | −8.824 | 20.156 | 1.00 | 5.57 | C |
| ATOM | 1691 | C | LEU | B | 657 | 36.009 | −8.820 | 21.465 | 1.00 | 6.67 | C |
| ATOM | 1692 | O | LEU | B | 657 | 36.065 | −9.905 | 20.924 | 1.00 | 6.08 | O |
| ATOM | 1693 | N | ALA | B | 658 | 37.004 | −7.943 | 21.405 | 1.00 | 7.27 | N |
| ATOM | 1694 | CA | ALA | B | 658 | 38.156 | −8.168 | 20.556 | 1.00 | 7.68 | C |
| ATOM | 1695 | CB | ALA | B | 658 | 39.093 | −7.045 | 20.658 | 1.00 | 9.00 | C |
| ATOM | 1696 | C | ALA | B | 658 | 38.893 | −9.422 | 20.884 | 1.00 | 8.43 | C |
| ATOM | 1697 | O | ALA | B | 658 | 39.271 | −10.196 | 19.981 | 1.00 | 8.28 | O |
| ATOM | 1698 | N | TYR | B | 659 | 39.143 | −9.615 | 22.174 | 1.00 | 8.68 | N |
| ATOM | 1699 | CA | TYR | B | 659 | 39.850 | −10.806 | 22.595 | 1.00 | 8.59 | C |
| ATOM | 1700 | CB | TYR | B | 659 | 40.177 | −10.769 | 24.087 | 1.00 | 8.82 | C |
| ATOM | 1701 | CG | TYR | B | 659 | 40.775 | −12.110 | 24.491 | 1.00 | 11.54 | C |
| ATOM | 1702 | CD1 | TYR | B | 659 | 42.087 | −12.435 | 24.133 | 1.00 | 7.96 | C |
| ATOM | 1703 | CE1 | TYR | B | 659 | 42.608 | −13.629 | 24.463 | 1.00 | 12.41 | C |
| ATOM | 1704 | CZ | TYR | B | 659 | 41.822 | −14.579 | 25.146 | 1.00 | 14.10 | C |
| ATOM | 1705 | OH | TYR | B | 659 | 42.353 | −15.803 | 25.433 | 1.00 | 10.91 | O |
| ATOM | 1706 | CE2 | TYR | B | 659 | 40.511 | −14.296 | 25.495 | 1.00 | 12.41 | C |
| ATOM | 1707 | CD2 | TYR | B | 659 | 39.996 | −13.070 | 25.164 | 1.00 | 11.29 | C |
| ATOM | 1708 | C | TYR | B | 659 | 39.053 | −12.090 | 22.229 | 1.00 | 8.10 | C |
| ATOM | 1709 | O | TYR | B | 659 | 39.591 | −13.017 | 21.663 | 1.00 | 7.91 | O |
| ATOM | 1710 | N | LEU | B | 660 | 37.773 | −12.143 | 22.548 | 1.00 | 8.11 | N |
| ATOM | 1711 | CA | LEU | B | 660 | 36.976 | −13.330 | 22.205 | 1.00 | 8.99 | C |
| ATOM | 1712 | CB | LEU | B | 660 | 35.494 | −13.149 | 22.601 | 1.00 | 8.89 | C |
| ATOM | 1713 | CG | LEU | B | 660 | 35.270 | −13.204 | 24.110 | 1.00 | 9.33 | C |
| ATOM | 1714 | CD1 | LEU | B | 660 | 33.943 | −12.684 | 24.547 | 1.00 | 9.61 | C |
| ATOM | 1715 | CD2 | LEU | B | 660 | 35.439 | −14.643 | 24.553 | 1.00 | 9.83 | C |
| ATOM | 1716 | C | LEU | B | 660 | 37.054 | −13.675 | 20.721 | 1.00 | 9.42 | C |
| ATOM | 1717 | O | LEU | B | 660 | 37.057 | −14.831 | 20.349 | 1.00 | 9.34 | O |
| ATOM | 1718 | N | ARG | B | 661 | 37.054 | −12.663 | 19.860 | 1.00 | 9.71 | N |
| ATOM | 1719 | CA | ARG | B | 661 | 37.050 | −12.968 | 18.462 | 1.00 | 10.45 | C |
| ATOM | 1720 | CB | ARG | B | 661 | 36.608 | −11.793 | 17.653 | 1.00 | 8.53 | C |
| ATOM | 1721 | CG | ARG | B | 661 | 35.229 | −11.304 | 17.915 | 1.00 | 7.97 | C |
| ATOM | 1722 | CD | ARG | B | 661 | 34.886 | −10.027 | 17.178 | 1.00 | 3.76 | C |
| ATOM | 1723 | NE | ARG | B | 661 | 33.472 | −9.762 | 16.983 | 1.00 | 2.70 | N |
| ATOM | 1724 | CZ | ARG | B | 661 | 32.973 | −8.629 | 16.467 | 1.00 | 3.60 | C |
| ATOM | 1725 | NH1 | ARG | B | 661 | 33.784 | −7.652 | 16.053 | 1.00 | 3.87 | N |
| ATOM | 1726 | NH2 | ARG | B | 661 | 31.652 | −8.468 | 16.330 | 1.00 | 2.00 | N |
| ATOM | 1727 | C | ARG | B | 661 | 38.478 | −13.448 | 18.096 | 1.00 | 12.78 | C |
| ATOM | 1728 | O | ARG | B | 661 | 38.625 | −14.338 | 17.273 | 1.00 | 13.90 | O |
| ATOM | 1729 | N | LEU | B | 662 | 39.508 | −12.879 | 18.735 | 1.00 | 13.10 | N |
| ATOM | 1730 | CA | LEU | B | 662 | 40.902 | −13.230 | 18.462 | 1.00 | 13.72 | C |
| ATOM | 1731 | CB | LEU | B | 662 | 41.803 | −12.308 | 19.265 | 1.00 | 13.06 | C |
| ATOM | 1732 | CG | LEU | B | 662 | 43.286 | −12.110 | 18.918 | 1.00 | 14.40 | C |
| ATOM | 1733 | CD1 | LEU | B | 662 | 44.132 | −12.325 | 20.086 | 1.00 | 9.20 | C |
| ATOM | 1734 | CD2 | LEU | B | 662 | 43.829 | −12.854 | 17.630 | 1.00 | 15.62 | C |
| ATOM | 1735 | C | LEU | B | 662 | 41.198 | −14.658 | 18.904 | 1.00 | 15.28 | C |
| ATOM | 1736 | O | LEU | B | 662 | 41.900 | −15.448 | 18.206 | 1.00 | 15.63 | O |
| ATOM | 1737 | N | ASN | B | 663 | 40.656 | −14.980 | 20.082 | 1.00 | 15.58 | N |
| ATOM | 1738 | CA | ASN | B | 663 | 40.829 | −16.268 | 20.666 | 1.00 | 15.36 | C |
| ATOM | 1739 | CB | ASN | B | 663 | 40.287 | −16.291 | 22.090 | 1.00 | 15.75 | C |
| ATOM | 1740 | CG | ASN | B | 663 | 40.059 | −17.727 | 22.606 | 1.00 | 16.02 | C |
| ATOM | 1741 | OD1 | ASN | B | 663 | 38.985 | −18.274 | 22.388 | 1.00 | 16.31 | O |
| ATOM | 1742 | ND2 | ASN | B | 663 | 41.057 | −18.320 | 23.284 | 1.00 | 9.15 | N |
| ATOM | 1743 | C | ASN | B | 663 | 40.143 | −17.296 | 19.840 | 1.00 | 15.24 | C |
| ATOM | 1744 | O | ASN | B | 663 | 40.592 | −18.403 | 19.771 | 1.00 | 15.75 | O |
| ATOM | 1745 | N | THR | B | 664 | 39.030 | −16.930 | 19.225 | 1.00 | 15.92 | N |
| ATOM | 1746 | CA | THR | B | 664 | 38.274 | −17.868 | 18.396 | 1.00 | 15.65 | C |
| ATOM | 1747 | CB | THR | B | 664 | 36.934 | −17.267 | 17.986 | 1.00 | 16.03 | C |
| ATOM | 1748 | OG1 | THR | B | 664 | 36.179 | −16.882 | 19.145 | 1.00 | 18.60 | O |
| ATOM | 1749 | CG2 | THR | B | 664 | 36.076 | −18.315 | 17.271 | 1.00 | 12.83 | C |
| ATOM | 1750 | C | THR | B | 664 | 39.059 | −18.201 | 17.126 | 1.00 | 15.89 | C |
| ATOM | 1751 | O | THR | B | 664 | 39.095 | −19.335 | 16.740 | 1.00 | 15.40 | O |
| ATOM | 1752 | N | LEU | B | 665 | 39.661 | −17.208 | 16.472 | 1.00 | 16.10 | N |
| ATOM | 1753 | CA | LEU | B | 665 | 40.453 | −17.463 | 15.290 | 1.00 | 17.99 | C |
| ATOM | 1754 | CB | LEU | B | 665 | 40.700 | −16.171 | 14.515 | 1.00 | 18.03 | C |
| ATOM | 1755 | CG | LEU | B | 665 | 39.504 | −15.473 | 13.840 | 1.00 | 17.81 | C |
| ATOM | 1756 | CD1 | LEU | B | 665 | 39.932 | −14.082 | 13.322 | 1.00 | 14.11 | C |
| ATOM | 1757 | CD2 | LEU | B | 665 | 38.978 | −16.273 | 12.715 | 1.00 | 15.62 | C |
| ATOM | 1758 | C | LEU | B | 665 | 41.816 | −18.142 | 15.599 | 1.00 | 18.90 | C |
| ATOM | 1759 | O | LEU | B | 665 | 42.290 | −18.978 | 14.843 | 1.00 | 18.38 | O |
| ATOM | 1760 | N | CYS | B | 666 | 42.452 | −17.769 | 16.702 | 1.00 | 19.38 | N |
| ATOM | 1761 | CA | CYS | B | 666 | 43.727 | −18.381 | 17.036 | 1.00 | 19.52 | C |
| ATOM | 1762 | CB | CYS | B | 666 | 44.435 | −17.649 | 18.192 | 1.00 | 20.43 | C |
| ATOM | 1763 | SG | CYS | B | 666 | 45.104 | −16.057 | 17.678 | 1.00 | 19.13 | S |
| ATOM | 1764 | C | CYS | B | 666 | 43.614 | −19.808 | 17.430 | 1.00 | 19.42 | C |
| ATOM | 1765 | O | CYS | B | 666 | 44.601 | −20.549 | 17.260 | 1.00 | 19.57 | O |
| ATOM | 1766 | N | GLU | B | 667 | 42.463 | −20.191 | 18.009 | 1.00 | 18.97 | N |
| ATOM | 1767 | CA | GLU | B | 667 | 42.273 | −21.576 | 18.478 | 1.00 | 18.84 | C |
| ATOM | 1768 | CB | GLU | B | 667 | 41.170 | −21.680 | 19.536 | 1.00 | 18.36 | C |
| ATOM | 1769 | CG | GLU | B | 667 | 40.892 | −23.085 | 20.048 | 1.00 | 17.77 | C |
| ATOM | 1770 | CD | GLU | B | 667 | 40.404 | −23.151 | 21.526 | 1.00 | 18.28 | C |
| ATOM | 1771 | OE1 | GLU | B | 667 | 39.851 | −24.235 | 21.937 | 1.00 | 12.51 | O |
| ATOM | 1772 | OE2 | GLU | B | 667 | 40.586 | −22.126 | 22.277 | 1.00 | 16.34 | O |
| ATOM | 1773 | C | GLU | B | 667 | 42.019 | −22.508 | 17.291 | 1.00 | 19.46 | C |
| ATOM | 1774 | O | GLU | B | 667 | 42.257 | −23.691 | 17.387 | 1.00 | 20.81 | O |
| ATOM | 1775 | N | ARG | B | 668 | 41.558 | −21.979 | 16.166 | 1.00 | 18.39 | N |
| ATOM | 1776 | CA | ARG | B | 668 | 41.330 | −22.817 | 15.012 | 1.00 | 18.41 | C |
| ATOM | 1777 | CB | ARG | B | 668 | 40.005 | −22.454 | 14.376 | 1.00 | 19.07 | C |
| ATOM | 1778 | CG | ARG | B | 668 | 38.775 | −22.369 | 15.341 | 1.00 | 20.80 | C |
| ATOM | 1779 | CD | ARG | B | 668 | 37.477 | −21.891 | 14.616 | 1.00 | 19.53 | C |
| ATOM | 1780 | NE | ARG | B | 668 | 36.292 | −21.858 | 15.484 | 1.00 | 23.37 | N |
| ATOM | 1781 | CZ | ARG | B | 668 | 35.058 | −21.419 | 15.127 | 1.00 | 22.67 | C |
| ATOM | 1782 | NH1 | ARG | B | 668 | 34.778 | −20.972 | 13.896 | 1.00 | 20.39 | N |
| ATOM | 1783 | NH2 | ARG | B | 668 | 34.094 | −21.407 | 16.035 | 1.00 | 20.67 | N |
| ATOM | 1784 | C | ARG | B | 668 | 42.450 | −22.719 | 13.950 | 1.00 | 18.34 | C |
| ATOM | 1785 | O | ARG | B | 668 | 42.651 | −23.613 | 13.122 | 1.00 | 17.26 | O |
| ATOM | 1786 | N | LEU | B | 669 | 43.206 | −21.630 | 13.988 | 1.00 | 18.42 | N |
| ATOM | 1787 | CA | LEU | B | 669 | 44.255 | −21.413 | 13.001 | 1.00 | 18.63 | C |
| ATOM | 1788 | CB | LEU | B | 669 | 44.105 | −19.999 | 12.412 | 1.00 | 19.29 | C |
| ATOM | 1789 | CG | LEU | B | 669 | 43.433 | −19.874 | 11.029 | 1.00 | 20.70 | C |
| ATOM | 1790 | CD1 | LEU | B | 669 | 42.191 | −20.771 | 10.861 | 1.00 | 18.65 | C |
| ATOM | 1791 | CD2 | LEU | B | 669 | 43.167 | −18.385 | 10.606 | 1.00 | 15.39 | C |
| ATOM | 1792 | C | LEU | B | 669 | 45.677 | −21.649 | 13.553 | 1.00 | 18.31 | C |
| ATOM | 1793 | O | LEU | B | 669 | 46.559 | −22.131 | 12.850 | 1.00 | 17.46 | O |
| ATOM | 1794 | N | LEU | B | 670 | 45.892 | −21.320 | 14.819 | 1.00 | 17.97 | N |
| ATOM | 1795 | CA | LEU | B | 670 | 47.206 | −21.450 | 15.382 | 1.00 | 18.50 | C |
| ATOM | 1796 | CB | LEU | B | 670 | 47.699 | −20.070 | 15.762 | 1.00 | 18.75 | C |
| ATOM | 1797 | CG | LEU | B | 670 | 47.828 | −19.118 | 14.568 | 1.00 | 17.36 | C |
| ATOM | 1798 | CD1 | LEU | B | 670 | 48.413 | −17.828 | 15.007 | 1.00 | 15.48 | C |
| ATOM | 1799 | CD2 | LEU | B | 670 | 48.698 | −19.778 | 13.477 | 1.00 | 14.62 | C |
| ATOM | 1800 | C | LEU | B | 670 | 47.340 | −22.401 | 16.552 | 1.00 | 19.33 | C |
| ATOM | 1801 | O | LEU | B | 670 | 48.121 | −22.162 | 17.448 | 1.00 | 19.41 | O |
| ATOM | 1802 | N | SER | B | 671 | 46.646 | −23.529 | 16.514 | 1.00 | 21.39 | N |
| ATOM | 1803 | CA | SER | B | 671 | 46.704 | −24.470 | 17.632 | 1.00 | 23.09 | C |
| ATOM | 1804 | CB | SER | B | 671 | 45.665 | −25.575 | 17.484 | 1.00 | 22.45 | C |
| ATOM | 1805 | OG | SER | B | 671 | 45.803 | −26.222 | 16.227 | 1.00 | 23.79 | O |
| ATOM | 1806 | C | SER | B | 671 | 48.081 | −25.084 | 17.765 | 1.00 | 24.29 | C |
| ATOM | 1807 | O | SER | B | 671 | 48.467 | −25.474 | 18.858 | 1.00 | 24.66 | O |
| ATOM | 1808 | N | GLU | B | 672 | 48.806 | −25.180 | 16.644 | 1.00 | 25.50 | N |
| ATOM | 1809 | CA | GLU | B | 672 | 50.152 | −25.749 | 16.637 | 1.00 | 25.94 | C |
| ATOM | 1810 | CB | GLU | B | 672 | 50.715 | −25.898 | 15.221 | 1.00 | 27.18 | C |
| ATOM | 1811 | CG | GLU | B | 672 | 49.983 | −26.842 | 14.267 | 1.00 | 31.37 | C |
| ATOM | 1812 | CD | GLU | B | 672 | 50.420 | −26.667 | 12.787 | 1.00 | 36.21 | C |
| ATOM | 1813 | OE1 | GLU | B | 672 | 50.113 | −25.634 | 12.135 | 1.00 | 34.47 | O |
| ATOM | 1814 | OE2 | GLU | B | 672 | 51.058 | −27.605 | 12.247 | 1.00 | 40.18 | O |
| ATOM | 1815 | C | GLU | B | 672 | 51.097 | −24.868 | 17.431 | 1.00 | 25.14 | C |
| ATOM | 1816 | O | GLU | B | 672 | 52.048 | −25.379 | 18.006 | 1.00 | 25.38 | O |
| ATOM | 1817 | N | HIS | B | 673 | 50.867 | −23.548 | 17.439 | 1.00 | 24.39 | N |
| ATOM | 1818 | CA | HIS | B | 673 | 51.727 | −22.613 | 18.226 | 1.00 | 23.25 | C |
| ATOM | 1819 | CB | HIS | B | 673 | 52.507 | −21.670 | 17.308 | 1.00 | 22.74 | C |
| ATOM | 1820 | CG | HIS | B | 673 | 52.982 | −22.304 | 16.035 | 1.00 | 24.74 | C |
| ATOM | 1821 | ND1 | HIS | B | 673 | 52.234 | −22.309 | 14.874 | 1.00 | 23.69 | N |
| ATOM | 1822 | CE1 | HIS | B | 673 | 52.909 | −22.931 | 13.920 | 1.00 | 23.68 | C |
| ATOM | 1823 | NE2 | HIS | B | 673 | 54.075 | −23.316 | 14.413 | 1.00 | 23.92 | N |
| ATOM | 1824 | CD2 | HIS | B | 673 | 54.142 | −22.947 | 15.737 | 1.00 | 25.13 | C |
| ATOM | 1825 | C | HIS | B | 673 | 50.904 | −21.811 | 19.255 | 1.00 | 21.86 | C |
| ATOM | 1826 | O | HIS | B | 673 | 50.723 | −20.598 | 19.131 | 1.00 | 21.72 | O |
| ATOM | 1827 | N | PRO | B | 674 | 50.422 | −22.506 | 20.274 | 1.00 | 20.75 | N |
| ATOM | 1828 | CA | PRO | B | 674 | 49.579 | −21.913 | 21.309 | 1.00 | 19.67 | C |
| ATOM | 1829 | CB | PRO | B | 674 | 49.540 | −23.021 | 22.379 | 1.00 | 19.92 | C |
| ATOM | 1830 | CG | PRO | B | 674 | 49.687 | −24.247 | 21.623 | 1.00 | 19.13 | C |
| ATOM | 1831 | CD | PRO | B | 674 | 50.691 | −23.934 | 20.547 | 1.00 | 20.60 | C |
| ATOM | 1832 | C | PRO | B | 674 | 50.126 | −20.630 | 21.901 | 1.00 | 18.69 | C |
| ATOM | 1833 | O | PRO | B | 674 | 49.369 | −19.848 | 22.443 | 1.00 | 18.47 | O |
| ATOM | 1834 | N | GLU | B | 675 | 51.420 | −20.396 | 21.773 | 1.00 | 18.13 | N |
| ATOM | 1835 | CA | GLU | B | 675 | 51.998 | −19.194 | 22.326 | 1.00 | 18.19 | C |
| ATOM | 1836 | CB | GLU | B | 675 | 53.517 | −19.319 | 22.414 | 1.00 | 18.97 | C |
| ATOM | 1837 | CG | GLU | B | 675 | 54.274 | −19.156 | 21.088 | 1.00 | 19.76 | C |
| ATOM | 1838 | CD | GLU | B | 675 | 54.239 | −20.376 | 20.186 | 1.00 | 18.67 | C |
| ATOM | 1839 | OE1 | GLU | B | 675 | 53.642 | −21.410 | 20.560 | 1.00 | 16.34 | O |
| ATOM | 1840 | OE2 | GLU | B | 675 | 54.824 | −20.283 | 19.085 | 1.00 | 18.78 | O |
| ATOM | 1841 | C | GLU | B | 675 | 51.657 | −17.940 | 21.548 | 1.00 | 17.58 | C |
| ATOM | 1842 | O | GLU | B | 675 | 51.660 | −16.886 | 22.121 | 1.00 | 18.20 | O |
| ATOM | 1843 | N | LEU | B | 676 | 51.382 | −18.030 | 20.250 | 1.00 | 16.44 | N |
| ATOM | 1844 | CA | LEU | B | 676 | 51.143 | −16.803 | 19.469 | 1.00 | 15.70 | C |
| ATOM | 1845 | CB | LEU | B | 676 | 51.006 | −17.104 | 17.965 | 1.00 | 15.18 | C |
| ATOM | 1846 | CG | LEU | B | 676 | 52.243 | −17.715 | 17.348 | 1.00 | 13.23 | C |
| ATOM | 1847 | CD1 | LEU | B | 676 | 52.008 | −17.973 | 15.866 | 1.00 | 12.22 | C |
| ATOM | 1848 | CD2 | LEU | B | 676 | 53.405 | −16.751 | 17.566 | 1.00 | 8.01 | C |
| ATOM | 1849 | C | LEU | B | 676 | 49.986 | −15.898 | 19.905 | 1.00 | 14.78 | C |
| ATOM | 1850 | O | LEU | B | 676 | 50.038 | −14.706 | 19.703 | 1.00 | 14.81 | O |
| ATOM | 1851 | N | GLU | B | 677 | 48.933 | −16.455 | 20.473 | 1.00 | 14.63 | N |
| ATOM | 1852 | CA | GLU | B | 677 | 47.758 | −15.636 | 20.830 | 1.00 | 14.03 | C |
| ATOM | 1853 | CB | GLU | B | 677 | 46.618 | −16.503 | 21.396 | 1.00 | 12.86 | C |
| ATOM | 1854 | CG | GLU | B | 677 | 45.378 | −15.737 | 21.847 | 1.00 | 14.72 | C |
| ATOM | 1855 | CD | GLU | B | 677 | 44.387 | −16.590 | 22.666 | 1.00 | 17.27 | C |
| ATOM | 1856 | OE1 | GLU | B | 677 | 43.557 | −17.338 | 22.098 | 1.00 | 15.74 | O |
| ATOM | 1857 | OE2 | GLU | B | 677 | 44.427 | −16.530 | 23.901 | 1.00 | 16.88 | O |
| ATOM | 1858 | C | GLU | B | 677 | 48.062 | −14.467 | 21.749 | 1.00 | 13.75 | C |
| ATOM | 1859 | O | GLU | B | 677 | 47.589 | −13.318 | 21.520 | 1.00 | 14.06 | O |
| ATOM | 1860 | N | HIS | B | 678 | 48.841 | −14.733 | 22.788 | 1.00 | 13.20 | N |
| ATOM | 1861 | CA | HIS | B | 678 | 49.142 | −13.689 | 23.765 | 1.00 | 14.08 | C |
| ATOM | 1862 | CB | HIS | B | 678 | 49.775 | −14.274 | 25.031 | 1.00 | 14.28 | C |
| ATOM | 1863 | CG | HIS | B | 678 | 49.611 | −13.422 | 26.257 | 1.00 | 16.75 | C |
| ATOM | 1864 | ND1 | HIS | B | 678 | 48.616 | −12.466 | 26.386 | 1.00 | 18.50 | N |
| ATOM | 1865 | CE1 | HIS | B | 678 | 48.698 | −11.904 | 27.580 | 1.00 | 17.30 | C |
| ATOM | 1866 | NE2 | HIS | B | 678 | 49.721 | −12.439 | 28.220 | 1.00 | 16.02 | N |
| ATOM | 1867 | CD2 | HIS | B | 678 | 50.306 | −13.393 | 27.418 | 1.00 | 15.51 | C |
| ATOM | 1868 | C | HIS | B | 678 | 50.019 | −12.585 | 23.184 | 1.00 | 13.46 | C |
| ATOM | 1869 | O | HIS | B | 678 | 49.895 | −11.445 | 23.578 | 1.00 | 13.27 | O |
| ATOM | 1870 | N | ILE | B | 679 | 50.907 | −12.922 | 22.248 | 1.00 | 13.66 | N |
| ATOM | 1871 | CA | ILE | B | 679 | 51.758 | −11.903 | 21.623 | 1.00 | 13.87 | C |
| ATOM | 1872 | CB | ILE | B | 679 | 52.940 | −12.548 | 20.884 | 1.00 | 14.37 | C |
| ATOM | 1873 | CG1 | ILE | B | 679 | 53.840 | −13.349 | 21.826 | 1.00 | 15.38 | C |
| ATOM | 1874 | CD1 | ILE | B | 679 | 54.959 | −14.175 | 21.105 | 1.00 | 11.31 | C |
| ATOM | 1875 | CG2 | ILE | B | 679 | 53.795 | −11.481 | 20.210 | 1.00 | 16.19 | C |
| ATOM | 1876 | C | ILE | B | 679 | 50.914 | −11.020 | 20.660 | 1.00 | 13.66 | C |
| ATOM | 1877 | O | ILE | B | 679 | 51.018 | −9.776 | 20.652 | 1.00 | 12.95 | O |
| ATOM | 1878 | N | ILE | B | 680 | 50.072 | −11.675 | 19.860 | 1.00 | 12.55 | N |
| ATOM | 1879 | CA | ILE | B | 680 | 49.162 | −10.987 | 18.958 | 1.00 | 11.61 | C |
| ATOM | 1880 | CB | ILE | B | 680 | 48.355 | −12.008 | 18.110 | 1.00 | 11.65 | C |
| ATOM | 1881 | CG1 | ILE | B | 680 | 49.280 | −12.924 | 17.344 | 1.00 | 9.78 | C |
| ATOM | 1882 | CD1 | ILE | B | 680 | 48.562 | −13.950 | 16.468 | 1.00 | 9.87 | C |
| ATOM | 1883 | CG2 | ILE | B | 680 | 47.473 | −11.289 | 17.076 | 1.00 | 12.42 | C |
| ATOM | 1884 | C | ILE | B | 680 | 48.199 | −10.085 | 19.727 | 1.00 | 11.07 | C |
| ATOM | 1885 | O | ILE | B | 680 | 47.921 | −8.939 | 19.315 | 1.00 | 10.14 | O |
| ATOM | 1886 | N | TRP | B | 681 | 47.693 | −10.596 | 20.844 | 1.00 | 10.25 | N |
| ATOM | 1887 | CA | TRP | B | 681 | 46.736 | −9.810 | 21.625 | 1.00 | 9.98 | C |
| ATOM | 1888 | CB | TRP | B | 681 | 46.107 | −10.594 | 22.784 | 1.00 | 10.30 | C |
| ATOM | 1889 | CG | TRP | B | 681 | 45.516 | −9.703 | 23.783 | 1.00 | 10.20 | C |
| ATOM | 1890 | CD1 | TRP | B | 681 | 45.943 | −9.489 | 25.083 | 1.00 | 8.14 | C |
| ATOM | 1891 | NE1 | TRP | B | 681 | 45.123 | −8.570 | 25.691 | 1.00 | 11.57 | N |
| ATOM | 1892 | CE2 | TRP | B | 681 | 44.174 | −8.153 | 24.780 | 1.00 | 10.57 | C |
| ATOM | 1893 | CD2 | TRP | B | 681 | 44.396 | −8.851 | 23.580 | 1.00 | 12.27 | C |
| ATOM | 1894 | CE3 | TRP | B | 681 | 43.548 | −8.595 | 22.486 | 1.00 | 12.34 | C |
| ATOM | 1895 | CZ3 | TRP | B | 681 | 42.549 | −7.671 | 22.623 | 1.00 | 8.26 | C |
| ATOM | 1896 | CH2 | TRP | B | 681 | 42.385 | −6.984 | 23.815 | 1.00 | 9.93 | C |
| ATOM | 1897 | CZ2 | TRP | B | 681 | 43.188 | −7.210 | 24.900 | 1.00 | 8.58 | C |
| ATOM | 1898 | C | TRP | B | 681 | 47.427 | −8.596 | 22.136 | 1.00 | 9.60 | C |
| ATOM | 1899 | O | TRP | B | 681 | 46.818 | −7.521 | 22.282 | 1.00 | 10.07 | O |
| ATOM | 1900 | N | THR | B | 682 | 48.710 | −8.757 | 22.431 | 1.00 | 9.03 | N |
| ATOM | 1901 | CA | THR | B | 682 | 49.523 | −7.629 | 22.897 | 1.00 | 7.57 | C |
| ATOM | 1902 | CB | THR | B | 682 | 50.887 | −8.117 | 23.316 | 1.00 | 7.89 | C |
| ATOM | 1903 | OG1 | THR | B | 682 | 50.760 | −8.912 | 24.499 | 1.00 | 7.20 | O |
| ATOM | 1904 | CG2 | THR | B | 682 | 51.776 | −6.939 | 23.723 | 1.00 | 9.26 | C |
| ATOM | 1905 | C | THR | B | 682 | 49.664 | −6.547 | 21.858 | 1.00 | 5.80 | C |
| ATOM | 1906 | O | THR | B | 682 | 49.522 | −5.428 | 22.192 | 1.00 | 5.05 | O |
| ATOM | 1907 | N | LEU | B | 683 | 50.010 | −6.900 | 20.621 | 1.00 | 5.57 | N |
| ATOM | 1908 | CA | LEU | B | 683 | 50.082 | −5.949 | 19.482 | 1.00 | 5.60 | C |
| ATOM | 1909 | CB | LEU | B | 683 | 50.485 | −6.672 | 18.233 | 1.00 | 4.78 | C |
| ATOM | 1910 | CG | LEU | B | 683 | 51.117 | −5.923 | 17.106 | 1.00 | 6.57 | C |
| ATOM | 1911 | CD1 | LEU | B | 683 | 50.996 | −6.590 | 15.661 | 1.00 | 4.62 | C |
| ATOM | 1912 | CD2 | LEU | B | 683 | 50.646 | −4.513 | 17.107 | 1.00 | 8.25 | C |
| ATOM | 1913 | C | LEU | B | 683 | 48.706 | −5.331 | 19.208 | 1.00 | 5.57 | C |
| ATOM | 1914 | O | LEU | B | 683 | 48.604 | −4.143 | 18.911 | 1.00 | 4.88 | O |
| ATOM | 1915 | N | PHE | B | 684 | 47.659 | −6.158 | 19.336 | 1.00 | 6.06 | N |
| ATOM | 1916 | CA | PHE | B | 684 | 46.291 | −5.766 | 19.089 | 1.00 | 6.35 | C |
| ATOM | 1917 | CB | PHE | B | 684 | 45.365 | −6.971 | 19.277 | 1.00 | 6.20 | C |
| ATOM | 1918 | CG | PHE | B | 684 | 43.963 | −6.775 | 18.749 | 1.00 | 5.04 | C |
| ATOM | 1919 | CD1 | PHE | B | 684 | 43.516 | −5.561 | 18.371 | 1.00 | 5.84 | C |
| ATOM | 1920 | CE1 | PHE | B | 684 | 42.232 | −5.363 | 17.912 | 1.00 | 9.04 | C |
| ATOM | 1921 | CZ | PHE | B | 684 | 41.368 | −6.426 | 17.831 | 1.00 | 10.79 | C |
| ATOM | 1922 | CE2 | PHE | B | 684 | 41.793 | −7.674 | 18.174 | 1.00 | 10.95 | C |
| ATOM | 1923 | CD2 | PHE | B | 684 | 43.096 | −7.845 | 18.653 | 1.00 | 10.41 | C |
| ATOM | 1924 | C | PHE | B | 684 | 45.885 | −4.661 | 20.010 | 1.00 | 6.76 | C |
| ATOM | 1925 | O | PHE | B | 684 | 45.351 | −3.634 | 19.545 | 1.00 | 7.52 | O |
| ATOM | 1926 | N | GLN | B | 685 | 46.183 | −4.856 | 21.293 | 1.00 | 6.72 | N |
| ATOM | 1927 | CA | GLN | B | 685 | 45.785 | −3.957 | 22.379 | 1.00 | 6.81 | C |
| ATOM | 1928 | CB | GLN | B | 685 | 45.903 | −4.668 | 23.756 | 1.00 | 7.65 | C |
| ATOM | 1929 | CG | GLN | B | 685 | 45.463 | −3.884 | 25.038 | 1.00 | 10.43 | C |
| ATOM | 1930 | CD | GLN | B | 685 | 46.325 | −2.667 | 25.299 | 1.00 | 16.01 | C |
| ATOM | 1931 | OE1 | GLN | B | 685 | 45.818 | −1.596 | 25.619 | 1.00 | 13.41 | O |
| ATOM | 1932 | NE2 | GLN | B | 685 | 47.650 | −2.813 | 25.090 | 1.00 | 17.77 | N |
| ATOM | 1933 | C | GLN | B | 685 | 46.597 | −2.708 | 22.317 | 1.00 | 6.15 | C |
| ATOM | 1934 | O | GLN | B | 685 | 46.095 | −1.608 | 22.604 | 1.00 | 5.65 | O |
| ATOM | 1935 | N | HIS | B | 686 | 47.843 | −2.870 | 21.897 | 1.00 | 5.79 | N |
| ATOM | 1936 | CA | HIS | B | 686 | 48.776 | −1.748 | 21.793 | 1.00 | 6.80 | C |
| ATOM | 1937 | CB | HIS | B | 686 | 50.212 | −2.230 | 21.429 | 1.00 | 6.29 | C |
| ATOM | 1938 | CG | HIS | B | 686 | 51.124 | −1.132 | 20.934 | 1.00 | 12.07 | C |
| ATOM | 1939 | ND1 | HIS | B | 686 | 51.758 | −0.238 | 21.781 | 1.00 | 17.34 | N |
| ATOM | 1940 | CE1 | HIS | B | 686 | 52.481 | 0.616 | 21.068 | 1.00 | 14.84 | C |
| ATOM | 1941 | NE2 | HIS | B | 686 | 52.355 | 0.303 | 19.791 | 1.00 | 13.76 | N |
| ATOM | 1942 | CD2 | HIS | B | 686 | 51.509 | −0.783 | 19.676 | 1.00 | 13.72 | C |
| ATOM | 1943 | C | HIS | B | 686 | 48.241 | −0.747 | 20.772 | 1.00 | 6.86 | C |
| ATOM | 1944 | O | HIS | B | 686 | 48.443 | 0.473 | 20.897 | 1.00 | 5.59 | O |
| ATOM | 1945 | N | THR | B | 687 | 47.531 | −1.302 | 19.791 | 1.00 | 6.24 | N |
| ATOM | 1946 | CA | THR | B | 687 | 47.058 | −0.557 | 18.692 | 1.00 | 7.58 | C |
| ATOM | 1947 | CB | THR | B | 687 | 46.784 | −1.518 | 17.502 | 1.00 | 8.36 | C |
| ATOM | 1948 | OG1 | THR | B | 687 | 48.012 | −2.196 | 17.143 | 1.00 | 6.50 | O |
| ATOM | 1949 | CG2 | THR | B | 687 | 46.448 | −0.754 | 16.252 | 1.00 | 6.00 | C |
| ATOM | 1950 | C | THR | B | 687 | 45.871 | 0.216 | 19.119 | 1.00 | 8.56 | C |
| ATOM | 1951 | O | THR | B | 687 | 45.839 | 1.429 | 19.017 | 1.00 | 10.02 | O |
| ATOM | 1952 | N | LEU | B | 688 | 44.898 | −0.482 | 19.673 | 1.00 | 9.41 | N |
| ATOM | 1953 | CA | LEU | B | 688 | 43.666 | 0.139 | 20.069 | 1.00 | 8.15 | C |
| ATOM | 1954 | CB | LEU | B | 688 | 42.762 | −0.907 | 20.687 | 1.00 | 7.39 | C |
| ATOM | 1955 | CG | LEU | B | 688 | 42.153 | −1.890 | 19.723 | 1.00 | 10.18 | C |
| ATOM | 1956 | CD1 | LEU | B | 688 | 41.445 | −3.058 | 20.415 | 1.00 | 7.27 | C |
| ATOM | 1957 | CD2 | LEU | B | 688 | 41.142 | −1.098 | 18.791 | 1.00 | 13.68 | C |
| ATOM | 1958 | C | LEU | B | 688 | 43.962 | 1.272 | 21.036 | 1.00 | 8.20 | C |
| ATOM | 1959 | O | LEU | B | 688 | 43.227 | 2.296 | 21.085 | 1.00 | 7.09 | O |
| ATOM | 1960 | N | GLN | B | 689 | 45.030 | 1.084 | 21.812 | 1.00 | 8.37 | N |
| ATOM | 1961 | CA | GLN | B | 689 | 45.360 | 2.012 | 22.883 | 1.00 | 9.31 | C |
| ATOM | 1962 | CB | GLN | B | 689 | 45.965 | 1.285 | 24.062 | 1.00 | 9.91 | C |
| ATOM | 1963 | CG | GLN | B | 689 | 46.488 | 2.136 | 25.193 | 1.00 | 11.42 | C |
| ATOM | 1964 | CD | GLN | B | 689 | 47.388 | 1.309 | 26.139 | 1.00 | 17.91 | C |
| ATOM | 1965 | OE1 | GLN | B | 689 | 47.428 | 1.509 | 27.368 | 1.00 | 16.41 | O |
| ATOM | 1966 | NE2 | GLN | B | 689 | 48.116 | 0.373 | 25.548 | 1.00 | 22.03 | N |
| ATOM | 1967 | C | GLN | B | 689 | 46.291 | 3.107 | 22.475 | 1.00 | 10.03 | C |
| ATOM | 1968 | O | GLN | B | 689 | 46.151 | 4.202 | 22.972 | 1.00 | 10.38 | O |
| ATOM | 1969 | N | ASN | B | 690 | 47.242 | 2.842 | 21.585 | 1.00 | 10.31 | N |
| ATOM | 1970 | CA | ASN | B | 690 | 48.195 | 3.873 | 21.243 | 1.00 | 10.87 | C |
| ATOM | 1971 | CB | ASN | B | 690 | 49.594 | 3.477 | 21.680 | 1.00 | 11.41 | C |
| ATOM | 1972 | CG | ASN | B | 690 | 49.655 | 3.038 | 23.163 | 1.00 | 14.58 | C |
| ATOM | 1973 | OD1 | ASN | B | 690 | 49.287 | 3.797 | 24.079 | 1.00 | 12.40 | O |
| ATOM | 1974 | ND2 | ASN | B | 690 | 50.129 | 1.806 | 23.395 | 1.00 | 13.56 | N |
| ATOM | 1975 | C | ASN | B | 690 | 48.200 | 4.283 | 19.791 | 1.00 | 11.32 | C |
| ATOM | 1976 | O | ASN | B | 690 | 48.675 | 5.346 | 19.472 | 1.00 | 12.34 | O |
| ATOM | 1977 | N | GLU | B | 691 | 47.670 | 3.471 | 18.897 | 1.00 | 11.49 | N |
| ATOM | 1978 | CA | GLU | B | 691 | 47.629 | 3.873 | 17.493 | 1.00 | 12.32 | C |
| ATOM | 1979 | CB | GLU | B | 691 | 48.418 | 2.895 | 16.589 | 1.00 | 13.24 | C |
| ATOM | 1980 | CG | GLU | B | 691 | 49.824 | 2.554 | 17.042 | 1.00 | 16.51 | C |
| ATOM | 1981 | CD | GLU | B | 691 | 50.774 | 3.763 | 16.964 | 1.00 | 27.49 | C |
| ATOM | 1982 | OE1 | GLU | B | 691 | 50.583 | 4.649 | 16.071 | 1.00 | 28.02 | O |
| ATOM | 1983 | OE2 | GLU | B | 691 | 51.722 | 3.845 | 17.795 | 1.00 | 27.57 | O |
| ATOM | 1984 | C | GLU | B | 691 | 46.173 | 3.941 | 17.054 | 1.00 | 11.32 | C |
| ATOM | 1985 | O | GLU | B | 691 | 45.826 | 3.491 | 15.982 | 1.00 | 9.71 | O |
| ATOM | 1986 | N | TYR | B | 692 | 45.346 | 4.520 | 17.925 | 1.00 | 12.17 | N |
| ATOM | 1987 | CA | TYR | B | 692 | 43.892 | 4.662 | 17.749 | 1.00 | 11.54 | C |
| ATOM | 1988 | CB | TYR | B | 692 | 43.246 | 5.296 | 18.968 | 1.00 | 10.36 | C |
| ATOM | 1989 | CG | TYR | B | 692 | 43.781 | 6.634 | 19.276 | 1.00 | 12.35 | C |
| ATOM | 1990 | CD1 | TYR | B | 692 | 43.456 | 7.724 | 18.465 | 1.00 | 13.10 | C |
| ATOM | 1991 | CE1 | TYR | B | 692 | 43.952 | 8.952 | 18.712 | 1.00 | 12.96 | C |
| ATOM | 1992 | CZ | TYR | B | 692 | 44.790 | 9.139 | 19.800 | 1.00 | 15.72 | C |
| ATOM | 1993 | OH | TYR | B | 692 | 45.273 | 10.416 | 20.038 | 1.00 | 18.87 | O |
| ATOM | 1994 | CE2 | TYR | B | 692 | 45.130 | 8.076 | 20.641 | 1.00 | 12.33 | C |
| ATOM | 1995 | CD2 | TYR | B | 692 | 44.643 | 6.833 | 20.368 | 1.00 | 10.45 | C |
| ATOM | 1996 | C | TYR | B | 692 | 43.456 | 5.375 | 16.481 | 1.00 | 11.74 | C |
| ATOM | 1997 | O | TYR | B | 692 | 42.342 | 5.147 | 16.015 | 1.00 | 12.21 | O |
| ATOM | 1998 | N | GLU | B | 693 | 44.334 | 6.181 | 15.881 | 1.00 | 11.26 | N |
| ATOM | 1999 | CA | GLU | B | 693 | 43.993 | 6.843 | 14.644 | 1.00 | 10.21 | C |
| ATOM | 2000 | CB | GLU | B | 693 | 45.024 | 7.880 | 14.262 | 1.00 | 10.93 | C |
| ATOM | 2001 | CG | GLU | B | 693 | 44.958 | 9.126 | 15.116 | 1.00 | 12.97 | C |
| ATOM | 2002 | CD | GLU | B | 693 | 43.629 | 9.809 | 15.027 | 1.00 | 16.35 | C |
| ATOM | 2003 | OE1 | GLU | B | 693 | 43.221 | 10.421 | 16.037 | 1.00 | 22.86 | O |
| ATOM | 2004 | OE2 | GLU | B | 693 | 42.974 | 9.751 | 13.961 | 1.00 | 15.98 | O |
| ATOM | 2005 | C | GLU | B | 693 | 43.839 | 5.846 | 13.532 | 1.00 | 9.33 | C |
| ATOM | 2006 | O | GLU | B | 693 | 43.184 | 6.125 | 12.529 | 1.00 | 10.07 | O |
| ATOM | 2007 | N | LEU | B | 694 | 44.370 | 4.664 | 13.729 | 1.00 | 7.38 | N |
| ATOM | 2008 | CA | LEU | B | 694 | 44.294 | 3.595 | 12.747 | 1.00 | 7.01 | C |
| ATOM | 2009 | CB | LEU | B | 694 | 45.194 | 2.459 | 13.250 | 1.00 | 8.21 | C |
| ATOM | 2010 | CG | LEU | B | 694 | 46.133 | 1.851 | 12.255 | 1.00 | 11.49 | C |
| ATOM | 2011 | CD1 | LEU | B | 694 | 46.625 | 2.906 | 11.273 | 1.00 | 11.62 | C |
| ATOM | 2012 | CD2 | LEU | B | 694 | 47.261 | 1.293 | 13.091 | 1.00 | 12.03 | C |
| ATOM | 2013 | C | LEU | B | 694 | 42.980 | 3.049 | 12.604 | 1.00 | 6.18 | C |
| ATOM | 2014 | O | LEU | B | 694 | 42.537 | 2.492 | 11.620 | 1.00 | 4.81 | O |
| ATOM | 2015 | N | MET | B | 695 | 42.079 | 3.163 | 13.645 | 1.00 | 6.55 | N |
| ATOM | 2016 | CA | MET | B | 695 | 40.705 | 2.688 | 13.639 | 1.00 | 6.27 | C |
| ATOM | 2017 | CB | MET | B | 695 | 40.166 | 2.596 | 15.082 | 1.00 | 7.54 | C |
| ATOM | 2018 | CG | MET | B | 695 | 40.634 | 1.332 | 15.836 | 1.00 | 9.37 | C |
| ATOM | 2019 | SD | MET | B | 695 | 39.938 | −0.166 | 15.166 | 1.00 | 7.22 | S |
| ATOM | 2020 | CE | MET | B | 695 | 38.323 | 0.077 | 15.495 | 1.00 | 2.00 | C |
| ATOM | 2021 | C | MET | B | 695 | 39.780 | 3.627 | 12.933 | 1.00 | 5.88 | C |
| ATOM | 2022 | O | MET | B | 695 | 38.662 | 3.212 | 12.616 | 1.00 | 5.11 | O |
| ATOM | 2023 | N | ARG | B | 696 | 40.219 | 4.874 | 12.705 | 1.00 | 5.14 | N |
| ATOM | 2024 | CA | ARG | B | 696 | 39.358 | 5.925 | 12.159 | 1.00 | 5.59 | C |
| ATOM | 2025 | CB | ARG | B | 696 | 40.136 | 7.200 | 11.947 | 1.00 | 6.29 | C |
| ATOM | 2026 | CG | ARG | B | 696 | 39.302 | 8.469 | 11.978 | 1.00 | 6.47 | C |
| ATOM | 2027 | CD | ARG | B | 696 | 40.166 | 9.744 | 11.848 | 1.00 | 8.31 | C |
| ATOM | 2028 | NE | ARG | B | 696 | 39.304 | 10.900 | 12.043 | 1.00 | 13.20 | N |
| ATOM | 2029 | CZ | ARG | B | 696 | 39.133 | 11.587 | 13.185 | 1.00 | 9.34 | C |
| ATOM | 2030 | NH1 | ARG | B | 696 | 39.826 | 11.314 | 14.278 | 1.00 | 3.82 | N |
| ATOM | 2031 | NH2 | ARG | B | 696 | 38.279 | 12.606 | 13.177 | 1.00 | 9.75 | N |
| ATOM | 2032 | C | ARG | B | 696 | 38.683 | 5.569 | 10.866 | 1.00 | 5.88 | C |
| ATOM | 2033 | O | ARG | B | 696 | 39.321 | 5.442 | 9.835 | 1.00 | 6.24 | O |
| ATOM | 2034 | N | ASP | B | 697 | 37.374 | 5.417 | 10.909 | 1.00 | 5.84 | N |
| ATOM | 2035 | CA | ASP | B | 697 | 36.620 | 5.019 | 9.689 | 1.00 | 6.16 | C |
| ATOM | 2036 | CB | ASP | B | 697 | 36.851 | 6.024 | 8.563 | 1.00 | 5.37 | C |
| ATOM | 2037 | CG | ASP | B | 697 | 36.238 | 7.416 | 8.830 | 1.00 | 5.53 | C |
| ATOM | 2038 | OD1 | ASP | B | 697 | 35.077 | 7.547 | 9.309 | 1.00 | 7.21 | O |
| ATOM | 2039 | OD2 | ASP | B | 697 | 36.823 | 8.461 | 8.482 | 1.00 | 3.96 | O |
| ATOM | 2040 | C | ASP | B | 697 | 36.989 | 3.586 | 9.232 | 1.00 | 4.52 | C |
| ATOM | 2041 | O | ASP | B | 697 | 36.893 | 3.257 | 8.060 | 1.00 | 2.10 | O |
| ATOM | 2042 | N | ARG | B | 698 | 37.458 | 2.779 | 10.173 | 1.00 | 2.27 | N |
| ATOM | 2043 | CA | ARG | B | 698 | 37.862 | 1.431 | 9.890 | 1.00 | 3.61 | C |
| ATOM | 2044 | CB | ARG | B | 698 | 39.393 | 1.335 | 9.864 | 1.00 | 2.00 | C |
| ATOM | 2045 | CG | ARG | B | 698 | 39.962 | 1.672 | 8.510 | 1.00 | 7.08 | C |
| ATOM | 2046 | CD | ARG | B | 698 | 41.414 | 1.300 | 8.239 | 1.00 | 10.31 | C |
| ATOM | 2047 | NE | ARG | B | 698 | 42.223 | 2.334 | 8.809 | 1.00 | 14.26 | N |
| ATOM | 2048 | CZ | ARG | B | 698 | 42.863 | 3.260 | 8.137 | 1.00 | 9.60 | C |
| ATOM | 2049 | NH1 | ARG | B | 698 | 42.877 | 3.272 | 6.818 | 1.00 | 8.71 | N |
| ATOM | 2050 | NH2 | ARG | B | 698 | 43.498 | 4.171 | 8.817 | 1.00 | 5.51 | N |
| ATOM | 2051 | C | ARG | B | 698 | 37.198 | 0.423 | 10.881 | 1.00 | 4.51 | C |
| ATOM | 2052 | O | ARG | B | 698 | 36.474 | 0.785 | 11.830 | 1.00 | 5.73 | O |
| ATOM | 2053 | N | HIS | B | 699 | 37.445 | −0.843 | 10.687 | 1.00 | 4.24 | N |
| ATOM | 2054 | CA | HIS | B | 699 | 36.758 | −1.862 | 11.467 | 1.00 | 4.34 | C |
| ATOM | 2055 | CB | HIS | B | 699 | 36.070 | −2.833 | 10.451 | 1.00 | 4.52 | C |
| ATOM | 2056 | CG | HIS | B | 699 | 35.058 | −3.760 | 11.034 | 1.00 | 2.22 | C |
| ATOM | 2057 | ND1 | HIS | B | 699 | 35.386 | −4.729 | 11.964 | 1.00 | 3.86 | N |
| ATOM | 2058 | CE1 | HIS | B | 699 | 34.311 | −5.460 | 12.229 | 1.00 | 2.00 | C |
| ATOM | 2059 | NE2 | HIS | B | 699 | 33.306 | −5.005 | 11.507 | 1.00 | 2.00 | N |
| ATOM | 2060 | CD2 | HIS | B | 699 | 33.746 | −3.935 | 10.758 | 1.00 | 2.00 | C |
| ATOM | 2061 | C | HIS | B | 699 | 37.706 | −2.621 | 12.402 | 1.00 | 3.78 | C |
| ATOM | 2062 | O | HIS | B | 699 | 38.796 | −2.946 | 12.041 | 1.00 | 2.51 | O |
| ATOM | 2063 | N | LEU | B | 700 | 37.226 | −2.906 | 13.608 | 1.00 | 3.99 | N |
| ATOM | 2064 | CA | LEU | B | 700 | 37.962 | −3.652 | 14.592 | 1.00 | 4.35 | C |
| ATOM | 2065 | CB | LEU | B | 700 | 37.015 | −4.031 | 15.713 | 1.00 | 3.43 | C |
| ATOM | 2066 | CG | LEU | B | 700 | 37.467 | −4.058 | 17.177 | 1.00 | 2.71 | C |
| ATOM | 2067 | CD1 | LEU | B | 700 | 36.714 | −5.193 | 17.919 | 1.00 | 2.00 | C |
| ATOM | 2068 | CD2 | LEU | B | 700 | 38.954 | −4.198 | 17.322 | 1.00 | 2.00 | C |
| ATOM | 2069 | C | LEU | B | 700 | 38.512 | −4.994 | 14.019 | 1.00 | 4.85 | C |
| ATOM | 2070 | O | LEU | B | 700 | 39.607 | −5.385 | 14.340 | 1.00 | 3.57 | O |
| ATOM | 2071 | N | ASP | B | 701 | 37.737 | −5.695 | 13.188 | 1.00 | 4.86 | N |
| ATOM | 2072 | CA | ASP | B | 701 | 38.202 | −6.958 | 12.691 | 1.00 | 5.27 | C |
| ATOM | 2073 | CB | ASP | B | 701 | 37.055 | −7.724 | 12.051 | 1.00 | 6.51 | C |
| ATOM | 2074 | CG | ASP | B | 701 | 35.965 | −8.161 | 13.051 | 1.00 | 8.63 | C |
| ATOM | 2075 | OD1 | ASP | B | 701 | 36.140 | −8.095 | 14.300 | 1.00 | 8.97 | O |
| ATOM | 2076 | OD2 | ASP | B | 701 | 34.860 | −8.568 | 12.634 | 1.00 | 12.11 | O |
| ATOM | 2077 | C | ASP | B | 701 | 39.392 | −6.779 | 11.683 | 1.00 | 5.92 | C |
| ATOM | 2078 | O | ASP | B | 701 | 40.241 | −7.654 | 11.524 | 1.00 | 4.87 | O |
| ATOM | 2079 | N | GLN | B | 702 | 39.449 | −5.650 | 10.990 | 1.00 | 6.01 | N |
| ATOM | 2080 | CA | GLN | B | 702 | 40.548 | −5.398 | 10.068 | 1.00 | 6.04 | C |
| ATOM | 2081 | CB | GLN | B | 702 | 40.262 | −4.162 | 9.205 | 1.00 | 5.99 | C |
| ATOM | 2082 | CG | GLN | B | 702 | 38.897 | −4.192 | 8.644 | 1.00 | 5.27 | C |
| ATOM | 2083 | CD | GLN | B | 702 | 38.418 | −2.903 | 8.079 | 1.00 | 4.96 | C |
| ATOM | 2084 | OE1 | GLN | B | 702 | 38.869 | −1.797 | 8.471 | 1.00 | 4.27 | O |
| ATOM | 2085 | NE2 | GLN | B | 702 | 37.475 | −3.020 | 7.135 | 1.00 | 3.80 | N |
| ATOM | 2086 | C | GLN | B | 702 | 41.896 | −5.243 | 10.829 | 1.00 | 6.46 | C |
| ATOM | 2087 | O | GLN | B | 702 | 42.908 | −5.756 | 10.357 | 1.00 | 6.12 | O |
| ATOM | 2088 | N | ILE | B | 703 | 41.899 | −4.594 | 12.002 | 1.00 | 5.52 | N |
| ATOM | 2089 | CA | ILE | B | 703 | 43.105 | −4.450 | 12.777 | 1.00 | 6.51 | C |
| ATOM | 2090 | CB | ILE | B | 703 | 42.911 | −3.462 | 13.974 | 1.00 | 8.27 | C |
| ATOM | 2091 | CG1 | ILE | B | 703 | 43.146 | −2.006 | 13.577 | 1.00 | 7.52 | C |
| ATOM | 2092 | CD1 | ILE | B | 703 | 42.183 | −1.541 | 12.512 | 1.00 | 10.76 | C |
| ATOM | 2093 | CG2 | ILE | B | 703 | 43.838 | −3.807 | 15.102 | 1.00 | 5.38 | C |
| ATOM | 2094 | C | ILE | B | 703 | 43.451 | −5.788 | 13.382 | 1.00 | 8.35 | C |
| ATOM | 2095 | O | ILE | B | 703 | 44.630 | −6.094 | 13.568 | 1.00 | 9.15 | O |
| ATOM | 2096 | N | MET | B | 704 | 42.427 | −6.586 | 13.720 | 1.00 | 8.61 | N |
| ATOM | 2097 | CA | MET | B | 704 | 42.616 | −7.906 | 14.325 | 1.00 | 7.76 | C |
| ATOM | 2098 | CB | MET | B | 704 | 41.284 | −8.558 | 14.722 | 1.00 | 7.41 | C |
| ATOM | 2099 | CG | MET | B | 704 | 41.418 | −9.941 | 15.451 | 1.00 | 9.47 | C |
| ATOM | 2100 | SD | MET | B | 704 | 39.832 | −10.902 | 15.795 | 1.00 | 8.72 | S |
| ATOM | 2101 | CE | MET | B | 704 | 39.200 | −10.805 | 14.093 | 1.00 | 2.92 | C |
| ATOM | 2102 | C | MET | B | 704 | 43.363 | −8.835 | 13.393 | 1.00 | 7.58 | C |
| ATOM | 2103 | O | MET | B | 704 | 44.404 | −9.378 | 13.736 | 1.00 | 8.07 | O |
| ATOM | 2104 | N | MET | B | 705 | 42.825 | −9.041 | 12.207 | 1.00 | 7.84 | N |
| ATOM | 2105 | CA | MET | B | 705 | 43.404 | −9.988 | 11.262 | 1.00 | 7.60 | C |
| ATOM | 2106 | CB | MET | B | 705 | 42.471 | −10.153 | 10.059 | 1.00 | 8.87 | C |
| ATOM | 2107 | CG | MET | B | 705 | 41.137 | −10.805 | 10.290 | 1.00 | 7.32 | C |
| ATOM | 2108 | SD | MET | B | 705 | 40.198 | −10.798 | 8.683 | 1.00 | 14.24 | S |
| ATOM | 2109 | CE | MET | B | 705 | 39.124 | −9.281 | 8.938 | 1.00 | 11.08 | C |
| ATOM | 2110 | C | MET | B | 705 | 44.763 | −9.464 | 10.826 | 1.00 | 8.08 | C |
| ATOM | 2111 | O | MET | B | 705 | 45.637 | −10.222 | 10.558 | 1.00 | 7.47 | O |
| ATOM | 2112 | N | CYS | B | 706 | 44.936 | −8.142 | 10.802 | 1.00 | 9.02 | N |
| ATOM | 2113 | CA | CYS | B | 706 | 46.226 | −7.550 | 10.487 | 1.00 | 9.66 | C |
| ATOM | 2114 | CB | CYS | B | 706 | 46.091 | −6.052 | 10.175 | 1.00 | 8.53 | C |
| ATOM | 2115 | SG | CYS | B | 706 | 45.622 | −5.697 | 8.466 | 1.00 | 10.44 | S |
| ATOM | 2116 | C | CYS | B | 706 | 47.299 | −7.794 | 11.592 | 1.00 | 10.66 | C |
| ATOM | 2117 | O | CYS | B | 706 | 48.520 | −7.837 | 11.297 | 1.00 | 10.39 | O |
| ATOM | 2118 | N | SER | B | 707 | 46.870 | −7.931 | 12.848 | 1.00 | 9.97 | N |
| ATOM | 2119 | CA | SER | B | 707 | 47.837 | −8.169 | 13.873 | 1.00 | 10.40 | C |
| ATOM | 2120 | CB | SER | B | 707 | 47.296 | −7.823 | 15.246 | 1.00 | 10.36 | C |
| ATOM | 2121 | OG | SER | B | 707 | 47.061 | −6.435 | 15.460 | 1.00 | 8.24 | O |
| ATOM | 2122 | C | SER | B | 707 | 48.273 | −9.654 | 13.807 | 1.00 | 12.62 | C |
| ATOM | 2123 | O | SER | B | 707 | 49.474 | −9.959 | 13.991 | 1.00 | 11.29 | O |
| ATOM | 2124 | N | MET | B | 708 | 47.321 | −10.563 | 13.507 | 1.00 | 14.70 | N |
| ATOM | 2125 | CA | MET | B | 708 | 47.649 | −12.000 | 13.401 | 1.00 | 16.47 | C |
| ATOM | 2126 | CB | MET | B | 708 | 46.449 | −12.858 | 13.083 | 1.00 | 16.13 | C |
| ATOM | 2127 | CG | MET | B | 708 | 45.246 | −12.592 | 13.968 | 1.00 | 17.64 | C |
| ATOM | 2128 | SD | MET | B | 708 | 43.770 | −13.570 | 13.497 | 1.00 | 19.35 | S |
| ATOM | 2129 | CE | MET | B | 708 | 44.439 | −15.271 | 13.483 | 1.00 | 14.93 | C |
| ATOM | 2130 | C | MET | B | 708 | 48.674 | −12.174 | 12.299 | 1.00 | 18.00 | C |
| ATOM | 2131 | O | MET | B | 708 | 49.789 | −12.726 | 12.501 | 1.00 | 19.56 | O |
| ATOM | 2132 | N | TYR | B | 709 | 48.316 | −11.675 | 11.129 | 1.00 | 18.18 | N |
| ATOM | 2133 | CA | TYR | B | 709 | 49.206 | −11.742 | 9.981 | 1.00 | 18.22 | C |
| ATOM | 2134 | CB | TYR | B | 709 | 48.600 | −11.027 | 8.799 | 1.00 | 18.00 | C |
| ATOM | 2135 | CG | TYR | B | 709 | 49.392 | −11.215 | 7.533 | 1.00 | 20.73 | C |
| ATOM | 2136 | CD1 | TYR | B | 709 | 49.134 | −12.285 | 6.686 | 1.00 | 20.65 | C |
| ATOM | 2137 | CE1 | TYR | B | 709 | 49.811 | −12.431 | 5.520 | 1.00 | 22.80 | C |
| ATOM | 2138 | CZ | TYR | B | 709 | 50.778 | −11.520 | 5.187 | 1.00 | 23.73 | C |
| ATOM | 2139 | OH | TYR | B | 709 | 51.492 | −11.706 | 4.041 | 1.00 | 23.11 | O |
| ATOM | 2140 | CE2 | TYR | B | 709 | 51.062 | −10.456 | 6.013 | 1.00 | 22.99 | C |
| ATOM | 2141 | CD2 | TYR | B | 709 | 50.372 | −10.300 | 7.161 | 1.00 | 21.61 | C |
| ATOM | 2142 | C | TYR | B | 709 | 50.587 | −11.163 | 10.224 | 1.00 | 17.84 | C |
| ATOM | 2143 | O | TYR | B | 709 | 51.559 | −11.656 | 9.676 | 1.00 | 17.96 | O |
| ATOM | 2144 | N | GLY | B | 710 | 50.665 | −10.107 | 11.024 | 1.00 | 17.95 | N |
| ATOM | 2145 | CA | GLY | B | 710 | 51.936 | −9.469 | 11.349 | 1.00 | 17.36 | C |
| ATOM | 2146 | C | GLY | B | 710 | 52.857 | −10.280 | 12.271 | 1.00 | 17.08 | C |
| ATOM | 2147 | O | GLY | B | 710 | 53.937 | −10.643 | 11.848 | 1.00 | 18.01 | O |
| ATOM | 2148 | N | ILE | B | 711 | 52.444 | −10.585 | 13.493 | 1.00 | 16.32 | N |
| ATOM | 2149 | CA | ILE | B | 711 | 53.288 | −11.312 | 14.417 | 1.00 | 16.83 | C |
| ATOM | 2150 | CB | ILE | B | 711 | 52.598 | −11.494 | 15.796 | 1.00 | 15.62 | C |
| ATOM | 2151 | CG1 | ILE | B | 711 | 53.286 | −10.673 | 16.876 | 1.00 | 17.22 | C |
| ATOM | 2152 | CD1 | ILE | B | 711 | 53.453 | −9.239 | 16.632 | 1.00 | 18.82 | C |
| ATOM | 2153 | CG2 | ILE | B | 711 | 52.745 | −12.930 | 16.319 | 1.00 | 13.88 | C |
| ATOM | 2154 | C | ILE | B | 711 | 53.705 | −12.666 | 13.783 | 1.00 | 18.91 | C |
| ATOM | 2155 | O | ILE | B | 711 | 54.827 | −13.122 | 13.926 | 1.00 | 17.41 | O |
| ATOM | 2156 | N | CYS | B | 712 | 52.808 | −13.303 | 13.049 | 1.00 | 20.88 | N |
| ATOM | 2157 | CA | CYS | B | 712 | 53.214 | −14.544 | 12.455 | 1.00 | 23.68 | C |
| ATOM | 2158 | CB | CYS | B | 712 | 52.091 | −15.179 | 11.615 | 1.00 | 23.93 | C |
| ATOM | 2159 | SG | CYS | B | 712 | 50.569 | −15.723 | 12.496 | 1.00 | 26.62 | S |
| ATOM | 2160 | C | CYS | B | 712 | 54.430 | −14.237 | 11.571 | 1.00 | 25.43 | C |
| ATOM | 2161 | O | CYS | B | 712 | 55.390 | −15.029 | 11.516 | 1.00 | 25.92 | O |
| ATOM | 2162 | N | LYS | B | 713 | 54.392 | −13.097 | 10.875 | 1.00 | 26.01 | N |
| ATOM | 2163 | CA | LYS | B | 713 | 55.467 | −12.746 | 9.970 | 1.00 | 27.09 | C |
| ATOM | 2164 | CB | LYS | B | 713 | 55.171 | −11.436 | 9.272 | 1.00 | 27.73 | C |
| ATOM | 2165 | CG | LYS | B | 713 | 54.261 | −11.607 | 8.055 | 1.00 | 31.65 | C |
| ATOM | 2166 | CD | LYS | B | 713 | 54.847 | −12.565 | 7.008 | 1.00 | 35.08 | C |
| ATOM | 2167 | CE | LYS | B | 713 | 54.103 | −12.463 | 5.640 | 1.00 | 37.06 | C |
| ATOM | 2168 | NZ | LYS | B | 713 | 54.896 | −13.092 | 4.488 | 1.00 | 37.63 | N |
| ATOM | 2169 | C | LYS | B | 713 | 56.825 | −12.677 | 10.631 | 1.00 | 26.49 | C |
| ATOM | 2170 | O | LYS | B | 713 | 57.790 | −13.241 | 10.157 | 1.00 | 25.93 | O |
| ATOM | 2171 | N | VAL | B | 714 | 56.900 | −11.968 | 11.736 | 1.00 | 26.93 | N |
| ATOM | 2172 | CA | VAL | B | 714 | 58.181 | −11.816 | 12.437 | 1.00 | 26.50 | C |
| ATOM | 2173 | CB | VAL | B | 714 | 58.141 | −10.659 | 13.427 | 1.00 | 26.43 | C |
| ATOM | 2174 | CG1 | VAL | B | 714 | 58.139 | −9.365 | 12.651 | 1.00 | 27.09 | C |
| ATOM | 2175 | CG2 | VAL | B | 714 | 56.913 | −10.776 | 14.334 | 1.00 | 23.75 | C |
| ATOM | 2176 | C | VAL | B | 714 | 58.631 | −13.072 | 13.160 | 1.00 | 26.16 | C |
| ATOM | 2177 | O | VAL | B | 714 | 59.795 | −13.206 | 13.464 | 1.00 | 26.08 | O |
| ATOM | 2178 | N | LYS | B | 715 | 57.697 | −13.978 | 13.431 | 1.00 | 26.17 | N |
| ATOM | 2179 | CA | LYS | B | 715 | 58.000 | −15.241 | 14.067 | 1.00 | 26.40 | C |
| ATOM | 2180 | CB | LYS | B | 715 | 56.935 | −15.601 | 15.080 | 1.00 | 25.70 | C |
| ATOM | 2181 | CG | LYS | B | 715 | 56.793 | −14.644 | 16.220 | 1.00 | 23.21 | C |
| ATOM | 2182 | CD | LYS | B | 715 | 57.950 | −14.735 | 17.093 | 1.00 | 22.85 | C |
| ATOM | 2183 | CE | LYS | B | 715 | 57.613 | −14.356 | 18.501 | 1.00 | 21.73 | C |
| ATOM | 2184 | NZ | LYS | B | 715 | 58.799 | −14.669 | 19.392 | 1.00 | 20.20 | N |
| ATOM | 2185 | C | LYS | B | 715 | 58.099 | −16.343 | 13.008 | 1.00 | 28.00 | C |
| ATOM | 2186 | O | LYS | B | 715 | 58.119 | −17.523 | 13.325 | 1.00 | 27.94 | O |
| ATOM | 2187 | N | ASN | B | 716 | 58.164 | −15.941 | 11.749 | 1.00 | 29.90 | N |
| ATOM | 2188 | CA | ASN | B | 716 | 58.255 | −16.882 | 10.639 | 1.00 | 32.18 | C |
| ATOM | 2189 | CB | ASN | B | 716 | 59.663 | −17.488 | 10.599 | 1.00 | 33.07 | C |
| ATOM | 2190 | CG | ASN | B | 716 | 60.627 | −16.647 | 9.764 | 1.00 | 34.01 | C |
| ATOM | 2191 | OD1 | ASN | B | 716 | 61.153 | −15.622 | 10.234 | 1.00 | 36.77 | O |
| ATOM | 2192 | ND2 | ASN | B | 716 | 60.847 | −17.067 | 8.516 | 1.00 | 30.24 | N |
| ATOM | 2193 | C | ASN | B | 716 | 57.185 | −17.991 | 10.504 | 1.00 | 32.52 | C |
| ATOM | 2194 | O | ASN | B | 716 | 57.395 | −18.965 | 9.773 | 1.00 | 32.68 | O |
| ATOM | 2195 | N | ILE | B | 717 | 56.060 | −17.846 | 11.204 | 1.00 | 32.92 | N |
| ATOM | 2196 | CA | ILE | B | 717 | 54.983 | −18.826 | 11.142 | 1.00 | 32.59 | C |
| ATOM | 2197 | CB | ILE | B | 717 | 54.031 | −18.641 | 12.311 | 1.00 | 32.11 | C |
| ATOM | 2198 | CG1 | ILE | B | 717 | 54.799 | −18.778 | 13.620 | 1.00 | 32.42 | C |
| ATOM | 2199 | CD1 | ILE | B | 717 | 55.411 | −20.122 | 13.859 | 1.00 | 31.30 | C |
| ATOM | 2200 | CG2 | ILE | B | 717 | 52.865 | −19.624 | 12.222 | 1.00 | 31.23 | C |
| ATOM | 2201 | C | ILE | B | 717 | 54.251 | −18.581 | 9.860 | 1.00 | 33.04 | C |
| ATOM | 2202 | O | ILE | B | 717 | 53.776 | −17.477 | 9.616 | 1.00 | 34.05 | O |
| ATOM | 2203 | N | ASP | B | 718 | 54.148 | −19.604 | 9.035 | 1.00 | 33.06 | N |
| ATOM | 2204 | CA | ASP | B | 718 | 53.501 | −19.452 | 7.739 | 1.00 | 33.60 | C |
| ATOM | 2205 | CB | ASP | B | 718 | 53.906 | −20.614 | 6.831 | 1.00 | 33.73 | C |
| ATOM | 2206 | CG | ASP | B | 718 | 53.353 | −20.482 | 5.434 | 1.00 | 34.30 | C |
| ATOM | 2207 | OD1 | ASP | B | 718 | 53.839 | −19.601 | 4.661 | 1.00 | 32.07 | O |
| ATOM | 2208 | OD2 | ASP | B | 718 | 52.432 | −21.237 | 5.035 | 1.00 | 33.96 | O |
| ATOM | 2209 | C | ASP | B | 718 | 51.974 | −19.329 | 7.795 | 1.00 | 33.35 | C |
| ATOM | 2210 | O | ASP | B | 718 | 51.249 | −20.326 | 7.935 | 1.00 | 34.12 | O |
| ATOM | 2211 | N | LEU | B | 719 | 51.469 | −18.114 | 7.672 | 1.00 | 32.76 | N |
| ATOM | 2212 | CA | LEU | B | 719 | 50.012 | −17.908 | 7.748 | 1.00 | 32.36 | C |
| ATOM | 2213 | CB | LEU | B | 719 | 49.590 | −17.409 | 9.138 | 1.00 | 32.31 | C |
| ATOM | 2214 | CG | LEU | B | 719 | 48.096 | −17.110 | 9.353 | 1.00 | 33.01 | C |
| ATOM | 2215 | CD1 | LEU | B | 719 | 47.220 | −18.253 | 8.871 | 1.00 | 31.76 | C |
| ATOM | 2216 | CD2 | LEU | B | 719 | 47.761 | −16.776 | 10.820 | 1.00 | 32.06 | C |
| ATOM | 2217 | C | LEU | B | 719 | 49.586 | −16.934 | 6.682 | 1.00 | 31.40 | C |
| ATOM | 2218 | O | LEU | B | 719 | 49.865 | −15.743 | 6.764 | 1.00 | 30.98 | O |
| ATOM | 2219 | N | LYS | B | 720 | 48.937 | −17.454 | 5.664 | 1.00 | 30.94 | N |
| ATOM | 2220 | CA | LYS | B | 720 | 48.521 | −16.604 | 4.573 | 1.00 | 31.42 | C |
| ATOM | 2221 | CB | LYS | B | 720 | 48.380 | −17.419 | 3.278 | 1.00 | 31.88 | C |
| ATOM | 2222 | CG | LYS | B | 720 | 49.634 | −18.216 | 2.944 | 1.00 | 33.94 | C |
| ATOM | 2223 | CD | LYS | B | 720 | 49.478 | −19.008 | 1.649 | 1.00 | 37.89 | C |
| ATOM | 2224 | CE | LYS | B | 720 | 49.501 | −18.082 | 0.379 | 1.00 | 39.26 | C |
| ATOM | 2225 | NZ | LYS | B | 720 | 49.418 | −18.855 | −0.944 | 1.00 | 38.62 | N |
| ATOM | 2226 | C | LYS | B | 720 | 47.223 | −15.858 | 4.896 | 1.00 | 30.27 | C |
| ATOM | 2227 | O | LYS | B | 720 | 46.347 | −16.374 | 5.592 | 1.00 | 30.09 | O |
| ATOM | 2228 | N | PHE | B | 721 | 47.117 | −14.644 | 4.368 | 1.00 | 29.04 | N |
| ATOM | 2229 | CA | PHE | B | 721 | 45.957 | −13.801 | 4.578 | 1.00 | 28.05 | C |
| ATOM | 2230 | CB | PHE | B | 721 | 46.162 | −12.422 | 3.958 | 1.00 | 28.24 | C |
| ATOM | 2231 | CG | PHE | B | 721 | 46.005 | −11.287 | 4.948 | 1.00 | 29.36 | C |
| ATOM | 2232 | CD1 | PHE | B | 721 | 44.870 | −11.178 | 5.715 | 1.00 | 31.20 | C |
| ATOM | 2233 | CE1 | PHE | B | 721 | 44.728 | −10.147 | 6.625 | 1.00 | 31.53 | C |
| ATOM | 2234 | CZ | PHE | B | 721 | 45.750 | −9.238 | 6.792 | 1.00 | 30.40 | C |
| ATOM | 2235 | CE2 | PHE | B | 721 | 46.885 | −9.353 | 6.050 | 1.00 | 29.38 | C |
| ATOM | 2236 | CD2 | PHE | B | 721 | 47.017 | −10.361 | 5.134 | 1.00 | 30.03 | C |
| ATOM | 2237 | C | PHE | B | 721 | 44.714 | −14.443 | 4.011 | 1.00 | 27.24 | C |
| ATOM | 2238 | O | PHE | B | 721 | 43.614 | −14.270 | 4.532 | 1.00 | 27.29 | O |
| ATOM | 2239 | N | LYS | B | 722 | 44.888 | −15.203 | 2.952 | 1.00 | 25.84 | N |
| ATOM | 2240 | CA | LYS | B | 722 | 43.762 | −15.855 | 2.337 | 1.00 | 25.19 | C |
| ATOM | 2241 | CB | LYS | B | 722 | 44.165 | −16.442 | 0.986 | 1.00 | 26.12 | C |
| ATOM | 2242 | CG | LYS | B | 722 | 43.008 | −16.961 | 0.169 | 1.00 | 30.46 | C |
| ATOM | 2243 | CD | LYS | B | 722 | 43.524 | −17.782 | −1.015 | 1.00 | 35.80 | C |
| ATOM | 2244 | CE | LYS | B | 722 | 44.265 | −19.065 | −0.529 | 1.00 | 37.91 | C |
| ATOM | 2245 | NZ | LYS | B | 722 | 43.363 | −20.138 | −0.003 | 1.00 | 36.85 | N |
| ATOM | 2246 | C | LYS | B | 722 | 43.224 | −16.916 | 3.276 | 1.00 | 23.23 | C |
| ATOM | 2247 | O | LYS | B | 722 | 42.052 | −17.236 | 3.235 | 1.00 | 23.64 | O |
| ATOM | 2248 | N | ILE | B | 723 | 44.086 | −17.465 | 4.117 | 1.00 | 21.64 | N |
| ATOM | 2249 | CA | ILE | B | 723 | 43.670 | −18.462 | 5.090 | 1.00 | 20.50 | C |
| ATOM | 2250 | CB | ILE | B | 723 | 44.905 | −19.242 | 5.626 | 1.00 | 20.86 | C |
| ATOM | 2251 | CG1 | ILE | B | 723 | 45.479 | −20.194 | 4.560 | 1.00 | 22.30 | C |
| ATOM | 2252 | CD1 | ILE | B | 723 | 44.530 | −21.368 | 4.187 | 1.00 | 25.70 | C |
| ATOM | 2253 | CG2 | ILE | B | 723 | 44.556 | −20.036 | 6.895 | 1.00 | 18.10 | C |
| ATOM | 2254 | C | ILE | B | 723 | 42.937 | −17.771 | 6.249 | 1.00 | 19.81 | C |
| ATOM | 2255 | O | ILE | B | 723 | 41.997 | −18.301 | 6.822 | 1.00 | 20.17 | O |
| ATOM | 2256 | N | ILE | B | 724 | 43.384 | −16.581 | 6.600 | 1.00 | 17.88 | N |
| ATOM | 2257 | CA | ILE | B | 724 | 42.789 | −15.874 | 7.676 | 1.00 | 16.10 | C |
| ATOM | 2258 | CB | ILE | B | 724 | 43.685 | −14.687 | 8.034 | 1.00 | 16.51 | C |
| ATOM | 2259 | CG1 | ILE | B | 724 | 45.116 | −15.191 | 8.240 | 1.00 | 13.98 | C |
| ATOM | 2260 | CD1 | ILE | B | 724 | 46.054 | −14.177 | 8.828 | 1.00 | 13.32 | C |
| ATOM | 2261 | CG2 | ILE | B | 724 | 43.137 | −13.949 | 9.255 | 1.00 | 16.09 | C |
| ATOM | 2262 | C | ILE | B | 724 | 41.407 | −15.432 | 7.288 | 1.00 | 15.97 | C |
| ATOM | 2263 | O | ILE | B | 724 | 40.475 | −15.523 | 8.079 | 1.00 | 15.53 | O |
| ATOM | 2264 | N | VAL | B | 725 | 41.247 | −14.975 | 6.058 | 1.00 | 16.45 | N |
| ATOM | 2265 | CA | VAL | B | 725 | 39.946 | −14.483 | 5.577 | 1.00 | 17.58 | C |
| ATOM | 2266 | CB | VAL | B | 725 | 40.049 | −14.015 | 4.145 | 1.00 | 18.51 | C |
| ATOM | 2267 | CG1 | VAL | B | 725 | 38.846 | −14.384 | 3.410 | 1.00 | 19.37 | C |
| ATOM | 2268 | CG2 | VAL | B | 725 | 40.339 | −12.541 | 4.079 | 1.00 | 20.64 | C |
| ATOM | 2269 | C | VAL | B | 725 | 38.878 | −15.563 | 5.615 | 1.00 | 17.09 | C |
| ATOM | 2270 | O | VAL | B | 725 | 37.783 | −15.368 | 6.169 | 1.00 | 17.94 | O |
| ATOM | 2271 | N | THR | B | 726 | 39.216 | −16.718 | 5.067 | 1.00 | 16.16 | N |
| ATOM | 2272 | CA | THR | B | 726 | 38.304 | −17.865 | 5.037 | 1.00 | 15.43 | C |
| ATOM | 2273 | CB | THR | B | 726 | 38.983 | −19.089 | 4.332 | 1.00 | 15.56 | C |
| ATOM | 2274 | OG1 | THR | B | 726 | 39.725 | −18.634 | 3.194 | 1.00 | 15.24 | O |
| ATOM | 2275 | CG2 | THR | B | 726 | 37.963 | −19.961 | 3.682 | 1.00 | 14.25 | C |
| ATOM | 2276 | C | THR | B | 726 | 37.769 | −18.254 | 6.428 | 1.00 | 14.32 | C |
| ATOM | 2277 | O | THR | B | 726 | 36.600 | −18.612 | 6.590 | 1.00 | 13.74 | O |
| ATOM | 2278 | N | ALA | B | 727 | 38.621 | −18.208 | 7.430 | 1.00 | 13.02 | N |
| ATOM | 2279 | CA | ALA | B | 727 | 38.182 | −18.547 | 8.785 | 1.00 | 12.57 | C |
| ATOM | 2280 | CB | ALA | B | 727 | 39.394 | −18.653 | 9.663 | 1.00 | 12.86 | C |
| ATOM | 2281 | C | ALA | B | 727 | 37.235 | −17.440 | 9.331 | 1.00 | 12.91 | C |
| ATOM | 2282 | O | ALA | B | 727 | 36.101 | −17.702 | 9.745 | 1.00 | 11.86 | O |
| ATOM | 2283 | N | TYR | B | 728 | 37.716 | −16.195 | 9.289 | 1.00 | 13.37 | N |
| ATOM | 2284 | CA | TYR | B | 728 | 36.915 | −15.040 | 9.660 | 1.00 | 13.34 | C |
| ATOM | 2285 | CB | TYR | B | 728 | 37.560 | −13.739 | 9.134 | 1.00 | 12.87 | C |
| ATOM | 2286 | CG | TYR | B | 728 | 36.773 | −12.497 | 9.455 | 1.00 | 11.24 | C |
| ATOM | 2287 | CD1 | TYR | B | 728 | 36.790 | −11.930 | 10.738 | 1.00 | 9.80 | C |
| ATOM | 2288 | CE1 | TYR | B | 728 | 36.061 | −10.794 | 11.018 | 1.00 | 5.74 | C |
| ATOM | 2289 | CZ | TYR | B | 728 | 35.323 | −10.222 | 10.030 | 1.00 | 7.00 | C |
| ATOM | 2290 | OH | TYR | B | 728 | 34.579 | −9.074 | 10.272 | 1.00 | 7.47 | O |
| ATOM | 2291 | CE2 | TYR | B | 728 | 35.308 | −10.766 | 8.777 | 1.00 | 5.96 | C |
| ATOM | 2292 | CD2 | TYR | B | 728 | 36.036 | −11.875 | 8.493 | 1.00 | 7.33 | C |
| ATOM | 2293 | C | TYR | B | 728 | 35.496 | −15.203 | 9.111 | 1.00 | 13.39 | C |
| ATOM | 2294 | O | TYR | B | 728 | 34.562 | −15.001 | 9.856 | 1.00 | 12.47 | O |
| ATOM | 2295 | N | LYS | B | 729 | 35.355 | −15.598 | 7.831 | 1.00 | 13.99 | N |
| ATOM | 2296 | CA | LYS | B | 729 | 34.034 | −15.706 | 7.199 | 1.00 | 15.28 | C |
| ATOM | 2297 | CB | LYS | B | 729 | 34.085 | −16.311 | 5.783 | 1.00 | 14.79 | C |
| ATOM | 2298 | CG | LYS | B | 729 | 34.768 | −15.480 | 4.748 | 1.00 | 18.29 | C |
| ATOM | 2299 | CD | LYS | B | 729 | 34.356 | −15.844 | 3.278 | 1.00 | 21.11 | C |
| ATOM | 2300 | CE | LYS | B | 729 | 35.021 | −17.144 | 2.752 | 1.00 | 25.75 | C |
| ATOM | 2301 | NZ | LYS | B | 729 | 34.801 | −18.458 | 3.578 | 1.00 | 24.25 | N |
| ATOM | 2302 | C | LYS | B | 729 | 33.100 | −16.568 | 8.043 | 1.00 | 15.73 | C |
| ATOM | 2303 | O | LYS | B | 729 | 31.884 | −16.377 | 7.977 | 1.00 | 14.88 | O |
| ATOM | 2304 | N | ASP | B | 730 | 33.659 | −17.558 | 8.770 | 1.00 | 16.21 | N |
| ATOM | 2305 | CA | ASP | B | 730 | 32.847 | −18.411 | 9.671 | 1.00 | 17.14 | C |
| ATOM | 2306 | CB | ASP | B | 730 | 33.624 | −19.640 | 10.136 | 1.00 | 17.31 | C |
| ATOM | 2307 | CG | ASP | B | 730 | 33.778 | −20.688 | 9.036 | 1.00 | 20.23 | C |
| ATOM | 2308 | OD1 | ASP | B | 730 | 32.778 | −20.962 | 8.350 | 1.00 | 22.19 | O |
| ATOM | 2309 | OD2 | ASP | B | 730 | 34.856 | −21.298 | 8.810 | 1.00 | 20.06 | O |
| ATOM | 2310 | C | ASP | B | 730 | 32.241 | −17.690 | 10.914 | 1.00 | 16.31 | C |
| ATOM | 2311 | O | ASP | B | 730 | 31.219 | −18.118 | 11.429 | 1.00 | 17.05 | O |
| ATOM | 2312 | N | LEU | B | 731 | 32.844 | −16.610 | 11.392 | 1.00 | 14.65 | N |
| ATOM | 2313 | CA | LEU | B | 731 | 32.243 | −15.945 | 12.522 | 1.00 | 15.22 | C |
| ATOM | 2314 | CB | LEU | B | 731 | 33.125 | −14.789 | 13.052 | 1.00 | 14.75 | C |
| ATOM | 2315 | CG | LEU | B | 731 | 34.636 | −15.126 | 13.196 | 1.00 | 13.31 | C |
| ATOM | 2316 | CD1 | LEU | B | 731 | 35.586 | −13.937 | 13.501 | 1.00 | 8.91 | C |
| ATOM | 2317 | CD2 | LEU | B | 731 | 34.775 | −16.150 | 14.223 | 1.00 | 11.15 | C |
| ATOM | 2318 | C | LEU | B | 731 | 30.846 | −15.507 | 12.109 | 1.00 | 15.53 | C |
| ATOM | 2319 | O | LEU | B | 731 | 30.649 | −15.085 | 10.986 | 1.00 | 15.48 | O |
| ATOM | 2320 | N | PRO | B | 732 | 29.874 | −15.635 | 13.008 | 1.00 | 16.47 | N |
| ATOM | 2321 | CA | PRO | B | 732 | 28.444 | −15.313 | 12.714 | 1.00 | 17.37 | C |
| ATOM | 2322 | CB | PRO | B | 732 | 27.732 | −15.685 | 14.022 | 1.00 | 17.22 | C |
| ATOM | 2323 | CG | PRO | B | 732 | 28.872 | −15.595 | 15.072 | 1.00 | 16.47 | C |
| ATOM | 2324 | CD | PRO | B | 732 | 30.066 | −16.164 | 14.367 | 1.00 | 15.27 | C |
| ATOM | 2325 | C | PRO | B | 732 | 28.075 | −13.853 | 12.326 | 1.00 | 18.51 | C |
| ATOM | 2326 | O | PRO | B | 732 | 27.003 | −13.592 | 11.762 | 1.00 | 17.81 | O |
| ATOM | 2327 | N | HIS | B | 733 | 28.931 | −12.894 | 12.620 | 1.00 | 19.79 | N |
| ATOM | 2328 | CA | HIS | B | 733 | 28.578 | −11.520 | 12.293 | 1.00 | 21.62 | C |
| ATOM | 2329 | CB | HIS | B | 733 | 28.964 | −10.622 | 13.462 | 1.00 | 22.24 | C |
| ATOM | 2330 | CG | HIS | B | 733 | 30.392 | −10.792 | 13.863 | 1.00 | 26.72 | C |
| ATOM | 2331 | ND1 | HIS | B | 733 | 31.403 | −9.962 | 13.400 | 1.00 | 31.50 | N |
| ATOM | 2332 | CE1 | HIS | B | 733 | 32.566 | −10.387 | 13.870 | 1.00 | 31.30 | C |
| ATOM | 2333 | NE2 | HIS | B | 733 | 32.343 | −11.463 | 14.614 | 1.00 | 32.86 | N |
| ATOM | 2334 | CD2 | HIS | B | 733 | 30.998 | −11.751 | 14.608 | 1.00 | 28.05 | C |
| ATOM | 2335 | C | HIS | B | 733 | 29.366 | −11.070 | 11.077 | 1.00 | 21.53 | C |
| ATOM | 2336 | O | HIS | B | 733 | 29.161 | −9.967 | 10.568 | 1.00 | 22.67 | O |
| ATOM | 2337 | N | ALA | B | 734 | 30.276 | −11.924 | 10.626 | 1.00 | 20.57 | N |
| ATOM | 2338 | CA | ALA | B | 734 | 31.179 | −11.610 | 9.525 | 1.00 | 19.72 | C |
| ATOM | 2339 | CB | ALA | B | 734 | 32.132 | −12.798 | 9.274 | 1.00 | 19.49 | C |
| ATOM | 2340 | C | ALA | B | 734 | 30.555 | −11.198 | 8.210 | 1.00 | 18.53 | C |
| ATOM | 2341 | O | ALA | B | 734 | 29.738 | −11.898 | 7.666 | 1.00 | 18.10 | O |
| ATOM | 2342 | N | VAL | B | 735 | 31.036 | −10.096 | 7.676 | 1.00 | 18.27 | N |
| ATOM | 2343 | CA | VAL | B | 735 | 30.646 | −9.624 | 6.357 | 1.00 | 18.38 | C |
| ATOM | 2344 | CB | VAL | B | 735 | 30.027 | −8.272 | 6.466 | 1.00 | 17.76 | C |
| ATOM | 2345 | CG1 | VAL | B | 735 | 29.805 | −7.694 | 5.093 | 1.00 | 19.37 | C |
| ATOM | 2346 | CG2 | VAL | B | 735 | 28.741 | −8.437 | 7.141 | 1.00 | 17.16 | C |
| ATOM | 2347 | C | VAL | B | 735 | 31.832 | −9.562 | 5.428 | 1.00 | 18.15 | C |
| ATOM | 2348 | O | VAL | B | 735 | 32.839 | −8.983 | 5.751 | 1.00 | 18.71 | O |
| ATOM | 2349 | N | GLN | B | 736 | 31.712 | −10.129 | 4.245 | 1.00 | 19.52 | N |
| ATOM | 2350 | CA | GLN | B | 736 | 32.851 | −10.140 | 3.292 | 1.00 | 20.24 | C |
| ATOM | 2351 | CB | GLN | B | 736 | 32.435 | −10.691 | 1.937 | 1.00 | 21.49 | C |
| ATOM | 2352 | CG | GLN | B | 736 | 32.023 | −12.190 | 1.994 | 1.00 | 26.42 | C |
| ATOM | 2353 | CD | GLN | B | 736 | 32.035 | −12.854 | 0.580 | 1.00 | 30.92 | C |
| ATOM | 2354 | OE1 | GLN | B | 736 | 33.109 | −13.248 | 0.047 | 1.00 | 30.86 | O |
| ATOM | 2355 | NE2 | GLN | B | 736 | 30.851 | −12.949 | −0.024 | 1.00 | 30.08 | N |
| ATOM | 2356 | C | GLN | B | 736 | 33.517 | −8.799 | 3.100 | 1.00 | 18.33 | C |
| ATOM | 2357 | O | GLN | B | 736 | 34.771 | −8.694 | 3.090 | 1.00 | 19.47 | O |
| ATOM | 2358 | N | GLU | B | 737 | 32.672 | −7.793 | 2.972 | 1.00 | 15.56 | N |
| ATOM | 2359 | CA | GLU | B | 737 | 33.095 | −6.424 | 2.769 | 1.00 | 14.71 | C |
| ATOM | 2360 | CB | GLU | B | 737 | 31.857 | −5.518 | 2.763 | 1.00 | 13.93 | C |
| ATOM | 2361 | CG | GLU | B | 737 | 32.132 | −4.035 | 2.789 | 1.00 | 20.16 | C |
| ATOM | 2362 | CD | GLU | B | 737 | 30.887 | −3.181 | 3.145 | 1.00 | 29.64 | C |
| ATOM | 2363 | OE1 | GLU | B | 737 | 30.162 | −3.607 | 4.089 | 1.00 | 34.03 | O |
| ATOM | 2364 | OE2 | GLU | B | 737 | 30.615 | −2.096 | 2.518 | 1.00 | 27.73 | O |
| ATOM | 2365 | C | GLU | B | 737 | 34.206 | −5.959 | 3.756 | 1.00 | 13.09 | C |
| ATOM | 2366 | O | GLU | B | 737 | 35.063 | −5.162 | 3.389 | 1.00 | 13.18 | O |
| ATOM | 2367 | N | THR | B | 738 | 34.224 | −6.472 | 4.984 | 1.00 | 11.57 | N |
| ATOM | 2368 | CA | THR | B | 738 | 35.257 | −6.065 | 5.951 | 1.00 | 10.08 | C |
| ATOM | 2369 | CB | THR | B | 738 | 34.999 | −6.653 | 7.332 | 1.00 | 8.74 | C |
| ATOM | 2370 | OG1 | THR | B | 738 | 33.648 | −6.403 | 7.711 | 1.00 | 9.41 | O |
| ATOM | 2371 | CG2 | THR | B | 738 | 35.757 | −5.946 | 8.356 | 1.00 | 2.00 | C |
| ATOM | 2372 | C | THR | B | 738 | 36.728 | −6.349 | 5.527 | 1.00 | 10.98 | C |
| ATOM | 2373 | O | THR | B | 738 | 37.616 | −5.661 | 5.995 | 1.00 | 9.61 | O |
| ATOM | 2374 | N | PHE | B | 739 | 36.976 | −7.314 | 4.634 | 1.00 | 12.04 | N |
| ATOM | 2375 | CA | PHE | B | 739 | 38.338 | −7.514 | 4.109 | 1.00 | 13.84 | C |
| ATOM | 2376 | CB | PHE | B | 739 | 38.926 | −8.893 | 4.455 | 1.00 | 13.62 | C |
| ATOM | 2377 | CG | PHE | B | 739 | 38.107 | −10.029 | 3.986 | 1.00 | 14.17 | C |
| ATOM | 2378 | CD1 | PHE | B | 739 | 38.063 | −10.348 | 2.633 | 1.00 | 14.68 | C |
| ATOM | 2379 | CE1 | PHE | B | 739 | 37.279 | −11.414 | 2.175 | 1.00 | 12.99 | C |
| ATOM | 2380 | CZ | PHE | B | 739 | 36.511 | −12.157 | 3.098 | 1.00 | 11.66 | C |
| ATOM | 2381 | CE2 | PHE | B | 739 | 36.550 | −11.832 | 4.439 | 1.00 | 12.82 | C |
| ATOM | 2382 | CD2 | PHE | B | 739 | 37.348 | −10.775 | 4.879 | 1.00 | 12.84 | C |
| ATOM | 2383 | C | PHE | B | 739 | 38.449 | −7.259 | 2.590 | 1.00 | 15.45 | C |
| ATOM | 2384 | O | PHE | B | 739 | 39.537 | −7.256 | 2.013 | 1.00 | 16.07 | O |
| ATOM | 2385 | N | LYS | B | 740 | 37.338 | −7.031 | 1.912 | 1.00 | 16.42 | N |
| ATOM | 2386 | CA | LYS | B | 740 | 37.424 | −6.723 | 0.479 | 1.00 | 16.15 | C |
| ATOM | 2387 | CB | LYS | B | 740 | 36.213 | −7.289 | −0.258 | 1.00 | 17.06 | C |
| ATOM | 2388 | CG | LYS | B | 740 | 36.071 | −8.841 | −0.313 | 1.00 | 18.60 | C |
| ATOM | 2389 | CD | LYS | B | 740 | 35.364 | −9.271 | −1.631 | 1.00 | 17.32 | C |
| ATOM | 2390 | CE | LYS | B | 740 | 34.757 | −10.655 | −1.542 | 1.00 | 22.65 | C |
| ATOM | 2391 | NZ | LYS | B | 740 | 35.750 | −11.740 | −1.288 | 1.00 | 26.48 | N |
| ATOM | 2392 | C | LYS | B | 740 | 37.470 | −5.213 | 0.201 | 1.00 | 15.61 | C |
| ATOM | 2393 | O | LYS | B | 740 | 37.968 | −4.795 | −0.822 | 1.00 | 14.70 | O |
| ATOM | 2394 | N | ARG | B | 741 | 36.920 | −4.416 | 1.127 | 1.00 | 15.05 | N |
| ATOM | 2395 | CA | ARG | B | 741 | 36.797 | −2.962 | 0.995 | 1.00 | 14.19 | C |
| ATOM | 2396 | CB | ARG | B | 741 | 35.330 | −2.620 | 0.730 | 1.00 | 14.64 | C |
| ATOM | 2397 | CG | ARG | B | 741 | 35.008 | −1.179 | 0.421 | 1.00 | 16.18 | C |
| ATOM | 2398 | CD | ARG | B | 741 | 33.588 | −0.966 | −0.148 | 1.00 | 24.70 | C |
| ATOM | 2399 | NE | ARG | B | 741 | 33.531 | 0.033 | −1.252 | 1.00 | 25.41 | N |
| ATOM | 2400 | CZ | ARG | B | 741 | 33.785 | 1.342 | −1.084 | 1.00 | 26.65 | C |
| ATOM | 2401 | NH1 | ARG | B | 741 | 34.096 | 1.807 | 0.123 | 1.00 | 25.62 | N |
| ATOM | 2402 | NH2 | ARG | B | 741 | 33.733 | 2.193 | −2.102 | 1.00 | 25.67 | N |
| ATOM | 2403 | C | ARG | B | 741 | 37.245 | −2.300 | 2.291 | 1.00 | 13.80 | C |
| ATOM | 2404 | O | ARG | B | 741 | 36.469 | −2.118 | 3.199 | 1.00 | 12.81 | O |
| ATOM | 2405 | N | VAL | B | 742 | 38.519 | −1.966 | 2.379 | 1.00 | 13.97 | N |
| ATOM | 2406 | CA | VAL | B | 742 | 39.089 | −1.379 | 3.586 | 1.00 | 14.00 | C |
| ATOM | 2407 | CB | VAL | B | 742 | 40.197 | −2.276 | 4.165 | 1.00 | 14.78 | C |
| ATOM | 2408 | CG1 | VAL | B | 742 | 40.864 | −1.627 | 5.411 | 1.00 | 12.67 | C |
| ATOM | 2409 | CG2 | VAL | B | 742 | 39.671 | −3.683 | 4.413 | 1.00 | 13.17 | C |
| ATOM | 2410 | C | VAL | B | 742 | 39.698 | −0.039 | 3.193 | 1.00 | 13.99 | C |
| ATOM | 2411 | O | VAL | B | 742 | 40.175 | 0.101 | 2.069 | 1.00 | 13.44 | O |
| ATOM | 2412 | N | LEU | B | 743 | 39.640 | 0.928 | 4.096 | 1.00 | 14.08 | N |
| ATOM | 2413 | CA | LEU | B | 743 | 40.118 | 2.281 | 3.834 | 1.00 | 16.23 | C |
| ATOM | 2414 | CB | LEU | B | 743 | 39.695 | 3.196 | 4.950 | 1.00 | 16.17 | C |
| ATOM | 2415 | CG | LEU | B | 743 | 39.291 | 4.624 | 4.687 | 1.00 | 17.47 | C |
| ATOM | 2416 | CD1 | LEU | B | 743 | 39.320 | 5.415 | 5.988 | 1.00 | 16.65 | C |
| ATOM | 2417 | CD2 | LEU | B | 743 | 40.177 | 5.284 | 3.687 | 1.00 | 19.65 | C |
| ATOM | 2418 | C | LEU | B | 743 | 41.622 | 2.365 | 3.695 | 1.00 | 17.49 | C |
| ATOM | 2419 | O | LEU | B | 743 | 42.360 | 1.710 | 4.400 | 1.00 | 17.60 | O |
| ATOM | 2420 | N | ILE | B | 744 | 42.072 | 3.160 | 2.744 | 1.00 | 20.53 | N |
| ATOM | 2421 | CA | ILE | B | 744 | 43.492 | 3.338 | 2.533 | 1.00 | 23.74 | C |
| ATOM | 2422 | CB | ILE | B | 744 | 43.902 | 2.930 | 1.102 | 1.00 | 24.04 | C |
| ATOM | 2423 | CG1 | ILE | B | 744 | 43.379 | 1.514 | 0.756 | 1.00 | 22.67 | C |
| ATOM | 2424 | CD1 | ILE | B | 744 | 43.896 | 0.426 | 1.560 | 1.00 | 19.74 | C |
| ATOM | 2425 | CG2 | ILE | B | 744 | 45.405 | 2.990 | 0.952 | 1.00 | 23.62 | C |
| ATOM | 2426 | C | ILE | B | 744 | 43.982 | 4.733 | 2.819 | 1.00 | 25.89 | C |
| ATOM | 2427 | O | ILE | B | 744 | 44.797 | 4.898 | 3.691 | 1.00 | 26.76 | O |
| ATOM | 2428 | N | LYS | B | 745 | 43.493 | 5.737 | 2.100 | 1.00 | 28.94 | N |
| ATOM | 2429 | CA | LYS | B | 745 | 44.043 | 7.102 | 2.253 | 1.00 | 31.98 | C |
| ATOM | 2430 | CB | LYS | B | 745 | 44.911 | 7.474 | 1.035 | 1.00 | 31.99 | C |
| ATOM | 2431 | CG | LYS | B | 745 | 46.346 | 6.997 | 1.095 | 1.00 | 33.87 | C |
| ATOM | 2432 | CD | LYS | B | 745 | 46.918 | 6.683 | −0.307 | 1.00 | 35.93 | C |
| ATOM | 2433 | CE | LYS | B | 745 | 47.283 | 7.943 | −1.086 | 1.00 | 33.52 | C |
| ATOM | 2434 | NZ | LYS | B | 745 | 46.120 | 8.507 | −1.826 | 1.00 | 32.74 | N |
| ATOM | 2435 | C | LYS | B | 745 | 43.011 | 8.218 | 2.510 | 1.00 | 33.73 | C |
| ATOM | 2436 | O | LYS | B | 745 | 43.033 | 8.889 | 3.574 | 1.00 | 35.26 | O |
| ATOM | 2437 | N | GLU | B | 746 | 42.125 | 8.457 | 1.542 | 1.00 | 34.28 | N |
| ATOM | 2438 | CA | GLU | B | 746 | 41.094 | 9.456 | 1.767 | 1.00 | 35.03 | C |
| ATOM | 2439 | CB | GLU | B | 746 | 41.370 | 10.733 | 0.995 | 1.00 | 35.68 | C |
| ATOM | 2440 | CG | GLU | B | 746 | 41.647 | 10.472 | −0.457 | 1.00 | 39.53 | C |
| ATOM | 2441 | CD | GLU | B | 746 | 42.782 | 9.502 | −0.614 | 1.00 | 42.55 | C |
| ATOM | 2442 | OE1 | GLU | B | 746 | 43.920 | 9.880 | −0.281 | 1.00 | 42.51 | O |
| ATOM | 2443 | OE2 | GLU | B | 746 | 42.519 | 8.364 | −1.034 | 1.00 | 47.78 | O |
| ATOM | 2444 | C | GLU | B | 746 | 39.726 | 8.925 | 1.439 | 1.00 | 34.47 | C |
| ATOM | 2445 | O | GLU | B | 746 | 38.844 | 8.974 | 2.266 | 1.00 | 36.86 | O |
| ATOM | 2446 | N | GLU | B | 747 | 39.526 | 8.424 | 0.234 | 1.00 | 33.35 | N |
| ATOM | 2447 | CA | GLU | B | 747 | 38.245 | 7.839 | −0.115 | 1.00 | 31.94 | C |
| ATOM | 2448 | CB | GLU | B | 747 | 37.426 | 8.821 | −0.936 | 1.00 | 32.95 | C |
| ATOM | 2449 | CG | GLU | B | 747 | 37.090 | 10.125 | −0.212 | 1.00 | 36.33 | C |
| ATOM | 2450 | CD | GLU | B | 747 | 35.781 | 10.735 | −0.705 | 1.00 | 39.89 | C |
| ATOM | 2451 | OE1 | GLU | B | 747 | 34.720 | 10.153 | −0.373 | 1.00 | 39.59 | O |
| ATOM | 2452 | OE2 | GLU | B | 747 | 35.812 | 11.772 | −1.431 | 1.00 | 40.99 | O |
| ATOM | 2453 | C | GLU | B | 747 | 38.504 | 6.586 | −0.928 | 1.00 | 30.15 | C |
| ATOM | 2454 | O | GLU | B | 747 | 37.614 | 6.091 | −1.625 | 1.00 | 29.18 | O |
| ATOM | 2455 | N | GLU | B | 748 | 39.747 | 6.109 | −0.820 | 1.00 | 28.29 | N |
| ATOM | 2456 | CA | GLU | B | 748 | 40.251 | 4.934 | −1.509 | 1.00 | 27.13 | C |
| ATOM | 2457 | CB | GLU | B | 748 | 41.666 | 5.225 | −2.007 | 1.00 | 27.78 | C |
| ATOM | 2458 | CG | GLU | B | 748 | 42.369 | 4.026 | −2.600 | 1.00 | 31.92 | C |
| ATOM | 2459 | CD | GLU | B | 748 | 41.560 | 3.391 | −3.728 | 1.00 | 39.42 | C |
| ATOM | 2460 | OE1 | GLU | B | 748 | 40.892 | 4.135 | −4.511 | 1.00 | 41.77 | O |
| ATOM | 2461 | OE2 | GLU | B | 748 | 41.576 | 2.138 | −3.828 | 1.00 | 41.89 | O |
| ATOM | 2462 | C | GLU | B | 748 | 40.192 | 3.607 | −0.701 | 1.00 | 25.04 | C |
| ATOM | 2463 | O | GLU | B | 748 | 40.724 | 3.480 | 0.411 | 1.00 | 23.53 | O |
| ATOM | 2464 | N | TYR | B | 749 | 39.506 | 2.621 | −1.270 | 1.00 | 23.64 | N |
| ATOM | 2465 | CA | TYR | B | 749 | 39.390 | 1.305 | −0.645 | 1.00 | 21.89 | C |
| ATOM | 2466 | CB | TYR | B | 749 | 37.939 | 1.027 | −0.350 | 1.00 | 21.59 | C |
| ATOM | 2467 | CG | TYR | B | 749 | 37.363 | 1.979 | 0.662 | 1.00 | 19.85 | C |
| ATOM | 2468 | CD1 | TYR | B | 749 | 37.125 | 3.290 | 0.327 | 1.00 | 17.50 | C |
| ATOM | 2469 | CE1 | TYR | B | 749 | 36.596 | 4.167 | 1.231 | 1.00 | 19.14 | C |
| ATOM | 2470 | CZ | TYR | B | 749 | 36.300 | 3.745 | 2.506 | 1.00 | 19.16 | C |
| ATOM | 2471 | OH | TYR | B | 749 | 35.764 | 4.657 | 3.419 | 1.00 | 18.64 | O |
| ATOM | 2472 | CE2 | TYR | B | 749 | 36.536 | 2.427 | 2.857 | 1.00 | 19.01 | C |
| ATOM | 2473 | CD2 | TYR | B | 749 | 37.063 | 1.563 | 1.946 | 1.00 | 17.38 | C |
| ATOM | 2474 | C | TYR | B | 749 | 39.964 | 0.192 | −1.504 | 1.00 | 20.89 | C |
| ATOM | 2475 | O | TYR | B | 749 | 39.916 | 0.259 | −2.716 | 1.00 | 21.97 | O |
| ATOM | 2476 | N | ASP | B | 750 | 40.494 | −0.837 | −0.866 | 1.00 | 20.13 | N |
| ATOM | 2477 | CA | ASP | B | 750 | 41.111 | −1.970 | −1.538 | 1.00 | 18.82 | C |
| ATOM | 2478 | CB | ASP | B | 750 | 42.567 | −1.679 | −1.874 | 1.00 | 19.07 | C |
| ATOM | 2479 | CG | ASP | B | 750 | 42.941 | −2.117 | −3.312 | 1.00 | 20.57 | C |
| ATOM | 2480 | OD1 | ASP | B | 750 | 42.687 | −3.314 | −3.677 | 1.00 | 15.59 | O |
| ATOM | 2481 | OD2 | ASP | B | 750 | 43.496 | −1.299 | −4.114 | 1.00 | 20.85 | O |
| ATOM | 2482 | C | ASP | B | 750 | 41.067 | −3.110 | −0.580 | 1.00 | 17.86 | C |
| ATOM | 2483 | O | ASP | B | 750 | 40.606 | −2.927 | 0.545 | 1.00 | 18.20 | O |
| ATOM | 2484 | N | SER | B | 751 | 41.534 | −4.277 | −1.023 | 1.00 | 16.66 | N |
| ATOM | 2485 | CA | SER | B | 751 | 41.594 | −5.478 | −0.198 | 1.00 | 15.69 | C |
| ATOM | 2486 | CB | SER | B | 751 | 42.277 | −6.578 | −0.976 | 1.00 | 14.91 | C |
| ATOM | 2487 | OG | SER | B | 751 | 43.683 | −6.392 | −0.953 | 1.00 | 15.07 | O |
| ATOM | 2488 | C | SER | B | 751 | 42.349 | −5.264 | 1.138 | 1.00 | 15.52 | C |
| ATOM | 2489 | O | SER | B | 751 | 43.138 | −4.346 | 1.310 | 1.00 | 15.02 | O |
| ATOM | 2490 | N | ILE | B | 752 | 42.142 | −6.147 | 2.083 | 1.00 | 15.22 | N |
| ATOM | 2491 | CA | ILE | B | 752 | 42.790 | −5.968 | 3.367 | 1.00 | 15.33 | C |
| ATOM | 2492 | CB | ILE | B | 752 | 42.236 | −6.937 | 4.387 | 1.00 | 14.48 | C |
| ATOM | 2493 | CG1 | ILE | B | 752 | 42.606 | −6.495 | 5.805 | 1.00 | 15.87 | C |
| ATOM | 2494 | CD1 | ILE | B | 752 | 41.941 | −7.320 | 6.917 | 1.00 | 12.60 | C |
| ATOM | 2495 | CG2 | ILE | B | 752 | 42.760 | −8.248 | 4.108 | 1.00 | 14.29 | C |
| ATOM | 2496 | C | ILE | B | 752 | 44.295 | −6.089 | 3.284 | 1.00 | 15.94 | C |
| ATOM | 2497 | O | ILE | B | 752 | 44.984 | −5.642 | 4.209 | 1.00 | 16.82 | O |
| ATOM | 2498 | N | ILE | B | 753 | 44.803 | −6.656 | 2.186 | 1.00 | 15.32 | N |
| ATOM | 2499 | CA | ILE | B | 753 | 46.219 | −6.847 | 2.011 | 1.00 | 15.05 | C |
| ATOM | 2500 | CB | ILE | B | 753 | 46.481 | −7.795 | 0.834 | 1.00 | 16.49 | C |
| ATOM | 2501 | CG1 | ILE | B | 753 | 46.016 | −9.193 | 1.137 | 1.00 | 17.73 | C |
| ATOM | 2502 | CD1 | ILE | B | 753 | 45.975 | −10.051 | −0.111 | 1.00 | 17.48 | C |
| ATOM | 2503 | CG2 | ILE | B | 753 | 47.962 | −7.928 | 0.502 | 1.00 | 17.13 | C |
| ATOM | 2504 | C | ILE | B | 753 | 46.830 | −5.512 | 1.672 | 1.00 | 14.92 | C |
| ATOM | 2505 | O | ILE | B | 753 | 47.876 | −5.155 | 2.189 | 1.00 | 15.50 | O |
| ATOM | 2506 | N | VAL | B | 754 | 46.222 | −4.772 | 0.756 | 1.00 | 14.19 | N |
| ATOM | 2507 | CA | VAL | B | 754 | 46.811 | −3.490 | 0.387 | 1.00 | 13.27 | C |
| ATOM | 2508 | CB | VAL | B | 754 | 45.977 | −2.757 | −0.669 | 1.00 | 13.73 | C |
| ATOM | 2509 | CG1 | VAL | B | 754 | 46.516 | −1.366 | −0.960 | 1.00 | 13.78 | C |
| ATOM | 2510 | CG2 | VAL | B | 754 | 45.829 | −3.593 | −1.938 | 1.00 | 13.40 | C |
| ATOM | 2511 | C | VAL | B | 754 | 46.863 | −2.657 | 1.656 | 1.00 | 13.15 | C |
| ATOM | 2512 | O | VAL | B | 754 | 47.807 | −1.884 | 1.862 | 1.00 | 12.76 | O |
| ATOM | 2513 | N | PHE | B | 755 | 45.863 | −2.832 | 2.528 | 1.00 | 12.25 | N |
| ATOM | 2514 | CA | PHE | B | 755 | 45.849 | −2.081 | 3.763 | 1.00 | 11.74 | C |
| ATOM | 2515 | CB | PHE | B | 755 | 44.564 | −2.286 | 4.554 | 1.00 | 12.24 | C |
| ATOM | 2516 | CG | PHE | B | 755 | 44.591 | −1.641 | 5.917 | 1.00 | 9.65 | C |
| ATOM | 2517 | CD1 | PHE | B | 755 | 44.648 | −0.250 | 6.030 | 1.00 | 11.69 | C |
| ATOM | 2518 | CE1 | PHE | B | 755 | 44.710 | 0.373 | 7.313 | 1.00 | 13.58 | C |
| ATOM | 2519 | CZ | PHE | B | 755 | 44.678 | −0.431 | 8.444 | 1.00 | 12.16 | C |
| ATOM | 2520 | CE2 | PHE | B | 755 | 44.603 | −1.833 | 8.307 | 1.00 | 8.49 | C |
| ATOM | 2521 | CD2 | PHE | B | 755 | 44.568 | −2.414 | 7.070 | 1.00 | 5.62 | C |
| ATOM | 2522 | C | PHE | B | 755 | 47.028 | −2.440 | 4.638 | 1.00 | 11.89 | C |
| ATOM | 2523 | O | PHE | B | 755 | 47.725 | −1.554 | 5.142 | 1.00 | 10.64 | O |
| ATOM | 2524 | N | TYR | B | 756 | 47.239 | −3.741 | 4.848 | 1.00 | 12.44 | N |
| ATOM | 2525 | CA | TYR | B | 756 | 48.369 | −4.170 | 5.674 | 1.00 | 13.71 | C |
| ATOM | 2526 | CB | TYR | B | 756 | 48.450 | −5.675 | 5.721 | 1.00 | 13.48 | C |
| ATOM | 2527 | CG | TYR | B | 756 | 49.697 | −6.181 | 6.414 | 1.00 | 16.30 | C |
| ATOM | 2528 | CD1 | TYR | B | 756 | 49.792 | −6.197 | 7.793 | 1.00 | 16.18 | C |
| ATOM | 2529 | CE1 | TYR | B | 756 | 50.933 | −6.683 | 8.441 | 1.00 | 18.26 | C |
| ATOM | 2530 | CZ | TYR | B | 756 | 52.012 | −7.161 | 7.687 | 1.00 | 21.98 | C |
| ATOM | 2531 | OH | TYR | B | 756 | 53.166 | −7.628 | 8.297 | 1.00 | 17.21 | O |
| ATOM | 2532 | CE2 | TYR | B | 756 | 51.935 | −7.156 | 6.293 | 1.00 | 20.27 | C |
| ATOM | 2533 | CD2 | TYR | B | 756 | 50.782 | −6.674 | 5.673 | 1.00 | 20.75 | C |
| ATOM | 2534 | C | TYR | B | 756 | 49.688 | −3.564 | 5.123 | 1.00 | 14.37 | C |
| ATOM | 2535 | O | TYR | B | 756 | 50.386 | −2.764 | 5.791 | 1.00 | 14.12 | O |
| ATOM | 2536 | N | ASN | B | 757 | 49.988 | −3.880 | 3.867 | 1.00 | 14.19 | N |
| ATOM | 2537 | CA | ASN | B | 757 | 51.213 | −3.374 | 3.300 | 1.00 | 15.33 | C |
| ATOM | 2538 | CB | ASN | B | 757 | 51.516 | −4.070 | 1.984 | 1.00 | 15.54 | C |
| ATOM | 2539 | CG | ASN | B | 757 | 51.770 | −5.537 | 2.177 | 1.00 | 18.19 | C |
| ATOM | 2540 | OD1 | ASN | B | 757 | 52.778 | −5.936 | 2.783 | 1.00 | 19.68 | O |
| ATOM | 2541 | ND2 | ASN | B | 757 | 50.851 | −6.363 | 1.680 | 1.00 | 20.50 | N |
| ATOM | 2542 | C | ASN | B | 757 | 51.320 | −1.860 | 3.141 | 1.00 | 14.85 | C |
| ATOM | 2543 | O | ASN | B | 757 | 52.419 | −1.337 | 3.198 | 1.00 | 15.14 | O |
| ATOM | 2544 | N | SER | B | 758 | 50.210 | −1.162 | 2.942 | 1.00 | 13.60 | N |
| ATOM | 2545 | CA | SER | B | 758 | 50.310 | 0.251 | 2.646 | 1.00 | 13.76 | C |
| ATOM | 2546 | CB | SER | B | 758 | 49.283 | 0.667 | 1.603 | 1.00 | 13.54 | C |
| ATOM | 2547 | OG | SER | B | 758 | 49.507 | −0.024 | 0.387 | 1.00 | 15.82 | O |
| ATOM | 2548 | C | SER | B | 758 | 50.167 | 1.178 | 3.823 | 1.00 | 14.24 | C |
| ATOM | 2549 | O | SER | B | 758 | 50.628 | 2.331 | 3.727 | 1.00 | 14.50 | O |
| ATOM | 2550 | N | VAL | B | 759 | 49.516 | 0.701 | 4.899 | 1.00 | 13.20 | N |
| ATOM | 2551 | CA | VAL | B | 759 | 49.204 | 1.515 | 6.035 | 1.00 | 12.81 | C |
| ATOM | 2552 | CB | VAL | B | 759 | 47.751 | 1.834 | 6.030 | 1.00 | 12.98 | C |
| ATOM | 2553 | CG1 | VAL | B | 759 | 47.457 | 2.869 | 7.066 | 1.00 | 12.46 | C |
| ATOM | 2554 | CG2 | VAL | B | 759 | 47.378 | 2.343 | 4.650 | 1.00 | 13.50 | C |
| ATOM | 2555 | C | VAL | B | 759 | 49.558 | 0.906 | 7.375 | 1.00 | 14.10 | C |
| ATOM | 2556 | O | VAL | B | 759 | 50.245 | 1.528 | 8.144 | 1.00 | 14.24 | O |
| ATOM | 2557 | N | PHE | B | 760 | 49.113 | −0.320 | 7.641 | 1.00 | 14.78 | N |
| ATOM | 2558 | CA | PHE | B | 760 | 49.305 | −0.964 | 8.916 | 1.00 | 15.29 | C |
| ATOM | 2559 | CB | PHE | B | 760 | 48.569 | −2.314 | 8.884 | 1.00 | 15.21 | C |
| ATOM | 2560 | CG | PHE | B | 760 | 48.414 | −2.965 | 10.234 | 1.00 | 14.87 | C |
| ATOM | 2561 | CD1 | PHE | B | 760 | 47.385 | −2.591 | 11.085 | 1.00 | 13.04 | C |
| ATOM | 2562 | CE1 | PHE | B | 760 | 47.237 | −3.188 | 12.302 | 1.00 | 13.09 | C |
| ATOM | 2563 | CZ | PHE | B | 760 | 48.118 | −4.129 | 12.712 | 1.00 | 13.72 | C |
| ATOM | 2564 | CE2 | PHE | B | 760 | 49.165 | −4.488 | 11.897 | 1.00 | 15.35 | C |
| ATOM | 2565 | CD2 | PHE | B | 760 | 49.304 | −3.918 | 10.660 | 1.00 | 13.80 | C |
| ATOM | 2566 | C | PHE | B | 760 | 50.785 | −1.162 | 9.246 | 1.00 | 16.61 | C |
| ATOM | 2567 | O | PHE | B | 760 | 51.333 | −0.642 | 10.217 | 1.00 | 16.43 | O |
| ATOM | 2568 | N | MET | B | 761 | 51.422 | −1.954 | 8.426 | 1.00 | 18.47 | N |
| ATOM | 2569 | CA | MET | B | 761 | 52.791 | −2.303 | 8.631 | 1.00 | 21.12 | C |
| ATOM | 2570 | CB | MET | B | 761 | 53.267 | −3.148 | 7.451 | 1.00 | 22.42 | C |
| ATOM | 2571 | CG | MET | B | 761 | 54.775 | −3.092 | 7.205 | 1.00 | 24.52 | C |
| ATOM | 2572 | SD | MET | B | 761 | 55.033 | −4.025 | 5.779 | 1.00 | 32.46 | S |
| ATOM | 2573 | CE | MET | B | 761 | 55.132 | −5.783 | 6.464 | 1.00 | 33.08 | C |
| ATOM | 2574 | C | MET | B | 761 | 53.689 | −1.091 | 8.816 | 1.00 | 21.28 | C |
| ATOM | 2575 | O | MET | B | 761 | 54.647 | −1.132 | 9.533 | 1.00 | 22.18 | O |
| ATOM | 2576 | N | GLN | B | 762 | 53.381 | −0.005 | 8.159 | 1.00 | 22.50 | N |
| ATOM | 2577 | CA | GLN | B | 762 | 54.223 | 1.171 | 8.259 | 1.00 | 22.90 | C |
| ATOM | 2578 | CB | GLN | B | 762 | 53.913 | 2.123 | 7.106 | 1.00 | 23.18 | C |
| ATOM | 2579 | CG | GLN | B | 762 | 53.783 | 1.393 | 5.714 | 1.00 | 27.77 | C |
| ATOM | 2580 | CD | GLN | B | 762 | 55.041 | 0.617 | 5.279 | 1.00 | 32.36 | C |
| ATOM | 2581 | OE1 | GLN | B | 762 | 55.947 | 1.172 | 4.658 | 1.00 | 34.37 | O |
| ATOM | 2582 | NE2 | GLN | B | 762 | 55.071 | −0.687 | 5.585 | 1.00 | 36.21 | N |
| ATOM | 2583 | C | GLN | B | 762 | 54.043 | 1.853 | 9.593 | 1.00 | 21.95 | C |
| ATOM | 2584 | O | GLN | B | 762 | 54.985 | 2.236 | 10.247 | 1.00 | 21.06 | O |
| ATOM | 2585 | N | ARG | B | 763 | 52.815 | 2.011 | 10.012 | 1.00 | 21.79 | N |
| ATOM | 2586 | CA | ARG | B | 763 | 52.622 | 2.728 | 11.232 | 1.00 | 22.55 | C |
| ATOM | 2587 | CB | ARG | B | 763 | 51.143 | 3.037 | 11.405 | 1.00 | 23.18 | C |
| ATOM | 2588 | CG | ARG | B | 763 | 50.785 | 3.636 | 12.785 | 1.00 | 27.01 | C |
| ATOM | 2589 | CD | ARG | B | 763 | 51.197 | 5.097 | 12.938 | 1.00 | 31.66 | C |
| ATOM | 2590 | NE | ARG | B | 763 | 50.298 | 5.888 | 12.126 | 1.00 | 36.41 | N |
| ATOM | 2591 | CZ | ARG | B | 763 | 49.055 | 6.188 | 12.498 | 1.00 | 39.77 | C |
| ATOM | 2592 | NH1 | ARG | B | 763 | 48.603 | 5.780 | 13.691 | 1.00 | 36.61 | N |
| ATOM | 2593 | NH2 | ARG | B | 763 | 48.273 | 6.913 | 11.691 | 1.00 | 40.01 | N |
| ATOM | 2594 | C | ARG | B | 763 | 53.158 | 1.947 | 12.436 | 1.00 | 22.17 | C |
| ATOM | 2595 | O | ARG | B | 763 | 53.538 | 2.537 | 13.438 | 1.00 | 19.77 | O |
| ATOM | 2596 | N | LEU | B | 764 | 53.190 | 0.617 | 12.300 | 1.00 | 22.80 | N |
| ATOM | 2597 | CA | LEU | B | 764 | 53.588 | −0.269 | 13.386 | 1.00 | 23.24 | C |
| ATOM | 2598 | CB | LEU | B | 764 | 52.500 | −1.301 | 13.594 | 1.00 | 23.48 | C |
| ATOM | 2599 | CG | LEU | B | 764 | 51.177 | −0.718 | 14.097 | 1.00 | 24.36 | C |
| ATOM | 2600 | CD1 | LEU | B | 764 | 50.013 | −1.676 | 13.885 | 1.00 | 24.21 | C |
| ATOM | 2601 | CD2 | LEU | B | 764 | 51.348 | −0.480 | 15.529 | 1.00 | 24.26 | C |
| ATOM | 2602 | C | LEU | B | 764 | 54.917 | −0.993 | 13.193 | 1.00 | 23.50 | C |
| ATOM | 2603 | O | LEU | B | 764 | 55.345 | −1.716 | 14.089 | 1.00 | 21.69 | O |
| ATOM | 2604 | N | LYS | B | 765 | 55.556 | −0.812 | 12.032 | 1.00 | 24.54 | N |
| ATOM | 2605 | CA | LYS | B | 765 | 56.800 | −1.510 | 11.719 | 1.00 | 26.74 | C |
| ATOM | 2606 | CB | LYS | B | 765 | 57.462 | −0.944 | 10.464 | 1.00 | 26.88 | C |
| ATOM | 2607 | CG | LYS | B | 765 | 58.915 | −1.493 | 10.240 | 1.00 | 28.72 | C |
| ATOM | 2608 | CD | LYS | B | 765 | 59.559 | −1.044 | 8.915 | 1.00 | 31.45 | C |
| ATOM | 2609 | CE | LYS | B | 765 | 58.982 | −1.767 | 7.693 | 1.00 | 34.41 | C |
| ATOM | 2610 | NZ | LYS | B | 765 | 57.496 | −1.689 | 7.597 | 1.00 | 37.61 | N |
| ATOM | 2611 | C | LYS | B | 765 | 57.796 | −1.462 | 12.879 | 1.00 | 28.01 | C |
| ATOM | 2612 | O | LYS | B | 765 | 58.254 | −2.494 | 13.377 | 1.00 | 27.53 | O |
| ATOM | 2613 | N | THR | B | 766 | 58.126 | −0.242 | 13.273 | 1.00 | 29.05 | N |
| ATOM | 2614 | CA | THR | B | 766 | 58.966 | 0.013 | 14.424 | 1.00 | 30.88 | C |
| ATOM | 2615 | CB | THR | B | 766 | 58.821 | 1.516 | 14.840 | 1.00 | 31.26 | C |
| ATOM | 2616 | OG1 | THR | B | 766 | 59.035 | 2.348 | 13.698 | 1.00 | 33.49 | O |
| ATOM | 2617 | CG2 | THR | B | 766 | 59.909 | 1.962 | 15.802 | 1.00 | 30.65 | C |
| ATOM | 2618 | C | THR | B | 766 | 58.615 | −0.872 | 15.610 | 1.00 | 30.99 | C |
| ATOM | 2619 | O | THR | B | 766 | 59.449 | −1.569 | 16.119 | 1.00 | 32.43 | O |
| ATOM | 2620 | N | ASN | B | 767 | 57.398 | −0.804 | 16.083 | 1.00 | 31.61 | N |
| ATOM | 2621 | CA | ASN | B | 767 | 56.992 | −1.570 | 17.232 | 1.00 | 33.54 | C |
| ATOM | 2622 | CB | ASN | B | 767 | 55.652 | −1.023 | 17.768 | 1.00 | 33.93 | C |
| ATOM | 2623 | CG | ASN | B | 767 | 55.048 | −1.892 | 18.837 | 1.00 | 36.39 | C |
| ATOM | 2624 | OD1 | ASN | B | 767 | 54.504 | −2.945 | 18.545 | 1.00 | 39.39 | O |
| ATOM | 2625 | ND2 | ASN | B | 767 | 55.142 | −1.456 | 20.094 | 1.00 | 40.65 | N |
| ATOM | 2626 | C | ASN | B | 767 | 56.926 | −3.092 | 16.985 | 1.00 | 34.59 | C |
| ATOM | 2627 | O | ASN | B | 767 | 57.394 | −3.895 | 17.816 | 1.00 | 34.71 | O |
| ATOM | 2628 | N | ILE | B | 768 | 56.354 | −3.511 | 15.863 | 1.00 | 35.47 | N |
| ATOM | 2629 | CA | ILE | B | 768 | 56.239 | −4.940 | 15.625 | 1.00 | 36.32 | C |
| ATOM | 2630 | CB | ILE | B | 768 | 55.621 | −5.237 | 14.268 | 1.00 | 36.49 | C |
| ATOM | 2631 | CG1 | ILE | B | 768 | 54.105 | −4.966 | 14.309 | 1.00 | 35.72 | C |
| ATOM | 2632 | CD1 | ILE | B | 768 | 53.429 | −5.168 | 12.981 | 1.00 | 35.45 | C |
| ATOM | 2633 | CG2 | ILE | B | 768 | 55.950 | −6.683 | 13.834 | 1.00 | 34.18 | C |
| ATOM | 2634 | C | ILE | B | 768 | 57.592 | −5.577 | 15.698 | 1.00 | 37.63 | C |
| ATOM | 2635 | O | ILE | B | 768 | 57.694 | −6.786 | 15.923 | 1.00 | 38.19 | O |
| ATOM | 2636 | N | LEU | B | 769 | 58.626 | −4.764 | 15.505 | 1.00 | 38.55 | N |
| ATOM | 2637 | CA | LEU | B | 769 | 59.996 | −5.257 | 15.523 | 1.00 | 40.19 | C |
| ATOM | 2638 | CB | LEU | B | 769 | 60.996 | −4.186 | 15.082 | 1.00 | 40.99 | C |
| ATOM | 2639 | CG | LEU | B | 769 | 61.863 | −4.615 | 13.896 | 1.00 | 42.49 | C |
| ATOM | 2640 | CD1 | LEU | B | 769 | 62.460 | −5.984 | 14.251 | 1.00 | 45.30 | C |
| ATOM | 2641 | CD2 | LEU | B | 769 | 61.061 | −4.723 | 12.578 | 1.00 | 42.90 | C |
| ATOM | 2642 | C | LEU | B | 769 | 60.375 | −5.753 | 16.890 | 1.00 | 40.93 | C |
| ATOM | 2643 | O | LEU | B | 769 | 61.022 | −6.789 | 16.999 | 1.00 | 41.56 | O |
| ATOM | 2644 | N | GLN | B | 770 | 59.987 | −5.024 | 17.936 | 1.00 | 41.51 | N |
| ATOM | 2645 | CA | GLN | B | 770 | 60.285 | −5.473 | 19.304 | 1.00 | 41.74 | C |
| ATOM | 2646 | CB | GLN | B | 770 | 59.366 | −4.794 | 20.343 | 1.00 | 41.53 | C |
| ATOM | 2647 | CG | GLN | B | 770 | 59.450 | −5.418 | 21.762 | 1.00 | 42.43 | C |
| ATOM | 2648 | CD | GLN | B | 770 | 58.133 | −5.425 | 22.587 | 1.00 | 42.74 | C |
| ATOM | 2649 | OE1 | GLN | B | 770 | 58.064 | −6.041 | 23.668 | 1.00 | 41.16 | O |
| ATOM | 2650 | NE2 | GLN | B | 770 | 57.116 | −4.740 | 22.090 | 1.00 | 43.74 | N |
| ATOM | 2651 | C | GLN | B | 770 | 60.125 | −7.004 | 19.380 | 1.00 | 41.82 | C |
| ATOM | 2652 | O | GLN | B | 770 | 61.077 | −7.747 | 19.610 | 1.00 | 41.81 | O |
| ATOM | 2653 | N | TYR | B | 771 | 58.911 | −7.467 | 19.144 | 1.00 | 42.24 | N |
| ATOM | 2654 | CA | TYR | B | 771 | 58.610 | −8.888 | 19.229 | 1.00 | 42.97 | C |
| ATOM | 2655 | CB | TYR | B | 771 | 57.180 | −9.140 | 18.775 | 1.00 | 41.87 | C |
| ATOM | 2656 | CG | TYR | B | 771 | 56.175 | −8.189 | 19.354 | 1.00 | 38.45 | C |
| ATOM | 2657 | CD1 | TYR | B | 771 | 55.729 | −8.307 | 20.662 | 1.00 | 37.30 | C |
| ATOM | 2658 | CE1 | TYR | B | 771 | 54.776 | −7.426 | 21.169 | 1.00 | 35.23 | C |
| ATOM | 2659 | CZ | TYR | B | 771 | 54.263 | −6.431 | 20.343 | 1.00 | 34.31 | C |
| ATOM | 2660 | OH | TYR | B | 771 | 53.309 | −5.491 | 20.752 | 1.00 | 34.71 | O |
| ATOM | 2661 | CE2 | TYR | B | 771 | 54.703 | −6.332 | 19.061 | 1.00 | 33.76 | C |
| ATOM | 2662 | CD2 | TYR | B | 771 | 55.641 | −7.191 | 18.576 | 1.00 | 35.17 | C |
| ATOM | 2663 | C | TYR | B | 771 | 59.572 | −9.775 | 18.441 | 1.00 | 44.15 | C |
| ATOM | 2664 | O | TYR | B | 771 | 59.703 | −10.968 | 18.720 | 1.00 | 43.36 | O |
| ATOM | 2665 | N | ALA | B | 772 | 60.235 | −9.181 | 17.454 | 1.00 | 46.14 | N |
| ATOM | 2666 | CA | ALA | B | 772 | 61.159 | −9.932 | 16.620 | 1.00 | 48.39 | C |
| ATOM | 2667 | CB | ALA | B | 772 | 61.590 | −9.113 | 15.432 | 1.00 | 48.34 | C |
| ATOM | 2668 | C | ALA | B | 772 | 62.368 | −10.356 | 17.425 | 1.00 | 50.30 | C |
| ATOM | 2669 | O | ALA | B | 772 | 62.956 | −11.413 | 17.175 | 1.00 | 50.02 | O |
| ATOM | 2670 | N | SER | B | 773 | 62.746 | −9.510 | 18.383 | 1.00 | 52.64 | N |
| ATOM | 2671 | CA | SER | B | 773 | 63.884 | −9.794 | 19.238 | 1.00 | 54.66 | C |
| ATOM | 2672 | CB | SER | B | 773 | 64.021 | −8.723 | 20.320 | 1.00 | 54.48 | C |
| ATOM | 2673 | OG | SER | B | 773 | 65.106 | −9.024 | 21.187 | 1.00 | 55.08 | O |
| ATOM | 2674 | C | SER | B | 773 | 63.691 | −11.173 | 19.879 | 1.00 | 56.17 | C |
| ATOM | 2675 | O | SER | B | 773 | 62.634 | −11.447 | 20.455 | 1.00 | 56.55 | O |
| ATOM | 2676 | N | THR | B | 774 | 64.709 | −12.031 | 19.768 | 1.00 | 57.80 | N |
| ATOM | 2677 | CA | THR | B | 774 | 64.694 | −13.397 | 20.328 | 1.00 | 59.18 | C |
| ATOM | 2678 | CB | THR | B | 774 | 65.990 | −14.164 | 19.901 | 1.00 | 59.51 | C |
| ATOM | 2679 | OG1 | THR | B | 774 | 67.155 | −13.432 | 20.324 | 1.00 | 60.23 | O |
| ATOM | 2680 | CG2 | THR | B | 774 | 66.138 | −14.219 | 18.355 | 1.00 | 59.89 | C |
| ATOM | 2681 | C | THR | B | 774 | 64.527 | −13.436 | 21.875 | 1.00 | 59.52 | C |
| ATOM | 2682 | O | THR | B | 774 | 64.836 | −14.440 | 22.533 | 1.00 | 59.47 | O |
| ATOM | 2683 | N | ARG | B | 775 | 64.043 | −12.330 | 22.433 | 1.00 | 59.58 | N |
| ATOM | 2684 | CA | ARG | B | 775 | 63.800 | −12.203 | 23.861 | 1.00 | 59.55 | C |
| ATOM | 2685 | CB | ARG | B | 775 | 64.540 | −10.968 | 24.393 | 1.00 | 59.96 | C |
| ATOM | 2686 | CG | ARG | B | 775 | 64.053 | −9.623 | 23.789 | 1.00 | 60.09 | C |
| ATOM | 2687 | CD | ARG | B | 775 | 64.791 | −8.402 | 24.311 | 1.00 | 59.27 | C |
| ATOM | 2688 | NE | ARG | B | 775 | 64.233 | −7.147 | 23.810 | 1.00 | 58.26 | N |
| ATOM | 2689 | CZ | ARG | B | 775 | 63.016 | −6.685 | 24.091 | 1.00 | 57.54 | C |
| ATOM | 2690 | NH1 | ARG | B | 775 | 62.186 | −7.364 | 24.871 | 1.00 | 56.28 | N |
| ATOM | 2691 | NH2 | ARG | B | 775 | 62.618 | −5.532 | 23.577 | 1.00 | 57.23 | N |
| ATOM | 2692 | C | ARG | B | 775 | 62.283 | −12.077 | 24.117 | 1.00 | 59.35 | C |
| ATOM | 2693 | O | ARG | B | 775 | 61.559 | −11.452 | 23.327 | 1.00 | 59.00 | O |
| ATOM | 2694 | N | PRO | B | 776 | 61.799 | −12.663 | 25.217 | 1.00 | 59.19 | N |
| ATOM | 2695 | CA | PRO | B | 776 | 60.358 | −12.614 | 25.546 | 1.00 | 58.52 | C |
| ATOM | 2696 | CB | PRO | B | 776 | 60.263 | −13.471 | 26.817 | 1.00 | 58.69 | C |
| ATOM | 2697 | CG | PRO | B | 776 | 61.659 | −13.372 | 27.413 | 1.00 | 59.27 | C |
| ATOM | 2698 | CD | PRO | B | 776 | 62.577 | −13.431 | 26.219 | 1.00 | 59.00 | C |
| ATOM | 2699 | C | PRO | B | 776 | 59.846 | −11.173 | 25.804 | 1.00 | 57.74 | C |
| ATOM | 2700 | O | PRO | B | 776 | 60.560 | −10.337 | 26.404 | 1.00 | 57.92 | O |
| ATOM | 2701 | N | PRO | B | 777 | 58.638 | −10.887 | 25.321 | 1.00 | 56.28 | N |
| ATOM | 2702 | CA | PRO | B | 777 | 58.011 | −9.569 | 25.464 | 1.00 | 55.27 | C |
| ATOM | 2703 | CB | PRO | B | 777 | 57.078 | −9.548 | 24.271 | 1.00 | 55.36 | C |
| ATOM | 2704 | CG | PRO | B | 777 | 56.561 | −10.950 | 24.291 | 1.00 | 56.03 | C |
| ATOM | 2705 | CD | PRO | B | 777 | 57.807 | −11.793 | 24.508 | 1.00 | 56.21 | C |
| ATOM | 2706 | C | PRO | B | 777 | 57.189 | −9.364 | 26.737 | 1.00 | 54.10 | C |
| ATOM | 2707 | O | PRO | B | 777 | 56.916 | −10.338 | 27.466 | 1.00 | 53.82 | O |
| ATOM | 2708 | N | THR | B | 778 | 56.810 | −8.103 | 26.983 | 1.00 | 52.28 | N |
| ATOM | 2709 | CA | THR | B | 778 | 55.960 | −7.751 | 28.117 | 1.00 | 51.05 | C |
| ATOM | 2710 | CB | THR | B | 778 | 56.128 | −6.263 | 28.526 | 1.00 | 52.62 | C |
| ATOM | 2711 | OG1 | THR | B | 778 | 57.514 | −5.930 | 28.693 | 1.00 | 52.75 | O |
| ATOM | 2712 | CG2 | THR | B | 778 | 55.525 | −6.018 | 29.916 | 1.00 | 51.07 | C |
| ATOM | 2713 | C | THR | B | 778 | 54.508 | −7.973 | 27.702 | 1.00 | 49.65 | C |
| ATOM | 2714 | O | THR | B | 778 | 53.774 | −7.020 | 27.460 | 1.00 | 49.64 | O |
| ATOM | 2715 | N | LEU | B | 779 | 54.098 | −9.228 | 27.595 | 1.00 | 47.40 | N |
| ATOM | 2716 | CA | LEU | B | 779 | 52.748 | −9.548 | 27.199 | 1.00 | 45.15 | C |
| ATOM | 2717 | CB | LEU | B | 779 | 52.486 | −11.020 | 27.483 | 1.00 | 44.67 | C |
| ATOM | 2718 | CG | LEU | B | 779 | 53.553 | −11.917 | 26.856 | 1.00 | 44.69 | C |
| ATOM | 2719 | CD1 | LEU | B | 779 | 53.599 | −13.324 | 27.488 | 1.00 | 45.78 | C |
| ATOM | 2720 | CD2 | LEU | B | 779 | 53.361 | −12.007 | 25.369 | 1.00 | 45.46 | C |
| ATOM | 2721 | C | LEU | B | 779 | 51.739 | −8.680 | 27.941 | 1.00 | 44.03 | C |
| ATOM | 2722 | O | LEU | B | 779 | 51.867 | −8.480 | 29.137 | 1.00 | 43.69 | O |
| ATOM | 2723 | N | SER | B | 780 | 50.729 | −8.184 | 27.219 | 1.00 | 42.70 | N |
| ATOM | 2724 | CA | SER | B | 780 | 49.645 | −7.367 | 27.780 | 1.00 | 41.09 | C |
| ATOM | 2725 | CB | SER | B | 780 | 48.967 | −6.594 | 26.642 | 1.00 | 41.11 | C |
| ATOM | 2726 | OG | SER | B | 780 | 48.015 | −5.655 | 27.119 | 1.00 | 41.62 | O |
| ATOM | 2727 | C | SER | B | 780 | 48.620 | −8.238 | 28.496 | 1.00 | 40.22 | C |
| ATOM | 2728 | O | SER | B | 780 | 48.381 | −9.353 | 28.080 | 1.00 | 39.24 | O |
| ATOM | 2729 | N | PRO | B | 781 | 47.997 | −7.730 | 29.555 | 1.00 | 40.30 | N |
| ATOM | 2730 | CA | PRO | B | 781 | 47.066 | −8.528 | 30.355 | 1.00 | 40.49 | C |
| ATOM | 2731 | CB | PRO | B | 781 | 46.795 | −7.626 | 31.569 | 1.00 | 40.18 | C |
| ATOM | 2732 | CG | PRO | B | 781 | 46.953 | −6.295 | 31.060 | 1.00 | 40.81 | C |
| ATOM | 2733 | CD | PRO | B | 781 | 48.136 | −6.369 | 30.103 | 1.00 | 40.81 | C |
| ATOM | 2734 | C | PRO | B | 781 | 45.775 | −8.859 | 29.620 | 1.00 | 40.94 | C |
| ATOM | 2735 | O | PRO | B | 781 | 45.134 | −7.961 | 29.063 | 1.00 | 41.63 | O |
| ATOM | 2736 | N | ILE | B | 782 | 45.408 | −10.140 | 29.606 | 1.00 | 40.96 | N |
| ATOM | 2737 | CA | ILE | B | 782 | 44.174 | −10.590 | 28.961 | 1.00 | 41.15 | C |
| ATOM | 2738 | CB | ILE | B | 782 | 44.169 | −12.125 | 28.886 | 1.00 | 41.28 | C |
| ATOM | 2739 | CG1 | ILE | B | 782 | 45.321 | −12.601 | 28.000 | 1.00 | 41.51 | C |
| ATOM | 2740 | CD1 | ILE | B | 782 | 45.027 | −12.473 | 26.509 | 1.00 | 42.88 | C |
| ATOM | 2741 | CG2 | ILE | B | 782 | 42.817 | −12.667 | 28.388 | 1.00 | 40.06 | C |
| ATOM | 2742 | C | ILE | B | 782 | 42.923 | −10.093 | 29.710 | 1.00 | 41.48 | C |
| ATOM | 2743 | O | ILE | B | 782 | 42.915 | −9.990 | 30.932 | 1.00 | 40.74 | O |
| ATOM | 2744 | N | PRO | B | 783 | 41.877 | −9.749 | 28.967 | 1.00 | 41.74 | N |
| ATOM | 2745 | CA | PRO | B | 783 | 40.617 | −9.334 | 29.579 | 1.00 | 42.10 | C |
| ATOM | 2746 | CB | PRO | B | 783 | 39.719 | −9.080 | 28.374 | 1.00 | 41.64 | C |
| ATOM | 2747 | CG | PRO | B | 783 | 40.666 | −8.777 | 27.311 | 1.00 | 41.40 | C |
| ATOM | 2748 | CD | PPO | B | 783 | 41.830 | −9.692 | 27.497 | 1.00 | 41.11 | C |
| ATOM | 2749 | C | PRO | B | 783 | 40.049 | −10.450 | 30.413 | 1.00 | 43.09 | C |
| ATOM | 2750 | O | PRO | B | 783 | 39.720 | −11.490 | 29.879 | 1.00 | 42.35 | O |
| ATOM | 2751 | N | HIS | B | 784 | 39.907 | −10.203 | 31.708 | 1.00 | 45.68 | N |
| ATOM | 2752 | CA | HIS | B | 784 | 39.388 | −11.196 | 32.656 | 1.00 | 48.44 | C |
| ATOM | 2753 | CB | HIS | B | 784 | 38.831 | −10.507 | 33.902 | 1.00 | 49.03 | C |
| ATOM | 2754 | CG | HIS | B | 784 | 38.870 | −11.356 | 35.137 | 1.00 | 51.46 | C |
| ATOM | 2755 | ND1 | HIS | B | 784 | 38.178 | −12.545 | 35.248 | 1.00 | 53.83 | N |
| ATOM | 2756 | CE1 | HIS | B | 784 | 38.390 | −13.068 | 36.446 | 1.00 | 54.70 | C |
| ATOM | 2757 | NE2 | HIS | B | 784 | 39.185 | −12.254 | 37.122 | 1.00 | 54.84 | N |
| ATOM | 2758 | CD2 | HIS | B | 784 | 39.506 | −11.180 | 36.322 | 1.00 | 53.76 | C |
| ATOM | 2759 | C | HIS | B | 784 | 38.331 | −12.109 | 32.048 | 1.00 | 49.08 | C |
| ATOM | 2760 | O | HIS | B | 784 | 38.559 | −13.299 | 31.879 | 1.00 | 49.44 | O |
| ATOM | 2761 | N | ILE | B | 785 | 37.169 | −11.552 | 31.746 | 1.00 | 50.08 | N |
| ATOM | 2762 | CA | ILE | B | 785 | 36.100 | −12.296 | 31.075 | 1.00 | 51.26 | C |
| ATOM | 2763 | CB | ILE | B | 785 | 36.409 | −12.469 | 29.563 | 1.00 | 51.67 | C |
| ATOM | 2764 | CG1 | ILE | B | 785 | 35.162 | −12.971 | 28.831 | 1.00 | 52.65 | C |
| ATOM | 2765 | CD1 | ILE | B | 785 | 35.332 | −13.007 | 27.347 | 1.00 | 54.35 | C |
| ATOM | 2766 | CG2 | ILE | B | 785 | 37.583 | −13.424 | 29.341 | 1.00 | 50.78 | C |
| ATOM | 2767 | C | ILE | B | 785 | 35.757 | −13.651 | 31.643 | 1.00 | 51.63 | C |
| ATOM | 2768 | O | ILE | B | 785 | 35.905 | −14.643 | 30.960 | 1.00 | 51.81 | O |
| ATOM | 2769 | N | PRO | B | 786 | 35.313 | −13.691 | 32.890 | 1.00 | 52.49 | N |
| ATOM | 2770 | CA | PRO | B | 786 | 34.882 | −14.933 | 33.536 | 1.00 | 52.77 | C |
| ATOM | 2771 | CB | PRO | B | 786 | 35.206 | −14.656 | 34.998 | 1.00 | 52.72 | C |
| ATOM | 2772 | CG | PRO | B | 786 | 34.865 | −13.198 | 35.163 | 1.00 | 52.87 | C |
| ATOM | 2773 | CD | PRO | B | 786 | 35.236 | −12.542 | 33.817 | 1.00 | 53.07 | C |
| ATOM | 2774 | C | PRO | B | 786 | 33.381 | −15.174 | 33.396 | 1.00 | 53.40 | C |
| ATOM | 2775 | O | PRO | B | 786 | 32.835 | −15.085 | 32.279 | 1.00 | 53.92 | O |
| ATOM | 2776 | N | ARG | B | 787 | 32.730 | −15.465 | 34.527 | 1.00 | 53.49 | N |
| ATOM | 2777 | CA | ARG | B | 787 | 31.274 | −15.694 | 34.591 | 1.00 | 53.49 | C |
| ATOM | 2778 | CB | ARG | B | 787 | 30.480 | −14.416 | 34.239 | 1.00 | 53.44 | C |
| ATOM | 2779 | CG | ARG | B | 787 | 30.063 | −13.565 | 35.449 | 1.00 | 52.26 | C |
| ATOM | 2780 | CD | ARG | B | 787 | 31.223 | −13.138 | 36.339 | 1.00 | 52.06 | C |
| ATOM | 2781 | NE | ARG | B | 787 | 30.807 | −12.649 | 37.659 | 1.00 | 53.17 | N |
| ATOM | 2782 | CZ | ARG | B | 787 | 30.457 | −13.428 | 38.706 | 1.00 | 51.86 | C |
| ATOM | 2783 | NH1 | ARG | B | 787 | 30.468 | −14.750 | 38.601 | 1.00 | 50.85 | N |
| ATOM | 2784 | NH2 | ARG | B | 787 | 30.090 | −12.880 | 39.864 | 1.00 | 49.91 | N |
| ATOM | 2785 | C | ARG | B | 787 | 30.800 | −16.882 | 33.745 | 1.00 | 53.54 | C |
| ATOM | 2789 | N | LEU | P | 1 | 20.931 | −14.544 | 31.252 | 1.00 | 47.69 | N |
| ATOM | 2790 | CA | LEU | P | 1 | 22.180 | −13.716 | 31.289 | 1.00 | 47.41 | C |
| ATOM | 2791 | CB | LEU | P | 1 | 23.030 | −14.044 | 32.535 | 1.00 | 47.97 | C |
| ATOM | 2792 | CG | LEU | P | 1 | 23.458 | −15.482 | 32.878 | 1.00 | 48.35 | C |
| ATOM | 2793 | CD1 | LEU | P | 1 | 24.423 | −15.489 | 34.063 | 1.00 | 49.77 | C |
| ATOM | 2794 | CD2 | LEU | P | 1 | 22.266 | −16.389 | 33.165 | 1.00 | 48.66 | C |
| ATOM | 2795 | C | LEU | P | 1 | 22.987 | −13.883 | 29.991 | 1.00 | 46.60 | C |
| ATOM | 2796 | O | LEU | P | 1 | 22.417 | −13.727 | 28.897 | 1.00 | 47.13 | O |
| ATOM | 2797 | N | ASP | P | 2 | 24.287 | −14.207 | 30.109 | 1.00 | 44.53 | N |
| ATOM | 2798 | CA | ASP | P | 2 | 25.177 | −14.357 | 28.935 | 1.00 | 42.24 | C |
| ATOM | 2799 | CB | ASP | P | 2 | 24.541 | −15.300 | 27.910 | 1.00 | 42.69 | C |
| ATOM | 2800 | CG | ASP | P | 2 | 25.522 | −15.772 | 26.866 | 1.00 | 44.51 | C |
| ATOM | 2801 | OD1 | ASP | P | 2 | 25.766 | −15.024 | 25.896 | 1.00 | 45.50 | O |
| ATOM | 2802 | OD2 | ASP | P | 2 | 26.093 | −16.887 | 26.932 | 1.00 | 46.89 | O |
| ATOM | 2803 | C | ASP | P | 2 | 25.516 | −12.983 | 28.288 | 1.00 | 39.49 | C |
| ATOM | 2804 | O | ASP | P | 2 | 26.077 | −12.893 | 27.200 | 1.00 | 39.39 | O |
| ATOM | 2805 | N | TYR | P | 3 | 25.145 | −11.911 | 28.970 | 1.00 | 35.91 | N |
| ATOM | 2806 | CA | TYR | P | 3 | 25.408 | −10.565 | 28.477 | 1.00 | 32.30 | C |
| ATOM | 2807 | CB | TYR | P | 3 | 24.129 | −9.724 | 28.549 | 1.00 | 31.47 | C |
| ATOM | 2808 | CG | TYR | P | 3 | 24.334 | −8.262 | 28.269 | 1.00 | 31.00 | C |
| ATOM | 2809 | CD1 | TYR | P | 3 | 24.418 | −7.805 | 26.965 | 1.00 | 29.48 | C |
| ATOM | 2810 | CE1 | TYR | P | 3 | 24.583 | −6.478 | 26.693 | 1.00 | 27.36 | C |
| ATOM | 2811 | CZ | TYR | P | 3 | 24.676 | −5.570 | 27.712 | 1.00 | 27.68 | C |
| ATOM | 2812 | OH | TYR | P | 3 | 24.888 | −4.218 | 27.370 | 1.00 | 28.65 | O |
| ATOM | 2813 | CE2 | TYR | P | 3 | 24.582 | −5.991 | 29.034 | 1.00 | 25.24 | C |
| ATOM | 2814 | CD2 | TYR | P | 3 | 24.407 | −7.315 | 29.309 | 1.00 | 27.91 | C |
| ATOM | 2815 | C | TYR | P | 3 | 26.471 | −10.014 | 29.386 | 1.00 | 29.47 | C |
| ATOM | 2816 | O | TYR | P | 3 | 26.400 | −10.230 | 30.588 | 1.00 | 28.95 | O |
| ATOM | 2817 | N | HIS | P | 4 | 27.456 | −9.330 | 28.820 | 1.00 | 26.50 | N |
| ATOM | 2818 | CA | HIS | P | 4 | 28.564 | −8.759 | 29.600 | 1.00 | 24.52 | C |
| ATOM | 2819 | CB | HIS | P | 4 | 29.803 | −8.571 | 28.690 | 1.00 | 24.64 | C |
| ATOM | 2820 | CG | HIS | P | 4 | 30.980 | −7.912 | 29.349 | 1.00 | 25.77 | C |
| ATOM | 2821 | ND1 | HIS | P | 4 | 31.453 | −8.268 | 30.598 | 1.00 | 30.26 | N |
| ATOM | 2822 | CE1 | HIS | P | 4 | 32.496 | −7.522 | 30.917 | 1.00 | 27.94 | C |
| ATOM | 2823 | NE2 | HIS | P | 4 | 32.732 | −6.707 | 29.910 | 1.00 | 26.64 | N |
| ATOM | 2824 | CD2 | HIS | P | 4 | 31.790 | −6.919 | 28.926 | 1.00 | 27.32 | C |
| ATOM | 2825 | C | HIS | P | 4 | 28.197 | −7.412 | 30.271 | 1.00 | 22.87 | C |
| ATOM | 2826 | O | HIS | P | 4 | 28.058 | −6.414 | 29.595 | 1.00 | 21.47 | O |
| ATOM | 2827 | N | PHE | P | 5 | 28.025 | −7.405 | 31.590 | 1.00 | 21.22 | N |
| ATOM | 2828 | CA | PHE | P | 5 | 27.773 | −6.153 | 32.298 | 1.00 | 20.74 | C |
| ATOM | 2829 | CB | PHE | P | 5 | 26.881 | −6.357 | 33.494 | 1.00 | 18.47 | C |
| ATOM | 2830 | CG | PHE | P | 5 | 25.501 | −6.754 | 33.152 | 1.00 | 16.23 | C |
| ATOM | 2831 | CD1 | PHE | P | 5 | 25.193 | −8.091 | 32.917 | 1.00 | 14.97 | C |
| ATOM | 2832 | CE1 | PHE | P | 5 | 23.887 | −8.475 | 32.631 | 1.00 | 13.89 | C |
| ATOM | 2833 | CZ | PHE | P | 5 | 22.881 | −7.505 | 32.578 | 1.00 | 11.09 | C |
| ATOM | 2834 | CE2 | PHE | P | 5 | 23.182 | −6.167 | 32.806 | 1.00 | 10.80 | C |
| ATOM | 2835 | CD2 | PHE | P | 5 | 24.471 | −5.790 | 33.087 | 1.00 | 11.98 | C |
| ATOM | 2836 | C | PHE | P | 5 | 29.109 | −5.609 | 32.769 | 1.00 | 21.26 | C |
| ATOM | 2837 | O | PHE | P | 5 | 29.724 | −6.195 | 33.664 | 1.00 | 21.67 | O |
| ATOM | 2838 | N | GLY | P | 6 | 29.556 | −4.502 | 32.170 | 1.00 | 21.32 | N |
| ATOM | 2839 | CA | GLY | P | 6 | 30.859 | −3.926 | 32.468 | 1.00 | 21.51 | C |
| ATOM | 2840 | C | GLY | P | 6 | 30.831 | −2.897 | 33.585 | 1.00 | 22.19 | C |
| ATOM | 2841 | O | GLY | P | 6 | 31.865 | −2.528 | 34.114 | 1.00 | 21.62 | O |
| ATOM | 2842 | N | LEU | P | 7 | 29.651 | −2.406 | 33.919 | 1.00 | 23.30 | N |
| ATOM | 2843 | CA | LEU | P | 7 | 29.543 | −1.497 | 35.032 | 1.00 | 25.46 | C |
| ATOM | 2844 | CB | LEU | P | 7 | 28.154 | −0.859 | 35.099 | 1.00 | 25.82 | C |
| ATOM | 2845 | CG | LEU | P | 7 | 27.814 | 0.211 | 34.061 | 1.00 | 26.11 | C |
| ATOM | 2846 | CD1 | LEU | P | 7 | 26.339 | 0.562 | 34.191 | 1.00 | 24.70 | C |
| ATOM | 2847 | CD2 | LEU | P | 7 | 28.709 | 1.422 | 34.234 | 1.00 | 21.99 | C |
| ATOM | 2848 | C | LEU | P | 7 | 29.817 | −2.254 | 36.323 | 1.00 | 26.52 | C |
| ATOM | 2849 | O | LEU | P | 7 | 29.437 | −3.421 | 36.472 | 1.00 | 25.30 | O |
| ATOM | 2850 | N | GLU | P | 8 | 30.492 | −1.567 | 37.240 | 1.00 | 28.49 | N |
| ATOM | 2851 | CA | GLU | P | 8 | 30.779 | −2.099 | 38.551 | 1.00 | 31.21 | C |
| ATOM | 2852 | CB | GLU | P | 8 | 32.215 | −1.763 | 38.887 | 1.00 | 32.54 | C |
| ATOM | 2853 | CG | GLU | P | 8 | 33.179 | −2.369 | 37.881 | 1.00 | 38.33 | C |
| ATOM | 2854 | CD | GLU | P | 8 | 34.639 | −2.164 | 38.254 | 1.00 | 44.38 | C |
| ATOM | 2855 | OE1 | GLU | P | 8 | 34.977 | −1.015 | 38.639 | 1.00 | 47.15 | O |
| ATOM | 2856 | OE2 | GLU | P | 8 | 35.437 | −3.145 | 38.151 | 1.00 | 44.55 | O |
| ATOM | 2857 | C | GLU | P | 8 | 29.830 | −1.509 | 39.593 | 1.00 | 30.87 | C |
| ATOM | 2858 | O | GLU | P | 8 | 29.277 | −0.443 | 39.374 | 1.00 | 30.40 | O |
| ATOM | 2859 | N | GLU | P | 9 | 29.655 | −2.193 | 40.726 | 1.00 | 31.54 | N |
| ATOM | 2860 | CA | GLU | P | 9 | 28.722 | −1.742 | 41.790 | 1.00 | 31.59 | C |
| ATOM | 2861 | CB | GLU | P | 9 | 28.841 | −2.607 | 43.057 | 1.00 | 32.17 | C |
| ATOM | 2862 | CG | GLU | P | 9 | 28.217 | −4.028 | 42.934 | 1.00 | 34.26 | C |
| ATOM | 2863 | CD | GLU | P | 9 | 28.018 | −4.747 | 44.294 | 1.00 | 33.72 | C |
| ATOM | 2864 | OE1 | GLU | P | 9 | 27.685 | −4.084 | 45.314 | 1.00 | 33.47 | O |
| ATOM | 2865 | OE2 | GLU | P | 9 | 28.185 | −5.978 | 44.350 | 1.00 | 31.42 | O |
| ATOM | 2866 | C | GLU | P | 9 | 28.826 | −0.254 | 42.133 | 1.00 | 30.99 | C |
| ATOM | 2867 | O | GLU | P | 9 | 27.826 | 0.429 | 42.361 | 1.00 | 31.00 | O |
| ATOM | 2868 | N | GLY | P | 10 | 30.033 | 0.276 | 42.153 | 1.00 | 30.01 | N |
| ATOM | 2869 | CA | GLY | P | 10 | 30.155 | 1.696 | 42.418 | 1.00 | 29.07 | C |
| ATOM | 2870 | C | GLY | P | 10 | 29.895 | 2.598 | 41.209 | 1.00 | 27.90 | C |
| ATOM | 2871 | O | GLY | P | 10 | 30.205 | 3.806 | 41.240 | 1.00 | 28.51 | O |
| ATOM | 2872 | N | GLU | P | 11 | 29.353 | 2.024 | 40.138 | 1.00 | 25.73 | N |
| ATOM | 2873 | CA | GLU | P | 11 | 29.073 | 2.791 | 38.922 | 1.00 | 23.64 | C |
| ATOM | 2874 | CB | GLU | P | 11 | 29.952 | 2.303 | 37.748 | 1.00 | 24.46 | C |
| ATOM | 2875 | CG | GLU | P | 11 | 31.459 | 2.612 | 37.875 | 1.00 | 26.02 | C |
| ATOM | 2876 | CD | GLU | P | 11 | 32.325 | 1.859 | 36.852 | 1.00 | 26.65 | C |
| ATOM | 2877 | OE1 | GLU | P | 11 | 33.486 | 2.296 | 36.631 | 1.00 | 24.35 | O |
| ATOM | 2878 | OE2 | GLU | P | 11 | 31.853 | 0.824 | 36.281 | 1.00 | 26.05 | O |
| ATOM | 2879 | C | GLU | P | 11 | 27.603 | 2.753 | 38.516 | 1.00 | 21.13 | C |
| ATOM | 2880 | O | GLU | P | 11 | 26.905 | 1.758 | 38.653 | 1.00 | 19.86 | O |
| ATOM | 2881 | N | GLY | P | 12 | 27.131 | 3.855 | 38.005 | 1.00 | 19.40 | N |
| ATOM | 2882 | CA | GLY | P | 12 | 25.766 | 3.895 | 37.535 | 1.00 | 18.44 | C |
| ATOM | 2883 | C | GLY | P | 12 | 25.582 | 4.880 | 36.401 | 1.00 | 17.74 | C |
| ATOM | 2884 | O | GLY | P | 12 | 26.404 | 5.785 | 36.161 | 1.00 | 16.52 | O |
| ATOM | 2885 | N | ILE | P | 13 | 24.458 | 4.734 | 35.719 | 1.00 | 17.35 | N |
| ATOM | 2886 | CA | ILE | P | 13 | 24.156 | 5.570 | 34.567 | 1.00 | 17.40 | C |
| ATOM | 2887 | CB | ILE | P | 13 | 22.680 | 5.451 | 34.230 | 1.00 | 16.80 | C |
| ATOM | 2888 | CG1 | ILE | P | 13 | 22.353 | 6.275 | 32.979 | 1.00 | 17.71 | C |
| ATOM | 2889 | CD1 | ILE | P | 13 | 23.048 | 5.763 | 31.684 | 1.00 | 18.51 | C |
| ATOM | 2890 | CG2 | ILE | P | 13 | 21.913 | 5.983 | 35.380 | 1.00 | 17.44 | C |
| ATOM | 2891 | C | ILE | P | 13 | 24.535 | 7.051 | 34.805 | 1.00 | 17.29 | C |
| ATOM | 2892 | O | ILE | P | 13 | 24.899 | 7.769 | 33.896 | 1.00 | 17.49 | O |
| ATOM | 2893 | N | ARG | P | 14 | 24.443 | 7.527 | 36.022 | 1.00 | 16.45 | N |
| ATOM | 2894 | CA | ARG | P | 14 | 24.749 | 8.917 | 36.228 | 1.00 | 17.18 | C |
| ATOM | 2895 | CB | ARG | P | 14 | 24.232 | 9.313 | 37.598 | 1.00 | 17.84 | C |
| ATOM | 2896 | CG | ARG | P | 14 | 24.580 | 10.693 | 38.068 | 1.00 | 17.51 | C |
| ATOM | 2897 | CD | ARG | P | 14 | 25.712 | 10.655 | 39.027 | 1.00 | 19.60 | C |
| ATOM | 2898 | NE | ARG | P | 14 | 25.496 | 11.615 | 40.079 | 1.00 | 21.69 | N |
| ATOM | 2899 | CZ | ARG | P | 14 | 26.127 | 11.610 | 41.237 | 1.00 | 22.46 | C |
| ATOM | 2900 | NH1 | ARG | P | 14 | 27.038 | 10.683 | 41.515 | 1.00 | 20.71 | N |
| ATOM | 2901 | NH2 | ARG | P | 14 | 25.840 | 12.557 | 42.113 | 1.00 | 22.29 | N |
| ATOM | 2902 | C | ARG | P | 14 | 26.228 | 9.319 | 36.051 | 1.00 | 17.89 | C |
| ATOM | 2903 | O | ARG | P | 14 | 26.533 | 10.465 | 35.749 | 1.00 | 18.28 | O |
| ATOM | 2904 | N | ASP | P | 15 | 27.153 | 8.388 | 36.249 | 1.00 | 17.97 | N |
| ATOM | 2905 | CA | ASP | P | 15 | 28.557 | 8.678 | 36.058 | 1.00 | 17.42 | C |
| ATOM | 2906 | CB | ASP | P | 15 | 29.419 | 7.620 | 36.731 | 1.00 | 17.17 | C |
| ATOM | 2907 | CG | ASP | P | 15 | 28.943 | 7.257 | 38.136 | 1.00 | 16.85 | C |
| ATOM | 2908 | OD1 | ASP | P | 15 | 28.567 | 8.168 | 38.917 | 1.00 | 14.72 | O |
| ATOM | 2909 | OD2 | ASP | P | 15 | 28.933 | 6.068 | 38.549 | 1.00 | 17.21 | O |
| ATOM | 2910 | C | ASP | P | 15 | 28.893 | 8.700 | 34.576 | 1.00 | 18.51 | C |
| ATOM | 2911 | O | ASP | P | 15 | 29.888 | 9.266 | 34.154 | 1.00 | 19.86 | O |
| ATOM | 2912 | N | LEU | P | 16 | 28.067 | 8.078 | 33.752 | 1.00 | 19.15 | N |
| ATOM | 2913 | CA | LEU | P | 16 | 28.383 | 8.009 | 32.351 | 1.00 | 19.27 | C |
| ATOM | 2914 | CB | LEU | P | 16 | 27.482 | 7.006 | 31.674 | 1.00 | 19.48 | C |
| ATOM | 2915 | CG | LEU | P | 16 | 27.817 | 5.590 | 32.121 | 1.00 | 18.49 | C |
| ATOM | 2916 | CD1 | LEU | P | 16 | 26.903 | 4.583 | 31.531 | 1.00 | 16.38 | C |
| ATOM | 2917 | CD2 | LEU | P | 16 | 29.205 | 5.334 | 31.714 | 1.00 | 18.61 | C |
| ATOM | 2918 | C | LEU | P | 16 | 28.287 | 9.373 | 31.691 | 1.00 | 20.34 | C |
| ATOM | 2919 | O | LEU | P | 16 | 28.964 | 9.619 | 30.688 | 1.00 | 18.95 | O |
| ATOM | 2920 | N | PHE | P | 17 | 27.423 | 10.216 | 32.264 | 1.00 | 22.07 | N |
| ATOM | 2921 | CA | PHE | P | 17 | 27.160 | 11.583 | 31.843 | 1.00 | 24.13 | C |
| ATOM | 2922 | CB | PHE | P | 17 | 25.668 | 11.799 | 31.553 | 1.00 | 23.78 | C |
| ATOM | 2923 | CG | PHE | P | 17 | 25.128 | 10.936 | 30.490 | 1.00 | 22.61 | C |
| ATOM | 2924 | CD1 | PHE | P | 17 | 24.659 | 9.689 | 30.789 | 1.00 | 21.60 | C |
| ATOM | 2925 | CE1 | PHE | P | 17 | 24.142 | 8.878 | 29.790 | 1.00 | 22.90 | C |
| ATOM | 2926 | CZ | PHE | P | 17 | 24.160 | 9.321 | 28.432 | 1.00 | 24.90 | C |
| ATOM | 2927 | CE2 | PHE | P | 17 | 24.662 | 10.542 | 28.122 | 1.00 | 23.17 | C |
| ATOM | 2928 | CD2 | PHE | P | 17 | 25.144 | 11.351 | 29.158 | 1.00 | 24.24 | C |
| ATOM | 2929 | C | PHE | P | 17 | 27.470 | 12.403 | 33.045 | 1.00 | 26.26 | C |
| ATOM | 2930 | O | PHE | P | 17 | 26.583 | 13.079 | 33.582 | 1.00 | 26.70 | O |
| ATOM | 2931 | N | ASP | P | 18 | 28.717 | 12.312 | 33.505 | 1.00 | 29.15 | N |
| ATOM | 2932 | CA | ASP | P | 18 | 29.152 | 12.999 | 34.732 | 1.00 | 31.05 | C |
| ATOM | 2933 | CB | ASP | P | 18 | 29.110 | 12.033 | 35.922 | 1.00 | 31.59 | C |
| ATOM | 2934 | CG | ASP | P | 18 | 29.079 | 12.741 | 37.254 | 1.00 | 35.19 | C |
| ATOM | 2935 | OD1 | ASP | P | 18 | 29.132 | 12.001 | 38.293 | 1.00 | 37.21 | O |
| ATOM | 2936 | OD2 | ASP | P | 18 | 29.007 | 14.012 | 37.355 | 1.00 | 37.60 | O |
| ATOM | 2937 | C | ASP | P | 18 | 30.566 | 13.540 | 34.586 | 1.00 | 31.76 | C |

1. A crystal comprising a pRb/E2F₍₄₀₉₋₄₂₆₎ complex, wherein the crystal structure is characterised by the atomic co-ordinates of Annex 1.

2. A crystal as claimed in claim 1, wherein the interactions between E2F₍₄₀₉₋₄₂₆₎ and pRb comprise one or more of the following interactions:

| E2F₍₄₀₉₋₄₂₆₎ residue | pRb residue |
|---|---|
| Leu₄₀₉ | Lys₅₄₈ |
| Tyr₄₁₁ | Glu₅₅₁ |
| Tyr₄₁₁ | Ile₅₃₂ |
| Tyr₄₁₁ | Glu₅₅₄ |
| His₄₁₂ | Arg₆₅₆ |
| His₄₁₂ | Lys₆₅₃ |
| Gly₄₁₄ | Glu₅₃₃ |
| Gly₄₁₄ | Lys₆₅₂ |
| Leu₄₁₅ | Leu₆₄₉ |
| Leu₄₁₅ | Glu₅₅₃ |
| Leu₄₁₅ | Lys₅₃₇ |
| Glu₄₁₇ | Lys₅₃₇ |
| Gly₄₁₈ | Arg₄₆₇ |
| Glu₄₁₉ | Thr₆₄₅ |
| Arg₄₂₂ | Glu₄₆₄ |
| Asp₄₂₃ | Arg₄₆₇ |
| Leu₄₂₄ | Lys₅₃₀ |
| Phe₄₂₅ | Phe₄₈₂ |
| Phe₄₂₅ | Lys₄₇₅ |

3. A method of identifying an agent which modulates the interaction between pRb and E2F₍₄₀₉₋₄₂₆₎, comprising:
a) combining pRb, E2F₍₄₀₉₋₄₂₆₎ and an agent, under conditions in which pRb and E2F₍₄₀₉₋₄₂₆₎ form a complex;
b) growing a crystal structure of any pRb/ E2F₍₄₀₉₋₄₂₆₎ complex; and
c) analyzing the crystal to determine whether the agent modulates the interaction between pRb and E2F₍₄₀₉₋₄₂₆₎.

4. The method of claim 3, wherein pRb is combined with the agent and E2F₍₄₀₉₋₄₂₆₎ is subsequently added.

5. The method in of claim 3, wherein E2F₍₄₀₉₋₄₂₆₎ is combined with the agent and pRb is subsequently added.

6. The method of claim 3, wherein he pRb is combined with E2F₍₄₀₉₋₄₂₆₎ and the agent is subsequently added.

7. The method of claim 3, wherein step c) comprises comparing the crystal structure to the crystal structure characterized by the atomic co-ordinates of Annex 1.

8. The method of claim 3, wherein the agent is selected using the three dimensional atomic co-ordinates of Annex 1.

9. A method of identifying an agent that modulates a pRb/E2F₍₄₀₉₋₄₂₆₎ complex, comprising selecting an agent using the three-dimensional atomic coordinates of Annex 1.

10. The method of claim 9, wherein said selecting is performed in conjunction with computer modeling.

11. The method of claim 9, further comprising the steps of:
a) contacting the selected agent with pRb and E2F₍₄₀₉₋₄₂₆₎ under conditions in which pRb and E2F₍₄₀₉₋₄₂₆₎ can form a complex; and
b) measuring the binding affinity of pRb to E2F₍₄₀₉₋₄₂₆₎ in the presence of the agent and comparing the binding affinity to that of pRb to E2F₍₄₀₉₋₄₂₆₎ when in the absence of the agent, wherein an agent modulates a pRb/E2F₍₄₀₉₋₄₂₆₎ complex when there is a change in the binding affinity of pRb to E2F₍₄₀₉₋₄₂₆₎ when in the presence of the agent.

12. The method of claim 11, further comprising:
a) growing a supplementary crystal from a solution containing pRb and E2F₍₄₀₉₋₄₂₆₎ and the selected agent where said agent changes the binding affinity of the pRb/E2F₍₄₀₉₋₄₂₆₎ complex under conditions in which pRb and E2F₍₄₀₉₋₄₂₆₎ can form a complex;
b) determining the three-dimensional atomic coordinates of the supplementary crystal by X-ray diffraction using molecular replacement analysis;
c) comparing the three dimensional coordinates with those for the crystal structure as characterized by the atomic co-ordinates of Annex 1; and
d) selecting a second generation agent using the three-dimensional atomic coordinates determined for the supplementary crystal.

13. The method of claim 12, wherein said selecting is performed in conjunction with computer modeling.

14. A method of identifying an agent that modulates a pRb/E2F₍₄₀₉₋₄₂₆₎ complex, comprising:
a) contacting an agent with pRb and E2F₍₄₀₉₋₄₂₆₎ under conditions in which pRb and E2F₍₄₀₉₋₄₂₆₎ can form a complex; and
b) measuring the binding affinity of pRb to E2F₍₄₀₉₋₄₂₆₎ in the presence of the agent and comparing the binding affinity to that of pRb to E2F₍₄₀₉₋₄₂₆₎ when in the absence of the agent, wherein an agent modulates a pRb/E2F₍₄₀₉₋₄₂₆₎ complex when there is a change in the binding affinity of pRb to E2F₍₄₀₉₋₄₂₆₎ when in the presence of the agent.

15. The method of claim 14, further comprising:
a) growing a supplementary crystal from a solution containing pRb and E2F₍₄₀₉₋₄₂₆₎ and the agent wherein said agent changes the binding affinity of the pRb/E2F₍₄₀₉₋₄₂₆₎ complex under conditions in which pRb and E2F₍₄₀₉₋₄₂₆₎ can form a complex;
b) determining the three-dimensional atomic coordinates of the supplementary crystal by X-ray diffraction using molecular replacement analysis;
c) comparing the three dimensional coordinates with those for the crystal structure characterized by the atomic co-ordinates of Annex 1; and
d) selecting a second generation agent using the three-dimensional atomic coordinates determined for the supplementary crystal.

16. The method as claimed in of claim 15, wherein said selecting is performed in conjunction with computer modeling.

17. A method of identifying an agent that modulates a pRb/E2F₍₄₀₉₋₄₂₆₎ complex, comprising:
a) co-crystallizing pRb with an agent;
b) determining the three dimensional coordinates of the pRb-agent association by X-ray diffraction using molecular replacement analysis; and
c) comparing the three dimensional coordinates with those of the crystal structure claimed in claim 1.

18. A method of identifying an agent that modulates a pRb/E2F₍₄₀₉₋₄₂₆₎ complex, comprising:
a) crystallizing pRb and soaking an agent into the crystal;
b) determining the three dimensional coordinates of the pRb-agent association by X-ray diffraction using molecular replacement analysis; and
c) comparing the three dimensional coordinates with those of the crystal structure claimed in claim 1.

19. A method of identifying an agent that modulates a pRb/E2F₍₄₀₉₋₄₂₆₎ complex, comprising:
a) co-crystallizing pRb, E2F₍₄₀₉₋₄₂₆₎ and an agent;
b) determining the three dimensional coordinates of the pRb-E2F-agent association by X-ray diffraction using molecular replacement analysis; and
c) comparing the three dimensional coordinates with those of the crystal structure claimed in claim 1.

20. A method of identifying an agent that modulates a pRb/E2F₍₄₀₉₋₄₂₆₎ complex, comprising:
a) co-crystalising pRb and E2F₍₄₀₉₋₄₂₆₎ and soaking an agent into the crystal;
b) determining the three dimensional coordinates of the pRb-E2F-agent association by X-ray diffraction using molecular replacement analysis; and
c) comparing the three dimensional coordinates with those of the crystal structure claimed in claim 1.

21. The method of claim 17, wherein the agent is selected using the three dimensional atomic co-ordinates of Annex 1.

22. The method of claim 17, further comprising selecting a second generation agent using the three dimensional atomic coordinates determined in step b).

23. The method of claim 22, wherein the second generation agent is selected using the three dimensional atomic coordinates of Annex 1.

24. The method of claim 22, wherein the selecting is performed in conjunction with computer modeling.

25. The method of claim 3, wherein the identified agent mimics a structural feature of E2F₍₄₀₉₋₄₂₆₎, when said E2F₍₄₀₉₋₄₂₆₎ is bound to pRb.

26. The method of claim 9, wherein further comprising the steps of,
a) contacting the agent with a pRb/E2F₍₄₀₉₋₄₂₆₎ complex; and
b) determining whether the agent affects the stability of the complex.

27. The method of claim 26, wherein the determining is with fluorescence polarization.

28. A method of identifying an agent that modulates a pRb/E2F₍₄₀₉₋₄₂₆₎ complex, comprising:
a) contacting a fluorescently tagged E2F₍₄₀₉₋₄₂₆₎ peptide (E2F-fluoropeptide) with pRb to allow pRb/E2F-fluoropeptide complex formation;
b) detecting the fluorescence polarization;
c) adding an agent; and
d) detecting the fluorescence polarization in the presence of the agent.

29. A method of identifying an agent that modulates a pRb/E2F₍₄₀₉₋₄₂₆₎ complex, comprising;
a) contacting a fluorescently tagged E2F₍₄₀₉₋₄₂₆₎ peptide (E2F-fluoropeptide) with pRb to allow pRb/E2F-fluoropeptide complex formation;
b) detecting the fluorescence polarization;
c) contacting an agent with pRb and E2F₍₄₀₉₋₄₂₆₎ peptide (E2F-fluoropeptide) under conditions in which pRb and E2F-fluoropeptide can form a complex;
d) detecting the fluorescence polarization; and
e) comparing the fluorescence polarization detected in b) and d).

30. The method of claim 28, wherein the fluorescently tagged E2F peptide is selected using the three dimensional atomic co-ordinates of Annex 1.

31. The method of claim 28, wherein a decrease in fluorescence polarization in the presence of the agent indicates that the agent destabilises the complex.

32. The method of claim 28, further comprising the step of adding untagged E2F₍₄₀₉₋₄₂₆₎ and detecting fluorescence polarization.

33. The method of claim 32, wherein a decrease in fluorescence polarization, upon addition of the untagged E2F₍₄₀₉₋₄₂₆₎ is indicative that the agent does not stabilise the complex.

34. The method of claim 32, wherein no substantial change in fluorescence polarization upon addition of the untagged E2F₍₄₀₉₋₄₂₆₎ is indicative that the agent stabilises the complex.

35. The method of claim 28, further comprising:
a) contacting a fluorescently tagged E7 peptide (E7-fluoropeptide) with pRb to allow pRb/E7-fluoropeptide complex formation;
b) detecting the fluorescence polarization;
c) adding an agent that modulates pRb/E2F₍₄₀₉4₂₆₎ complex; and
d) detecting the fluorescence polarization in the presence of the agent.

36. The method of claim 28, further comprising:
a) contacting a fluorescently tagged E7 peptide (E7-fluoropeptide) with pRb to allow pRb/E7-fluoropeptide complex formation;
b) detecting the fluorescence polarization;
c) contacting an agent that modulates pRb/E2F₍₄₀₉₋₄₂₆₎ complex with pRb and E7-fluoropeptide under conditions in which pRb and E7-fluoropeptide can from a complex;
d) detecting the fluorescence polarization; and
e) comparing the fluorescence polarization detected in b) and d).

37. The method in of claim 35, wherein a decrease in fluorescence polarization indicates that the agent also inhibits E7 binding to pRb.

38. The method of claim 11, wherein the binding affinity is measured by isothermal titration calorimetry.

39. The method of claim 11, wherein the binding affinity is measured by Surface Plasmon Resonance (SPR).

40. A method of modulating the interaction between pRb and E2F₍₄₀₉₋₄₂₆₎ comprising contacting an agent identified by the method of claim 3 with pRb and E2F₍₄₀₉₋₄₂₆₎ under conditions in which pRb and E2F₍₄₀₉₋₄₂₆₎ form a complex.

41. A method for the prevention or treatment of proliferative diseases comprising contacting a cell with an agent identified by the method as claimed in claim 3, wherein the agent is an apoptosis promoting factor.

42. A method for preventing or treating cancer, comprising contacting a cancer cell with an agent identified by the method as claimed in claim 3.

43. A pharmaceutical composition comprising an agent which modulates the formation of a pRb/E2F₍₄₀₉₋₄₂₆₎ complex as identified by the method as claimed in any one of claim 3.

44. (canceled)

45. The method of claim 40, wherein the agent is identified by use of the atomic co-ordinates of Annex 1.

46. Computer readable media comprising a data storage material encoded with computer readable data, wherein said computer readable data comprises a set of atomic co-ordinates of the pRb/E2F₍₄₀₉₋₄₂₆₎ crystal structure of Annex I recorded thereon.

47. The method of claim 42, wherein the cancer is pancreatic cancer.
